(19) United States
(12) Patent Application Publication
(10) Pub. No.: US 2009/0215742 A1
(43) Pub. Date: Aug. 27, 2009

(54) AMIDE RESORCINOL COMPOUNDS

(75) Inventors: Lee Andrew Funk, Oceanside, CA (US); Mary Catherine Johnson, San Diego, CA (US); Pei-Pei Kung, San Diego, CA (US); Zhongxiang Zhou, San Diego, CA (US)

Correspondence Address: PFIZER INC, 10555 SCIENCE CENTER DRIVE, SAN DIEGO, CA, 92121, US

(73) Assignee: PFIZER, INC.

(21) Appl. No.: 11/913,427

(22) PCT Filed: Apr. 21, 2006
(86) PCT No.: PCT/IB06/01178

§ 371 Date: Aug. 12, 2008

Related U.S. Application Data
(60) Provisional application No. 60/677,268, filed on May 3, 2005.
(60) Provisional application No. 60/772,626, filed on Feb. 13, 2006.

(51) Int. Cl. A61K 31/553; C07D 209/44; A61K 31/4035; C07D 267/10; C07D 498/10

(52) U.S. Cl. 514/211.1; 548/482; 514/416; 540/544; 514/211.01; 540/543

(57) ABSTRACT

The present invention is directed to compounds of formula (I),

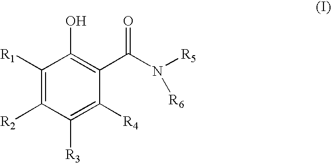

and pharmaceutically acceptable salts and solvates thereof, their synthesis, and their use as HSP-90 inhibitors.

This application is the national stage filing under 35 U.S.C. 371, of Patent Cooperation Treaty Patent Application No. PCT/IB2006/001178, filed Apr. 21, 2006, which claims the benefit of U.S. Provisional Application Nos. 60/677,268 filed May 3, 2005 and 60/772,626, filed Feb. 13, 2006 the disclosures of which are incorporated herein by reference in their entireties.

### FIELD OF THE INVENTION

The present invention is directed to compounds, and pharmaceutically acceptable salts and solvates thereof, their synthesis, and their use as modulators or inhibitors of HSP-90. The compounds of the present invention are useful for modulating (e.g. inhibiting) HSP-90 activity and for treating diseases or conditions mediated by HSP-90, such as for example, disease states associated with abnormal cell growth such as cancer.

### BACKGROUND

Molecular chaperones play important roles in cellular function by ensuring proper folding of proteins upon synthesis as well as their refolding under conditions of denaturing stress. By regulating the balance between protein synthesis and degradation, molecular chaperones are a significant part of the cellular response to stress. In addition, by regulating the proper folding of various cellular proteins, chaperones play an important role in regulating cellular functions such as cell proliferation and apoptosis. (See, e.g. Jolly, et al., J. Natl. Cancer Inst. 92: 1564-1572 (2000)). Heat shock proteins (HSPs) are a class of chaperones that accumulate in the cell in response to various environmental stresses, such as heat shock, oxidative stress, or the presence of alcohols or heavy metals. In addition to their role in protecting the cell from such environmental stresses, HSPs may also play a significant role as chaperones for a variety of cellular proteins under stress-free conditions. Members of the HSP family are classified according to their molecular weight (e.g. HSP-27, HSP-70, and HSP-90). Evidence of differential expression of HSPs in various stages of tumor progression suggests HSPs play a role in cancer. (See, e.g. Martin, et al., Cancer Res. 60:2232-2238 (2000)).

HSP-90 is a homodimer with ATPase activity and functions in a series of complex interactions with a variety of substrate proteins (Young, et al., J. Cell Biol. 154: 267-273 (2001)). HSP-90 is unique with regard to other chaperones, however, since most of its known substrate proteins are signal transduction proteins. Thus, HSP-90 plays an essential role in regulating cellular signal transduction networks. (See, e.g. Xu, et al., Proc. Natl. Acad. Sci 90:7074-7078 (1993)). In particular, substrate proteins of HSP-90 include many mutated or over-expressed proteins implicated in cancer such as p53, Bcr-Ab1 kinase, Raf-1 kinase, Akt kinase, Npm-Alk kinase p185^{ErbB2} transmembrane kinase, Cdk4, Cdk6, Wee1 (a cell cycle-dependent kinase), HER2/Neu (ErbB2), and hypoxia inducible factor-1α (HIF-1α). Thus inhibition of HSP-90 results in selective degradation of these important signaling proteins involved in apoptosis, cell proliferation, and cell cycle regulation (Holstein, et al., Cancer Res. 61:4003-4009 (2001)). Accordingly, HSP-90 is an attractive therapeutic target because of the important roles played by these signaling proteins in disease states involving abnormal cell growth, such as cancer. It is thus desirable to discover and develop new inhibitors of HSP-90 activity that can provide a therapeutic benefit to patients suffering from disease states related to abnormal cell growth such as cancer.

### SUMMARY

The present invention provides compounds of formula (I)

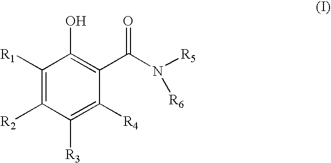

wherein: R¹ is H, —CH₃, or halogen; R², R³ and R⁴ are each independently H, —OH, (C₁ to C₆) alkyl, (C₂ to C₈) alkenyl, (C₂ to C₈) alkynyl, (C₁ to C₈) alkoxy, (C₁ to C₈) heteroalkyl, halogen, —CF₃, cyano, —Xₘ—C(O)R⁷, —Xₘ—S(O)₂R⁷, —Xₘ—(NR^{8a})—S(O)₂R^{8b}, —Xₘ—(NR^{8a})—C(O)R^{8b}, (C₂ to C₉) cycloheteroalkyl, (C₃ to C₈) cycloalkyl, (C₆ to C₁₄) aryl, or (C₂ to C₉) heteroaryl, each of which is optionally substituted with at least one R⁹ group; or R³ together with either R² or R⁴, together with the atoms to which they are attached, form a (C₆ to C₁₄) aryl, (C₂ to C₉) heteroaryl, (C₂ to C₉) cycloheteroalkyl, or a (C₃ to C₈) cycloalkyl group, each of which is optionally substituted with at least one R⁹ group;

R⁵ and R⁶ are each independently —(CR^{10a}R^{10b})—R¹¹ or R⁵ and R⁶ taken together with the nitrogen atom to which they are attached in formula (I) form a (C₂ to C₉) heteroaryl, or a (C₂ to C₉) cycloheteroalkyl group, wherein each of said (C₂ to C₉) heteroaryl and (C₂ to C₉) cycloheteroalkyl is optionally substituted with at least one R¹² group, and each is optionally spiro-fused to an R¹³ group;

each R⁷ is independently H, halogen, —CF₃, cyano, —N(R^{8a}R^{8b}), (C₁ to C₆) alkyl, (C₂ to C₈) alkenyl, (C₂ to C₈) alkynyl, (C₁ to C₈) alkoxy, (C₆ to C₁₄) aryl, (C₂ to C₉) heteroaryl, (C₂ to C₉) cycloheteroalkyl, or (C₃ to C₈) cycloalkyl;

each R^{8a} and R^{8b} is independently H, (C₁ to C₆) alkyl, (C₂ to C₈) alkenyl, (C₂ to C₈) alkynyl, —(CH₂)ₙCN, —(CH₂)ₙN(R^{10a}R^{10b}), —(CH₂)ₙCF₃, —(CH₂)ₙCHF₂, (C₃ to C₈) cycloalkyl, (C₂ to C₉) cycloheteroalkyl, or (C₁ to C₈) heteroalkyl, wherein said (C₃ to C₈) cycloalkyl and (C₂ to C₉) cycloheteroalkyl are each optionally substituted with at least one R^{10a} group; or when R^{8a} and R^{8b} are both bound to a nitrogen atom, R^{8a} and R^{8b} together with the nitrogen atom to which they are attached, can form a (C₂ to C₉) cycloheteroalkyl group;

each R⁹ is independently —OH, halogen, —CF₃, cyano, (C₁ to C₆) alkyl, (C₂ to C₈) alkenyl, (C₂ to C₈) alkynyl, (C₁ to C₈) heteroalkyl, (C₁ to C₈) alkoxy, —Xₘ—S(O)₂R⁷, —Xₘ—(NR^{8a})—S(O)₂R^{8b}, —N(R^{8a}R^{8b}), —NR^{8a}C(O)₂R^{8b}, —(CH₂)ₙC(O)₂R^{8a}, —C(O)N(R^{8a}R^{8b}), —Xₘ—(C₆ to C₁₄) aryl, —Xₘ—(C₂ to C₉) heteroaryl, —Xₘ—(C₂ to C₉) cycloheteroalkyl, or —Xₘ—(C₃ to C₈) cycloalkyl, wherein said (C₆ to C₁₄) aryl, (C₂ to C₉) heteroaryl, (C₂ to C₉) cycloheteroalkyl, or (C₃ to C₈) cycloalkyl is optionally further substituted with at least one R¹⁴ group;

each R^{10a} and R^{10b} is independently H, —OH, (C₁ to C₆) alkyl, (C₂ to C₈) alkenyl, (C₂ to C₈) alkynyl, (C₁ to C₈) heteroalkyl, or (C₁ to C₈) alkoxy;

each R¹¹ is independently (C₁ to C₆) alkyl, (C₂ to C₈) alkenyl, (C₂ to C₈) alkynyl, (C₁ to C₈) alkoxy, (C₁ to C₈) heteroalkyl, cyano, —CF₃, halogen, —N(R^{8a}R^{8b}), —(CH₂)ₙC(O)₂R^{8a} —(CH₂)ₙ—S(CH₂)ₙR⁹, —C(O)N(R^{8a}R^{8b}), (C₆ to C₁₄) aryl, (C₂ to C₉) heteroaryl, (C₂ to C₉) cycloheteroalkyl, or (C₃ to C₈) cycloalkyl, each of which is optionally substituted with at least one R⁹ group;

each R¹² is independently —OH, halogen, —CF₃, cyano, (C₁ to C₆) alkyl, (C₂ to C₈) alkenyl, (C₂ to C₈) alkynyl, (C₁ to C₈) heteroalkyl, (C₁ to C₈) alkoxy, —(CH₂)ₙ—SCH₃, —N(R^{8a}R^{8b}), —NR^{8a}C(O)₂R^{8b}, —C(O)N(R^{8a}R^{8b}), —(CH₂)ₙC(O)₂R^{8a}, —(CH₂)ₙC(O)₂R¹¹, —XₘS(O)₂R⁷, —Xₘ—(C₆ to C₁₄) aryl, —Xₘ—(C₂ to C₉) heteroaryl, —Xₘ—(C₂ to C₉) cycloheteroalkyl, or —Xₘ—(C₃ to C₈) cycloalkyl, wherein said (C₆ to C₁₄) aryl, (C₂ to C₉) heteroaryl, (C₂ to C₉) cycloheteroalkyl, and (C₃ to C₈) cycloalkyl are optionally further substituted with at least one R¹¹ group;

each R¹³ is independently (C₆ to C₁₄) aryl, (C₂ to C₉) heteroaryl, (C₂ to C₉) cycloheteroalkyl, or (C₃ to C₈) cycloalkyl, each of which is optionally substituted with at least one R¹² group;

each R¹⁴ is independently —OH, halogen, —CF₃, cyano, —NO₂, (C₁ to C₆) alkyl, (C₂ to C₈) alkenyl, (C₂ to C₈) alkynyl, (C₁ to C₈) heteroalkyl, (C₁ to C₈) alkoxy, —(CH₂)ₙ—SCH₃, —N(R^{8a}R^{8b}), —NR^{8a}C(O)₂R^{8b}, —(CH₂)ₙC(O)₂R^{8a}, —Xₘ—(C₆ to C₁₄) aryl, —Xₘ—(C₂ to C₉) heteroaryl, —Xₘ—(C₂ to C₉) cycloheteroalkyl, or —Xₘ—(C₃ to C₈) cycloalkyl;

X is —O—, —S—, —NH—, (C₁ to C₈) heteroalkyl, (C₁ to C₈) alkoxy, (C₁ to C₆) alkyl, (C₂ to C₈) alkenyl, or (C₂ to C₈) alkynyl; each m is independently 0 or 1; and each n is independently 0, 1, 2, 3, or 4; or pharmaceutically acceptable salts or solvates thereof.

The present invention also provides compounds of formula (I) as described above, wherein: R² is —OH; and R³ and R⁴ are each independently H, halogen, (C₁ to C₆) alkyl, (C₂ to C₈) alkenyl, (C₂ to C₈) alkynyl, or (C₁ to C₈) heteroalkyl.

The present invention also provides compounds of formula (I) as described above, wherein R² is —OH or —CF₃, and R³ and R⁴ are both H.

The present invention also provides compounds of formula (I) as described above, wherein R³ is a (C₆ to C₁₄) aryl, or (C₂ to C₉) heteroaryl group, which is optionally substituted by at least one R⁹ group.

The present invention also provides compounds of formula (I) as described above, wherein: R⁵ and R⁶ are each independently —(CR^{10a}R^{10b})ₙ—R¹¹. In one embodiment R⁵ is —CH₃; R⁶ is —(CR^{10a}R^{10b})ₙ—R¹¹; and n is 1, 2, or 3. In a further embodiment R¹¹ is C₆ aryl, and is optionally substituted with at least one R⁹ group.

The present invention also provides compounds of formula (I) as described above wherein R⁵ and R⁶ taken together with the nitrogen atom to which they are attached in formula (I) form a (C₂ to C₉) heteroaryl, or a (C₂ to C₉) cycloheteroalkyl group, wherein each of said (C₂ to C₉) heteroaryl and (C₂ to C₉) cycloheteroalkyl is optionally substituted with at least one R¹² group, and each is optionally spiro-fused to an R¹³ group. In one embodiment R² is —OH; and R³ and R⁴ are each independently H, —OH, (C₁ to C₆) alkyl, (C₁ to C₈) alkoxy, (C₁ to C₈) heteroalkyl, halogen, —CF₃, cyano, (C₂ to C₉) cycloheteroalkyl, (C₃ to C₈) cycloalkyl, (C₆ to C₁₄) aryl, or (C₂ to C₉) heteroaryl, each of which is optionally substituted with at least one R⁹ group. In a further embodiment R⁵ and R⁶ taken together with the nitrogen atom to which they are attached in formula (I) form a group selected from:

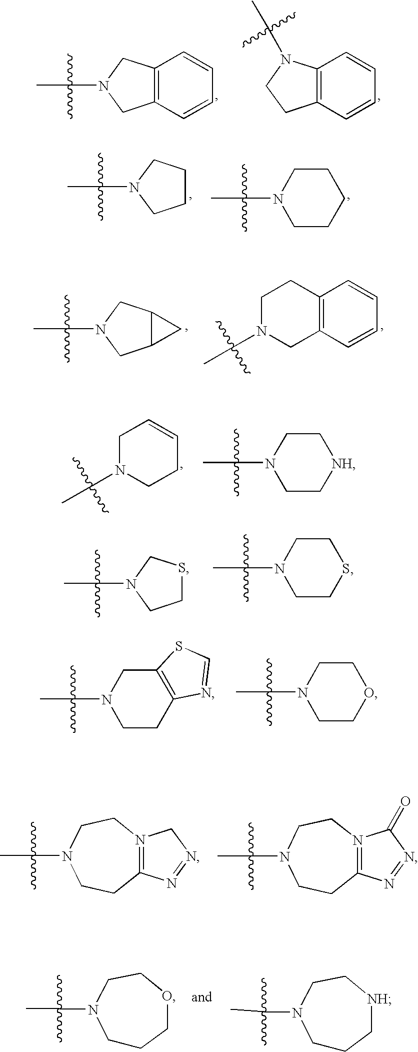

wherein each of said groups is optionally substituted with at least one R¹² group, and each of which is optionally spiro-fused to an R¹³ group.

In a further embodiment m is 0 and R¹² is (C₆ to C₁₄) aryl, (C₂ to C₉) heteroaryl, (C₂ to C₉) cycloheteroalkyl, (C₃ to C₈) cycloalkyl, or —C(O)N(R^{8a}R^{8b}), where each of said (C₆ to C₁₄) aryl, (C₂ to C₉) heteroaryl, (C₂ to C₉) cycloheteroalkyl, and (C₃ to C₈) cycloalkyl is optionally substituted with at least one R¹¹ group.

In a further embodiment R² is —OH; and R³ and R⁴ are each independently H, —OH, (C₁ to C₆) alkyl, (C₁ to C₈) alkoxy, (C₁ to C₈) heteroalkyl, halogen, —CF₃, cyano, (C₂ to C₉) cycloheteroalkyl, (C₃ to C₈) cycloalkyl, (C₆ to C₁₄) aryl, or (C₂ to C₉) heteroaryl, each of which is optionally substituted with at least one R⁹ group; or R³ together with R⁴, together with the atoms to which they are attached, form a (C₆ to C₁₄) aryl, (C₂ to C₉) heteroaryl, (C₂ to C₉) cycloheteroalkyl, or a (C₃ to C₈) cycloalkyl group, each of which is optionally substituted with at least one R⁹ group.

In another embodiment R³ and R⁴ are each independently H, —OH, (C₁ to C₄) alkyl, (C₁ to C₄) alkoxy, (C₁ to C₄) heteroalkyl, halogen, —CF₃, cyano, (C₂ to C₉) cycloheteroalkyl, (C₃ to C₈) cycloalkyl, (C₆ to C₁₄) aryl, or (C₂ to C₉) heteroaryl, each of which is optionally substituted with at least one R⁹ group. In a further embodiment R³ is halogen and R⁴ is H. In a further embodiment R³ is (C₂ to C₉) cycloheteroalkyl, (C₃ to C₈) cycloalkyl, (C₆ to C₁₄) aryl, or (C₂ to C₉) heteroaryl, each of which is optionally substituted with at least one R⁹ group; and R⁴ is H.

The present invention further relates to compounds of formula (I) as described above, wherein the compound of formula (I) has the following structure:

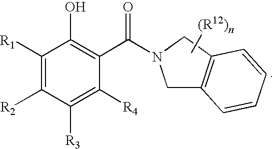

In one embodiment R² is —OH; and R³ and R⁴ are each independently H, —OH, (C₁ to C₆) alkyl, (C₁ to C₈) alkoxy, (C₁ to C₈) heteroalkyl, halogen, —CF₃, cyano, (C₂ to C₉) cycloheteroalkyl, (C₃ to C₈) cycloalkyl, (C₆ to C₁₄) aryl, or (C₂ to C₉) heteroaryl, each of which is optionally substituted with at least one R⁹ group; or R³ together with R⁴, together with the atoms to which they are attached, form a (C₆ to C₁₄) aryl, (C₂ to C₉) heteroaryl, (C₂ to C₉) cycloheteroalkyl, or a (C₃ to C₈) cycloalkyl group, each of which is optionally substituted with at least one R⁹ group.

In a further embodiment R³ and R⁴ are each independently H, —OH, (C₁ to C₄) alkyl, (C₁ to C₄) alkoxy, (C₁ to C₄) heteroalkyl, halogen, —CF₃, cyano, (C₂ to C₉) cycloheteroalkyl, (C₃ to C₈) cycloalkyl, (C₆ to C₁₄) aryl, or (C₂ to C₉) heteroaryl, each of which is optionally substituted with at least one R⁹ group. In a still further embodiment R³ is halogen and R⁴ is H.

In another embodiment R³ is (C₂ to C₉) cycloheteroalkyl, (C₃ to C₈) cycloalkyl, (C₆ to C₁₄) aryl, or (C₂ to C₉) heteroaryl, each of which is optionally substituted with at least one R⁹ group; and R⁴ is H.

The present invention further relates to compounds of formula (I) as described above, wherein the compound of formula (I) has the following structure:

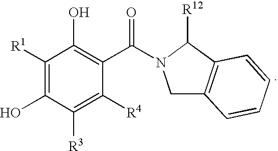

The present invention further relates to compounds of formula (I) as described above, wherein the compound of formula (I) has the following structure:

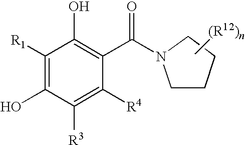

The present invention also provides a compound selected from the group consisting of: 3-bromo-2-(1,3-dihydro-2H-isoindol-2-ylcarbonyl)phenol; 4-(1,3-dihydro-2H-isoindol-2-ylcarbonyl)benzene-1,3-diol; tert-butyl [3-(2,4-dihydroxybenzoyl)-3-azabicyclo[3.1.0]hex-6-yl]carbamate; 4-{[2-(2-methylphenyl)pyrrolidin-1-yl]carbonyl}benzene-1,3-diol; 4-{[2-(1-naphthyl)pyrrolidin-1-yl]carbonyl}benzene-1,3-diol; 4-{[2-(3,5-dichlorophenyl)pyrrolidin-1-yl]carbonyl}benzene-1,3-diol; 4-{[2-(2-chlorophenyl)pyrrolidin-1-yl]carbonyl}benzene-1,3-diol; 4-[(2-phenylpyrrolidin-1-yl)carbonyl]benzene-1,3-diol; 2-(1,3-dihydro-2H-isoindol-2-ylcarbonyl)-4-fluorophenol; 4-bromo-2-(1,3-dihydro-2H-isoindol-2-ylcarbonyl)phenol; 2-(1,3-dihydro-2H-isoindol-2-ylcarbonyl)-5-(trifluoromethyl)phenol; 4-chloro-2-(1,3-dihydro-2H-isoindol-2-ylcarbonyl)phenol; 4-[(6-amino-3-azabicyclo[3.1.0]hex-3-yl)carbonyl]benzene-1,3-diol; 4-bromo-6-(1,3-dihydro-2H-isoindol-2-ylcarbonyl)benzene-1,3-diol; 3-(1,3-dihydro-2H-isoindol-2-ylcarbonyl)-2′-(trifluoromethyl)biphenyl-4-ol; 3-(1,3-dihydro-2H-isoindol-2-ylcarbonyl)biphenyl-4-ol; and 2-(1,3-dihydro-2H-isoindol-2-ylcarbonyl)-4-(1-methyl-1H-pyrazol-4-yl)phenol; 2-(1,3-dihydro-2H-isoindol-2-ylcarbonyl)-4-methoxyphenol; 2-(1,3-dihydro-2H-isoindol-2-ylcarbonyl)-5-methoxyphenol; 4-(1,3-dihydro-2H-isoindol-2-ylcarbonyl)-2-methylbenzene-1,3-diol; 4-(2,3-dihydro-1H-indol-1-ylcarbonyl)benzene-1,3-diol; 4-(1,3-dihydro-2H-isoindol-2-ylcarbonyl)benzene-1,3-diol; 1-(1,3-dihydro-2H-isoindol-2-ylcarbonyl)-2-naphthol; 3-(1,3-dihydro-2H-isoindol-2-ylcarbonyl)-2-naphthol; 3-(1,3-dihydro-2H-isoindol-2-ylcarbonyl)-6-methoxy-2-naphthol; 5-chloro-2-(1,3-dihydro-2H-isoindol-2-ylcarbonyl)phenol; 2-(1,3-dihydro-2H-isoindol-2-ylcarbonyl)-5-methylphenol; 3-(1,3-dihydro-2H-isoindol-2-ylcarbonyl)-2′,4′-difluorobiphenyl-4-ol; 2-(1,3-dihydro-2H-isoindol-2-ylcarbonyl)-5-fluorophenol; 2-(1,3-dihydro-2H-isoindol-2-ylcarbonyl)-4-methylphenol; 4-(2-aminopyrimidin-4-yl)-2-(1,3-dihydro-2H-isoindol-2-ylcarbonyl)phenol; 2-(1,3-dihydro-2H-isoindol-2-ylcarbonyl)-4-pyrimidin-4-ylphenol; 2-(1,3-dihydro-2H-isoindol-2-ylcarbonyl)-4-(1H-pyrazol-3-yl)phenol; 2-(1,3-dihydro-2H-isoindol-2-ylcarbonyl)-4-(2-ethylpyrimidin-4-yl)phenol; 2-(1,3-dihydro-2H-isoindol-2-ylcarbonyl)-4-(2-methylpyrimidin-4-yl)phenol; 2-(1,3-dihydro-2H-isoindol-2-ylcarbonyl)phenol; 4-({6-[(cyclopropylmethoxy)methyl]-1,4-oxazepan-4-yl}carbonyl)benzene-1,3-diol; 4-({6-[(cyclopropylmethoxy)methyl]-6-hydroxy-1,4-oxazepan-4-yl}carbonyl)benzene-1,3-diol; 2,4-dihydroxy-N-isobutyl-N-methylbenzamide; 2,4-dihydroxy-N-(2-hydroxycyclohexyl)-N-methylbenzamide; 4-{[2-(2,2-dimethylpropyl)piperidin-1-yl]carbonyl}benzene-1,3-diol; 4-[(4-methylpiperidin-1-yl)carbonyl]benzene-1,3-diol; 4-{[4-(4-chlorobenzyl)piperidin-1-yl]carbonyl}benzene-1,3-diol; 4-{[2-(2-cyclopentylethyl)piperidin-1-yl]carbonyl}benzene-1,3-diol; 2,4-dihydroxy-N-(2-hydroxycyclohexyl)-N-methylbenzamide; 4-{[4-(2-pyrimidin-2-ylethyl)piperidin-1-yl]carbonyl}benzene-1,3-diol; 4-{[4-(2-pyrazin-2-ylethyl)piperidin-1-yl]carbonyl}benzene-1,3-diol; 4-({4-[2-(3-chlorophenyl)ethyl]piperidin-1-yl}carbonyl)benzene-1,3-diol; 4-{[4-(2-phenylethyl)piperidin-1-yl]carbonyl}benzene-1,3-diol; 4-{[4-(2-pyrimidin-5-ylethyl)piperidin-1-yl]carbonyl}benzene-1,3-diol; 4-({3-[(2-fluorobenzyl)oxy]-1-oxa-8-azaspiro[4.5]dec-8-yl}carbonyl)benzene-1,3-diol; ethyl 1-(2,4-dihydroxybenzoyl)piperidine-3-carboxylate; 2,4-dihydroxy-N-(2-hydroxy-1-methyl-2-phenylethyl)-N-methylbenzamide; 4-[(4,4-diphenylpiperidin-1-yl)carbonyl]benzene-1,3-diol; 4-{[3-(2-phenoxyethyl)piperidin-1-yl]carbonyl}benzene-1,3-diol; 4-[(6,7-diethoxy-3,4-dihydroisoquinolin-2(1H)-yl)carbonyl]benzene-1,3-diol; 4-{[2-(3-fluorophenyl)piperidin-1-yl]carbonyl}benzene-1,3-diol; 4-[(3-{[(5-ethyl-1,2,4-oxadiazol-3-yl)methoxy]methyl}pyrrolidin-1-yl)carbonyl]benzene-1,3-diol; 4-({3-[(3-chlorophenoxy)methyl]piperidin-1-yl}carbonyl)benzene-1,3-diol; 4-[(3-{[(6-methylpyridin-3-yl)oxy]methyl}piperidin-1-yl)carbonyl]benzene-1,3-diol; 4-({6-[(cyclopentyloxy)methyl]-6-hydroxy-1,4-oxazepan-4-yl}carbonyl)benzene-1,3-diol; 4-{[2-(1,3-thiazol-2-yl)piperidin-1-yl]carbonyl}benzene-1,3-diol; 4-(piperidin-1-ylcarbonyl)benzene-1,3-diol; 4-({3-[2-(methoxymethyl)pyrimidin-4-yl]piperidin-1-yl}carbonyl)benzene-1,3-diol; 4-{[3-(2-phenylethyl)pyrrolidin-1-yl]carbonyl}benzene-1,3-diol; 2,4-dihydroxy-N-methyl-N-(1-phenylethyl)benzamide; 2,4-dihydroxy-N-methyl-N-(1-phenylethyl)benzamide; 4-{[4-(4-chlorophenoxy)piperidin-1-yl]carbonyl}benzene-1,3-diol; 4-{[4-(3-chlorophenoxy)piperidin-1-yl]carbonyl}benzene-1,3-diol; 4-{[4-(4-fluorophenyl)piperidin-1-yl]carbonyl}benzene-1,3-diol; 4-[(4-phenoxypiperidin-1-yl)carbonyl]benzene-1,3-diol; 4-{[4-(2-methoxyphenoxy)piperidin-1-yl]carbonyl}benzene-1,3-diol; 4-{[2-(2-methylphenyl)pyrrolidin-1-yl]carbonyl}benzene-1,3-diol; 4-{[4-(4-methylphenoxy)piperidin-1-yl]carbonyl}benzene-1,3-diol; 4-[(6,8-dimethyl-3,4-dihydro-1′H-spiro[chromene-2,4′-piperidin]-1′-yl)carbonyl]benzene-1,3-diol; 4-{[4-(4-chlorophenyl)-4-hydroxypiperidin-1-yl]carbonyl}benzene-1,3-diol; 4-[(4-hydroxy-4-phenylpiperidin-1-yl)carbonyl]benzene-1,3-diol; 4-({4-[4-chloro-3-(trifluoromethyl)phenyl]-4-hydroxypiperidin-1-yl}carbonyl)benzene-1,3-diol; 4-[(6-chloro-1′H-spiro[chromene-2,4′-piperidin]-1′-yl)carbonyl]benzene-1,3-diol; 4-(1′H,3H-spiro[2-benzofuran-1,4′-piperidin]-1′-ylcarbonyl)benzene-1,3-diol; 2,4-dihydroxy-N-methyl-N-[1-(1-naphthyl)ethyl]benzamide; 4-[(6-methyl-1′H-spiro[chromene-2,4′-piperidin]-1′-yl)carbonyl]benzene-1,3-diol; 4-({4-[2-(trifluoromethyl)pyrimidin-4-yl]piperidin-1-yl}carbonyl)benzene-1,3-diol; 4-{[2-(4-chlorophenyl)pyrrolidin-1-yl]carbonyl}benzene-1,3-diol; 1′-(2,4-dihydroxybenzoyl)spiro[chromene-2,4′-piperidin]-4(3H)-one; 4-[(4-pyrimidin-2-ylpiperidin-1-yl)carbonyl]benzene-1,3-diol; 4-({3-[(3-chlorophenoxy)methyl]pyrrolidin-1-yl}carbonyl)benzene-1,3-diol; 4-{[4-(5-chloro-1,3-benzoxazol-2-yl)-1,4-diazepan-1-yl]carbonyl}benzene-1,3-diol; 4-({3-[(2-chloro-4-fluorophenoxy)methyl]pyrrolidin-1-yl}carbonyl)benzene-1,3-diol; 4-(pyrrolidin-1-ylcarbonyl)benzene-1,3-diol; 1-{[7-(2,4-dihydroxybenzoyl)-6,7,8,9-tetrahydro-5H-[1,2,4]triazolo[4,3-d][1,4]diazepin-3-yl]methyl}pyrrolidin-2-one; 4-({2-[5-(2-methoxyphenyl)-1,3,4-oxadiazol-2-yl]pyrrolidin-1-yl}carbonyl)benzene-1,3-diol; 4-[(3-phenylpyrrolidin-1-yl)carbonyl]benzene-1,3-diol; methyl 1-(2,4-dihydroxybenzoyl)pyrrolidine-3-carboxylate; 4-{[3-(4-fluorophenyl)pyrrolidin-1-yl]carbonyl}benzene-1,3-diol; 4-{[3-(3-chlorophenyl)pyrrolidin-1-yl]carbonyl}benzene-1,3-diol; 4-(3,6-dihydropyridin-1(2H)-ylcarbonyl)benzene-1,3-diol; 4-[(6-{[(3,5-dimethylisoxazol-4-yl)methoxy]methyl}-1,4-oxazepan-4-yl)carbonyl]benzene-1,3-diol; 4-{[6-(3,4-dichlorobenzyl)-1,4-oxazepan-4-yl]carbonyl}benzene-1,3-diol; 4-{[3-hydroxy-3-(2-methylphenyl)pyrrolidin-1-yl]carbonyl}benzene-1,3-diol; 4-{[4-(6-methyl pyridin-2-yl)piperazin-1-yl]carbonyl}benzene-1,3-diol; N-[2-(3,4-dimethoxyphenyl)ethyl]-2,4-dihydroxy-N-methylbenzamide; (2,4-Dihydroxy-phenyl)-(1-phenyl-3,4-dihydro-1H-isoquinolin-2-yl)-methanone; 4-[(2-methyl-6,7-dihydro[1,3]thiazolo[5,4-c]pyridin-5(4H)-yl)carbonyl]benzene-1,3-diol; N-(4-fluorobenzyl)-2,4-dihydroxy-N-methylbenzamide; N-(4-chlorobenzyl)-2,4-dihydroxy-N-methylbenzamide; 2,4-dihydroxy-N-methyl-N-(4-phenoxybenzyl)benzamide; 2,4-dihydroxy-N-methyl-N-(2-phenylethyl)benzamide; 4-(3,4-dihydroisoquinolin-2(1H)-ylcarbonyl)benzene-1,3-diol; 4-({2-[(3-chlorophenoxy)methyl]morpholin-4-yl}carbonyl)benzene-1,3-diol; 4-[(4-pyrazin-2-ylpiperazin-1-yl)carbonyl]benzene-1,3-diol; 4-{[2-(phenoxymethyl)morpholin-4-yl]carbonyl}benzene-1,3-diol; N-(2-chlorobenzyl)-2,4-dihydroxy-N-methylbenzamide; 4-({2-[(3,5-difluorophenoxy)methyl]morpholin-4-yl}carbonyl)benzene-1,3-diol; 4-({2-[(2-chloro-4-fluorophenoxy)methyl]morpholin-4-yl}carbonyl)benzene-1,3-diol; 4-{[3-(4-methoxybenzyl)-5,6,8,9-tetrahydro-7H-[1,2,4]triazolo[4,3-d][1,4]diazepin-7-yl]carbonyl}benzene-1,3-diol; 4-(1,3-thiazolidin-3-ylcarbonyl)benzene-1,3-diol; 2,4-dihydroxy-N-{[5-(2-methoxyphenyl)-1,3,4-oxadiazol-2-yl]methyl}-N-methyl benzamide; N-{[5-(4-fluorophenyl)-1,3,4-oxadiazol-2-yl]methyl}-2,4-dihydroxy-N-methylbenzamide; 4-{[3-(4-fluorophenyl)-1,7-dioxa-2,10-diazaspiro[4.6]undec-2-en-10-yl]carbonyl}benzene-1,3-diol; N-{[5-(3-cyanophenyl)-1,3,4-oxadiazol-2-yl]methyl}-2,4-dihydroxy-N-methylbenzamide; 4-[(6-{[(2,6-dichlorobenzyl)oxy]methyl}-1,4-oxazepan-4-yl)carbonyl]benzene-1,3-diol; N-(1,3-benzoxazol-2-ylmethyl)-2,4-dihydroxy-N-methylbenzamide; 4-{[6-(hydroxymethyl)-4-pyrazin-2-yl-1,4-diazepan-1-yl]carbonyl}benzene-1,3-diol; 4-{[4-(6-chloro-1,3-benzoxazol-2-yl)piperazin-1-yl]carbonyl}benzene-1,3-diol; 2-[4-(2,4-dihydroxybenzoyl)piperazin-1-yl]-6-[2-(trifluoromethyl)phenyl]pyrimidin-4(3H)-one; 4-{[4-(5-methoxy-1,3-benzoxazol-2-yl)piperazin-1-yl]carbonyl}benzene-1,3-diol; 4-{[3-(3,5-difluorophenyl)-1,7-dioxa-2,10-diazaspiro[4.6]undec-2-en-10-yl]carbonyl}benzene-1,3-diol; 4-{[3-(4-fluoro-3-methoxyphenyl)-1,7-dioxa-2,10-diazaspiro[4.6]undec-2-en-10-yl]carbonyl}benzene-1,3-diol; 4-[(3-phenyl-1,7-dioxa-2,10-diazaspiro[4.6]undec-2-en-10-yl)carbonyl]benzene-1,3-diol; 4-{[3-(2,6-difluorophenyl)-1,7-dioxa-2,10-diazaspiro[4.6]undec-2-en-10-yl]carbonyl}benzene-1,3-diol; 4-{[3-(2-fluoro-5-methoxyphenyl)-1,7-dioxa-2,10-diazaspiro[4.6]undec-2-en-10-yl]carbonyl}benzene-1,3-diol; 4-(thiomorpholin-4-ylcarbonyl)benzene-1,3-diol; (2,4-Dihydroxy-phenyl)-(3-naphthalen-1-yl-4,5,7,8-tetrahydro-1,2,3a,6-tetraaza-azulen-6-yl)-methanone; 4-[(3-phenylmorpholin-4-yl)carbonyl]benzene-1,3-diol; 4-(5,6-dihydro[1,2,4]triazolo[4,3-a]pyrazin-7(8H)-ylcarbonyl)benzene-1,3-diol; 4-bromo-6-[(2-{4-[(dimethylamino)methyl]phenyl}pyrrolidin-1-yl)carbonyl]benzene-1,3-diol; 4-{[3-(4-bromophenyl)piperazin-1-yl]carbonyl}-6-chlorobenzene-1,3-diol; 4-chloro-6-{[5-(hydroxymethyl)-1,3-dihydro-2H-isoindol-2-yl]carbonyl}benzene-1,3-diol; 4-tert-butyl-2-(1,3-dihydro-2H-isoindol-2-ylcarbonyl)phenol; 3-bromo-2-(1,3-dihydro-2H-isoindol-2-ylcarbonyl)phenol; -(1,3-dihydro-2H-isoindol-2-ylcarbonyl)-2′-(trifluoromethyl)biphenyl-4-ol; 3-(1,3-dihydro-2H-isoindol-2-ylcarbonyl)biphenyl-4-ol; 2-(1,3-dihydro-2H-isoindol-2-ylcarbonyl)-4-(1-methyl-1H-pyrazol-4-yl)phenol; 2-(5-chloro-2,4-dihydroxybenzoyl)-N-ethylisoindoline-1-carboxamide; 1-[3-(1,3-dihydro-2H-isoindol-2-ylcarbonyl)-4-hydroxyphenyl]ethanone; 2-(1,3-dihydro-2H-isoindol-2-ylcarbonyl)-4-(1-hydroxyethyl)phenol; 3-(1,3-dihydro-2H-isoindol-2-ylcarbonyl)-4-hydroxybenzonitrile; 4-chloro-6-(1,3-dihydro-2H-isoindol-2-ylcarbonyl)-5-methylbenzene-1,3-diol; 4-chloro-6-{[(2R)-2-(pyrrolidin-1-ylmethyl)pyrrolidin-1-yl]carbonyl}benzene-1,3-diol; 2-(5-chloro-2,4-dihydroxybenzoyl)-N-ethylisoindoline-5-carboxamide; 3-chloro-4,6-dihydroxy-N,N-dimethyl-2-(2-oxo-2-piperidin-1-ylethyl)benzamide; 2-(5-chloro-2,4-dihydroxybenzoyl)-N-ethylisoindoline-1-carboxamide; 2-(5-chloro-2,4-dihydroxybenzoyl)-N-ethylisoindoline-1-carboxamide; 2-(5-chloro-2,4-dihydroxybenzoyl)-N-cyclobutylisoindoline-1-carboxamide; 2-(5-chloro-2,4-dihydroxybenzoyl)-N-(2-isocyanoethyl)isoindoline-1-carboxamide; 2-(5-chloro-2,4-dihydroxybenzoyl)-N-cyclopropylisoindoline-1-carboxamide; 2-(5-chloro-2,4-dihydroxybenzoyl)-N-(2,2,2-trifluoroethyl)isoindoline-1-carboxamide; N-allyl-2-(5-chloro-2,4-dihydroxybenzoyl)isoindoline-1-carboxamide; 2-(5-chloro-2,4-dihydroxybenzoyl)-N-isopropylisoindoline-1-carboxamide; 2-(5-chloro-2,4-dihydroxybenzoyl)-N-[2-(dimethylamino)ethyl]isoindoline-1-carboxamide; 2-(5-chloro-2,4-dihydroxybenzoyl)-N-(2,2-difluoroethyl)isoindoline-1-carboxamide; 2-(5-chloro-2,4-dihydroxybenzoyl)-N-(4-hydroxycyclohexyl)isoindoline-1-carboxamide; 2-(5-chloro-2,4-dihydroxybenzoyl)-N-propylisoindoline-1-carboxamide; 4-{[2-(2-methoxyphenyl)pyrrolidin-1-yl]carbonyl}benzene-1,3-diol; 4-[(2-phenylpyrrolidin-1-yl)carbonyl]benzene-1,3-diol; 4-(1,3-dihydro-2H-isoindol-2-ylsulfonyl)benzene-1,3-diol; 4-{[(2S)-2-(2-methylphenyl)pyrrolidin-1-yl]carbonyl}benzene-1,3-diol; 4-{[(2R)-2-(2-methylphenyl)pyrrolidin-1-yl]carbonyl}benzene-1,3-diol; 4-[(2-{4-[(dimethylamino)methyl]phenyl}pyrrolidin-1-yl)carbonyl]benzene-1,3-diol; 4-[(2-biphenyl-4-ylpyrrolidin-1-yl)carbonyl]benzene-1,3-diol; 4-{[2-(3-bromophenyl)pyrrolidin-1-yl]carbonyl}benzene-1,3-diol; 4-[(2-biphenyl-3-ylpyrrolidin-1-yl)carbonyl]benzene-1,3-diol; 4-{[2-(2′-chlorobiphenyl-3-yl)pyrrolidin-1-yl]carbonyl}benzene-1,3-diol; 3′-[1-(2,4-dihydroxybenzoyl)pyrrolidin-2-yl]-N,N-dimethylbiphenyl-4-carboxamide; 3′-[1-(2,4-dihydroxybenzoyl)pyrrolidin-2-yl]-N,N-dimethylbiphenyl-3-carboxamide; 4-({2-[3′-(piperidin-1-ylmethyl)biphenyl-3-yl]pyrrolidin-1-yl}carbonyl)benzene-1,3-diol; 4-[1-(2,4-dihydroxybenzoyl)pyrrolidin-2-yl]-N,N-dimethylbenzamide; 4-{[2-(4-bromophenyl)pyrrolidin-1-yl]carbonyl}benzene-1,3-diol; 4-({2-[4-(1-methyl-1H-pyrazol-4-yl)phenyl]pyrrolidin-1-yl}carbonyl)benzene-1,3-diol; 4-{[3-(phenylsulfonyl)pyrrolidin-1-yl]carbonyl}benzene-1,3-diol; 4-chloro-6-{[2-(2-hydroxyethyl)piperidin-1-yl]carbonyl}-5-methylbenzene-1,3-diol; 4-chloro-5-methyl-6-{[2-(2-piperidin-1-ylethyl)piperidin-1-yl]carbonyl}benzene-1,3-diol; 4-chloro-6-{[2-(2-cyclopentylethyl)piperidin-1-yl]carbonyl}-5-methylbenzene-1,3-diol; 4-chloro-5-methyl-6-[(2-pyridin-2-yl piperidin-1-yl)carbonyl]benzene-1,3-diol; 4-chloro-5-methyl-6-(piperazin-1-ylcarbonyl)benzene-1,3-diol; 4-{[3-(methylsulfonyl)pyrrolidin-1-yl]carbonyl}benzene-1,3-diol; methyl 1-(2,4-dihydroxybenzoyl) pyrrolidine-3-carboxylate; 4-{[3-(pyrazin-2-ylmethyl)pyrrolidin-1-yl]carbonyl}benzene-1,3-diol; tert-butyl 1-(2,4-dihydroxybenzoyl)-D-prolinate; 4-{[3-(hydroxymethyl)pyrrolidin-1-yl]carbonyl}benzene-1,3-diol; benzyl 1-(2,4-dihydroxybenzoyl)-L-prolinate; 4-nitrobenzyl 1-(2,4-dihydroxybenzoyl)-L-prolinate; 4-[(3-benzylpyrrolidin-1-yl)carbonyl]benzene-1,3-diol; 4-[(2-phenylpyrrolidin-1-yl)carbonyl]benzene-1,3-diol; 4-{[2-(3-fluorophenyl)pyrrolidin-1-yl]carbonyl}benzene-1,3-diol; benzyl 1-(2,4-dihydroxybenzoyl)-D-prolinate; 4-[(2-{5-[(cyclopropylmethyl)thio]-4-methyl-4H-1,2,4-triazol-3-yl}pyrrolidin-1-yl)carbonyl]benzene-1,3-diol; 4-({2-[4-(trifluoromethyl)phenyl]pyrrolidin-1-yl}carbonyl)benzene-1,3-diol; 2,4-dichloro-6-(1,3-dihydro-2H-isoindol-2-ylcarbonyl)phenol; 4-{[3-(4-bromophenyl)piperazin-1-yl]carbonyl}-6-chlorobenzene-1,3-diol; 4-chloro-6-{[5-(hydroxymethyl)-1,3-dihydro-2H-isoindol-2-yl]carbonyl}benzene-1,3-diol; methyl 4-(1-(5-chloro-2,4-dihydroxybenzoyl)pyrrolidin-2-yl)-3-methylbenzoate; 3-[1-(5-chloro-2,4-dihydroxybenzoyl)pyrrolidin-2-yl]-N-ethyl-4-methylbenzamide; methyl 3-[1-(5-chloro-2,4-dihydroxybenzoyl)pyrrolidin-2-yl]-4-methylbenzoate; and 4-(1-(5-chloro-2,4-dihydroxybenzoyl)pyrrolidin-2-yl)-N-ethyl-3-methylbenzamide; or a pharmaceutically acceptable salt or solvate thereof.

It should be understood that the present invention encompasses compounds of formula (I) as described herein formed by any and all combinations of the definitions of R¹, R², R³, R⁴, R⁵, R⁶, R⁷, R^{8a}, R^{8b}, R⁹, R^{10a}, R^{10b}, R¹¹, R¹², R¹³, R¹⁴, X, m, and n as described herein, and further includes pharmaceutically acceptable salts and solvates thereof.

In a further aspect of the present invention are pharmaceutical compositions, comprising a therapeutically effective amount of at least one of the compounds described herein, or a pharmaceutically acceptable salt or solvate thereof, and a pharmaceutically acceptable carrier or diluent.

The present invention further relates to methods of reducing abnormal cell growth in a mammal in need thereof, comprising the step of administering to said mammal a therapeutically effective amount of at least one compound described herein, or a pharmaceutically acceptable salt or solvate thereof. For example, in one embodiment, the abnormal cell growth is cancerous.

The present invention also relates to methods of treating cancer in a mammal, comprising the step of administering to said mammal a therapeutically effective amount of at least one compound described herein, or a pharmaceutically acceptable salt or solvate thereof.

In another aspect of the present invention are methods of inhibiting HSP-90 enzymatic activity, comprising contacting said HSP-90 enzyme with an HSP-90-inhibiting amount of at least one compound described herein, or a pharmaceutically acceptable salt or solvate thereof.

In a further aspect of the present invention are uses of any of the compounds described herein, or a pharmaceutically acceptable salt or solvate thereof, in the manufacture of a medicament for the treatment of abnormal cell growth in a mammal.

As used herein, the terms “comprising” and “including” are used in their open, non-limiting sense.

The terms “halo” and/or “halogen” refer to fluorine, chlorine, bromine or iodine.

The term “(C₁ to C₆)” alkyl refers to a saturated aliphatic hydrocarbon radical including straight chain and branched chain groups of 1 to 6 carbon atoms. Examples of (C₁ to C₆) alkyl groups include methyl, ethyl, propyl, 2-propyl, n-butyl, iso-butyl, tert-butyl, pentyl, and the like.

The term “(C₂ to C₈) alkenyl”, as used herein, means an alkyl moiety comprising 2 to 8 carbons having at least one carbon-carbon double bond. The carbon-carbon double bond in such a group may be anywhere along the 2 to 8 carbon chain that will result in a stable compound. Such groups include both the E and Z isomers of said alkenyl moiety. Examples of such groups include, but are not limited to, ethenyl, propenyl, butenyl, allyl, and pentenyl. The term “allyl,” as used herein, means a —CH₂CH═CH₂ group. The term, “C(R)═C(R),” as used herein, represents a carbon-carbon double bond in which each carbon is substituted by an R group.

As used herein, the term “(C₂ to C₈) alkynyl” means an alkyl moiety comprising from 2 to 8 carbon atoms and having at least one carbon-carbon triple bond. The carbon-carbon triple bond in such a group may be anywhere along the 2 to 8 carbon chain that will result in a stable compound. Examples of such groups include, but are not limited to, ethyne, propyne, 1-butyne, 2-butyne, 1-pentyne, 2-pentyne, 1-hexyne, 2-hexyne, and 3-hexyne.

The term “(C₁ to C₈) alkoxy”, as used herein, means an O-alkyl group wherein said alkyl group contains from 1 to 8 carbon atoms and is straight, branched, or cyclic. Examples of such groups include, but are not limited to, methoxy, ethoxy, n-propyloxy, iso-propyloxy, n-butoxy, iso-butoxy, tert-butoxy, cyclopentyloxy, and cyclohexyloxy.

The term “(C₁ to C₈) heteroalkyl” refers to a straight- or branched-chain alkyl group having a total of from 2 to 12 atoms in the chain, including from 1 to 8 carbon atoms, and one or more atoms of which is a heteroatom selected from S, O, and N, with the proviso that said chain may not contain two adjacent O atoms or two adjacent S atoms. The S atoms in said chains may be optionally oxidized with one or two oxygen atoms, to afford sulfides and sulfones, respectively. Furthermore, the (C₁ to C₈) heteroalkyl groups in the compounds of the present invention can contain an oxo group at any carbon or heteroatom that will result in a stable compound. Exemplary (C₁ to C₈) heteroalkyl groups include, but are not limited to, alcohols, alkyl ethers, primary, secondary, and tertiary alkyl amines, amides, ketones, esters, sulfides, and sulfones.

The term “(C₆ to C₁₄) aryl”, as used herein, means a group derived from an aromatic hydrocarbon containing from 6 to 14 carbon atoms. Examples of such groups include, but are not limited to, phenyl or naphthyl. The terms “Ph” and “phenyl,” as used herein, mean a —C₆H₅ group. The term “benzyl,” as used herein, means a —CH₂C₆H₅ group.

“(C₂ to C₉) heteroaryl”, as used herein, means an aromatic heterocyclic group having a total of from 5 to 10 atoms in its ring, and containing from 2 to 9 carbon atoms and from one to four heteroatoms each independently selected from O, S and N, and with the proviso that the ring of said group does not contain two adjacent O atoms or two adjacent S atoms. The heterocyclic groups include benzo-fused ring systems. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The C₂ to C₉ heteroaryl groups may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

“(C₂ to C₉) cycloheteroalkyl”, as used herein, means a non-aromatic, monocyclic, bicyclic, tricyclic, spirocyclic, or tetracyclic group having a total of from 4 to 13 atoms in its ring system, and containing from 2 to 9 carbon atoms and from 1 to 4 heteroatoms each independently selected from O, S and N, and with the proviso that the ring of said group does not contain two adjacent O atoms or two adjacent S atoms. Furthermore, such C₂ to C₉ cycloheteroalkyl groups may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a C₂ to C₉ cycloheteroalkyl group contains a sulfur atom, said sulfur atom may be oxidized with one or two oxygen atoms to afford either a sulfoxide or sulfone. An example of a 4 membered cycloheteroalkyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such C₂ to C₉ cycloheteroalkyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl quinolizinyl, 3-oxopiperazinyl, 4-methylpiperazinyl, 4-ethylpiperazinyl, and 1-oxo-2,8,diazaspiro[4.5]dec-8-yl.

The term “(C₃ to C₈) cycloalkyl group” means a saturated, monocyclic, fused, spirocyclic, or polycyclic ring structure having a total of from 3 to 8 carbon ring atoms. Examples of such groups include, but are not limited to, cyclopropyl, cyclobutyl, cyclopentyl, cyclopentenyl, cyclohexyl, cycloheptyl, and adamantyl.

The term “cyano” refers to a —C≡N group.

The term “substituted,” means that the specified group or moiety bears one or more substituents. The term “unsubstituted,” means that the specified group bears no substituents. The term “optionally substituted” means that the specified group is unsubstituted or substituted by one or more substituents. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one to which the remainder of the compound of the present invention is bonded, minus an additional substituent, to leave 4). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

The term “solvate”, as used herein, means a pharmaceutically acceptable solvate form of a compound of the present invention that retains the biological effectiveness of such compound. Examples of solvates include, but are not limited to, compounds of the invention in combination with water, isopropanol, ethanol, methanol, dimethylsulfoxide (DMSO), ethyl acetate, acetic acid, ethanolamine, or mixtures thereof. It is specifically contemplated that in the present invention one solvent molecule can be associated with one molecule of the compounds of the present invention, such as a hydrate. Furthermore, it is specifically contemplated that in the present invention, more than one solvent molecule may be associated with one molecule of the compounds of the present invention, such as a dihydrate. Additionally, it is specifically contemplated that in the present invention less than one solvent molecule may be associated with one molecule of the compounds of the present invention, such as a hemihydrate. Furthermore, solvates of the present invention are contemplated as solvates of compounds of the present invention that retain the biological effectiveness of the non-hydrate form of the compounds.

The term “pharmaceutically acceptable salt,” as used herein, means a salt of a compound of the present invention that retains the biological effectiveness of the free acids and bases of the specified derivative and that is not biologically or otherwise undesirable.

The term “pharmaceutically acceptable formulation,” as used herein, means a combination of a compound of the invention, or a pharmaceutically acceptable salt or solvate thereof, and a carrier, diluent, and/or excipient(s) that are compatible with a compound of the present invention, and is not deleterious to the recipient thereof. Pharmaceutical formulations can be prepared by procedures known to those of ordinary skill in the art. For example, the compounds of the present invention can be formulated with common excipients, diluents, or carriers, and formed into tablets, capsules, and the like. Examples of excipients, diluents, and carriers that are suitable for such formulations include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as povidone, sodium starch glycolate, sodium carboxymethylcellulose, agar, calcium carbonate, and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate and solid polyethylene glycols. Final pharmaceutical forms may be pills, tablets, powders, lozenges, saches, cachets, or sterile packaged powders, and the like, depending on the type of excipient used. Additionally, it is specifically contemplated that pharmaceutically acceptable formulations of the present invention can contain more than one active ingredient. For example, such formulations may contain more than one compound according to the present invention. Alternatively, such formulations may contain one or more compounds of the present invention and one or more additional agents that reduce abnormal cell growth.

The term “HSP-90-inhibiting amount” as used herein, refers to the amount of a compound of the present invention, or a pharmaceutically acceptable salt or solvate thereof, required to inhibit the enzymatic activity of HSP-90 in vivo, such as in a mammal, or in vitro. The amount of such compounds required to cause such inhibition can be determined without undue experimentation using methods described herein and those known to those of ordinary skill in the art.

The term “inhibiting HSP-90 enzyme activity,” as used herein, means decreasing the activity or functioning of the HSP-90 enzyme either in vitro or in vivo, such as in a mammal, such as a human, by contacting the enzyme with a compound of the present invention.

The term “therapeutically effective amount,” as used herein, means an amount of a compound of the present invention, or a pharmaceutically acceptable salt or solvate thereof, that, when administered to a mammal in need of such treatment, is sufficient to effect treatment, as defined herein. Thus, a therapeutically effective amount of a compound of the present invention, or a pharmaceutically acceptable salt or solvate thereof, is a quantity sufficient to modulate or inhibit the activity of the HSP-90 enzyme such that a disease condition that is mediated by activity of the HSP-90 enzyme is reduced or alleviated.

The terms “treat”, “treating”, and “treatment” refer to any treatment of an HSP-90 mediated disease or condition in a mammal, particularly a human, and include: (i) preventing the disease or condition from occurring in a subject which may be predisposed to the condition, such that the treatment constitutes prophylactic treatment for the pathologic condition; (ii) modulating or inhibiting the disease or condition, i.e., arresting its development; (iii) relieving the disease or condition, i.e., causing regression of the disease or condition; or (iv) relieving and/or alleviating the disease or condition or the symptoms resulting from the disease or condition, e.g., relieving an inflammatory response without addressing the underlying disease or condition. With regard to cancer, these terms simply mean that the life expectancy of an individual affected with a cancer will be increased or that one or more of the symptoms of the disease will be reduced.

The term “compound of the present invention” refers to any of the above-mentioned compounds, as well as those in the Examples that follow, and include those generically described or those described as species. The term also refers to pharmaceutically acceptable salts or solvates of these compounds.

“Abnormal cell growth”, as used herein, unless otherwise indicated, refers to cell growth that is independent of normal regulatory mechanisms (e.g., loss of contact inhibition), including the abnormal growth of normal cells and the growth of abnormal cells. This includes, but is not limited to, the abnormal growth of: tumor cells (tumors) that proliferate by expressing a mutated tyrosine kinase or overexpression of a receptor tyrosine kinase; benign and malignant cells of other proliferative diseases in which aberrant tyrosine kinase activation occurs; any tumors that proliferate by receptor tyrosine kinases; any tumors that proliferate by aberrant serine/threonine kinase activation; benign and malignant cells of other proliferative diseases in which aberrant serine/threonine kinase activation occurs; tumors, both benign and malignant, expressing an activated Ras oncogene; tumor cells, both benign and malignant, in which the Ras protein is activated as a result of oncogenic mutation in another gene; benign and malignant cells of other proliferative diseases in which aberrant Ras activation occurs. Examples of such benign proliferative diseases are psoriasis, benign prostatic hypertrophy, human papilloma virus (HPV), and restinosis. “Abnormal cell growth” also refers to and includes the abnormal growth of cells, both benign and malignant, resulting from activity of the enzyme farnesyl protein transferase.

The terms “abnormal cell growth” and “hyperproliferative disorder” are used interchangeably in this application.

### DETAILED DESCRIPTION

The compounds of the present invention are useful for modulating or inhibiting HSP-90 activity. Accordingly, these compounds are useful for the prevention and/or treatment of disease states associated with abnormal cell growth such as cancer, alone or in combination with other anti-cancer agents.

In accordance with a convention used in the art, the symbol

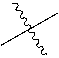

is used in structural formulas herein to depict the bond that is the point of attachment of the moiety or substituent to the core or backbone structure. In accordance with another convention, in some structural formulae herein the carbon atoms and their bound hydrogen atoms are not explicitly depicted, e.g.,

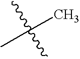

represents a methyl group,

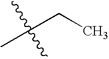

represents an ethyl group,

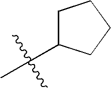

represents a cyclopentyl group, etc.

The compounds of the present invention may have asymmetric carbon atoms. The carbon-carbon bonds of the compounds of the present invention may be depicted herein using a solid line ( [symbol] ), a solid wedge ( [symbol] ), or a dotted wedge ( [symbol] ). The use of a solid line to depict bonds to asymmetric carbon atoms is meant to indicate that all possible stereoisomers at that carbon atom are included. The use of either a solid or dotted wedge to depict bonds to asymmetric carbon atoms is meant to indicate that only the stereoisomer shown is meant to be included. It is possible that compounds of the invention may contain more than one asymmetric carbon atom. In those compounds, the use of a solid line to depict bonds to asymmetric carbon atoms is meant to indicate that all possible stereoisomers are meant to be included. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present. Unless otherwise stated, all possible stereoisomers of the compounds of the present invention are meant to be included herein.

The term “stereoisomers” refers to compounds that have identical chemical constitution, but differ with regard to the arrangement of their atoms or groups in space. In particular, the term “enantiomers” refers to two stereoisomers of a compound that are non-superimposable mirror images of one another. The terms “racemic” or “racemic mixture,” as used herein, refer to a 1:1 mixture of enantiomers of a particular compound. The term “diastereomers”, on the other hand, refers to the relationship between a pair of stereoisomers that comprise two or more asymmetric centers and are not mirror images of one another.

A “solvate” is intended to mean a pharmaceutically acceptable solvate form of a specified compound that retains the biological effectiveness of such compound. Examples of solvates include, but are not limited to, compounds of the invention in combination with water, isopropanol, ethanol, methanol, dimethylsulfoxide (DMSO), ethyl acetate, acetic acid, ethanolamine, or mixtures thereof.

A “pharmaceutically acceptable salt” is intended to mean a salt that retains the biological effectiveness of the free acids and bases of the specified derivative, containing pharmacologically acceptable anions, and is not biologically or otherwise undesirable. Examples of pharmaceutically acceptable salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edisylate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, γ-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phosphate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

The compounds of the present invention that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

Those compounds of the present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts can be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds, agents and salts may exist in different crystal or polymorphic forms, all of which are intended to be within the scope of the present invention and specified formulas.

The compounds of the present invention may be formulated into pharmaceutical compositions as described below in any pharmaceutical form recognizable to the skilled artisan as being suitable. Pharmaceutical compositions of the invention comprise a therapeutically effective amount of at least one compound of the present invention and an inert, pharmaceutically acceptable carrier or diluent.

To treat or prevent diseases or conditions mediated by HSP-90, a pharmaceutical composition of the invention is administered in a suitable formulation prepared by combining a therapeutically effective amount (i.e., an HSP-90 modulating, regulating, or inhibiting amount effective to achieve therapeutic efficacy) of at least one compound of the present invention (as an active ingredient) with one or more pharmaceutically suitable carriers, which may be selected, for example, from diluents, excipients and auxiliaries that facilitate processing of the active compounds into the final pharmaceutical preparations.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol, Gelucire or the like, or formulator, such as CMC (carboxy-methylcellulose), PG (propyleneglycol), or PEG (polyethyleneglycol), may be added. Gelucire®, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g., parenteral or oral administration.

To obtain a stable water-soluble dose form, a pharmaceutically acceptable salt of a compound of the present invention may be dissolved in an aqueous solution of an organic or inorganic acid, such as a 0.3 M solution of succinic acid or citric acid. If a soluble salt form is not available, the agent may be dissolved in a suitable co-solvent or combinations of co-solvents. Examples of suitable co-solvents include alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds of the present invention may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use.

In addition to the formulations described above, the compounds of the present invention may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a co-solvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire®, Capryol®, Labrafil®, Labrasol®, Lauroglycol®, Plurol®, Peceol® Transcutol® and the like may be used.

Further, the pharmaceutical composition may be incorporated into a skin patch for delivery of the drug directly onto the skin.

It will be appreciated that the actual dosages of the agents of this invention will vary according to the particular agent being used, the particular composition formulated, the mode of administration, and the particular site, host, and disease being treated. Those skilled in the art using conventional dosage-determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

Furthermore, the pharmaceutically acceptable formulations of the present invention may contain a compound of the present invention, or a pharmaceutically acceptable salt or solvate thereof, in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg.

Additionally, the pharmaceutically acceptable formulations of the present invention may contain a compound of the present invention, or a pharmaceutically acceptable salt or solvate thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

The compounds of the present invention, or pharmaceutically acceptable salts or solvates thereof, may be administered to a mammal suffering from abnormal cell growth, such as a human, either alone or as part of a pharmaceutically acceptable formulation, once a day, twice a day, or three times a day.

Those of ordinary skill in the art will understand that with respect to the compounds of the present invention, the particular pharmaceutical formulation, the dosage, and the number of doses given per day to a mammal requiring such treatment, are all choices within the knowledge of one of ordinary skill in the art and can be determined without undue experimentation.

This invention also relates to a method for the treatment of abnormal cell growth in a mammal, including a human, comprising administering to said mammal an amount of a compound of the Formula I, as defined above, or a pharmaceutically acceptable salt or solvate thereof, that is effective in treating abnormal cell growth.

In one embodiment of this method, the abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In one embodiment of the present invention the cancer is selected from lung cancer (NSCLC and SCLC), cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non hodgkins's lymphoma, spinal axis tumors, or a combination of one or more of the foregoing cancers.

In another embodiment of the present invention the cancer is selected from lung cancer (NSCLC and SCLC), ovarian cancer, colon cancer, rectal cancer, cancer of the anal region, or a combination of one or more of the foregoing cancers.

In another embodiment of the present invention the cancer is selected from lung cancer (NSCLC and SCLC), ovarian cancer, colon cancer, rectal cancer, or a combination of one or more of the foregoing cancers.

In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of a compound of the present invention, or a pharmaceutically acceptable salt or solvate thereof, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a pharmaceutical composition for the treatment of abnormal cell growth in a mammal, including a human, comprising an amount of a compound of the present invention, as defined above, or a pharmaceutically acceptable salt or solvate thereof, that is effective in treating abnormal cell growth, and a pharmaceutically acceptable carrier. In one embodiment of said composition, said abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

The invention also relates to a pharmaceutical composition for the treatment of abnormal cell growth in a mammal, including a human, which comprises an amount of a compound of the present invention, as defined above, or a pharmaceutically acceptable salt or solvate thereof, that is effective in treating abnormal cell growth in combination with a pharmaceutically acceptable carrier and an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

The invention also relates to a method for the treatment of a hyperproliferative disorder in a mammal which comprises administering to said mammal a therapeutically effective amount of a compound of the present invention, or a pharmaceutically acceptable salt or hydrate thereof, in combination with an anti-tumor agent selected from the group consisting antiproliferative agents, kinase inhibitors, angiogenesis inhibitors, growth factor inhibitors, cox-I inhibitors, cox-II inhibitors, mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, statins, and anti-androgens.

In one embodiment of the present invention the anti-tumor agent used in conjunction with a compound of the present invention and pharmaceutical compositions described herein is an anti-angiogenesis agent, kinase inhibitor, pan kinase inhibitor or growth factor inhibitor.

Preferred pan kinase inhibitors include SU-11248, described in U.S. Pat. No. 6,573,293 (Pfizer, Inc, NY, USA).

Anti-angiogenesis agents, include but are not limited to the following agents, such as EGF inhibitor, EGFR inhibitors, VEGF inhibitors, VEGFR inhibitors, TIE2 inhibitors, IGF1R inhibitors, COX-II (cyclooxygenase II) inhibitors, MMP-2 (matrix-metalloprotienase 2) inhibitors, and MMP-9 (matrix-metalloprotienase 9) inhibitors.

Preferred VEGF inhibitors, include for example, Avastin (bevacizumab), an anti-VEGF monoclonal antibody of Genentech, Inc. of South San Francisco, Calif.

Additional VEGF inhibitors include CP-547,632 (Pfizer Inc., NY, USA), AG13736 (Pfizer Inc.), ZD-6474 (AstraZeneca), AEE788 (Novartis), AZD-2171), VEGF Trap (Regeneron/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof. VEGF inhibitors useful in the practice of the present invention are disclosed in U.S. Pat. Nos. 6,534,524 and 6,235,764, both of which are incorporated in their entirety for all purposed.

Particularly preferred VEGF inhibitors include CP-547,632, AG13736, Vatalanib, Macugen and combinations thereof.

Additional VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 6,534,524 (discloses AG13736), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following United States patent applications: Ser. No. 09/221,946 (filed Dec. 28, 1998); Ser. No. 09/454,058 (filed Dec. 2, 1999); Ser. No. 09/501,163 (filed Feb. 9, 2000); Ser. No. 09/539,930 (filed Mar. 31, 2000); Ser. No. 09/202,796 (filed May 22, 1997); Ser. No. 09/384,339 (filed Aug. 26, 1999); and Ser. No. 09/383,755 (filed Aug. 26, 1999); and the compounds disclosed and claimed in the following United States provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

PDGRr inhibitors include but not limited to those disclosed international patent application publication number WO01/40217, published Jul. 7, 2001 and international patent application publication number WO2004/020431, published Mar. 11, 2004, the contents of which are incorporated in their entirety for all purposes.

Preferred PDGFr inhibitors include Pfizer's CP-673,451 and CP-868,596 and its pharmaceutically acceptable salts.

Preferred GARF inhibitors include Pfizer's AG-2037 (pelitrexol and its pharmaceutically acceptable salts). GARF inhibitors useful in the practice of the present invention are disclosed in U.S. Pat. No. 5,608,082 which is incorporated in its entirety for all purposed.

Examples of useful COX-II inhibitors which can be used in conjunction with a compound of Formula I and pharmaceutical compositions described herein include CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additionally, COX-II inhibitors are disclosed in U.S. patent application Ser. Nos. 10/801,446 and 10/801,429, the contents of which are incorporated in their entirety for all purposes.

In one embodiment the anti-tumor agent is celecoxib as disclosed in U.S. Pat. No. 5,466,823, the contents of which are incorporated by reference in its entirety for all purposes. The structure for Celecoxib is shown below:

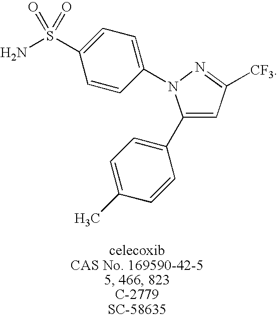

In one embodiment the anti-tumor agent is valecoxib as disclosed in U.S. Pat. No. 5,633,272, the contents of which are incorporated by reference in its entirety for all purposes. The structure for valdecoxib is shown below:

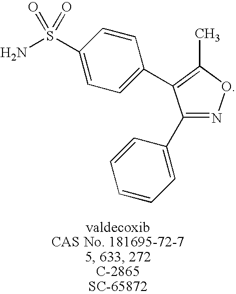

In one embodiment the anti-tumor agent is parecoxib as disclosed in U.S. Pat. No. 5,932,598, the contents of which are incorporated by reference in its entirety for all purposes. The structure for paracoxib is shown below:

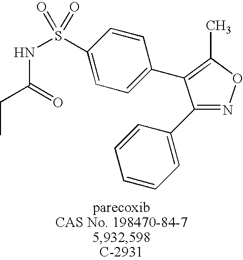

In one embodiment the anti-tumor agent is deracoxib as disclosed in U.S. Pat. No. 5,521,207, the contents of which are incorporated by reference in its entirety for all purposes. The structure for deracoxib is shown below:

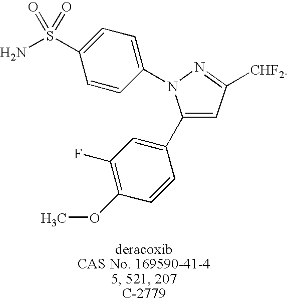

In one embodiment the anti-tumor agent is SD-8381 as disclosed in U.S. Pat. No. 6,034,256, the contents of which are incorporated by reference in its entirety for all purposes. The structure for SD-8381 is shown below:

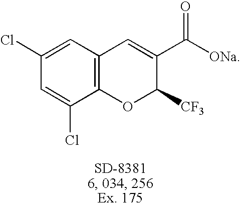

In one embodiment the anti-tumor agent is ABT-963 as disclosed in International Publication Number WO 2002/24719, the contents of which are incorporated by reference in its entirety for all purposes. The structure for ABT-963 is shown below:

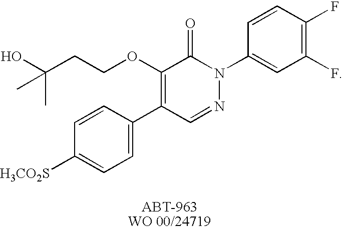

In one embodiment the anti-tumor agent is rofecoxib as shown below:

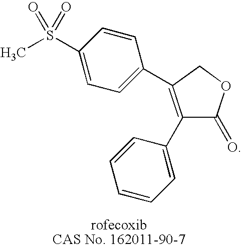

In one embodiment the anti-tumor agent is MK-663 (etoricoxib) as disclosed in International Publication Number WO 1998/03484, the contents of which are incorporated by reference in its entirety for all purposes. The structure for etoricoxib is shown below:

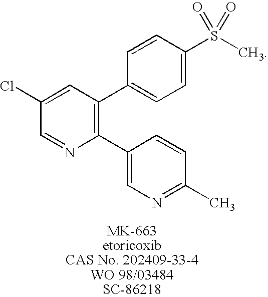

In one embodiment the anti-tumor agent is COX-189 (Lumiracoxib) as disclosed in International Publication Number WO 1999/11605, the contents of which are incorporated by reference in its entirety for all purposes. The structure for Lumiracoxib is shown below:

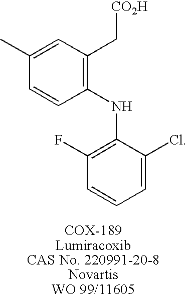

In one embodiment the anti-tumor agent is BMS-347070 as disclosed in U.S. Pat. No. 6,180,651, the contents of which are incorporated by reference in its entirety for all purposes. The structure for BMS-347070 is shown below:

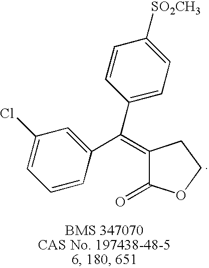

In one embodiment the anti-tumor agent is NS-398 (CAS 123653-11-2). The structure for NS-398 (CAS 123653-11-2) is shown below:

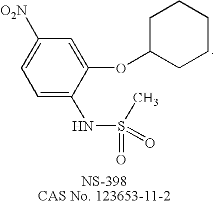

In one embodiment the anti-tumor agent is RS 57067 (CAS 17932-91-3). The structure for RS-57067 (CAS 17932-91-3) is shown below:

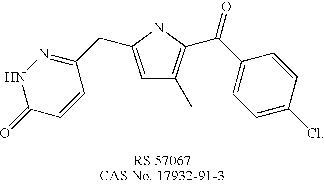

In one preferred embodiment the anti-tumor agent is 4-Methyl-2-(3,4-dimethylphenyl)-1-(4-sulfamoyl-phenyl)-1H-pyrrole. The structure for 4-Methyl-2-(3,4-dimethylphenyl)-1-(4-sulfamoyl-phenyl)-1H-pyrrole is shown below:

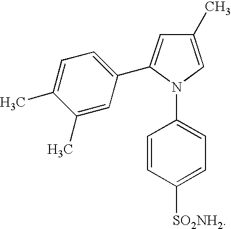

In one embodiment the anti-tumor agent is 2-(4-Ethoxyphenyl)-4-methyl-1-(4-sulfamoylphenyl)-1H-pyrrole. The structure for 2-(4-Ethoxyphenyl)-4-methyl-1-(4-sulfamoylphenyl)-1H-pyrrole is shown below:

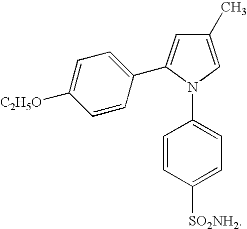

In one embodiment the anti-tumor agent is meloxicam. The structure for meloxicam is shown below:

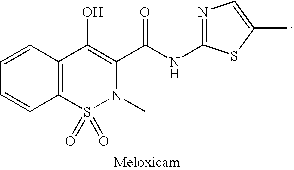

Other useful inhibitors as anti-tumor agents used in conjunction with a compound of the present invention and pharmaceutical compositions described herein include aspirin, and non-steroidal anti-inflammatory drugs (NSAIDs) which inhibit the enzyme that makes prostaglandins (cyclooxygenase I and II), resulting in lower levels of prostaglandins, include but are not limited to the following, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Preferred COX-I inhibitors include ibuprofen (Motrin), nuprin, naproxen (Aleve), indomethacin (Indocin), nabumetone (Relafen) and combinations thereof.

Targeted agents used in conjunction with a compound of the present invention and pharmaceutical compositions described herein include EGFr inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof.

Preferred EGFr inhibitors include Iressa, Erbitux, Tarceva and combinations thereof.

Other anti-tumor agents include those selected from pan erb receptor inhibitors or ErbB2 receptor inhibitors, such as CP-724,714 (Pfizer, Inc.), CI-1033 (canertinib, Pfizer, Inc.), Herceptin (trastuzumab, Genentech Inc.), Omitarg (2C4, pertuzumab, Genentech Inc.), TAK-165 (Takeda), GW-572016 (Ionafarnib, GlaxoSmithKline), GW-282974 (GlaxoSmithKline), EKB-569 (Wyeth), PKI-166 (Novartis), dHER2 (HER2 Vaccine, Corixa and GlaxoSmithKline), APC8024 (HER2 Vaccine, Dendreon), anti-HER2/neu bispecific antibody (Decof Cancer Center), B7.her2.IgG3 (Agensys), AS HER2 (Research Institute for Rad Biology & Medicine), trifunctional bispecific antibodies (University of Munich) and mAB AR-209 (Aronex Pharmaceuticals Inc) and mAB 2B-1 (Chiron) and combinations thereof.

Preferred erb selective anti-tumor agents include Herceptin, TAK-165, CP-724,714, ABX-EGF, HER3 and combinations thereof.

Preferred pan erbb receptor inhibitors include GW572016, CI-1033, EKB-569, and Omitarg and combinations thereof.

Additional erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Pat. Nos. 6,465,449, and 6,284,764, and International Application No. WO 2001/98277 each of which are herein incorporated by reference in their entirety.

Additionally, other anti-tumor agents may be selected from the following agents, BAY-43-9006 (Onyx Pharmaceuticals Inc.), Genasense (augmerosen, Genta), Panitumumab (Abgenix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aeterna), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antisoma), Thymitaq (Eximias), Temodar (temozolomide, Schering Plough) and Revilimd (Celegene) and combinations thereof.

Other anti-tumor agents may be selected from the following agents, CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE), ABI-007 (paclitaxel), Evista (raloxifene), Atamestane (Biomed-777), Xyotax (polyglutamate paclitaxel), Targetin (bexarotine) and combinations thereof.

Additionally, other anti-tumor agents may be selected from the following agents, Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpirnase), BEC2 (mitumoab), Xcytrin (motexafin gadolinium) and combinations thereof.

Further anti-tumor agents may selected from the following agents, CeaVac (CEA), NeuTrexin (trimetresate glucuronate) and combinations thereof.

Additional anti-tumor agents may selected from the following agents, OvaRex (oregovomab), Osidem (IDM-1), and combinations thereof.

Additional anti-tumor agents may selected from the following agents, Advexin (ING 201), Tirazone (tirapazamine), and combinations thereof.

Additional anti-tumor agents may selected from the following agents, RSR13 (efaproxiral), Cotara (131I chTNT 1/b), NBI-3001 (IL-4) and combinations thereof.

Additional anti-tumor agents may selected from the following agents, Canvaxin, GMK vaccine, PEG Interon A, Taxoprexin (DHA/paciltaxel) and combinations thereof.

Other anti-tumor agents include Pfizer's MEK1/2 inhibitor PD325901, Array Biopharm's MEK inhibitor ARRY-142886, Bristol Myers' CDK2 inhibitor BMS-387,032, Pfizer's CDK inhibitor PD0332991 and AstraZeneca's AXD-5438 and combinations thereof.

Additionally, mTOR inhibitors may also be utilized such as CCI-779 (Wyeth) and rapamycin derivatives RAD001 (Novartis) and AP-23573 (Ariad), HDAC inhibitors SAHA (Merck Inc./Aton Pharmaceuticals) and combinations thereof.

Additional anti-tumor agents include aurora 2 inhibitor VX-680 (Vertex), Chk1/2 inhibitor XL844 (Exilixis).

The following cytotoxic agents, e.g., one or more selected from the group consisting of epirubicin (Ellence), docetaxel (Taxotere), paclitaxel, Zinecard (dexrazoxane), rituximab (Rituxan) imatinib mesylate (Gleevec), and combinations thereof, may be used in conjunction with a compound of the present invention and pharmaceutical compositions described herein.

The invention also contemplates the use of the compounds of the present invention together with hormonal therapy, including but not limited to, exemestane (Aromasin, Pfizer Inc.), leuprorelin (Lupron or Leuplin, TAP/Abbott/Takeda), anastrozole (Arimidex, Astrazeneca), gosrelin (Zoladex, AstraZeneca), doxercalciferol, fadrozole, formestane, tamoxifen citrate (tamoxifen, Nolvadex, AstraZeneca), Casodex (AstraZeneca), Abarelix (Praecis), Trelstar, and combinations thereof.

The invention also relates to hormonal therapy agents such as anti-estrogens including, but not limited to fulvestrant, toremifene, raloxifene, lasofoxifene, letrozole (Femara, Novartis), anti-androgens such as bicalutamide, flutamide, mifepristone, nilutamide, Casodex® (4′-cyano-3-(4-fluorophenylsulphonyl)-2-hydroxy-2-methyl-3′-(trifluoromethyl)propionanilide, bicalutamide) and combinations thereof.

Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

Particularly preferred cytotoxic agents include Camptosar, Erbitux, Iressa, Gleevec, Taxotere and combinations thereof.

The following topoisomerase I inhibitors may be utilized as anti-tumor agents: camptothecin; irinotecan HCl (Camptosar); edotecarin; orathecin (Supergen); exatecan (Daiichi); BN-80915 (Roche); and combinations thereof.

Particularly preferred toposimerase II inhibitors include epirubicin (Ellence).

The compounds of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers.

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, Paraplatin (carboplatin), eptaplatin, lobaplatin, nedaplatin, Eloxatin (oxaliplatin, Sanofi) or satrplatin and combinations thereof. Particularly preferred alkylating agents include Eloxatin (oxaliplatin).

Antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, Alimta (premetrexed disodium, LY231514, MTA), Gemzar (gemcitabine, Eli Lilly), fludarabin, 5-azacitidine, capecitabine, cladribine, clofarabine, decitabine, eflornithine, ethynylcytidine, cytosine arabinoside, hydroxyurea, TS-1, melphalan, nelarabine, nolatrexed, ocfosfate, disodium premetrexed, pentostatin, pelitrexol, raltitrexed, triapine, trimetrexate, vidarabine, vincristine, vinorelbine; or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

Antibiotics include intercalating antibiotics but are not limited to: aclarubicin, actinomycin D, amrubicin, annamycin, adriamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin, zinostatin and combinations thereof.

Plant derived anti-tumor substances include for example those selected from mitotic inhibitors, for example vinblastine, docetaxel (Taxotere), paclitaxel and combinations thereof.

Cytotoxic topoisomerase inhibiting agents include one or more agents selected from the group consisting of aclarubicin, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, topotecan, and combinations thereof.

Preferred cytotoxic topoisomerase inhibiting agents include one or more agents selected from the group consisting of camptothecin, 10-hydroxycamptothecin, 9-aminocamptothecin, irinotecan HCl (Camptosar), edotecarin, epirubicin (Ellence), etoposide, SN-38, topotecan, and combinations thereof.

Immunologicals include interferons and numerous other immune enhancing agents. Interferons include interferon alpha, interferon alpha-2a, interferon, alpha-2b, interferon beta, interferon gamma-1a, interferon gamma-1b (Actimmune), or interferon gamma-n1 and combinations thereof. Other agents include filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab (Y-muHMFG1), Provenge (Dendreon) and combinations thereof.

Biological response modifiers are agents that modify defense mechanisms of living organisms or biological responses, such as survival, growth, or differentiation of tissue cells to direct them to have anti-tumor activity. Such agents include krestin, lentinan, sizofuran, picibanil, ubenimex and combinations thereof.

Other anticancer agents include alitretinoin, ampligen, atrasentan bexarotene, bortezomib. Bosentan, calcitriol, exisulind, finasteride, fotemustine, ibandronic acid, miltefosine, mitoxantrone, 1-asparaginase, procarbazine, dacarbazine, hydroxycarbamide, pegaspargase, pentostatin, tazarotne, Telcyta (TLK-286, Telik Inc.), Velcade (bortemazib, Millenium), tretinoin, and combinations thereof.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain, Vitaxin and combinations thereof.

Platinum-coordinated compounds include but are not limited to, cisplatin, carboplatin, nedaplatin, oxaliplatin, and combinations thereof.

Camptothecin derivatives include but are not limited to camptothecin, 10-hydroxycamptothecin, 9-aminocamptothecin, irinotecan, SN-38, edotecarin, topotecan and combinations thereof.

Other antitumor agents include mitoxantrone, I-asparaginase, procarbazine, dacarbazine, hydroxycarbamide, pentostatin, tretinoin and combinations thereof.

Anti-tumor agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736; and anti-proliferative agents such as other farnesyl protein transferase inhibitors, for example the farnesyl protein transferase inhibitors. Additionally, specific CTLA4 antibodies that can be used in the present invention include those described in U.S. Provisional Application 60/113,647 (filed Dec. 23, 1998), U.S. Pat. No. 6,682,736 both of which are herein incorporated by reference in their entirety.

Specific IGF1R antibodies that can be used in the present invention include those described in International Patent Application No. WO 2002/053596, which is herein incorporated by reference in its entirety.

Specific CD40 antibodies that can be used in the present invention include those described in International Patent Application No. WO 2003/040170 which is herein incorporated by reference in its entirety.

Gene therapy agents may also be employed as anti-tumor agents such as TNFerade (GeneVec), which express TNFalpha in response to radiotherapy.

In one embodiment of the present invention statins may be used in conjunction with a compound of the present invention and pharmaceutical compositions thereof. Statins (HMG-CoA reducatase inhibitors) may be selected from the group consisting of Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin and Niacin (Advicor, Kos Pharmaceuticals), derivatives and combinations thereof.

In a preferred embodiment the statin is selected from the group consisting of Atovorstatin and Lovastatin, derivatives and combinations thereof.

Other agents useful as anti-tumor agents include Caduet.

### Methods of Preparation

Compounds of the present invention may be prepared using the reaction routes and synthetic schemes described below, employing the techniques available in the art using starting materials that are readily available. The preparation of certain embodiments of the present invention is described in detail in the following examples, but those of ordinary skill in the art will recognize that the preparations described may be readily adapted to prepare other embodiments of the present invention. For example, the synthesis of non-exemplified compounds according to the invention may be performed by modifications apparent to those skilled in the art, e.g., by appropriately protecting interfering groups, by changing to other suitable reagents known in the art, or by making routine modifications of reaction conditions. Alternatively, other reactions disclosed herein or known in the art will be recognized as having adaptability for preparing other compounds of the invention.

In the following examples and preparations, “DMF” means di-methyl formamide, “Me” means methyl, “TEA” means tri-ethyl amine, “i-PrOH” means isopropyl alcohol, “HATU” means O-(7-azabenzotriazol-1-yl)-N,N,N′,N′-tetramethyluronium phosphorus pentafluoride, “DMSO” means di-methyl sulfoxide, “EtOAc” means ethyl acetate, “Boc” means t-butyloxycarbonyl, “DCM” means di-chloro methane, “DME” means di-methyl ether, “MeOH” means methanol, “t-BuLi” means tert-butyl lithium, “THF” means tetrahydrofuran, “DIEA” means diisopropyl ethylamine, “TBDMSCI” means tert-butyl di-methyl silyl chloride, “Pd(dppf)₂Cl₂” means dichloro (1,1 bis(diphenylphosphino) ferrocene) palladium(II).

### General Procedure G1

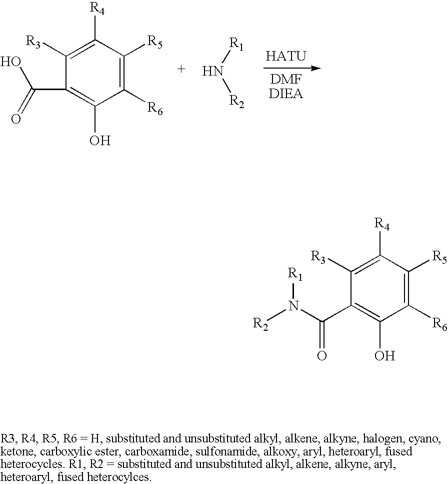

In a glovebox, a solution of the appropriate carboxylic acid solution (0.2 M, 400 mL, 0.08 mmole), a solution of the appropriate amine in DMF (0.2 M, 400 mL, 0.08 mmole), a solution of TEA (1.0 M, 80 mL, 0.08 mmole), and a solution of HATU in DMF (0.5 M, 160 mL, 0.08 mmole) are combined in a test tube, and the reactions are allowed to stir for 70° C. for 2 hours. The solvents are removed, and the residues are reconstituted in DMSO and then purified using reverse phase HPLC to give the desired product.

### General Procedure G2

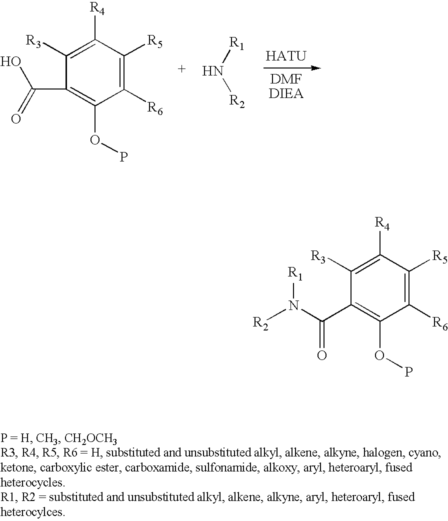

Amine (1.3 molar equivalent) is added to a solution of acid (0.17 mmole), diisopropylethyl amine (5 molar equivalent), and O-(7-azabenzotriazol-1-yl)-N,N,N′,N′-tetramethyluronium phosphorus pentafloride (HATU) (1.1 molar equivalent) in 3 mL of DMF under a nitrogen atmosphere. The reaction is allowed to stir at room temperature for 12 hours. Saturated NaHCO₃ is added to the reaction mixture to quench the reaction. EtOAc is then added to extract the aqueous solution. Dry EtOAc layer over Na₂SO₄. The Na₂SO₄ is filtered off and the filtrate is evaporated to give a brown oil residue. The residue is purified by silica gel chromatography (eluting with EtOAc and hexanes) to give the desired amide product.

### General Procedure G2a

### Boc Group Deprotection Step

Hydrogen chloride (5 to 30 molar equivalent, 4 M in dioxane) is added to a solution of the product obtained from G2 (0.15 mmole) in DCM or MeOH (4 mL). The reaction is stirred at room temperature for 12 hours. The reaction mixture is neutralized with NaHCO₃ and then EtOAc was added to extract the aqueous layer. The organic layer is dried, filtered, and concentrated to give the desired product.

### General Procedure G2b

### Methoxy Deprotection Step

Boron tribromide (3 molar equivalent, 1 M in DCM) is added to a solution of the product obtained from G2 (1 mmole) at −78° C. under N₂ atmosphere. The reaction mixture is allowed to warm up to room temperature and then stirred at room temperature for 12 hours. In an ice bath, NaHCO₃ (aq) is added to neutralize the reaction mixture and EtOAc (2×50 mL) is added to extract the aqueous solution. The organic layers are combined, dried, filtered, and concentrated to give a residue. The residue is purified by silica gel chromatography (eluting with EtOAc and hexanes) to give the desired product.

### General Procedure G4

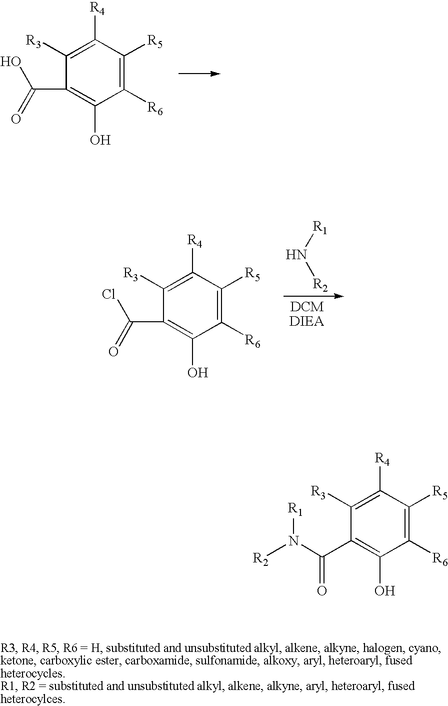

Oxalyl chloride (1.1 molar equivalent) is added to a solution of acid (1 mmol), DMF (0.1 mL) in 5 mL of DCM at 0° C. Under N₂ atmosphere, the reaction mixture is allowed to warm up to room temperature and then stirred for 12 hours. The solvent is evaporated to obtain a residue. Diisopropylethyl amine (5 molar equivalent) and DCM (5 mL) are added sequentially to the residue. Isoindoline (1 molar equivalent, in 2 mL of DMF) is added to the reaction mixture and the resulting mixture is stirred at room temperature for 12 hours. Water (20 mL) is added to quench the reaction and EtOAc was added to extract the aqueous solution. The organic layer is dried, filtered, and concentrated to obtain a residue. The residue is purified by silica gel chromatography (eluting with EtOAc and hexanes) to give the desired amide product.

### General Procedure G5

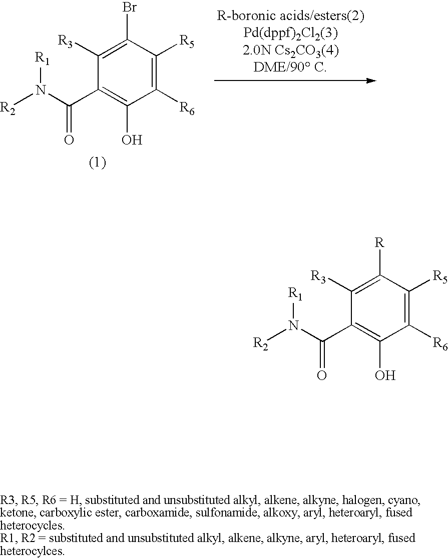

A reaction solution of Bromo-2-(1,3-dihydro-2H-isoindol-2-ylcarbonyl)phenol (1) (1.0 eq) and corresponding boronic acid (2) (1.3 eq) in 4 mL of DME is purged with N₂ for 15 minutes, then Pd(dppf)₂Cl₂ (3) (0.04 eq) is added, then another 2.0N Cs₂CO₃ solution (4) after being purged with N₂ for 15 minutes (3.0 eq) is added to the mixture. The resulting mixture is stirred at 90° C. for 4 hours. The reaction is completed by LCMS & TLC.

Work-up: The reaction mixture is filtered through Celite pad and washed well with MeOH. The filtrate is concentrated by vacuum. The residue is partitioned between EtOAc (200 mL) and sat. NaHCO₃ solution (2×50 mL) and brine (50 mL). The organic layer is dried (Na₂SO₄) and then concentrated by vacuum. The residue is purified by Biotage system (silica chromatography) to collect desired fractions to afford corresponding products.

### General Procedure G6

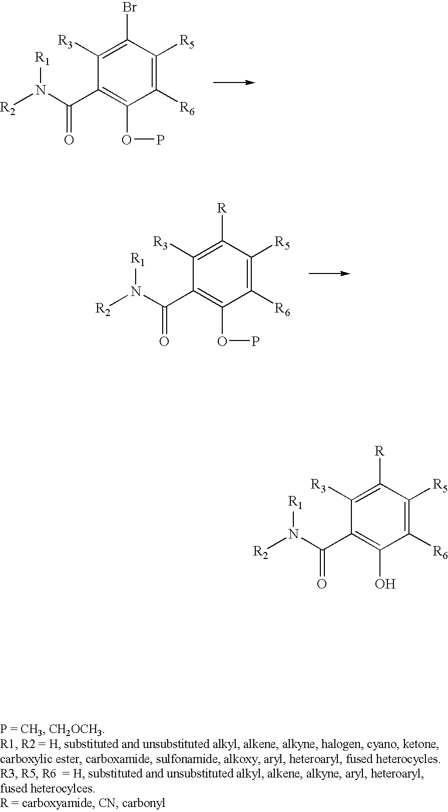

### General Procedure G7

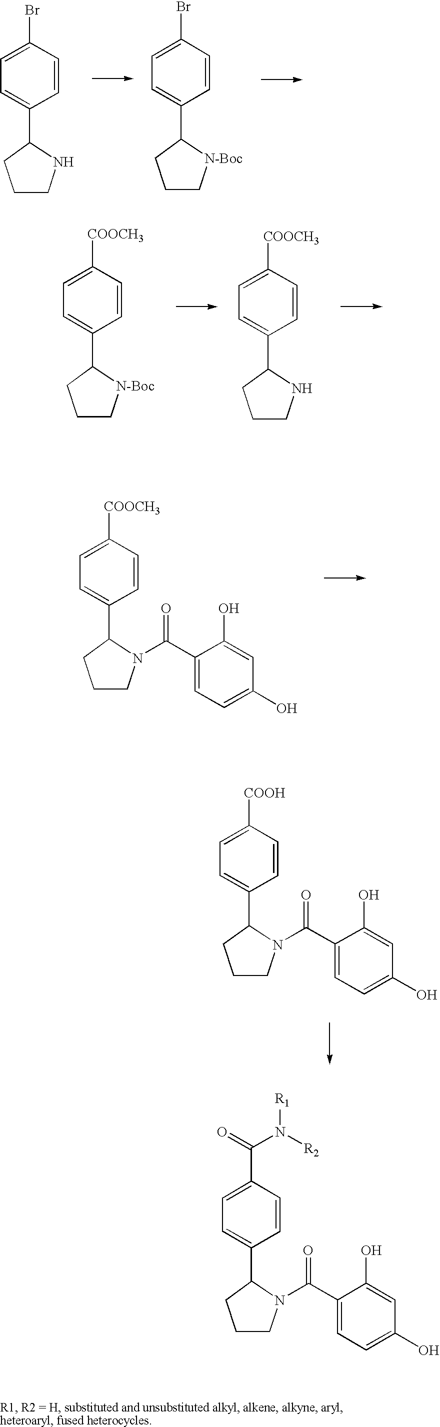

### General Procedure G8

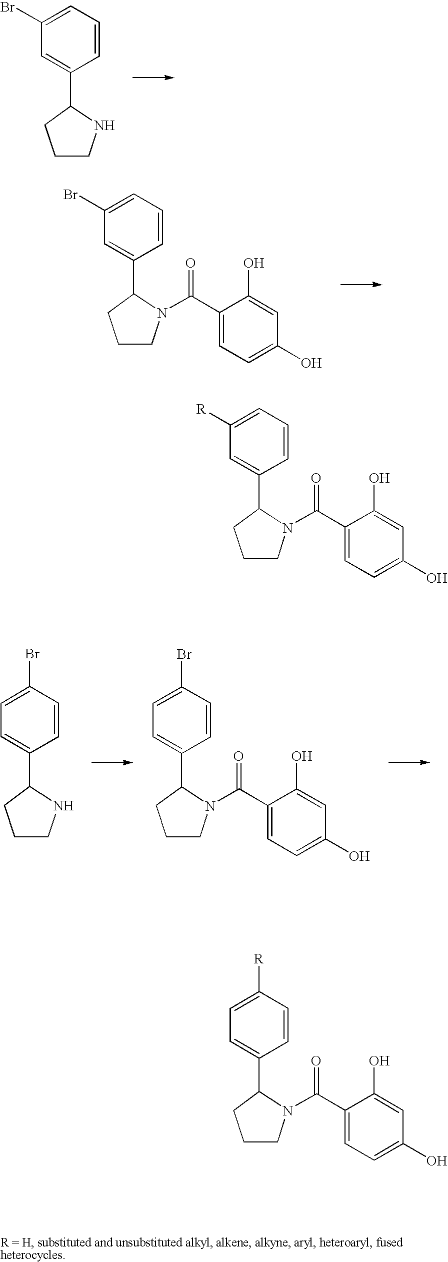

### General Procedure G9a

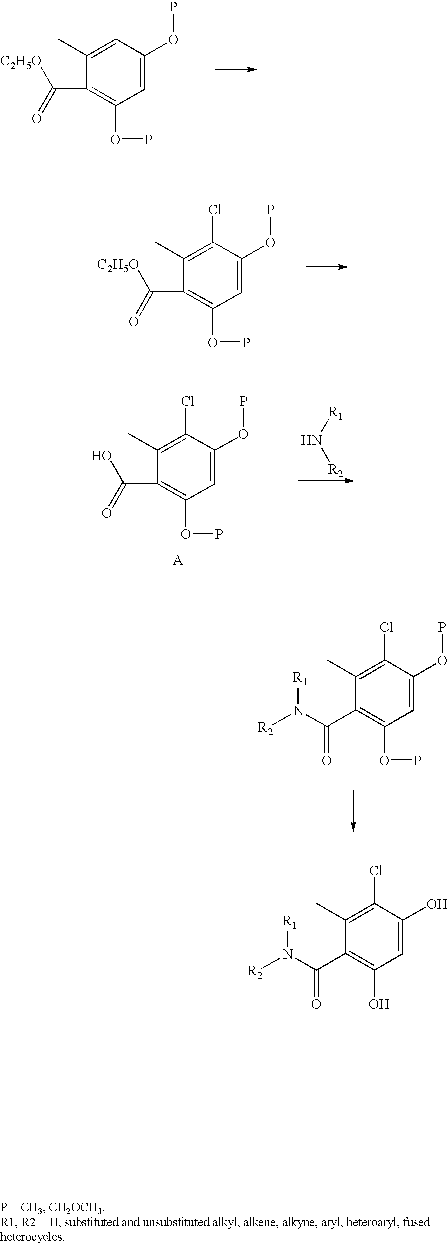

### General Procedure G9b

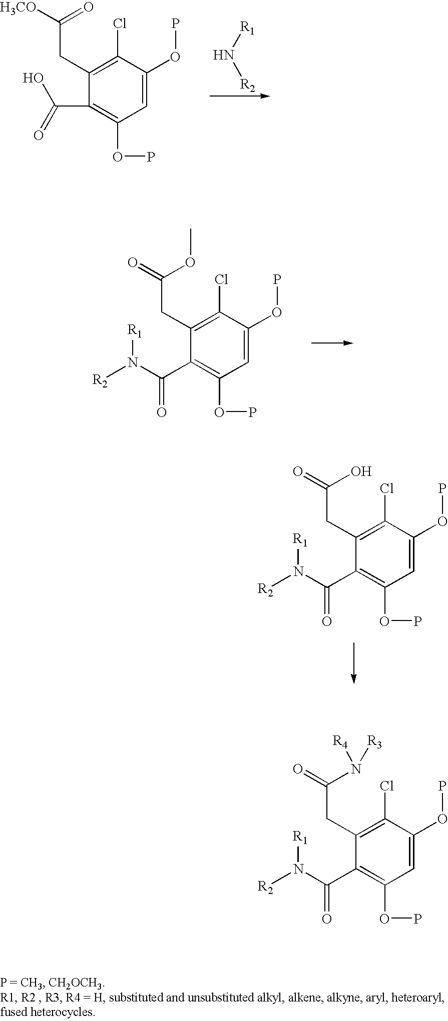

### General Procedure G10

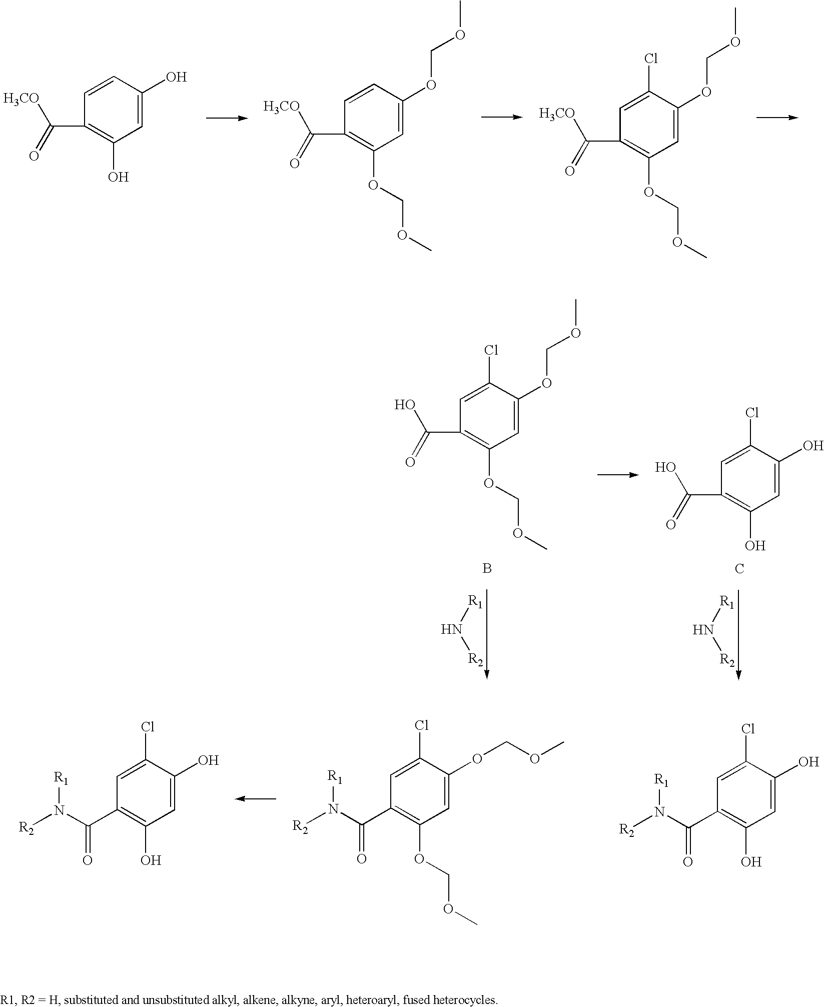

### General Procedure G11a

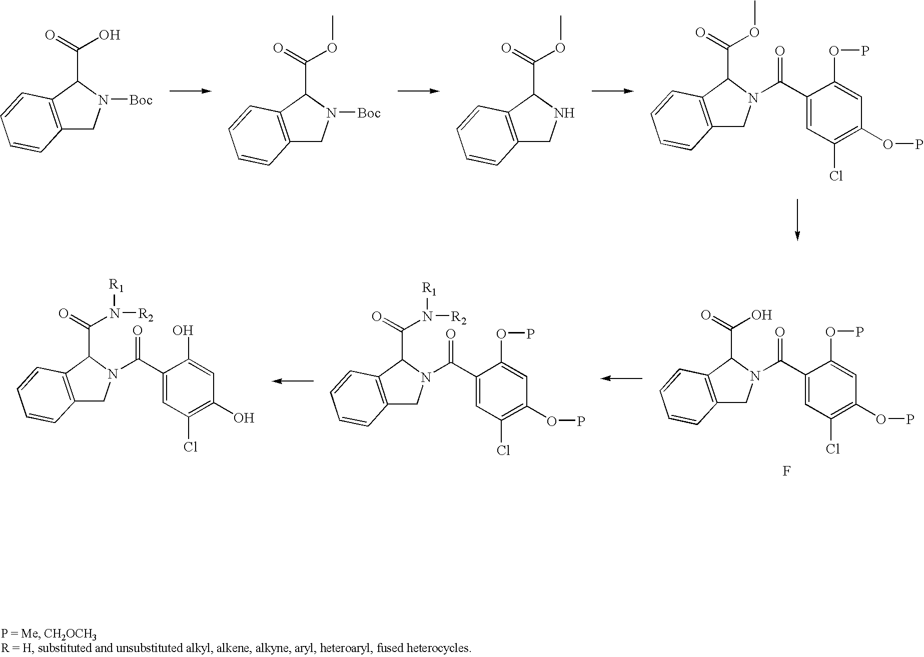

### General Procedure G11b

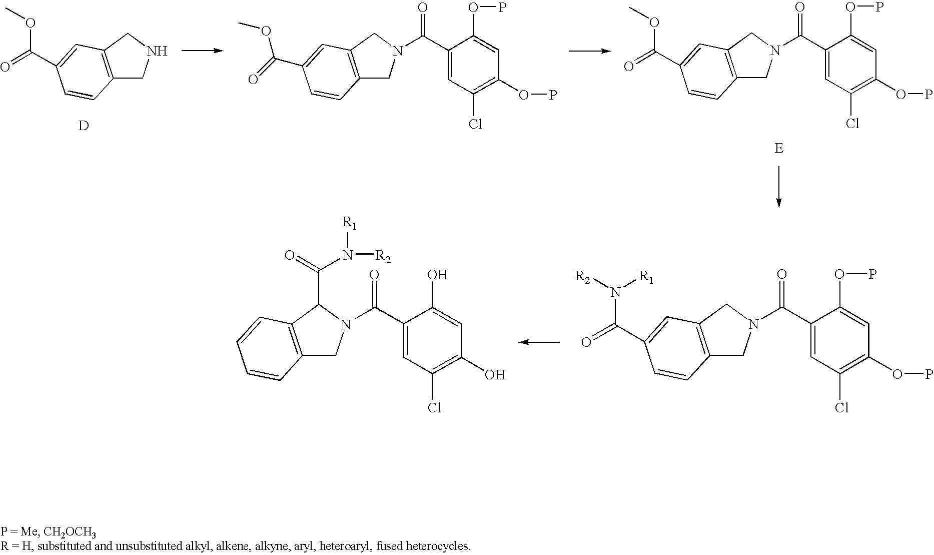

### General Procedure G12

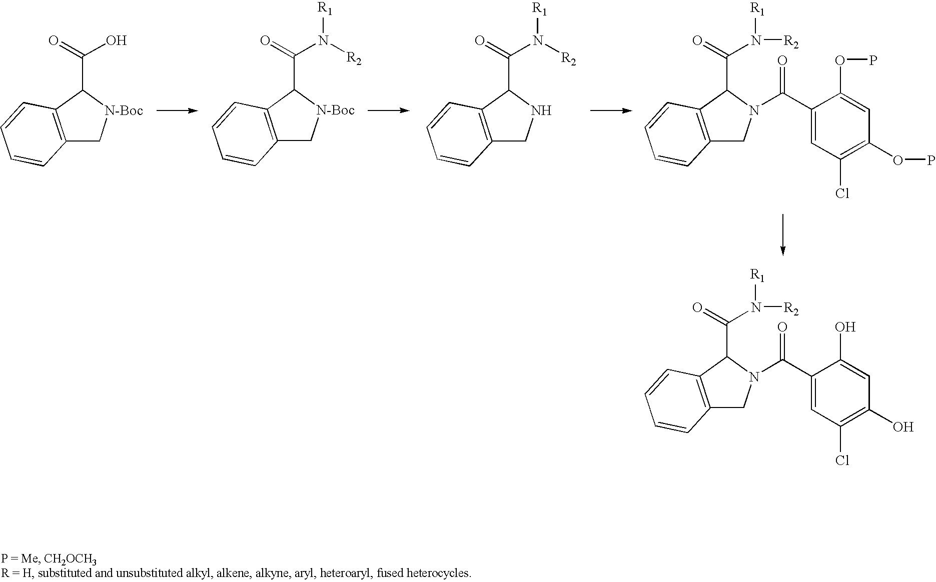

### General Procedure G13

Chiral chromatography to separate two enantiomers.

### General Procedure G14a

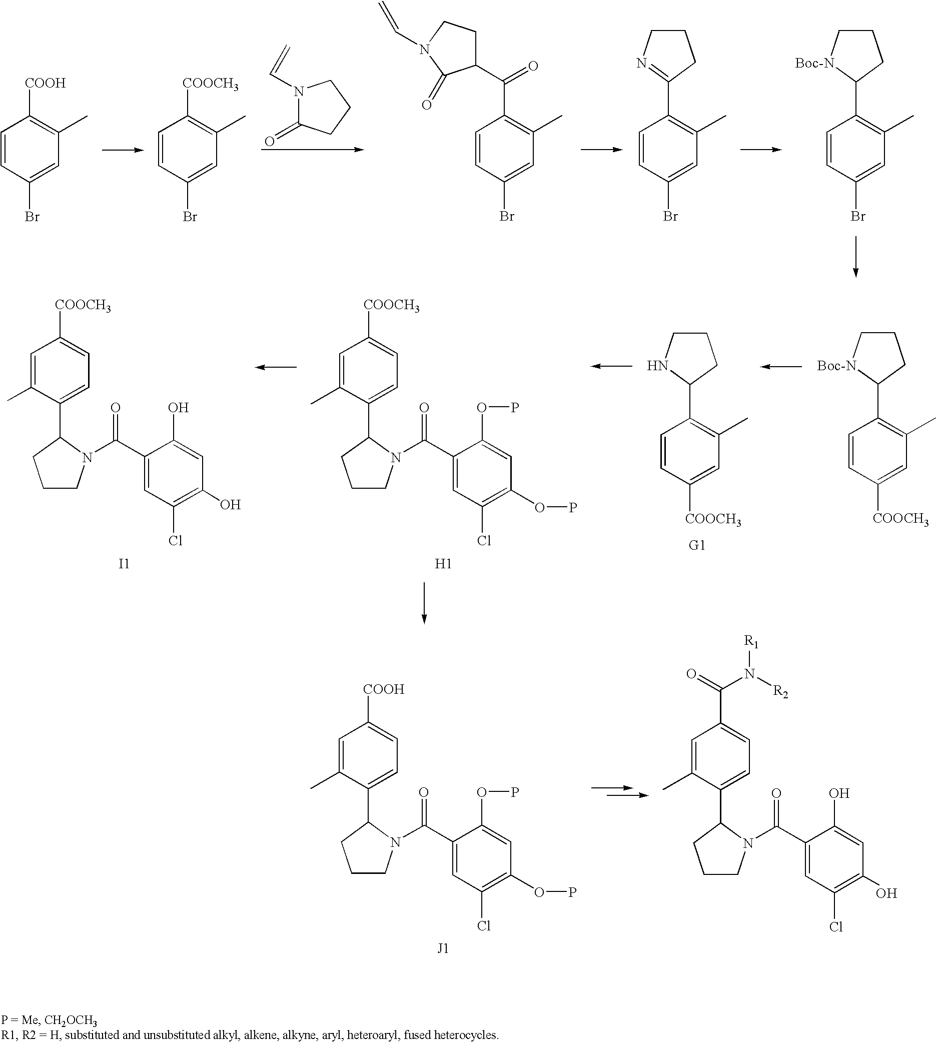

### General Procedure G14b

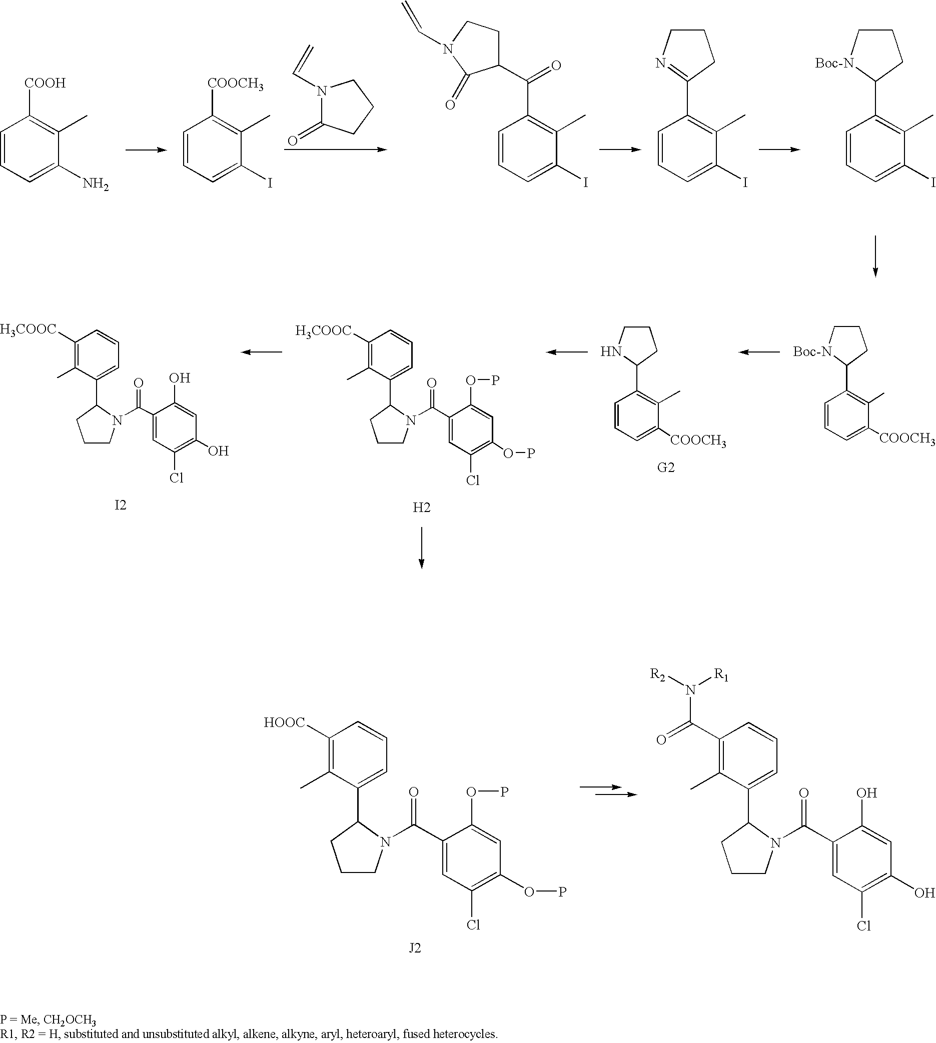

### General Procedure G14c

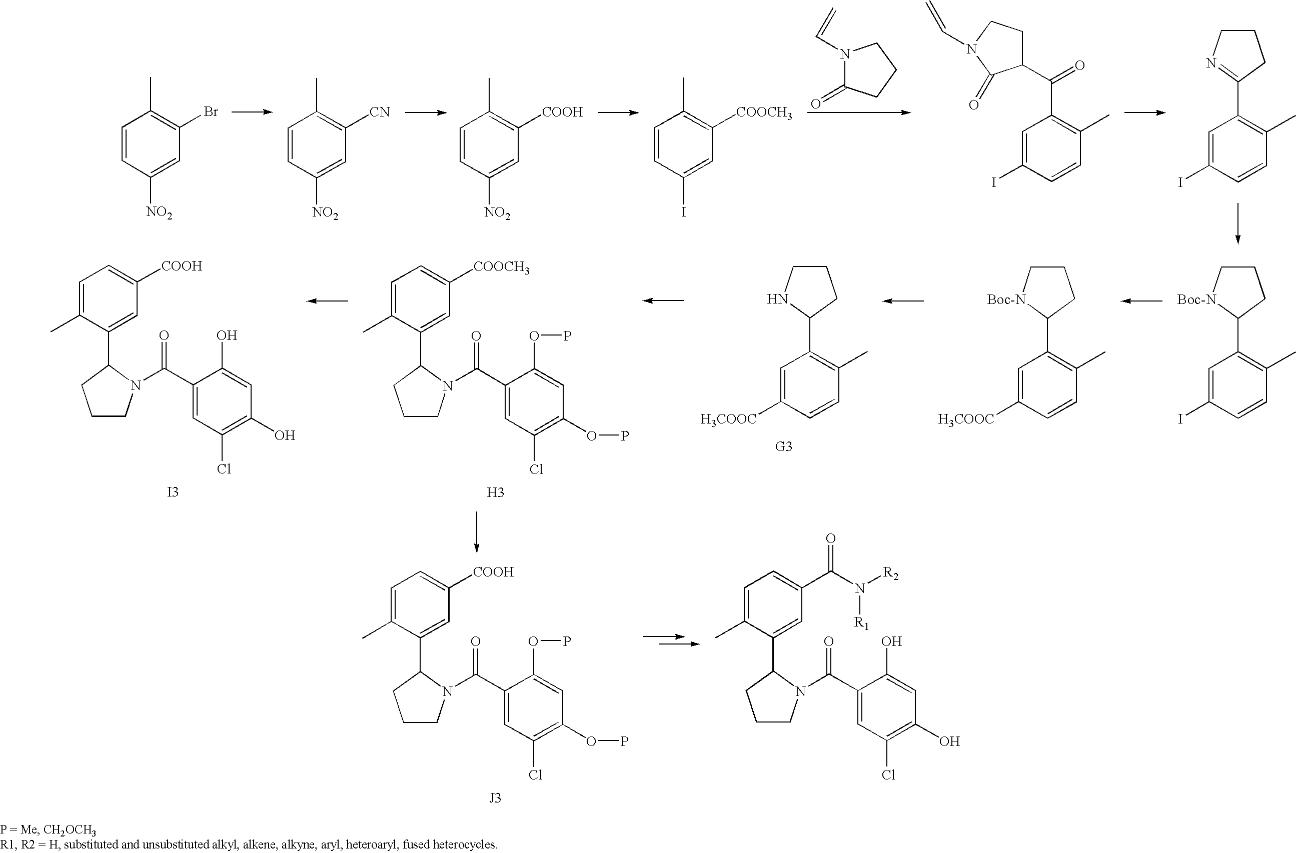

### EXAMPLES

The examples below are intended to illustrate particular embodiments of the present invention and are not meant to limit the scope of the invention in any way. 1 to 17 and Examples 137 to 167 provide detailed synthetic steps for preparing several specific compounds of the present invention. Table 1 shows additional compounds that were prepared as Examples 18 to 136 according to the general reaction schemes as described herein. Table 2 shows additional compounds that were prepared as Examples 168 to 214 according to the general reaction schemes as described herein. Examples 215 to 218 show detailed synthetic steps for several of the General Procedures described herein. Examples 219 to 221 describe and show the biochemical assay data from the compounds of Examples 1 to 214, and 217-218.

In the examples described below, unless indicated otherwise, all temperatures are in degrees Celsius (° C.) and all parts and percentages are by weight.

Various starting materials and other reagents were purchased from commercial suppliers, such as Aldrich Chemical Company, and used without further purification, unless indicated otherwise.

The reactions set forth below were performed under a positive pressure of nitrogen, argon or with a drying tube, at ambient temperature (unless otherwise stated), in anhydrous solvents. Analytical thin-layer chromatography was performed on glass-backed silica gel 60° F. 254 plates (Analtech (0.25 mm)) and eluted with the appropriate solvent ratios (v/v). The reactions were assayed by high-pressure liquid chromotagraphy (HPLC) or thin-layer chromatography (TLC) and terminated as judged by the consumption of starting material. The TLC plates were visualized by UV, phosphomolybdic acid stain, or iodine stain.

¹H-NMR spectra were recorded on a Bruker instrument operating at 400 or 500 MHz. NMR spectra are obtained as DMSO-d₆ or CDCl₃ solutions (reported in ppm), using chloroform as the reference standard (7.25 ppm and 77.00 ppm) or DMSO-d₆ (2.50 ppm and 39.52 ppm). Other NMR solvents were used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz.

Infrared spectra were recorded on a Perkin-Elmer FT-IR Spectrometer as neat oils, as KBr pellets, or as CDCl₃ solutions, and when reported are in wave numbers (cm⁻¹). The mass spectra were obtained using LC/MS or APCI. All melting points are uncorrected.

All final products had greater than 95% purity (by HPLC at wavelengths of 220 nm and 254 nm).

All elemental analyses for compounds herein, unless otherwise specified, provided values for C, H, and N analysis that were within 0.4% of the theoretical value, and are reported as “C, H, N.”

### Example 1

### 3-bromo-2-(1,3-dihydro-2H-isoindol-2-ylcarbonyl)phenol

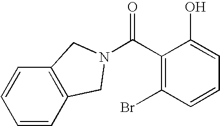

The above compound was made using the General Procedure G2 to afford product that was 90% pure (221 mg, 48%) and then re-purified to afford pure 3-bromo-2-(1,3-dihydro-2H-isoindol-2-ylcarbonyl)phenol (118 mg). ¹H NMR (400 MHz, DMSO-d₆) δ ppm 4.41-4.52 (m, 2H) 4.76-4.85 (m, 2H) 6.94 (d, J=8.08 Hz, 1H) 7.09-7.14 (m, 1H) 7.19 (t, J=8.08 Hz, 1H) 7.26-7.34 (m, 3H) 7.40 (d, J=7.07 Hz, 1H) 10.31 (s, 1H); Anal. Calc'd for C₁₇H₁₆BrNO₃.0.25H₂O: C, 55.01; H, 4.62; N, 3.77. Found: C, 55.07; H, 4.57; N, 3.60.

### 2-bromo-6-hydroxybenzoic acid

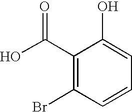

To a solution of methyl 2-bromo-6-hydroxybenzoate (547 mg, 2.01 mmol) in MeOH (10 mL) was added 1M NaOH (10 mL). Excess solid NaOH was added to saturate the solution. The resulting mixture was heated to 80° C. for 5 h and then allowed to cool to ambient temperature. The solution was concentrated to half of its volume and then carefully acidified with 1M HCl to a pH ˜1.0. The product was extracted with EtOAc (3×50 mL), dried (Na₂SO₄), and concentrated to afford 2-bromo-6-hydroxybenzoic acid as a white solid (409 mg, 94%). ¹H NMR (400 MHz, DMSO-d₆) δ ppm 6.87 (dd, J=8.34, 0.76 Hz, 1H) 7.03 (dd, J=8.08, 0.76 Hz, 1H) 7.13 (t, J=8.08 Hz, 1H) 11.75 (s, 1H).

### methyl 2-bromo-6-hydroxybenzoate

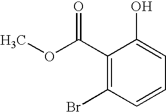

To a solution of 2-bromo-6-{[tert-butyl(dimethyl)silyl]oxy}-N,N-diethylbenzamide (2.905 g, 7.52 mmol) in anhydrous CH₃CN (50 mL) under a nitrogen atmosphere was added Na₂HPO₄ (1.601 g, 11.28 mmol) and then followed by Me₃OBF₄ (3.337 g, 22.56 mmol). The resulting solution was stirred for 16 h at ambient temperature and then saturated aqueous NaHCO₃ (50 mL) was added followed by solid NaHCO₃. The resulting solution was then stirred at ambient temperature for 16 h. The product was then extracted with EtOAc (3×200 mL), dried (Na₂SO₄), concentrated, and purified by flash chromatography (0%→20% EtOAc/hexanes) to afford methyl 2-bromo-6-hydroxybenzoate as a clear liquid (1.362 g, 78%). ¹H NMR (400 MHz, DMSO-d₆) δ ppm 3.80 (s, 3H) 6.91 (d, J=8.34 Hz, 1H) 7.07 (d, J=7.07 Hz, 1H) 7.19 (t, J=8.08 Hz, 1H) 10.45 (s, 1H).

### 2-bromo-6-{[tert-butyl(dimethyl)silyl]oxy}-N,N-diethylbenzamide

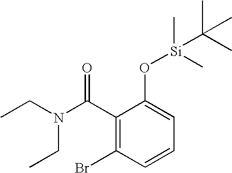

A solution of 2-{[tert-butyl(dimethyl)silyl]oxy}-N,N-diethylbenzamide (2.791 g, 9.08 mmol) in anhydrous THF (100 mL) under a nitrogen atmosphere was cooled to −78° C. and then t-BuLi (1.7M in pentane, 8.01 mL, 13.62 mmol) was slowly added dropwise over 30 minutes. The solution was stirred at −78° C. for an additional 30 minutes and then bromine (0.7 mL, 13.62 mmol) was slowly added over 30 additional minutes. The bath was removed and the solution allowed to warm to ambient temperature. After 20 h, saturated aqueous sodium thiosulfate and EtOAc was added and the organic layer separated. The organic layer was then washed with brine, dried (Na₂SO₄), concentrated, and purified by flash chromatography (0%→15% EtOAc/hexanes) to afford 2-bromo-6-{[tert-butyl(dimethyl)silyl]oxy}-N,N-diethylbenzamide as a white solid (2.905 g, 83%). ¹H NMR (400 MHz, DMSO-d₆) δ ppm 0.17 (s, 3H) 0.24 (s, 3H) 0.91 (s, 9H) 1.01 (t, J=7.07 Hz, 3H) 1.15 (t, J=7.07 Hz, 3H) 3.00-3.11 (m, 2H) 3.23-3.33 (m, J=7.20, 7.01, 6.80, 6.80, 6.80 Hz, 1H) 3.53-3.63 (m, J=13.64, 7.04, 7.04, 6.88 Hz, 1H) 6.93 (dd, J=6.95, 2.15 Hz, 1H) 7.18-7.24 (m, 2H).

### 2-{[tert-butyl(dimethyl)silyl]oxy}-N,N-diethylbenzamide

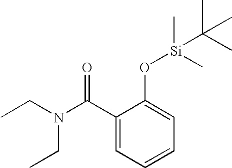

To a solution of N,N-diethyl-2-hydroxybenzamide (2.0 g, 10.35 mmol) in DMF (50 mL) was added DIEA (6.49 mL, 37.26 mmol) and TBDMSCI (3.9 g, 25.87 mmol) under a nitrogen atmosphere at ambient temperature. After 16 h, the solution was washed with H₂O (3×), dried (Na₂SO₄), concentrated, and purified by flash chromatography (0% 10% EtOAc/hexanes) to afford quantitative yield of 2-{[tert-butyl(dimethyl)silyl]oxy}-N,N-diethylbenzamide. ¹H NMR (400 MHz, DMSO-d₆) δ ppm 0.19 (d, J=17.18 Hz, 5H) 0.84 (s, 5H) 0.90-0.97 (m, 12H) 1.13 (t, J=7.07 Hz, 3H) 6.89 (d, J=8.34 Hz, 1H) 6.98 (t, J=7.45 Hz, 1H) 7.12 (dd, J=7.58, 1.77 Hz, 1H) 7.27 (td, J=7.83, 1.77 Hz, 1H).

### Example 2

### 4-(1,3-dihydro-2H-isoindol-2-ylcarbonyl)benzene-1,3-diol

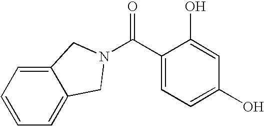

The above compound was made as follows using the General Procedure G2. Isoindoline (200 mg, 1.6 mmol) was added to a solution of 2,4-dihyrdoxybenzoic acid (200 mg, 1.3 mmol), diisopropylethyl amine (1.1 ml, 6.5 mmol), and O-(7-azabenzotriazol-1-yl)-N,N,N′,N′-tetramethyluronium phosphorus pentafloride (HATU) (550 mg, 1.4 mmol) in 5 mL of DMF under a nitrogen atmosphere. The reaction was allowed to stir at room temperature for 12 h. Saturated NaHCO₃ (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 30→40% EtOAc in hexanes) to give the desired product (115 mg, 34.8% yield). ¹H NMR (400 MHz, MeOD) δ ppm 4.87-4.94 (m, 4H) 6.14-6.53 (m, 2H) 7.00-7.52 (m, 5H); Anal. Calc'd for C₁₅H₁₃NO₃.0.15H₂O: C, 67.89; H, 5.00; N, 5.23. Found: C, 68.06; H, 4.99; N, 5.25.

### Example 3

### tert-butyl [3-(2,4-dihydroxybenzoyl)-3-azabicyclo[3.1.0]hex-6-yl]carbamate

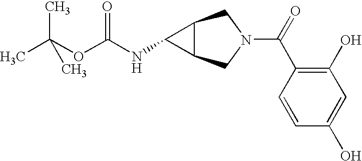

The above compound was made as follows using the General Procedure G2. (1A,5A,6A)-6-tert-amino-3-butoxycarbonylamine-3-azobicyclo {3,1,0}-hexane (290 mg, 1.4 mmol) (which was prepared as reported by Brighty et al. J. Synlett 11: 1097-1099 (1996)), the disclosure of which is incorporated herein by reference, was added to a solution of 2,4-dihydroxybenzoic acid (200 mg, 1.3 mmol), diisopropylethyl amine (1.1 mL, 6.5 mmol), and O-(7-azabenzotriazol-1-yl)-N,N,N′,N′-tetramethyluronium phosphorus pentafloride (HATU) (550 mg, 1.4 mmol) in 5 mL of DMF under a nitrogen atmosphere. The reaction was allowed to stir at room temperature for 12 h. Saturated NaHCO₃ (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 50 →60% EtOAc in hexanes) to give the desired product (145.8 mg, 33.8% yield). ¹H NMR (400 MHz, DMSO-D6) δ ppm 1.35 (s, 9H) 1.56-1.69 (m, 2H) 2.05-2.15 (m, 1H) 3.38-3.51 (m, 4H) 6.21 (dd, J=8.3, 2.3 Hz, 1H) 6.26 (d, J=2.0 Hz, 1H) 6.98 (d, J=8.6 Hz, 1H) 7.06 (s, 1H) 9.62 (s, 1H) 10.16 (s, 1H); Anal. Calc'd for C₁₇H₂₂N₂O₅.0.75 PF₆.0.75H₂O: C, 44.72; H, 5.19; N, 6.14. Found: C, 44.77; H, 4.80; N, 6.19.

### Example 4

### 4-{[2-(2-methylphenyl)pyrrolidin-1-yl]carbonyl}benzene-1,3-diol

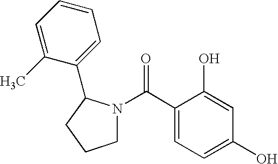

The above compound was made as follows using the General Procedure G2. O-(7-azabenzotriazol-1-yl)-N,N,N′,N′-tetramethyluronium phosphorus pentafloride (HATU) (319 mg, 0.84 mmol), diisopropylethyl amine (0.29 mL, 1.68 mmol) and 2,4-dihydroxybenzoic acid (87 mg, 0.56 mmol) were mixed with 2 mL of DMF under a nitrogen atmosphere. After 30 min, a DMF solution (1 mL) of 2-(2-methylphenyl)pyrrolidine (100 mg, 0.62 mmol) was then added to the solution. The reaction was allowed to stir at room temperature for 16 h. Saturated aqueous NaHCO₃ was added and the product extracted with EtOAc. The organic extract was then washed with H₂O and brine. The organic layer was then dried (Na₂SO₄), concentrated, and purified by flash chromatography (20%→40% EtOAc/hexanes) to afford product (35 mg, 21%) as a white solid. ¹H NMR (400 MHz, DMSO-d₆) δ ppm 1.54-1.65 (m, 1H) 1.78-1.90 (m, 2H) 2.31-2.42 (m, 4H) 3.55-3.65 (m, 1H) 3.81-3.92 (m, 1H) 5.20-5.31 (m, 1H) 6.23-6.33 (m, 2H) 7.05-7.16 (m, J=6.6 Hz, 3H) 7.20-7.31 (m, 2H) 9.70-9.81 (m, 1H) 10.66-10.76 (m, 1H). Elemental Analysis Calc'd for C₁₈H₁₉NO₃ C, 72.71; H, 6.44; N, 4.71. Found: C, 72.48; H, 6.54; N, 4.67.

### Example 5

### 4-{[2-(1-naphthyl)pyrrolidin-1-yl]carbonyl}benzene-1,3-diol

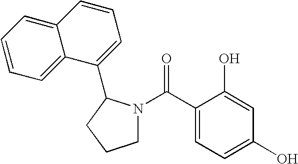

The above compound was made as follows using the General Procedure G2. O-(7-azabenzotriazol-1-yl)-N,N,N′,N′-tetramethyluronium phosphorus pentafluoride (HATU) (370 mg, 0.972 mmol), diisopropylethyl amine (0.34 mL, 1.94 mmol) and 2,4-dihydroxybenzoic acid (100 mg, 0.648 mmol) were mixed with 4 mL of DMF under a nitrogen atmosphere. After 30 min, a DMF solution (1 mL) of 2-(1-naphthyl)pyrrolidine (142 mg, 0.72 mmol) was then added to the solution. The reaction was allowed to stir at room temperature for 16 h. Saturated aqueous NaHCO₃ was added and the product extracted with EtOAc. The organic extract was then washed with H₂O and brine. The organic layer was then dried (Na₂SO₄), concentrated, and purified by flash chromatography (10%→40% EtOAc/hexanes) to afford product (21 mg, 10%) as a white solid. ¹H NMR (400 MHz, DMSO-d₆) δ ppm 1.63-1.95 (m, 3H) 2.53-2.66 (m, 2H) 3.53-3.69 (m, 1H) 3.78-3.98 (m, 1H) 5.84-6.38 (m, 2H) 7.20-7.32 (m, 1H) 7.40-7.64 (m, 4H) 7.80 (d, J=8.3 Hz, 1H) 7.88-8.00 (m, 1H) 8.09-8.24 (m, 1H) 9.28-9.92 (m, 1H) 10.37-10.96 (m, 1H). Elemental Analysis: Calc'd for C₂₁H₁₉NO₃ C, 75.66; H, 5.74; N, 4.20. Found: C, 75.18; H, 6.22; N, 4.00.

### Example 6

### 4-{[2-(3,5-dichlorophenyl)pyrrolidin-1-yl]carbonyl}benzene-1,3-diol

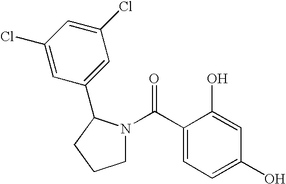

The above compound was made as follows using the General Procedure G2. O-(7-azabenzotriazol-1-yl)-N,N,N′,N′-tetramethyluronium phosphorus pentafluoride (HATU) (370 mg, 0.972 mmol), diisopropylethyl amine (34 mL, 1.94 mmol) and 2,4-dihydroxybenzoic acid (100 mg, 0.648 mmol) were mixed with 4 mL of DMF under a nitrogen atmosphere. After 30 min, a DMF solution (1 mL) of 2-(3,5-dichlorophenyl)pyrrolidine (175 mg, 0.811 mmol) was then added to the solution. The reaction was allowed to stir at room temperature for 16 h. Saturated aqueous NaHCO₃ was added and the product extracted with EtOAc. The organic extract was then washed with H₂O and brine. The organic layer was then dried (Na₂SO₄), concentrated, and purified by flash chromatography (10%→40% EtOAc/hexanes) to afford product (54 mg, 24%) as a white solid. ¹H NMR (400 MHz, DMSO-d₆) δ ppm 1.66-1.76 (m, 1H) 1.76-1.86 (m, 2H) 2.31-2.41 (m, 1H) 3.42-3.53 (m, 1H) 3.74-3.85 (m, 1H) 5.05-5.16 (m, J=5.6 Hz, 1H) 6.24-6.35 (m, 2H) 7.14-7.23 (m, 1H) 7.42 (d, J=7.8 Hz, 3H) 9.73 (s, 1H) 10.49 (s, 1H). Elemental Analysis: Calc'd for C₁₇H₁₅Cl₂NO₃ C, 57.97; H, 4.29; N, 3.98. Found: C, 57.77; H, 4.28; N, 3.92.

### Example 7

### 4-{[2-(2-chlorophenyl)pyrrolidin-1-yl]carbonyl}benzene-1,3-diol

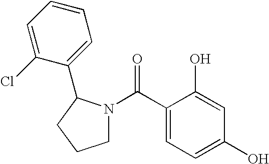

The above compound was made as follows using the General Procedure G2. O-(7-azabenzotriazol-1-yl)-N,N,N′,N′-tetramethyluronium phosphorus pentafluoride (HATU) (370 mg, 0.972 mmol), diisopropylethyl amine (0.34 mL, 1.944 mmol) and 2,4-dihydroxybenzoic acid (100 mg, 0.648 mmol) were mixed with 4 mL of DMF under a nitrogen atmosphere. After 30 min, a DMF solution (1 mL) of 2-(2-chlorophenyl)pyrrolidine (147 mg, 0.811 mmol) was then added to the solution. The reaction was allowed to stir at room temperature for 16 h. Saturated aqueous NaHCO₃ was added and the product extracted with EtOAc. The organic extract was then washed with H₂O and brine. The organic layer was then dried (Na₂SO₄), concentrated, and purified by flash chromatography (10%→40% EtOAc/hexanes) to afford product (58 mg, 28%) as a white solid. ¹H NMR (400 MHz, DMSO-d₆) δ ppm 1.50-1.75 (m, 1H) 1.73-1.95 (m, 2H) 2.29-2.47 (m, 1H) 3.48-3.63 (m, 1H) 3.75-3.90 (m, 1H) 5.37 (t, J=6.4 Hz, 1H) 6.18-6.39 (m, 2H) 7.11-7.34 (m, 3H) 7.34-7.51 (m, 2H) 9.38-9.82 (m, 1H) 10.09-10.60 (m, 1H). Elemental Analysis: Calc'd for C₁₇H₁₆ClNO₃ C, 64.26; H, 5.08; N, 4.41. Found: C, 64.37; H, 5.11; N, 4.37.

### Example 8

### 4-[(2-phenylpyrrolidin-1-yl)carbonyl]benzene-1,3-diol

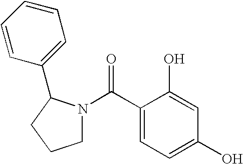

The above compound was made as follows using the General Procedure G2. O-(7-azabenzotriazol-1-yl)-N,N,N′,N′-tetramethyluronium phosphorus pentafluoride (HATU) (406 mg, 1.07 mmol), diisopropylethyl amine (0.34 mL, 1.94 mmol) and 2,4-dihydroxybenzoic acid (149 mg, 0.97 mmol) were mixed with 4 mL of DMF under a nitrogen atmosphere. After 30 min, a DMF solution (1 mL) of 2-phenylpyrrolidine (150 mg, 1.02 mmol) was then added to the solution. The reaction was allowed to stir at room temperature for 16 h. Saturated aqueous NaHCO₃ was added and the product extracted with EtOAc. The organic extract was then washed with H₂O and brine. The organic layer was then dried (Na₂SO₄), concentrated, and purified by flash chromatography (20%→40% EtOAc/hexanes) to afford product (32 mg, 12%) as a white solid. ¹H NMR (400 MHz, DMSO-d₆) δ ppm 1.67-1.77 (m, 1H) 1.77-1.87 (m, 2H) 2.29-2.38 (m, 1H) 3.53-3.64 (m, 1H) 3.75-3.86 (m, 1H) 5.14 (s, 1H) 6.22-6.32 (m, 2H) 7.15-7.24 (m, J=5.1 Hz, 2H) 7.24-7.35 (m, 4H) 9.76 (s, 1H) 10.68 (s, 1H). Elemental Analysis: Calc'd for C₁₇H₁₇ClNO₃ C, 72.07; H, 6.05; N, 4.94. Found: C, 71.89; H, 6.17; N, 4.76.

### Example 9

### 2-(1,3-dihydro-2H-isoindol-2-ylcarbonyl)-4-fluorophenol

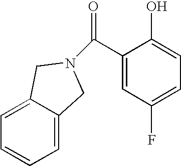

The above compound was made as follows using the General Procedure G2. O-(7-azabenzotriazol-1-yl)-N,N,N′,N′-tetramethyluronium phosphorus pentafluoride (HATU) (418 mg, 1.1 mmol), diisopropylethyl amine (0.35 mL, 2.0 mmol) and isoindoline (0.12 mL, 1.05 mmol) were added to a solution of 5-fluorosalicylic acid (156 mg, 1.0 mmol) in 1 mL of DMF under a nitrogen atmosphere. The reaction was allowed to stir at room temperature for 12 h. Saturated NaHCO₃ was added to the reaction mixture to quench the reaction. EtOAc was then added to extract the aqueous solution. Dry EtOAc layer over Na₂SO₄. The Na₂SO₄ was filtered off and the filtrated was evaporated to give a brown oil residue. The residue was purified by silica gel chromatography (eluting with EtOAc and hexanes) to give the desired product of 2-(1,3-dihydro-2H-isoindol-2-ylcarbonyl)-4-fluorophenol as off-white solid (235 mg, 91% yield). ¹H NMR (400 MHz, DMSO-D6) δ ppm 4.62 (s, 2H) 4.79 (s, 2H) 6.82-6.99 (m, J=8.72, 4.42 Hz, 1H) 7.01-7.18 (m, 2H) 7.20-7.34 (m, 3H) 7.34-7.45 (m, J=6.82 Hz, 1H) 9.97 (s, 1H). LCMS: Calc'd. For C₁₅H₁₂FNO₂: MW: 257; found: (M+1): 258. Anal. Calc'd for C₁₅H₁₂FNO₂.0.04EtOAc: C, 69.62; H, 4.76; N, 5.37. Found: C, 69.75; H, 4.71; N, 5.39.

### Example 10

### 4-bromo-2-(1,3-dihydro-2H-isoindol-2-ylcarbonyl)phenol

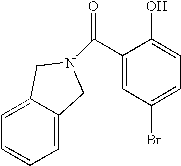

The above compound was made as follows using the General Procedure G2. O-(7-azabenzotriazol-1-yl)-N,N,N′,N′-tetramethyluronium phosphorus pentafluoride (HATU) (836 mg, 2.2 mmol), diisopropylethyl amine (0.7 mL, 4.0 mmol) and isoindoline (0.24 mL, 2.1 mmol) were added to a solution of 5-bromosalicylic acid (434 mg, 2.0 mmol) in 2 mL of DMF under a nitrogen atmosphere. The reaction was allowed to stir at room temperature for 12 h. Saturated NaHCO₃ was added to the reaction mixture to quench the reaction. EtOAc was then added to extract the aqueous solution. Dry EtOAc layer over Na₂SO₄. The Na₂SO₄ was filtered off and the filtrated was evaporated to give a brown oil residue. The residue was purified by silica gel chromatography (eluting with EtOAc and hexanes) to give the desired product of 4-bromo-2-(1,3-dihydro-2H-isoindol-2-ylcarbonyl)phenol as a yellowish solid (419 mg, 66% yield). ¹H NMR (400 MHz, DMSO-D6) δ ppm 4.61 (s, 2H) 4.78 (s, 2H) 6.89 (d, J=8.8 Hz, 1H) 7.22-7.31 (m, 3H) 7.33-7.46 (m, 3H) 10.29 (s, 1H). LCMS: Calc'd. For C₁₅H₁₂BrNO₂: MW: 318; found: (M+1): 319. Anal. Calc'd for C₁₅H₁₂BrNO₂.0.11CH₂Cl₂: C, 55.41; H, 3.76; N, 4.26. Found: C, 55.43; H, 3.69; N, 3.93.

### Example 11

### 2-(1,3-dihydro-2H-isoindol-2-ylcarbonyl)-5-(trifluoromethyl)phenol

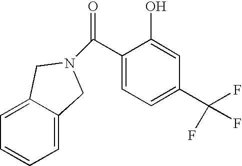

The above compound was made as follows using the General Procedure G2. O-(7-azabenzotriazol-1-yl)-N,N,N′,N′-tetramethyluronium phosphorus pentafluoride (HATU) (380 mg, 1.0 mmol), diisopropylethyl amine (0.35 mL, 2.0 mmol) and isoindoline (0.12 mL, 1.05 mmol) were added to a solution of 2-hydroxy-4-(trifluoromethyl)benzoic acid (206 mg, 1.0 mmol) in 1.0 mL of DMF under a nitrogen atmosphere. The reaction was allowed to stir at room temperature for 12 h. Saturated NaHCO₃ was added to the reaction mixture to quench the reaction. EtOAc was then added to extract the aqueous solution. Dry EtOAc layer over Na₂SO₄. The Na₂SO₄ was filtered off and the filtrate was evaporated to give a brown oil residue. The residue was purified by silica gel chromatography (eluting with EtOAc and hexanes) to give the desired product of 2-(1,3-dihydro-2H-isoindol-2-ylcarbonyl)-5-(trifluoromethyl)phenol as yellowish solid (288 mg, 94% yield). ¹H NMR (400 MHz, DMSO-D6) δ ppm 4.58 (s, 2H) 4.81 (s, 2H) 7.13-7.34 (m, 5H) 7.38 (d, J=7.07 Hz, 1H) 7.46 (d, J=8.08 Hz, 1H) 10.69 (s, 1H). LCMS: Calc'd. For C₁₆H₁₂F₃NO₂: MW: 307; found: (M+1): 308. Anal. Calc'd for C₁₆H₁₂F₃NO₂: C, 62.54; H, 3.94; N, 4.56. Found: C, 62.63; H, 3.79; N, 4.59.

### Example 12

### 4-chloro-2-(1,3-dihydro-2H-isoindol-2-ylcarbonyl)phenol

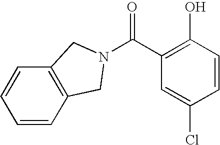

The above compound was made as follows using the General Procedure G2. Isoindoline (185 mg, 1.5 mmol) was added to a solution of 5-chloro-salicylic acid (200 mg, 1.2 mmol), diisopropylethyl amine (1 ml, 6 mmol), and O-(7-azabenzotriazol-1-yl)-N,N,N′,′-tetramethyluronium phosphorus pentafloride (HATU) (510 mg, 1.3 mmol) in 5 mL of DMF under a nitrogen atmosphere. The reaction was allowed to stir at room temperature for 12 h. Saturated NaHCO₃ (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrated was evaporated to give a brown oil residue. The residue was purified by silica gel chromatography (gradient elution 25→30% EtOAc in hexanes) to give the desired product (277 mg, 87.3% yield). ¹H NMR (400 MHz, DMSO-D6) δ ppm 4.61 (s, 2H) 4.78 (s, 2H) 6.94 (d, J=8.8 Hz, 1H) 7.20-7.34 (m, 5H) 7.38 (d, J=6.8 Hz, 1H) 10.26 (s, 1H); Anal. Calc'd for C₁₅H₁₂ClNO₂: C, 65.82; H, 4.42; N, 5.12. Found: C, 65.91; H, 4.38; N, 5.07.

### Example 13

### 4-[(6-amino-3-azabicyclo[3.1.0]hex-3-yl)carbonyl]benzene-1,3-diol

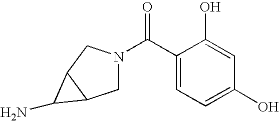

Hydrogen chloride (1 mL, 4 mmol, 4 M in dioxane) was added to a solution of the product obtained from Example 3 (50 mg, 0.15 mmol) in MeOH (4 mL). The reaction was stirred at room temperature for 12 h. The reaction mixture was neutralized with NaHCO₃ and then EtOAc was added to extract the aqueous layer. The organic layer was dried, filtered, and concentrated to give the desired product. ¹H NMR (400 MHz, DMSO-D6) δ ppm 1.90-2.06 (m, 2H) 2.20-2.36 (m, 1H) 3.48-3.60 (m, 4H) 6.23 (d, J=8.3 Hz, 1H) 6.30 (s, 1H) 6.99 (d, J=8.3 Hz, 1H) 8.36 (s, 3H) 9.69 (s, 1H) 10.16 (s, 1H); Anal. Calc'd for C₁₂H₁₄N₂O₃.0.75 PF₆.1H₂O.1.5 CH₃OH: C, 39.64; H, 5.42; N, 6.85. Found: C, 40.00; H, 5.22; N, 6.58.

### Example 14

### 4-bromo-6-(1,3-dihydro-2H-isoindol-2-ylcarbonyl)benzene-1,3-diol

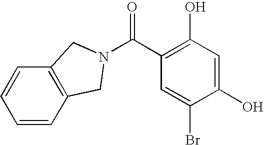

The above compound was made as follows using the General Procedure G4. Oxalyl chloride (0.11 mL, 1.1 mmol) was added to a solution of 5-bromo-2,4-dihydroxybenzoic acid (233 mg, 1 mmol), DMF (0.1 mL) in 5 mL of DCM at 0° C. Under N₂ atmosphere, the reaction mixture was allowed to warm up to room temperature and then stirred for 12 h. The solvent was evaporated to obtain a residue. Diisopropylethyl amine (1 mL, 6 molar equivalent) and DCM (5 mL) was added sequentially to the residue. Isoindoline (200 mg, 1.6 mmol, in 2 mL of DMF) was added to the reaction mixture and the resulting mixture was stirred at room temperature for 12 h. Water (20 mL) was added to quench the reaction and EtOAc was added to extract the aqueous solution. The residue was purified by silica gel chromatography (gradient elution 30→35% EtOAc in hexanes) to give the desired product (146.5 mg, 43.8% yield). ¹H NMR (400 MHz, DMSO-D6) δ ppm 4.73 (d, J=25.0 Hz, 4H) 6.58 (s, 1H) 7.23-7.31 (m, 3H) 7.31-7.44 (m, 2H) 10.32 (s, 1H) 10.47 (s, 1H); Anal. Calc'd for C₁₅H₁₂NO₃Br: C, 53.91; H, 3.62; N, 4.19. Found: C, 54.06; H, 3.70; N, 4.23.

### Example 15

### 3-(1,3-dihydro-2H-isoindol-2-ylcarbonyl)-2′-(trifluoromethyl)biphenyl-4-ol

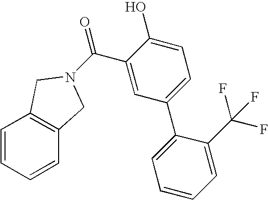

The above compound was made as follows using the General Procedures G2 and G5. A reaction solution of 4-bromo-2-(1,3-dihydro-2H-isoindol-2-ylcarbonyl)phenol (145 mg, 0.46 mmol, product from Example 10) and 2-(trifluoromethyl)benzeneboronic acid (113 mg, 0.59 mmol) in 4 mL of DME was purged with N₂ for 15 min, then Pd(dppf)₂Cl₂ (15 mg, 0.02 mmol) was added, then another 2.0 N Cs₂CO₃ solution after being purged with N₂ for 15 min. (0.7 mL, 1.38 mmol) was added to mixture. The resulting mixture was stirred at 90° C. for 4 hours. The reaction was brought to completion with LCMS & TLC. The reaction mixture was filtered through a Celite pad and washed well with MeOH. The filtrate was concentrated by vacuum. The residue was partitioned between EtOAc (200 mL) and sat. NaHCO₃ solution (2×50 mL) and brine (50 mL). The organic layer was dried (Na₂SO₄) and then concentrated by vacuum. The residue was purified by silica gel chromatography (eluting with EtOAc and hexanes) to give the desired product of 3-(1,3-dihydro-2H-isoindol-2-ylcarbonyl)-2′-(trifluoromethyl)biphenyl-4-ol as white solid (122 mg, 69% yield). ¹H NMR (400 MHz, DMSO-D6) δ ppm 4.58-4.68 (m, 2H) 4.76-4.85 (m, 2H) 6.94-7.05 (m, J=8.59 Hz, 1H) 7.11-7.16 (m, J=2.0 Hz, 1H) 7.18-7.34 (m, 4H) 7.35-7.46 (m, 2H) 7.51-7.62 (m, J=7.7, 7.7 Hz, 1H) 7.64-7.74 (m, J=7.6, 7.6 Hz, 1H) 7.75-7.85 (m, J=7.8 Hz, 1H) 10.22 (s, 1H). LCMS: Calc'd. For C₂₂H₁₆F₃NO₂: MW: 383; found: (M+1): 384. Anal. Calc'd for C₂₂H₁₆F₃NO₂×0.18CH₂Cl₂.0.08EtOAc: C, 66.61; H, 4.22; N, 3.45. Found: C, 66.57; H, 4.20; N, 3.69.

### Example 16

### 3-(1,3-dihydro-2H-isoindol-2-ylcarbonyl)biphenyl-4-ol

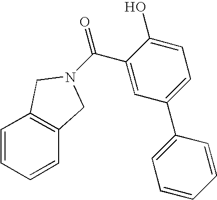

The above compound was made as follows using the General Procedures G2 and G5. A reaction solution of 4-bromo-2-(1,3-dihydro-2H-isoindol-2-ylcarbonyl)phenol (100 mg, 0.3 mmol, product from Example 10) and phenylboronic acid (50 mg, 0.4 mmol) in 3 mL of DME was purged with N₂ for 15 min, then Pd(dppf)₂Cl₂ (10 mg, 0.01 mmol) was added, then another 2.0 N Cs₂CO₃ solution after purged with N₂ for 15 min (0.5 mL, 1.0 mmol) was added to the mixture. The resulting mixture was stirred at 90° C. for 4 hours. The reaction was brought to completion with LCMS & TLC. The reaction mixture was filtered through Celite pad and washed well with MeOH. The filtrate was concentrated by vacuum. The residue was partitioned between EtOAc (200 mL) and sat. NaHCO₃ solution (2×50 mL) and brine (50 mL). The organic layer was dried (Na₂SO₄) and then concentrated by vacuum. The residue was purified by silica gel chromatography (eluting with EtOAc and hexanes) to give the desired product of 3-(1,3-dihydro-2H-isoindol-2-ylcarbonyl)biphenyl-4-ol as a white solid (64 mg, 66% yield). ¹H NMR (400 MHz, DMSO-D6) δ ppm 4.61-4.72 (m, 2H) 4.76-4.91 (m, 2H) 7.02 (d, J=8.34 Hz, 1H) 7.21-7.34 (m, 4H) 7.34-7.47 (m, 4H) 7.48-7.67 (m, 3H) 10.13 (s, 1H). LCMS: Calc'd. For C₂₂H₁₆NO₂: MW: 315; found: (M+1): 316. Anal. Calc'd for C₂₂H₁₆F₃NO₂.0.29CH₂Cl₂: C, 75.21; H, 5.21; N, 4.12. Found: C, 75.14; H, 5.14; N, 4.36.

### Example 17

### 2-(1,3-dihydro-2H-isoindol-2-ylcarbonyl)-4-(1-methyl-1H-pyrazol-4-yl)phenol

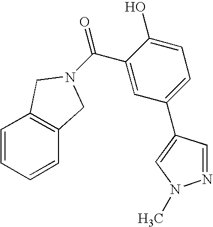

The above compound was made as follows using the General Procedures G2 and G5. A reaction solution of 4-bromo-2-(1,3-dihydro-2H-isoindol-2-ylcarbonyl)phenol (100 mg, 0.3 mmol, product from Example 10) and 1-methyl-4-(4,4,5,5-tetratmethyl-1,3,2-dioxaborolan-2yl)-1H-pyrazole (85 mg, 0.4 mmol) in 3 mL of DME was purged with N₂ for 15 min, then Pd(dppf)₂Cl₂ (10 mg, 0.01 mmol) was added, then another 2.0 N Cs₂CO₃ solution after purged with N₂ for 15 min (0.5 mL, 1.0 mmol) was added to mixture. The resulting mixture was stirred at 90° C. for 4 hours. The reaction was brought to completion with LCMS & TLC. The reaction mixture was filtered through Celite pad and washed well with MeOH. The filtrate was concentrated by vacuum. The residue was partitioned between EtOAc (200 mL) and sat. NaHCO₃ solution (2×50 mL) and brine (50 mL). The organic layer was dried (Na₂SO₄) and then concentrated by vacuum. The residue was purified by silica gel chromatography (eluting with EtOAc and hexanes) to give the desired product of 2-(1,3-dihydro-2H-isoindol-2-ylcarbonyl)-4-(1-methyl-1H-pyrazol-4-yl)phenol as a white solid (35 mg, 35% yield). ¹H NMR (400 MHz, DMSO-D6) δ ppm 3.82 (s, 3H) 4.65 (s, 2H) 4.81 (s, 2H) 6.91 (d, J=8.1 Hz, 1H) 7.20-7.34 (m, 3H) 7.36-7.50 (m, J=14.0, 14.0 Hz, 3H) 7.76 (s, 1H) 8.02 (s, 1H) 9.90 (s, 1H). LCMS: Calc'd. For C₁₉H₁₇N₃O₂: MW: 319; found: (M+1): 320. Anal. Calc'd for C₂₂H₁₆F₃NO₂.0.28EtOAc: C, 70.24; H, 5.44; N, 12.21. Found: C, 70.25; H, 5.50; N, 12.22.

TABLE 1

| No. | Structure | Name | Proc. | ¹H NMR |
|---|---|---|---|---|
| 18 | 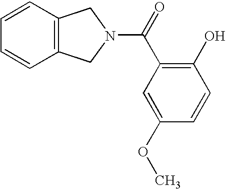 | 2-(1,3-dihydro-2H- isoindol-2- ylcarbonyl)-4- methoxyphenol | G2 | (400 MHz, DMSO-d₆) δ ppm 3.69 (s, 3 H) 4.63 (s, 2 H) 4.79 (s, 2 H) 6.79 (s, 1 H) 6.84-6.86 (m, 2 H) 7.24-7.33 (m, 3 H) 7.39 (d, J = 6.82 Hz, 1 H) 9.48 (s, 1 H) |
| 19 | 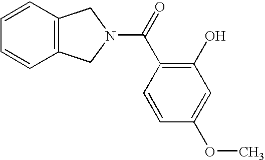 | 2-(1,3-dihydro-2H- isoindol-2- ylcarbonyl)-5- methoxyphenol | G2 | (400 MHz, DMSO-D6) δ ppm 3.74 (s, 3 H) 4.74 (s, 2 H) 4.80 (s, 2 H) 6.42-6.52 (m, 2 H) 7.20-7.45 (m, 5 H) 10.49 (s, 1 H) |
| 20 | 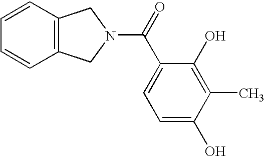 | 4-(1,3-dihydro-2H- isoindol-2- ylcarbonyl)-2- methylbenzene-1,3-diol | G2 | (400 MHz, DMSO-d₆) δ ppm 1.99 (s, 3 H) 4.98 (s, 4 H) 6.43 (d, J = 8.59 Hz, 1 H) 7.27-7.33 (m, 2 H) 7.33-7.40 (m, 2 H) 7.43 (d, J = 8.59 Hz, 1 H) 9.90 (s, 1 H) 11.64 (s, 1 H) |
| 21 | 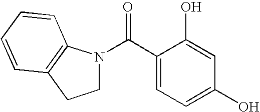 | 4-(2,3-dihydro-1H- indol-1- ylcarbonyl)benzene- 1,3-diol | G2 | (400 MHz, DMSO-d₆) δ ppm 3.05 (t, J = 8.46 Hz, 2 H) 3.95 (t, J = 8.34 Hz, 2 H) 6.29 (dd, J = 8.34, 2.27 Hz, 1 H) 6.35 (d, J = 2.27 Hz, 1 H) 6.98 (t, J = 7.33 Hz, 1 H) 7.07 (d, J = 8.34 Hz, 1 H) 7.12 (t, J = 7.58 Hz, 1 H) 7.23 (d, J = 7.33 Hz, 1 H) 7.68 (s, 1 H) 9.67 (s, 1 H) 9.91 (s, 1 H) |
| 22 | 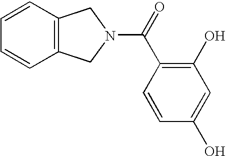 | 4-(1,3-dihydro-2H- isoindol-2- ylcarbonyl)benzene- 1,3-diol | G2 | (400 MHz, MeOD) δ ppm 4.87-4.94 (m, 4 H) 6.14-6.53 (m, 2 H) 7.00-7.52 (m, 5 H) |
| 23 | 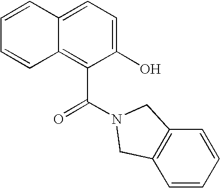 | 1-(1,3-dihydro-2H- isoindol-2- ylcarbonyl)-2- naphthol | G1 | (400 MHz, DMSO-d6) δ ppm 4.20 (d, J = 14.28 Hz, 1 H) 4.57 (d, J = 12.91 Hz, 1 H) 4.86 (d, J = 17.31 Hz, 1 H) 4.96 (d, 1 H) 7.13-7.23 (m, J = 6.59 Hz, 3 H) 7.24-7.30 (m, 2 H) 7.39 (d, J = 6.32 Hz, 2 H) 7.53 (t, J = 7.42 Hz, 1 H) 7.79-7.83 (m, 2 H) 10.07 (s, 1 H) |
| 24 | 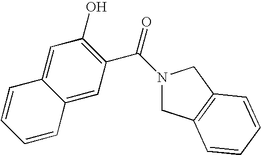 | 3-(1,3-dihydro-2H- isoindol-2- ylcarbonyl)-2- naphthol | G1 | (500 MHz, DMSO-d6) δ ppm 4.57 (s, 2 H) 4.82 (s, 2 H) 7.17-7.25 (m, 3 H) 7.27 (t, 2 H) 7.35-7.39 (m, 1 H) 7.41 (d, J = 6.59 Hz, 1 H) 7.70 (t, J = 4.94, 4.40 Hz, 1 H) 7.77-7.82 (m, 2 H) 10.18 (s, 1 H) |
| 25 | 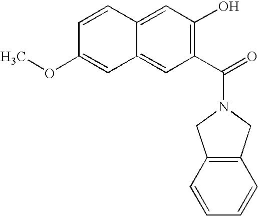 | 3-(1,3-dihydro-2H- isoindol-2- ylcarbonyl)-6- methoxy-2-naphthol | G1 | (500 MHz, DMSO-d6) δ ppm 3.79 (s, 3 H) 4.57 (s, 2 H) 4.82 (s, 2 H) 7.08 (t, J = 6.59 Hz, 1 H) 7.17-7.29 (m, 4 H) 7.37 (t, J = 5.49 Hz, 1 H) 7.51 (s, 1 H) 7.63 (d, J = 7.97 Hz, 1 H) 7.70 (s, 1 H) 9.91 (s, 1 H) |
| 26 | 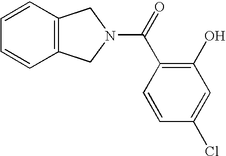 | 5-chloro-2-(1,3- dihydro-2H-isoindol-2-ylcarbonyl)phenol | G1 | (500 MHz, DMSO-d6) δ ppm 4.57 (s, 2 H) 4.76 (s, 2 H) 6.88-6.94 (m, J = 4.67 Hz, 2 H) 7.20-7.28 (m, 4 H) 7.35 (d, 1 H) 10.46 (s, 1 H) |
| 27 | 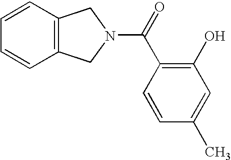 | 2-(1,3-dihydro-2H- isoindol-2- ylcarbonyl)-5- methylphenol | G1 | (500 MHz, DMSO-d6) δ ppm 2.23 (s, 3 H) 4.61 (s, 2 H) 4.76 (s, 2 H) 6.65 (d, J = 10.71 Hz, 1 H) 6.69 (d, J = 2.75 Hz, 1 H) 7.11 (d, J = 11.26 Hz, 1 H) 7.20-7.28 (m, 3 H) 7.34 (br. s., 1 H) 9.93 (s, 1 H) |
| 28 | 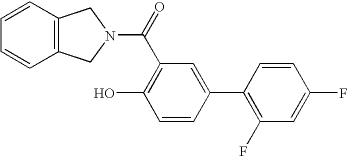 | 3-(1,3-dihydro-2H- isoindol-2- ylcarbonyl)-2′,4′- difluorobiphenyl-4-ol | G1 | (500 MHz, DMSO-d₆) δ ppm 4.63 (s, 2 H) 4.78 (s, 2 H) 6.99 (d, J = 9.34 Hz, 1 H) 7.08-7.14 (m, 1 H) 7.21-7.30 (m, 4 H) 7.35 (s, 2 H) 7.38-7.44 (m, 1 H) 7.49-7.56 (m, J = 7.69 Hz, 1 H) 10.20 (s, 1 H) |
| 29 | 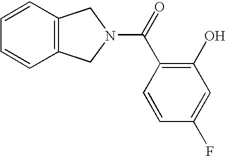 | 2-(1,3-dihydro-2H- isoindol-2- ylcarbonyl)-5- fluorophenyl | G1 | (500 MHz, DMSO-d6) δ ppm 4.59 (s, 2 H) 4.76 (s, 2 H) 6.67 (d, J = 6.59 Hz, 2 H) 7.21-7.29 (m, 4 H) 7.35 (t, J = 3.85 Hz, 1 H) 10.51 (s, 1 H) |
| 30 | 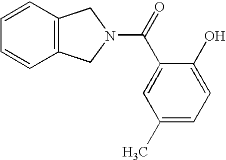 | 2-(1,3-dihydro-2H- isoindol-2- ylcarbonyl)-4- methylphenyl | G1 | (500 MHz, DMSO-d6) δ ppm 2.18 (s, 3 H) 4.58 (s, 2 H) 4.75 (s, 2 H) 6.77 (d, J = 10.71 Hz, 1 H) 6.99 (s, 1 H) 7.02 (d, J = 10.44 Hz, 1 H) 7.20-7.28 (m, 3 H) 7.31-7.38 (m, J = 4.40 Hz, 1 H) 9.66 (s, 1 H) |
| 31 | 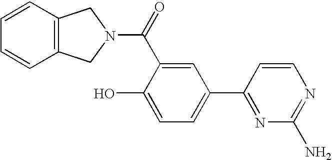 | 4-(2- aminopyrimidin-4- yl)-2-(1,3-dihydro- 2H-isoindol-2- ylcarbonyl)phenol | G1 | (500 MHz, DMSO-d6) δ ppm 4.61 (s, 2 H) 4.80 (s, 2 H) 7.04 (d, J = 11.26 Hz, 1 H) 7.21-7.31 (m, 4 H) 7.34-7.38 (m, 1 H) 8.10 (s, 2 H) 8.26 (d, J = 12.09 Hz, 1 H) 10.82 (s, 1 H) |
| 32 | 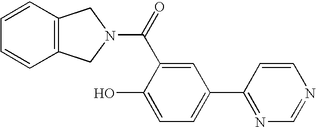 | 2-(1,3-dihydro-2H- isoindol-2- ylcarbonyl)-4- pyrimidin-4-ylphenyl | G1 | (500 MHz, DMSO-d6) δ ppm 4.62 (s, 2 H) 4.81 (s, 2 H) 7.05 (d, J = 8.52 Hz, 1 H) 7.21-7.29 (m, 3 H) 7.37 (d, J = 11.81 Hz, 1 H) 7.99 (d, 1 H) 8.11 (s, 1 H) 8.15 (d, J = 7.69 Hz, 1 H) 8.72 (d, J = 11.26 Hz, 1 H) 9.12 (s, 1 H) 10.62 (s, 1 H) |
| 33 | 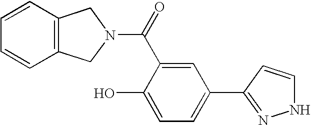 | 2-(1,3-dihydro-2H- isoindol-2- ylcarbonyl)-4-(1H- pyrazol-3-yl)phenol | G1 | (500 MHz, DMSO-d6) δ ppm 4.63 (s, 2 H) 4.79 (s, 2 H) 6.57 (d, J = 3.85 Hz, 1 H) 6.92 (d, J = 6.87 Hz, 1 H) 7.20-7.29 (m, 3 H) 7.36 (d, J = 7.14 Hz, 1 H) 7.59-7.63 (m, 2 H) 7.66 (d, J = 9.61 Hz, 1 H) 10.04 (s, 1 H) |
| 34 | 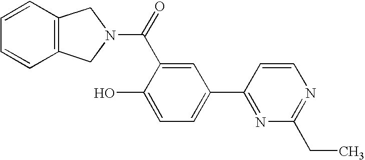 | 2-(1,3-dihydro-2H- isoindol-2- ylcarbonyl)-4-(2- ethylpyrimidin-4- yl)phenol | G1 | (500 MHz, DMSO-d6) δ ppm 1.27 (t, J = 9.89 Hz, 3 H) 2.87 (q, J = 7.22 Hz, 2 H) 4.61 (s, 2 H) 4.80 (s, 2 H) 7.04 (d, J = 7.42 Hz, 1 H) 7.20-7.28 (m, 3 H) 7.36 (d, J = 9.34 Hz, 1 H) 7.78 (d, 1 H) 8.09-8.15 (m, 2 H) 8.65 (d, 1 H) 10.60 (s, 1 H) |
| 35 | 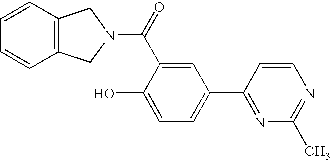 | 2-(1,3-dihydro-2H- isoindol-2- ylcarbonyl)-4-(2- methylpyrimidin-4- yl)phenol | G1 | (500 MHz, DMSO-d6) δ ppm 2.61 (s, 3 H) 4.62 (s, 2 H) 4.81 (s, 2 H) 7.04 (d, J = 11.54 Hz, 1 H) 7.21-7.29 (m, 3 H) 7.36 (d, J = 8.24 Hz, 1 H) 7.80 (d, J = 13.19 Hz, 1 H) 8.09-8.14 (m, 2 H) 8.62 (d, 1 H) 10.63 (s, 1 H) |
| 36 | 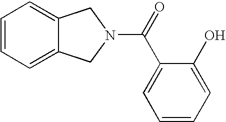 | 2-(1,3-dihydro-2H- isoindol-2- ylcarbonyl)phenol | G2 | (400 MHz, DMSO-d₆) δ ppm 4.63 (s, 2 H) 4.81 (s, 2 H) 6.88 (t, J = 7.45 Hz, 1 H) 6.93 (d, J = 7.83 Hz, 1 H) 7.23-7.32 (m, 5 H) 7.39 (d, J = 7.07 Hz, 1 H) 10.00 (s, 1 H) |
| 37 | 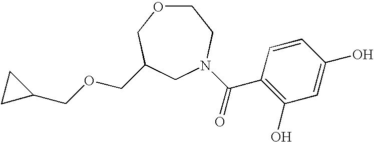 | 4-({6- [cyclopropylmethoxy) methyl]-1,4- oxazepan-4- yl}carbonyl)benzene- 1,3-diol | G1 | (500 MHz, DMSO) δ ppm 0.01-0.09 (m, 2 H) 0.37 (d, J = 5.77 Hz, 2 H) 0.81-0.90 (m, 1 H) 2.06 (br. s., 1 H) 3.50 (dd, J = 12.36, 6.04 Hz, 2 H) 3.56-3.65 (m, 3 H) 6.18 (dd, J = 8.24, 1.92 Hz, 1 H) 6.24 (d, J = 1.92 Hz, 1 H) 6.86 (d, J = 8.24 Hz, 1 H) 9.42 (s, 1 H) 9.59 (s, 1 H) |
| 38 | 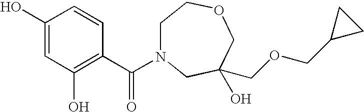 | 4-({6- [cyclopropylmethoxy) methyl]-6-hydroxy- 1,4-oxazepan-4- yl}carbonyl)benzene- 1,3-diol | G1 | (500 MHz, DMSO) δ ppm 0.08 (s, 2 H) 0.37 (d, J = 7.42 Hz, 2 H) 0.83-0.92 (m, 1 H) 3.51-3.61 (m, 3 H) 3.64-3.70 (m, 1 H) 4.60-4.69 (m, 1 H) 6.19 (dd, J = 8.24, 1.92 Hz, 2 H) 6.24 (d, J = 1.65 Hz, 1 H) 6.90 (d, J = 8.24 Hz, 1 H) 9.45 (s, 1 H) 9.62 (s, 1 H) |
| 39 | 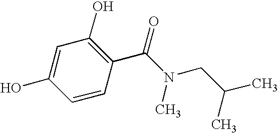 | 2,4-dihydroxy-N- isobutyl-N- methylbenzamide | G1 | (500 MHz, DMSO) δ ppm 0.75 (d, 6 H) 1.82- 1.90 (m, 1 H) 2.79 (s, 3 H) 6.18 (dd, 1 H) 6.23 (d, 1 H) 6.84 (d, 1 H) 9.42 (s, 1 H) 9.59 (s, 1 H) |
| 40 | 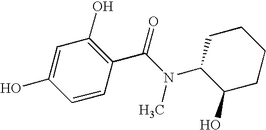 | 2,4-dihydroxy-N-(2- hydroxycyclohexyl)- N-methylbenzamide | G1 | (500 MHz, DMSO) δ ppm 0.80-1.23 (m, 3 H) 1.32-1.48 (m, 1 H) 1.53-1.62 (m, 3 H) 1.79-1.86 (m, 1 H) 2.74 (s, 3 H) 3.98-4.07 (m, 1 H) 4.47-4.77 (m, 1 H) 6.18 (d, 1 H), 6.21 (s, 1 H) 6.91 (d, 1 H) 9.44 (s, 1 H) 9.61 (s, 1 H) |
| 41 | 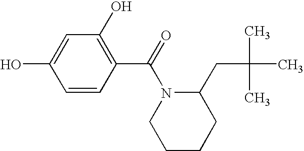 | 4-{[2-(2,2- dimethylpropyl) piperidin-1- yl]carbonyl}benzene- 1,3-diol | G1 | (500 MHz, DMSO) δ ppm 0.82 (s, 9 H) 1.23-1.32 (m, J = 12.36 Hz, 2 H) 1.42-1.51 (m, 4 H) 1.53-1.60 (m, 2 H) 6.17 (dd, J = 8.24, 1.92 Hz, 1 H) 6.22 (d, J = 1.65 Hz, 1 H) 6.77 (d, J = 7.97 Hz, 1 H) 9.40 (s, 1 H) 9.51 (s, 1 H) |
| 42 | 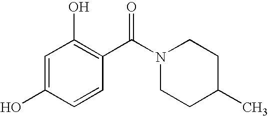 | 4-[(4- methylpiperidin-1- yl)carbonyl]benzene- 1,3-diol | G1 | (500 MHz, DMSO) δ ppm 0.85 (d, 3 H) 0.92-1.01 (m, 2 H) 1.49 -1.57 (m, 3 H) 2.74 (t, 2 H) 3.85-3.95 (m, 2 H) 6.18 (dd, 1 H) 6.23 (d, 1 H) 6.85 (d, 1 H) 9.44 (s, 1 H) 9.57 (s, 1 H) |
| 43 | 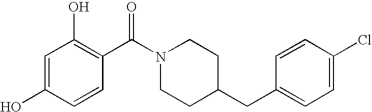 | 4-{[4-(4- chlorobenzyl) piperidin-1- yl]carbonyl}benzene- 1,3-diol | G1 | (500 MHz, DMSO) δ ppm 0.95-1.14 (m, 4 H) 1.49 (d, 2 H) 1.68 (br. s., 1 H) 2.70 (t, 2 H) 3.90 (br. s., 2 H) 6.18 (dd, 1 H) 6.22 (d, 1 H) 6.85 (d, 1 H) 7.15 (d, 2 H) 7.27 (d, 2 H) 9.45 (s, 1 H) 9.57 (s, 1 H) |
| 44 | 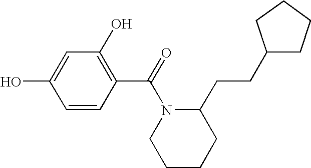 | 4-{[2-(2- cyclopentylethyl) piperidin-1- yl]carbonyl}benzene- 1,3-diol | G1 | (500 MHz, DMSO) δ ppm 0.96 (s, 2 H) 1.11 (s, 1 H) 1.21-1.29 (m, 1 H) 1.39 (s, 3 H) 1.45-1.55 (m, 8 H) 1.58-1.66 (m, 4 H) 2.76-2.84 (m, 1 H) 6.17 (dd, J = 8.24, 1.92 Hz, 1 H) 6.22 (d, J = 1.92 Hz, 1 H) 6.79 (d, J = 8.24 Hz, 1 H) 9.39 (s, 1 H) 9.49 (s, 1 H) |
| 45 | 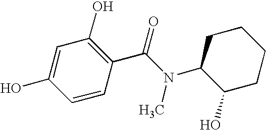 | 2,4-dihydroxy-N-(2- hydroxycyclohexyl)- N-methylbenzamide | G1 | (500 MHz, DMSO) δ ppm 0.98-1.06 (m, 1 H) 1.07-1.16 1.16 (m, J = 15.11 Hz, 2 H) 1.43 (q, J = 12.09 Hz, 1 H) 1.54-1.62 (m, 3 H) 1.78-1.86 (m, 1 H) 2.74 (s, 3 H) 4.42-4.76 (m, 1 H) 6.18 (d, J = 8.24 Hz, 1 H) 6.21 (s, 1 H) 6.91 (d, J = 8.24 Hz, 1 H) 9.44 (s, 1 H) 9.53-9.72 (m, 1 H) |
| 46 | 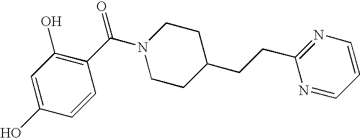 | 4-{[4-(2-pyrimidin-2- ylethyl)piperidin-1- yl]carbonyl}benzene- 1,3-diol | G1 | (500 MHz, DMSO) δ ppm 1.02 (dq, 2 H) 1.40-1.49 (m, 1 H) 1.59-1.69 (m, 4 H) 2.72 (t, 2 H) 2.84 (t, 2 H) 3.88-3.97 (m, 2 H) 6.18 (dd, 1 H) 6.23 (d, 1 H) 6.86 (d, 1 H) 7.27 (t, 1 H) 8.66 (d, 2 H) 9.45 (s, 1 H) 9.58 (s, 1 H) |
| 47 | 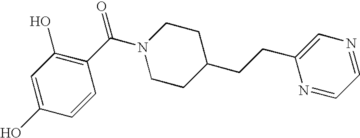 | 4-{[4-(2-pyrazin-2- ylethyl)piperidin-1- yl]carbonyl}benzene- 1,3-diol | G1 | (500 MHz, DMSO) δ ppm 1.03 (dq, 2 H) 1.40-1.49 (m, 1 H) 1.58 (q, 2 H) 1.66 (d, 2 H) 2.69-2.77 (m, 4 H) 3.88-3.97 (m, 2 H) 6.18 (dd, 1 H) 6.23 (d, 1 H) 6.86 (d, 1 H) 8.41 (d, 1 H) 8.48-8.50 (m, 1 H) 8.52 (s, 1 H) 9.45 (s, 1 H) 9.58 (s, 1 H) |
| 48 | 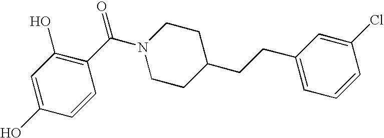 | 4-({4-[2-(3- chlorophenyl)ethyl] piperidin-1- yl}carbonyl)benzene- 1,3-diol | G1 | (500 MHz, DMSO) δ ppm 1.03 (dq, 2 H) 1.42-1.48 (m, 3 H) 1.65 (d, 2 H) 2.73 (t, 2 H) 2.98-3.11 (m, 2 H) 3.87-3.97 (m, 2 H) 6.18 (dd, 1 H) 6.23 (d, 1 H) 6.86 (d, 1 H) 7.12 (d, 1 H) 7.17 (d, 1 H) 7.23 (d, 1 H) 7.25 (t, 1 H) 9.45 (s, 1 H) 9.57 (s, 1 H) |
| 49 | 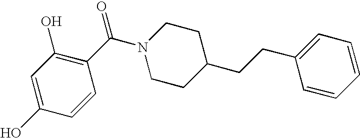 | 4-{[4-(2- phenylethyl) piperidin-1- yl]carbonyl}benzene- 1,3-diol | G1 | (500 MHz, DMSO) δ ppm 1.03 (q, 2 H) 1.40-1.49 (m, 3 H) 1.65 (d, 2 H) 2.72 (t, 2 H) 3.93 (br. s., 2 H) 6.18 (dd, 1 H) 6.23 (d, 1 H) 6.86 (d, 1 H) 7.11 (t, 1 H) 7.14 (d, 2 H) 7.22 (t, 2 H) 9.45 (s, 1 H) 9.58 (s, 1 H) |
| 50 | 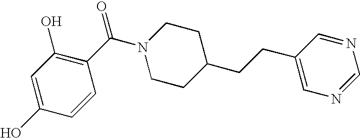 | 4-{[4-(2-pyrimidin-5- ylethyl)piperidin-1- yl]carbonyl}benzene- 1,3-diol | G1 | (500 MHz, DMSO) δ ppm 1.04 (qd, J = 3.57 Hz, 2 H) 1.42-1.45 (m, 1 H) 1.47-1.52 (m, 2 H) 1.66 (d, J = 12.91 Hz, 2 H) 2.57 (t, J = 8.24 Hz, 2 H) 2.74 (t, J = 11.95 Hz, 2 H) 3.88-3.98 (m, 2 H) 6.18 (dd, J = 8.24, 1.92 Hz, 1 H) 6.23 (d, J = 1.92 Hz, 1 H) 6.86 (d, J = 8.24 Hz, 1 H) 8.64 (s, 2 H) 8.96 (s, 1 H) 9.45 (s, 1 H) 9.58 (s, 1 H) |
| 51 | 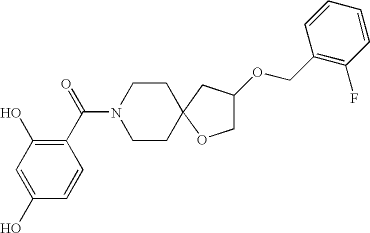 | 4-({3-[(2- fluorobenzyl)oxy]-1- oxa-8- azaspiro[4.5]dec-8- yl}carbonyl)benzene- 1,3-diol | G1 | (500 MHz, DMSO) δ ppm 1.06-1.12 (m, 1 H) 1.47 (t, J = 5.49 Hz, 2 H) 1.55 (dt, J = 9.89, 4.40, 3.30 Hz, 1 H) 1.65 (dd, J = 9.61, 4.40 Hz, 1 H) 2.99 (t, J = 12.22 Hz, 2 H) 3.48 (br. s., 2 H) 3.75-3.83 (m, 3 H) 4.17-4.22 (m, 1 H) 4.40-4.48 (m, 2 H) 6.18 (dd, J = 8.24, 1.92 Hz, 1 H) 6.23 (d, J = 1.65 Hz, 1 H) 6.87 (d, J = 8.24 Hz, 1 H) 7.10-7.17 (m, 2 H) 7.31 (dd, J = 7.42, 6.04 Hz, 1 H) 7.38 (t, J = 7.55 Hz, 1 H) 9.45 (s, 1 H) 9.58 (s, 1 H) |
| 52 | 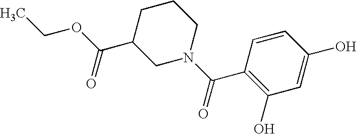 | ethyl 1-(2,4- dihydroxybenzoyl) piperidine-3- carboxylate | G1 | (500 MHz, DMSO) δ ppm 1.10 (t, 3 H) 1.31-1.40 (m, 1 H) 1.52-1.62 (m, 2 H) 1.88-1.93 (m, 1 H) 2.89 (t, 2 H) 2.96-3.05 (m, 1 H) 3.67-3.75 (m, 1 H) 3.94-4.03 (m, 3 H) 6.19 (dd, 1 H) 6.23 (s, 1 H) 6.87 (d, 1 H) 9.47 (s, 1 H) 9.59 (s, 1 H) |
| 53 | 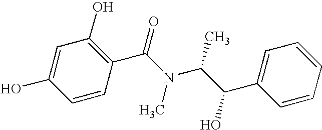 | 2,4-dihydroxy-N-(2- hydroxy-1-methyl-2- phenylethyl)-N- methylbenzamide | G1 | (500 MHz, DMSO) δ ppm 1.12 (d, 3 H) 2.78 (s, 3 H) 4.03 (q, 1 H) 4.52-4.62 (m, 1 H) 5.38 (d, 1 H) 6.07 (dd, 1 H) 6.20 (s, 1 H) 7.01 (d, 1 H) 7.14-7.23 (m, 5 H) 9.37 (s, 1 H) 9.47 (s, 1 H) |
| 54 | 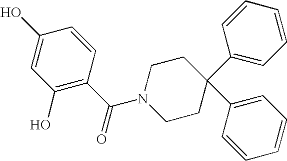 | 4-[(4,4- diphenylpiperidin-1- yl)carbonyl]benzene- 1,3-diol | G1 | (500 MHz, DMSO) δ ppm 1.12 (t, 2 H) 2.34 (s, 4 H) 6.18 (dd, 1 H) 6.22 (d, 1 H) 6.87 (d, 1 H) 7.10 (t, 2 H) 7.24 (t, 4 H) 7.30 (d, 4 H) 9.45 (s, 1 H) 9.54 (s, 1 H) |
| 55 | 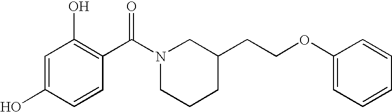 | 4-{[3-(2- phenoxyethyl) piperidin-1- yl]carbonyl}benzene- 1,3-diol | G1 | (500 MHz, DMSO) δ ppm 1.19 (q, 1 H) 1.33 (q, 1 H) 1.54-1.64 (m, 4 H) 1.79 (d, 1 H) 2.68 (dd, 1 H) 2.87 (t, 1 H) 3.68 (s, 1 H) 3.86-3.95 (m, 3 H) 6.16 (dd, 1 H) 6.23 (d, 1 H) 6.80-6.88 (m, 4 H) 7.22 (t, 2 H) 9.44 (s, 1 H) 9.58 (s, 1 H) |
| 56 | 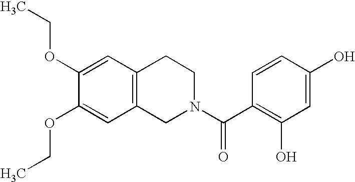 | 4-[(6,7-diethoxy- 3,4- dihydroisoquinolin- 2(1H)- yl)carbonyl]benzene- 1,3-diol | G1 | (500 MHz, DMSO) δ ppm 1.20-1.28 (m, 6 H) 2.65 (t, 2 H) 3.00-3.06 (m, 1 H) 3.48-3.56 (m, 1 H) 3.87-3.95 (m, 4 H) 4.47 (s, 2 H) 6.21 (dd, 1 H) 6.27 (d, 1 H) 6.65 (s, 2 H) 6.91 (d, 1 H) 9.52 (s, 1 H) 9.68 (s, 1 H) |
| 57 | 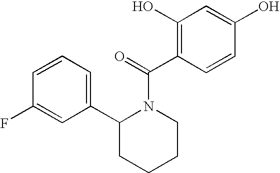 | 4-{[2-(3- fluorophenyl) piperidin-1- yl]carbonyl}benzene- 1,3-diol | G1 | (500 MHz, DMSO) δ ppm 1.22-1.28 (m, 2 H) 1.29-1.39 (m, 1 H) 1.39-1.45 (m, 1 H) 1.54 (d, 1 H) 1.77 (t, 1 H) 2.36 (d, 1 H) 2.77 (br. s., 1 H) 6.21 (d, 1 H) 6.27 (s, 1 H) 6.94 (d, 1 H) 7.02 (t, 1 H) 7.16 (d, 2 H) 7.37 (q, 1 H) 9.46 (s, 1 H) 9.79 (s, 1 H) |
| 58 | 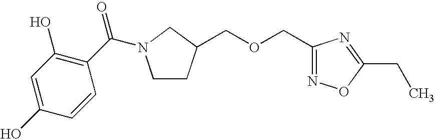 | 4-[(3-{[(5-ethyl- 1,2,4-oxadiazol-3- yl)methoxy]methyl} pyrrolidin-1- yl)carbonyl]benzene 1,3-diol | G1 | (500 MHz, DMSO) δ ppm 1.22 (t, J = 7.55 Hz, 3 H) 1.57 (ddd, J = 20.40, 8.17, 7.97 Hz, 1 H) 1.89 (td, J = 11.95, 6.59 Hz, 1 H) 2.88 (q, J = 7.69 Hz, 2 H) 3.45-3.53 (m, 2 H) 4.52 (s, 2 H) 6.17-6.22 (m, 2 H) 7.13 (d, J = 8.24 Hz, 1 H) 9.69 (s, 1 H) 10.81 (s, 1 H) |
| 59 | 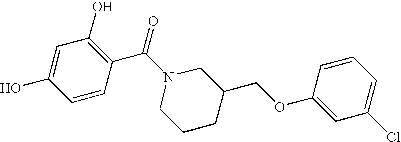 | 4-({3-[(3- chlorophenoxy) methyl]piperidin-1- yl}carbonyl)benzene- 1,3-diol | G1 | (500 MHz, DMSO) δ ppm 1.27-1.40 (m, 2 H) 1.61 (d, 1 H) 1.79 (d, 1 H) 1.89 (q, 1 H) 2.78 (dd, 2 H) 2.88 (dt, 1 H) 3.00-3.07 (m, 1 H) 3.76-3.86 (m, 2 H) 6.17 (dd, 1 H) 6.24 (d, 1 H) 6.81 (d, 1 H) 6.85 (d, 1 H) 6.90-6.94 (m, 2 H) 7.24 (t, 1 H) 9.45 (s, 1 H) 9.58 (s, 1 H) |
| 60 | 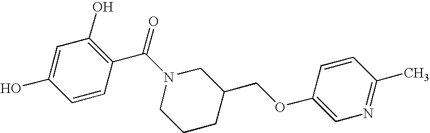 | 4-[(3-{[(6- methylpyridin-3- yl)oxy]methyl} piperidin-1- yl)carbonyl]benzene 1,3-diol | G1 | (500 MHz, DMSO) δ ppm 1.29-1.39 (m, 3 H) 1.61 (d, 1 H), 1.79 (d, 1 H) 1.86-1.93 (m, 1 H) 2.33 (s, 3 H) 2.80 (dd, 1 H) 2.89 (t, 1 H) 3.77-3.87 (m, 3 H) 6.16 (dd, 1 H) 6.24 (d, 1 H) 6.85 (d, 1 H) 7.09 (d, 1 H) 7.18 (d, 1 H) 8.04 (s, 1 H) 9.45 (s, 1 H) 9.58 (s, 1 H) |
| 61 | 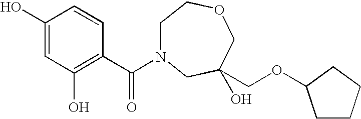 | 4-({6- [(cyclopentyloxy) methyl]-6-hydroxy-1,4- oxazepan-4- yl}carbonyl)benzene 1,3-diol | G1 | (500 MHz, DMSO) δ ppm 1.36-1.45 (m, J = 4.67 Hz, 3 H) 1.45-1.55 (m, 5 H) 3.04 (br. s., 1 H) 3.45-3.80 (m, 5 H) 4.55 (br. s., 1 H) 6.19 (dd, J = 8.38, 1.51 Hz, 1 H) 6.24 (d, J = 1.37 Hz, 1 H) 6.89 (d, J = 8.24 Hz, 1 H) 9.45 (s, 1 H) 9.62 (s, 1 H) |
| 62 | 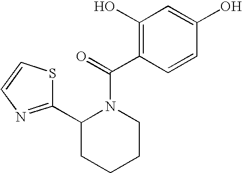 | 4-{[2-(1,3-thiazol-2- yl)piperidin-1- yl]carbonyl}benzene 1,3-diol | G1 | (500 MHz, DMSO) δ ppm 1.37-1.44 (m, 2 H) 1.50-1.56 (m, 1 H) 1.58-1.67 (m, 2 H) 1.77 (br. s., 1 H) 2.08 (d, 1 H) 2.81-2.90 (m, 1 H) 4.42 (br. s., 1 H) 6.21 (d, 1 H) 6.26 (d, 1 H) 6.93 (d, 1 H) 7.65 (d, 1 H) 7.71 (d, 1 H) 9.51 (s, 1 H) 9.70 (s, 1 H) |
| 63 | 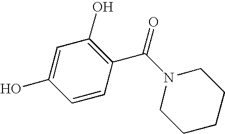 | 4-(piperidin-1- ylcarbonyl)benzene- 1,3-diol | G1 | (500 MHz, DMSO) δ ppm 1.37-1.44 (m, 6 H) 1.50-1.55 (m, 2 H) 2.81-2.84 (m, 2 H) 6.18 (dd, 1 H) 6.23 (d, 1 H) 6.85 (d, 1 H) 9.44 (s, 1 H) 9.57 (s, 1 H) |
| 64 | 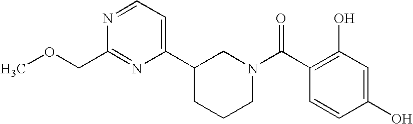 | 4-({3-[2- (methoxymethyl) pyrimidin-4-yl]piperidin- 1- yl}carbonyl)benzene- 1,3-diol | G1 | (500 MHz, DMSO) δ ppm 1.42-1.51 (m, 1 H) 1.65-1.73 (m, 2 H) 1.97 (d, J = 12.64 Hz, 1 H) 2.79-2.88 (m, J = 3.85 Hz, 3 H) 3.32 (s, 3 H) 4.03 (s, 1 H) 4.47 (s, 2 H) 6.19 (d, J = 8.24 Hz, 2 H) 6.23 (s, 1 H) 6.89 (d, J = 8.24 Hz, 1 H) 7.30 (d, J = 4.12 Hz, 1 H) 8.65 (d, J = 4.94 Hz, 1 H) 9.46 (s, 1 H) 9.61 (s, 1 H) |
| 65 | 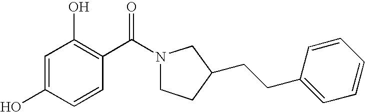 | 4-{[3-(2- phenylethyl) pyrrolidin-1- yl]carbonyl}benzene- 1,3-diol | G1 | (500 MHz, DMSO) δ ppm 1.44-1.52 (m, J = 11.54, 9.34, 8.79 Hz, 1 H) 1.57-1.66 (m, 3 H) 1.93-1.99 (m, 1 H) 2.01-2.09 (m, J = 9.34, 7.42, 6.87 Hz, 1 H) 3.53-3.58 (m, 2 H) 6.17-6.22 (m, 2 H) 7.08-7.18 (m, 5 H) 7.22 (t, J = 7.42 Hz, 2 H) 9.70 (s, 1 H) 10.95 (s, 1 H) |
| 66 | 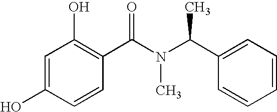 | 2,4-dihydroxy-N- methyl-N-(1- phenylethyl) benzamide | G1 | (500 MHz, DMSO) δ ppm 1.46 (d, 3 H) 6.20 (dd, 1 H) 6.26 (s, 1 H) 6.92 (d, 1 H) 7.21 (t, 1 H) 7.25-7.32 (m, 4 H) 9.44 (s, 1 H) 9.70 (s, 1 H) |
| 67 | 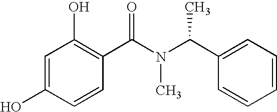 | 2,4-dihydroxy-N- methyl-N-(1- phenylethyl) benzamide | G1 | (500 MHz, DMSO) δ ppm 1.46 (d, J = 7.14 Hz, 3 H) 6.20 (dd, J = 8.38, 1.79 Hz, 1 H) 6.27 (d, J = 1.37 Hz, 1 H) 6.92 (d, J = 8.24 Hz, 1 H) 7.21 (t, J = 7.00 Hz, 1 H) 7.25-7.32 (m, 4 H) 9.44 (s, 1 H) 9.70 (s, 1 H) |
| 68 | 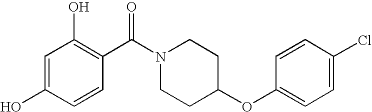 | 4-{[4-(4- chlorophenoxy) piperidin-1- yl]carbonyl}benzene- 1,3-diol | G1 | (500 MHz, DMSO) δ ppm 1.48-1.55 (m, 2 H) 1.88 (d, 2 H) 3.01-3.07 (m, 2 H) 3.66 (br. s., 2 H) 4.53-4.58 (m, 1 H) 6.19 (dd, 1 H) 6.24 (d, 1 H) 6.89 (d, 1 H) 6.96 (d, 2 H) 7.26 (d, 2 H) 9.47 (s, 1 H) 9.61 (s, 1 H) |
| 69 | 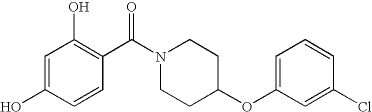 | 4-{[4-(3- chlorophenoxy) piperidin-1- yl]carbonyl}benzene- 1,3-diol | G1 | (500 MHz, DMSO) δ ppm 1.48-1.56 (m, 2 H) 1.85-1.91 (m, 2 H) 3.01-3.07 (m, 2 H) 3.62-3.72 (m, 2 H) 4.60-4.65 (m, 1 H) 6.19 (dd, 1 H) 6.24 (d, 1 H) 6.87-6.94 (m, 3 H) 7.03 (s, 1 H) 7.25 (t, 1 H) 9.47 (s, 1 H) 9.61 (s, 1 H) |
| 70 | 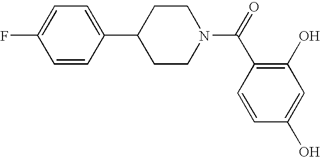 | 4-{[4-(4- fluorophenyl) piperidin-1- yl]carbonyl}benzene- 1,3-diol | G1 | (500 MHz, DMSO) δ ppm 1.48 (dq, 2 H) 1.71 (d, 2 H) 2.74 (t, 1 H) 2.86 (t, 2 H) 4.01-4.11 (m, 2 H) 6.20 (dd, 1 H) 6.24 (d, 1 H) 6.92 (d, 1 H) 7.06 (t, 2 H) 7.23 (dd, 2 H) 9.46 (s, 1 H) 9.61 (s, 1 H) |
| 71 | 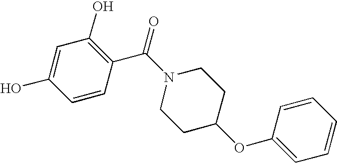 | 4-[(4- phenoxypiperidin-1- yl)carbonyl]benzene- 1,3-diol | G1 | (500 MHz, DMSO) δ ppm 1.49-1.56 (m, 2 H) 1.85-1.91 (m, 2 H) 2.94-3.03 (m, 2 H) 3.63-3.72 (m, 2 H) 4.54-4.59 (m, 1 H) 6.19 (dd, 1 H) 6.24 (d, 2 H) 6.86-6.94 (m, 3 H) 7.20-7.27 (m, 2 H) 9.47 (s, 1 H) 9.61 (s, 1 H) |
| 72 | 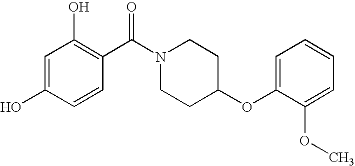 | 4-{[4-(2- methoxyphenoxy) piperidin-1- yl]carbonyl}benzene- 1,3-diol | G1 | (500 MHz, DMSO) δ ppm 1.49-1.57 (m, 2 H) 1.79-1.86 (m, 2 H) 3.03 (br. s., 2 H) 3.63-3.72 (m, 5 H) 4.41-4.46 (m, 1 H) 6.19 (dd, 1 H) 6.24 (d, 1 H) 6.81 (t, 1 H) 6.85-6.91 (m, 2 H) 6.93 (d, 1 H) 6.98 (d, 1 H) 9.47 (s, 1 H) 9.61 (s, 1 H) |
| 73 | 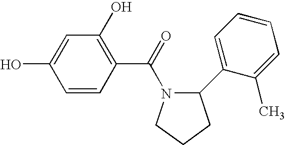 | 4-{[2-(2- methylphenyl) pyrrolidin-1- yl]carbonyl}benzene- 1,3-diol | G1 | (500 MHz, DMSO) δ ppm 1.50-1.59 (m, 1 H) 1.74-1.83 (m, 2 H) 2.26-2.35 (m, 4 H) 3.51-3.61 (m, 1 H) 3.77-3.85 (m, 1 H) 5.16-5.25 (m, 1 H), 6.17-6.26 (m, 2 H) 7.01-7.09 (m, J = 8.52 Hz, 3 H) 7.15-7.24 (m, 2 H) 9.69 (s, 1 H) 10.65 (s, 1 H) |
| 74 | 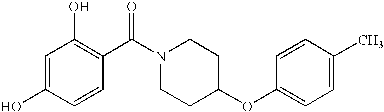 | 4-{[4-(4- methylphenoxy) piperidin-1- yl]carbonyl}benzene- 1,3-diol | G1 | (500 MHz, DMSO) δ ppm 1.50 (ddd, 2 H) 1.82-1.88 (m, 2 H) 2.17 (s, 3 H) 3.62-3.71 (m, 2 H) 4.49 (ddd, 1 H) 6.19 (dd, 1 H) 6.24 (d, 1 H) 6.81 (d, 2 H) 6.89 (d, 1 H) 7.02 (d, 2 H) 9.47 (s, 1 H) 9.61 (s, 1 H) |
| 75 | 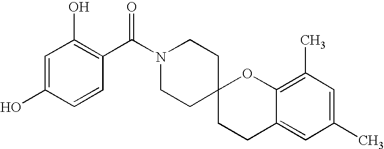 | 4-[(6,8-dimethyl- 3,4-dihydro-1′H- spiro[chromene-2,4′- piperidin]-1′- yl)carbonyl]benzene 1,3-diol | G1 | (500 MHz, DMSO) δ ppm 1.50 (td, 2 H) 1.65 (d, 2 H) 1.70 (t, 2 H) 2.07 (s, 3 H) 2.09 (s, 3 H) 2.62 (t, 2 H) 2.97-3.08 (m, 2 H) 3.68-3.93 (m, 2 H) 6.19 (dd, 1 H) 6.24 (d, 1 H) 6.64 (s, 1 H) 6.70 (s, 1 H) 6.91 (d, 1 H) 9.47 (s, 1 H) 9.62 (s, 1 H) |
| 76 | 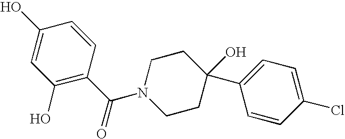 | 4-{[4-(4- chlorophenyl)-4- hydroxypiperidin-1- yl]carbonyl}benzene- 1,3-diol | G1 | (500 MHz, DMSO) δ ppm 1.54 (d, 2 H) 1.81 (td, 2 H) 3.83-3.91 (m, 2 H) 4.03 (q, 2 H) 5.17 (s, 1 H) 6.20 (dd, 1 H) 6.24 (s, 1 H) 6.94 (d, 1 H) 7.32 (d, 2 H) 7.44 (d, 2 H) 9.45 (s, 1 H) 9.62 (s, 1 H) |
| 77 | 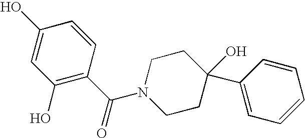 | 4-[(4-hydroxy-4- phenylpiperidin-1- yl)carbonyl]benzene- 1,3-diol | G1 | (500 MHz, DMSO) δ ppm 1.55 (d, 2 H) 1.83 (td, 2 H) 3.82-3.91 (m, 2 H) 3.95-4.04 (m, 2 H) 5.05 (s, 1 H) 6.20 (dd, 1 H) 6.24 (d, 1 H) 6.93 (d, 1 H) 7.17 (t, 1 H) 7.27 (t, 2 H) 7.42 (d, 2 H) 9.45 (s, 1 H) 9.62 (s, 1 H) |
| 78 | 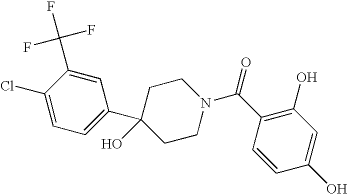 | 4-({4-[4-chloro-3- (trifluoromethyl) phenyl]-4- hydroxypiperidin-1- yl}carbonyl)benzene- 1,3-diol | G1 | (500 MHz, DMSO) δ ppm 1.55 (d, 2 H) 1.87 (td, 2 H) 3.86-3.93 (m, 2 H) 4.00-4.06 (m, 2 H) 5.42 (s, 1 H) 6.20 (dd, 1 H) 6.24 (d, 1 H) 6.96 (d, 1 H) 7.63 (d, 1 H) 7.70-7.74 (m, 1 H) 7.88 (s, 1 H) 9.46 (s, 1 H) 9.62 (s, 1 H) |
| 79 | 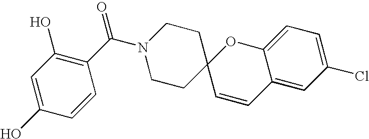 | 4-[(6-chloro-1′H- spiro[chromene-2,4′- piperidin]-1′- yl)carbonyl]benzene- 1,3-diol | G1 | (500 MHz, DMSO) δ ppm 1.59-1.66 (m, 2 H) 1.80 (d, 2 H) 2.99 (none, 1 H) 3.67-3.77 (m, 2 H) 5.82 (d, 1 H) 6.20 (dd, 1 H) 6.24 (d, 1 H) 6.44 (d, 1 H) 6.83 (d, 1 H) 6.90 (d, 1 H) 7.10 (dd, 1 H) 7.14 (d, 1 H) 9.48 (s, 1 H) 9.62 (s, 1 H) |
| 80 | 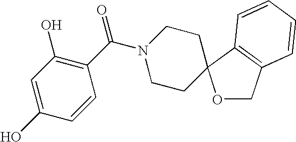 | 4-(1′H,3H-spiro[2- benzofuran-1,4′- piperidin]-1′- ylcarbonyl)benzene- 1,3-diol | G1 | (500 MHz, DMSO) δ ppm 1.59 (d, 2 H) 1.82 (td, 2 H) 2.94 (br. s., 2 H) 3.96 (br. s., 2 H) 4.97 (s, 2 H) 6.21 (d, 1 H) 6.25 (d, 1 H) 6.96 (d, 1 H) 7.18-7.23 (m, 2 H) 7.24 (d, 2 H) 9.47 (s, 1 H) 9.65 (s, 1 H) |
| 81 | 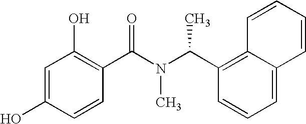 | 2,4-dihydroxy-N- methyl-N-[1-(1- naphthyl)ethyl] benzamide | G1 | 500 MHz, DMSO) δ ppm 1.59 (d, 3 H) 2.27 (s, 3 H) 3.05 (q, 1 H) 6.16 (d, 1 H) 6.21 (s, 1 H) 6.82 (d, 1 H) 7.45-7.52 (m, 3 H) 7.59 (d, 1 H) 7.85 (d, 1 H) 7.88-7.92 (m, 1 H) 8.02 (br. s., 1 H) 9.42 (s, 1 H) 9.56 (s, 1 H) |
| 82 | 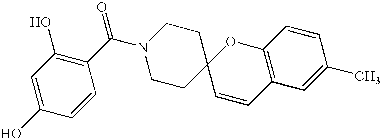 | 4-[(6-methyl-1′H- spiro[chromene-2,4′- piperidin]-1′- yl)carbonyl]benzene- 1,3-diol | G1 | (500 MHz, DMSO) δ ppm 1.59 (m, 2 H) 1.78 (d, 2 H) 2.14 (s, 3 H) 3.00-3.07 (m, 2 H) 3.70 (s, 2 H) 5.70 (d, 1 H) 6.19 (dd, 1 H) 6.24 (d, 1 H) 6.38 (d, 1 H) 6.68 (d, 1 H) 6.84 (s, 1 H) 6.87 (d, 1 H) 6.90 (d, 1 H) 9.47 (s, 1 H) 9.62 (s, 1 H) |
| 83 | 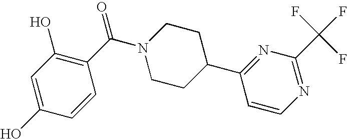 | 4-({4-[2- (trifluoromethyl) pyrimidin-4-yl]piperidin- 1- yl}carbonyl)benzene- 1,3-diol | G1 | (500 MHz, DMSO) δ ppm 1.60 (ddd, 2 H) 1.87 (d, 2 H) 2.93 (t, 2 H) 3.01-3.07 (m, 1 H) 4.03-4.12 (m, 2 H) 6.20 (dd, 1 H) 6.25 (d, 1 H) 6.91 (d, 1 H) 7.74 (d, 1 H) 8.92 (d, 1 H) 9.47 (s, 1 H) 9.63 (s, 1 H) |
| 84 | 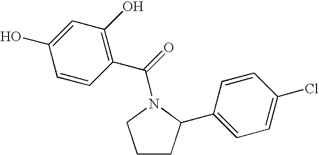 | 4-{[2-(4- chlorophenyl) pyrrolidin-1- yl]carbonyl}benzene- 1,3-diol | G1 | (500 MHz, DMSO) δ ppm 1.62-1.70 (m, 1 H) 1.71-1.81 (m, 2 H) 2.25-2.32 (m, 1 H) 3.48-3.55 (m, 1 H) 3.70-3.79 (m, 1 H) 5.00-5.09 (m, 1 H) 6.16-6.26 (m, 2 H) 7.19 (d, 1 H) 7.29 (s, 4 H) 9.69 (s, 1 H) 10.58 (s, 1 H) |
| 85 | 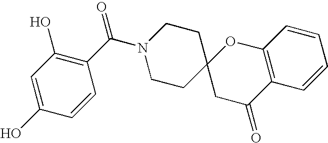 | 1′-(2,4- dihydroxybenzoyl) spiro]chromene-2,4′- piperidin]-4(3H)-one | G1 | (500 MHz, DMSO) δ ppm 1.63 (dt, 2 H) 1.87 (d, 2 H) 2.80 (s, 2 H) 2.97-3.10 (m, 2 H) 3.69-3.78 (m, 2 H) 6.19 (dd, 1 H) 6.24 (d, 1 H) 6.90 (d, 1 H) 7.00 (t, 1 H) 7.04 (d, 1 H) 7.53 (t, 1 H) 7.68 (d, 1 H) 9.48 (s, 1 H) 9.63 (s, 1 H) |
| 86 | 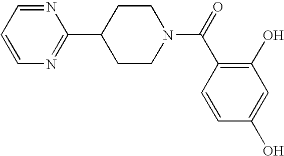 | 4-[(4-pyrimidin-2- ylpiperidin-1- yl)carbonyl]benzene- 1,3-diol | G1 | (500 MHz, DMSO) δ ppm 1.66 (ddd, 2 H) 1.89 (d, 2 H) 2.94 (t, 2 H) 3.97-4.07 (m, 2 H) 6.20 (dd, 1 H) 6.24 (d, 1 H) 6.89 (d, 1 H) 7.30 (t, 1 H) 8.70 (d, 2 H) 9.46 (s, 1 H) 9.61 (s, 1 H) |
| 87 | 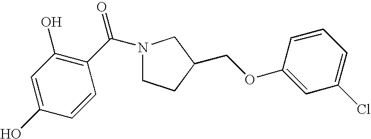 | 4-({3-[(3- chlorophenoxy) methyl]pyrrolidin-1- yl}carbonyl)benzene- 1,3-diol | G1 | (500 MHz, DMSO) δ ppm 1.67-1.75 (m, 1 H) 2.00 (s, 1 H) 2.53-2.62 (m, 1 H) 3.00-3.07 (m, 1 H) 3.52-3.57 (m, 1 H) 3.61 (dd, 1 H) 3.91-3.99 (m, 3 H) 6.18-6.23 (m, 2 H) 6.87 (d, 1 H) 6.94 (d, 1 H) 6.98 (s, 1 H) 7.15 (d, 1 H) 7.25 (t, 1 H) 9.69 (s, 1 H) 10.79 (s, 1 H) |
| 88 | 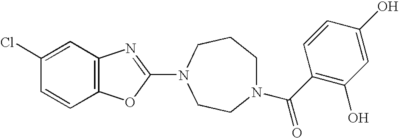 | 4-{[4-(5-chloro-1,3- benzoxazol-2-yl)- 1,4-diazepan-1- yl]carbonyl}benzene- 1,3-diol | G1 | (500 MHz, DMSO) δ ppm 1.72-1.81 (m, 2 H) 3.64-3.73 (m, 6 H) 6.07 (br. s., 1 H) 6.23 (d, J = 1.92 Hz, 1 H) 6.52 (br. s., 1 H) 6.97 (dd, J = 8.38, 1.79 Hz, 1 H) 7.26 (s, 1 H) 7.34 (d, J = 6.87 Hz, 1 H) 9.42 (s, 1 H) 9.54 (s, 1 H) |
| 89 | 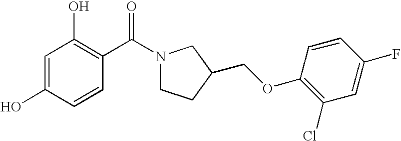 | 4-({3-[(2-chloro-4- fluorophenoxy) methyl]pyrrolidin-1- yl}carbonyl)benzene- 1,3-diol | G1 | (500 MHz, DMSO) δ ppm 1.75 (td, 1 H) 2.02 (td, 1 H) 2.58-2.65 (m, 2 H) 3.02-3.07 (m, 1 H) 3.54-3.59 (m, 1 H) 3.63 (dd, 1 H) 3.95-4.04 (m, 2 H) 6.18-6.23 (m, 2 H) 7.13 (d, 2 H) 7.15 (d, 1 H) 7.37 (d, 1 H) 9.69 (s, 1 H) 10.82 (s, 1 H) |
| 90 | 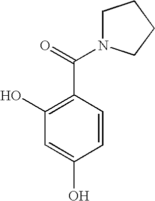 | 4-(pyrrolidin-1- ylcarbonyl)benzene- 1,3-diol | G1 | (500 MHz, DMSO) δ ppm 1.77 (t, 4 H) 2.80-2.85 (m, 4 H) 6.17-6.24 (m, 2 H) 7.18 (d, 1 H) 9.70 (s, 1 H) 10.99 (s, 1 H) |
| 91 | 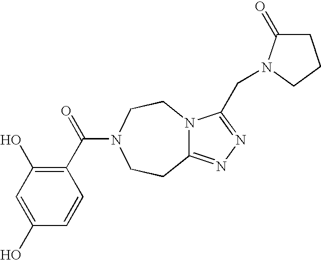 | 1-{[7-(2,4- dihydroxybenzoyl)- 6,7,8,9-tetrahydro- 5H- [1,2,4]triazolo[4,3- d][1,4]diazepin-3- yl]methyl}pyrrolidin- 2-one | G1 | (500 MHz, DMSO) δ ppm 1.84 (t, J = 7.42 Hz, 2 H) 2.15-2.22 (m, 2 H) 2.94-3.00 (m, 1 H) 3.02-3.07 (m, 1 H) 3.57 (br. s., 2 H) 4.03 (s, 2 H) 4.45 (s, 2 H) 6.22 (dd, J = 8.24, 1.92 Hz, 1 H) 6.28 (d, J = 1.65 Hz, 1 H) 6.92 (d, J = 8.24 Hz, 1 H) 9.51 (s, 1 H) 9.67 (s, 1 H) |
| 92 | 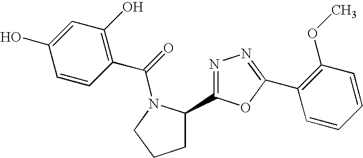 | 4-({2-[5-(2- methoxyphenyl)- 1,3,4-oxadiazol-2- yl]pyrrolidin-1- yl}carbonyl)benzene- 1,3-diol | G1 | (500 MHz, DMSO) δ ppm 1.90-1.96 (m, 1 H) 1.98-2.04 (m, 2 H) 2.30-2.37 (m, 1 H) 3.55-3.60 (m, 1 H) 3.62-3.67 (m, 1 H) 3.81 (s, 3 H) 5.30 (s, 1 H) 6.16-6.25 (m, 2 H) 7.02-7.10 (m, 2 H) 7.20 (d, J = 8.52 Hz, 1 H) 7.54 (t, J = 7.28 Hz, 1 H) 7.73 (br. s., 1 H) 9.67 (s, 1 H) 10.32 (s, 1 H) |
| 93 | 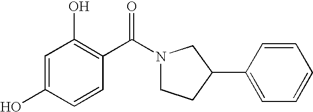 | 4-[(3- phenylpyrrolidin-1- yl)carbonyl]benzene- 1,3-diol | G1 | (500 MHz, DMSO) δ ppm 1.90-1.99 (m, 1 H) 2.18-2.23 (m, 1 H) 3.54-3.63 (m, 2 H) 3.81 (br. s., 1 H) 6.18-6.23 (m, 2 H) 7.20 (d, 2 H) 7.24-7.29 (m, 5 H) 9.71 (s, 1 H) 10.91 (s, 1 H) |
| 94 | 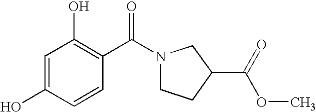 | methyl 1-(2,4- dihydroxybenzoyl) pyrrolidine-3- carboxylate | G1 | (500 MHz, DMSO) δ ppm 1.93-2.00 (m, 1 H) 2.05-2.11 (m, 1 H) 3.47 (br. s., 1 H) 3.55-3.63 (m, 5 H) 6.19-6.23 (m, 2 H) 7.11 (d, 1 H) 9.69 (s, 1 H) 10.66 (s, 1 H) |
| 95 | 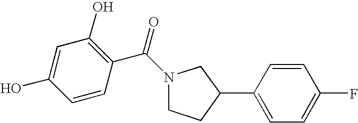 | 4-{[3-(4- fluorophenyl)pyrrolidin- 1-yl]carbonyl}benzene- 1,3-diol | G1 | (500 MHz, DMSO) δ ppm 1.93 (t, J = 10.16 Hz, 1 H) 2.16-2.22 (m, 1 H) 3.58 (d, J = 6.87 Hz, 2 H) 3.76-3.84 (m, 1 H) 6.18-6.23 (m, 2 H) 7.09 (t, J = 8.79 Hz, 2 H) 7.19 (d, J = 7.97 Hz, 1 H) 7.26-7.33 (m, 2 H) 9.71 (s, 1 H) 10.90 (s, 1 H) |
| 96 | 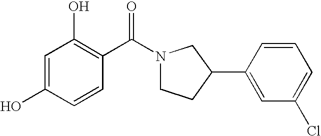 | 4-{[3-(3- chlorophenyl)pyrrolidin- 1-yl]carbonyl}benzene- 1,3-diol | G1 | (500 MHz, DMSO) δ ppm 1.95 (t, 1 H) 2.18-2.24 (m, 1 H) 2.26-2.37 (m, 1 H) 2.97-3.04 (m, 1 H) 3.52-3.61 (m, 2 H) 3.81 (br. s., 1 H) 6.19-6.24 (m, 2 H) 7.19 (d, 1 H) 7.23-7.28 (m, 2 H) 7.30 (d, 1 H) 7.35 (s, 1 H) 9.71 (s, 1 H) 10.88 (s, 1 H) |
| 97 | 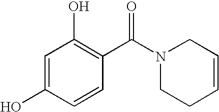 | 4-(3,6- dihydropyridin- 1(2H)- ylcarbonyl)benzene- 1,3-diol | G1 | (500 MHz, DMSO) δ ppm 2.05 (s, 2 H) 3.88 (s, 2 H) 5.64 (d, 1 H) 5.77 (d, 1 H) 6.20 (dd, 1 H) 6.25 (d, 1 H) 6.89 (d, 1 H) 9.50 (s, 1 H) 9.67 (s, 1 H) |
| 98 | 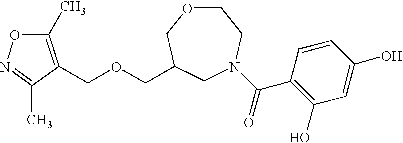 | 4-[(6-{[(3,5- dimethylisoxazol-4- yl)methoxy]methyl}- 1,4-oxazepan-4- yl)carbonyl]benzene- 1,3-diol | G1 | (500 MHz, DMSO) δ ppm 2.08 (br. s., 3 H) 2.26 (br. s., 3 H) 3.49 (dd, J = 12.91, 6.32 Hz, 1 H) 3.60 (t, J = 11.95 Hz, 2 H) 4.14 (br. s., 2 H) 6.18 (dd, J = 8.38, 2.06 Hz, 1 H) 6.24 (d, J = 1.65 Hz, 1 H) 6.85 (d, J = 8.24 Hz, 1 H) 9.43 (s, 1 H) 9.59 (s, 1 H) |
| 99 | 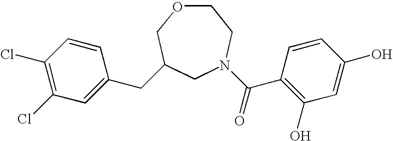 | 4-{[6-(3,4- dichlorobenzyl)-1,4- oxazepan-4- yl]carbonyl}benzene- 1,3-diol | G1 | (500 MHz, DMSO) δ ppm 2.12 (s, 1 H) 3.60 (s, 4 H) 6.17 (d, J = 8.52 Hz, 1 H) 6.22 (s, 1 H) 6.82 (s, 1 H) 7.15 (br. s., 1 H) 7.22 (br. s., 1 H) 7.42 (s, 1 H) 9.42 (s, 1 H) 9.56 (s, 1 H) |
| 100 | 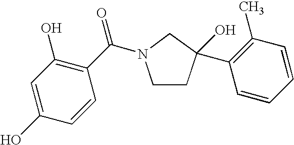 | 4-{[3-hydroxy-3-(2- methylphenyl) pyrrolidin-1- yl]carbonyl}benzene- 1,3-diol | G1 | (500 MHz, DMSO) δ ppm 2.22-2.27 (m, 1 H) 2.29-2.34 (m, 1 H) 2.87 (s, 3 H) 3.51-3.58 (m, 1 H) 3.70 (br. s., 1 H) 3.85 (d, 2 H) 5.29 (s, 1 H) 6.19-6.24 (m, 2 H) 7.08-7.15 (m, 3 H) 7.24 (d, 1 H) 7.29 (d, 1 H) 9.75 (s, 1 H) 11.06 (s, 1 H) |
| 101 | 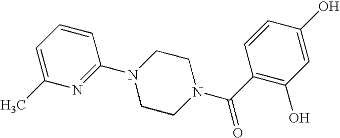 | 4-{[4-(6- methylpyridin-2- yl)piperazin-1- yl]carbonyl}benzene- 1,3-diol | G1 | (500 MHz, DMSO) δ ppm 2.25 (s, 3 H) 2.26 (s, 2 H) 2.97-3.01 (m, 2 H) 3.42-3.47 (m, 2 H) 3.50-3.54 (m, 2 H) 6.21 (dd, 1 H) 6.26 (d, 1 H) 6.48 (d, 1 H) 6.56 (d, 1 H) 6.93 (d, 1 H) 7.36-7.43 (m, 1 H) |
| 102 | 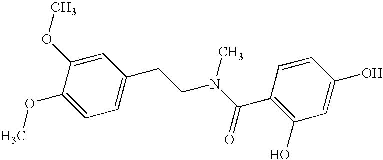 | N-[2-(3,4- dimethoxyphenyl) ethyl]-2,4-dihydroxy-N- methylbenzamide | G1 | (500 MHz, DMSO) δ ppm 2.60-2.69 (m, 2 H) 2.82 (s, 3 H) 3.63 (s, 3 H) 3.65 (s, 3 H) 6.16 (d, J = 8.24 Hz, 1 H) 6.24 (d, J = 1.65 Hz, 1 H) 6.57 (s, 2 H) 6.71 (s, 1 H) 6.77 (d, J = 7.97 Hz, 1 H) 9.43 (s, 1 H) 9.62 (s, 1 H) |
| 103 | 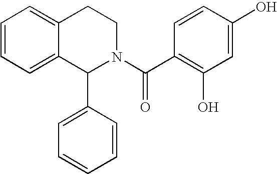 | (2,4-Dihydroxy- phenyl)-(1-phenyl- 3,4-dihydro-1H- isoquinolin-2-yl)- methanone | G1 | (500 MHz, DMSO) δ ppm 2.67 (dt, J = 16.21, 4.12 Hz, 1 H) 2.87 (ddd, J = 16.14, 9.82, 6.73 Hz, 1 H) 3.04 (br. s., 1 H) 6.20 (dd, J = 8.24, 1.65 Hz, 1 H) 6.26 (d, J = 1.65 Hz, 1 H) 6.84 (d, J = 8.24 Hz, 1 H) 7.12-7.21 (m, 6 H) 7.25 (t, J = 7.55 Hz, 2 H) 9.48 (s, 1 H) 9.61 (s, 1 H) |
| 104 | 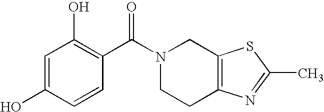 | 4-[(2-methyl-6,7- dihydro[1,3]thiazolo [5,4-c]pyridin-5(4H)- yl)carbonyl]benzene- 1,3-diol | G1 | (500 MHz, DMSO) δ ppm 2.69 (s, 3 H) 3.62 (br. s., 2 H) 4.59 (s, 2 H) 6.21 (dd, 1 H) 6.27 (s, 1 H) 6.92 (d, 1 H) 9.54 (s, 1 H) 9.70 (s, 1 H) |
| 105 | 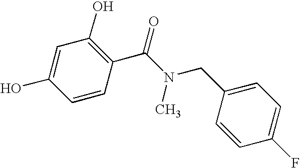 | N-(4-fluorobenzyl)- 2,4-dihydroxy-N- methylbenzamide | G1 | (500 MHz, DMSO) δ ppm 2.72 (s, 3 H) 4.48 (s, 2 H) 6.20 (dd, 1 H) 6.26 (d, 1 H) 6.93 (d, 1 H) 7.11 (t, 2 H) 7.26 (br. s., 2 H) 9.48 (s, 1 H) 9.75 (s, 1 H) |
| 106 | 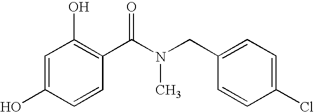 | N-(4-chlorobenzyl)- 2,4-dihydroxy-N- methylbenzamide | G1 | (500 MHz, DMSO) δ ppm 2.73 (s, 3 H) 4.49 (s, 2 H) 6.20 (dd, 1 H) 6.26 (d, 1 H) 6.93 (d, 1 H) 7.25 (d, 2 H) 7.35 (d, 2 H) 9.48 (s, 1 H) 9.75 (s, 1 H) |
| 107 | 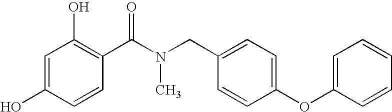 | 2,4-dihydroxy-N- methyl-N-(4- phenoxybenzyl) benzamide | G1 | (500 MHz, DMSO) δ ppm 2.74 (s, 3 H) 4.48 (s, 2 H) 6.20 (dd, 1 H) 6.26 (d, 1 H) 6.89-6.96 (m, 5 H) 7.08 (t, 1 H) 7.24 (d, 2 H) 7.34 (t, 2 H) 9.47 (s, 1 H) 9.74 (s, 1 H) |
| 108 | 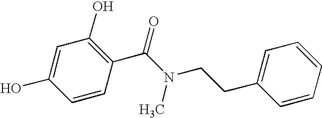 | 2,4-dihydroxy-N- methyl-N-(2- phenylethyl) benzamide | G1 | (500 MHz, DMSO) δ ppm 2.74 (t, 2 H) 2.81 (s, 3 H) 6.15 (d, 1 H) 6.24 (d, 1 H) 6.66-6.75 (m, 1 H) 7.05-7.10 (m, 1 H) 7.14 (t, 2 H) 7.18-7.23 (m, 2 H) 9.43 (s, 1 H) 9.62 (s, 1 H) |
| 109 | 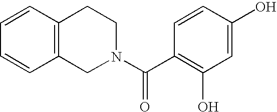 | 4-(3,4- dihydroisoquinolin- 2(1H)- ylcarbonyl)benzene- 1,3-diol | G1 | (500 MHz, DMSO) δ ppm 2.76 (t, 2 H) 3.52-3.59 (m, 2 H) 4.57 (s, 2 H) 6.21 (dd, 1 H) 6.27 (d, 1 H) 6.92 (d, 1 H) 7.05-7.13(m, 4 H) 9.52 (s, 1 H) 9.69 (s, 1 H) |
| 110 | 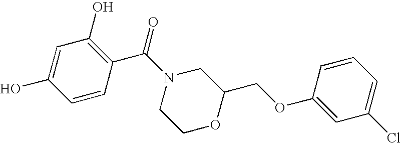 | 4-({2-[(3- chlorophenoxy) methyl]morpholin-4- yl}carbonyl)benzene- 1,3-diol | G1 | (500 MHz, DMSO) δ ppm 2.80-2.90 (m, 2 H) 2.94-3.03 (m, 1 H) 3.63-3.71 (m, 1 H) 3.82 (d, 2 H) 3.94-4.03 (m, 3 H) 6.21 (dd, 1 H) 6.25 (d, 1 H) 6.86 (d, 1 H) 6.92 (d, 1 H) 6.95 (d, 1 H) 6.97 (s, 1 H) 7.25 (t, 1 H) 9.51 (s, 1 H) 9.67 (s, 1 H) |
| 111 | 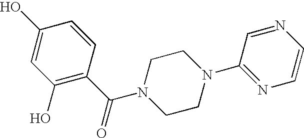 | 4-[(4-pyrazin-2- ylpiperazin-1- yl)carbonyl]benzene- 1,3-diol | G1 | (500 MHz, DMSO) δ ppm 2.81-2.87 (m, 4 H) 3.47-3.56 (m, 4 H) 6.22 (dd, 1 H) 6.27 (d, 1 H) 6.94 (d, 1 H) 7.81 (d, 1 H) 8.02-8.05 (m, 2 H) 8.25-8.28 (m, 2 H) |
| 112 | 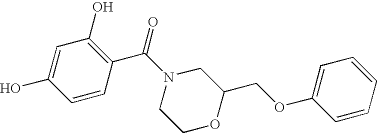 | 4-{[2- (phenoxymethyl) morpholin-4- yl]carbonyl}benzene- 1,3-diol | G1 | (500 MHz, DMSO) δ ppm 2.81-2.90 (m, 2 H) 2.92-3.00 3.00 (m, 1 H) 3.66-3.72 (m, 2 H) 3.82 (d, 2 H) 3.89-3.97 (m, 3 H) 6.21 (dd, 1 H) 6.25 (d, 1 H) 6.87 (d, 2 H) 6.91 (t, 1 H) 7.23 (t, 2 H) 9.51 (s, 1 H) 9.68 (s, 1 H) |
| 113 | 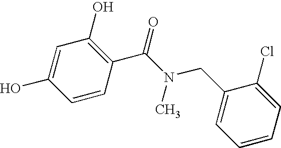 | N-(2-chlorobenzyl)- 2,4-dihydroxy-N- methylbenzamide | G1 | (500 MHz, DMSO) δ ppm 2.81 (s, 3 H) 4.59 (s, 2 H) 6.20 (d, 1 H) 6.27 (s, 1 H) 6.94 (d, 1 H) 7.26-7.34 (m, 3 H) 7.41 (d, 1 H) 9.50 (s, 1 H) 9.83 (s, 1 H) |
| 114 | 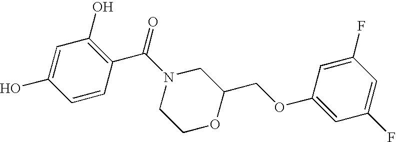 | 4-({2-[(3,5- difluorophenoxy) methyl]morpholin-4- yl}carbonyl)benzene- 1,3-diol | G1 | (500 MHz, DMSO) δ ppm 2.84 (s, 1 H) 2.97 (s, 1 H) 3.68 (s, 1 H) 3.81 (s, 2 H) 4.02 (s, 3 H) 6.20 (dd, 1 H) 6.25 (d, 1 H) 6.68 (d, 2 H) 6.73 (t, 1 H) 6.92 (d, 1 H) 6.92 (d, 1 H) 9.51 (s, 1 H) 9.67 (s, 1 H) |
| 115 | 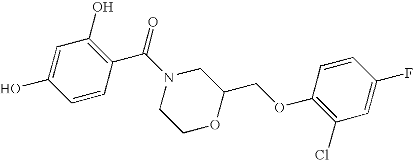 | 4-({2-[(2-chloro-4- fluorophenoxy) methyl]morpholin-4- yl}carbonyl)benzene- 1,3-diol | G1 | (500 MHz, DMSO) δ ppm 2.87-2.96 (m, 2 H) 3.70 (d, J = 7.69 Hz, 2 H) 3.83 (d, J = 12.09 Hz, 2 H) 3.96-4.02 (m, 1 H) 4.06 (dd, J = 10.16, 5.49 Hz, 1 H) 6.20 (dd, J = 8.38, 1.79 Hz, 1 H) 6.25 (d, J = 1.92 Hz, 1 H) 6.92 (d, J = 8.24 Hz, 1 H) 7.12 (d, J = 6.04 Hz, 2 H) 7.36 (d, J = 9.34 Hz, 1 H) 9.49 (s, 1 H) 9.65 (s, 1 H) |
| 116 | 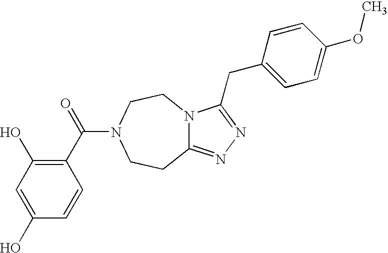 | 4-{[3-(4- methoxybenzyl)- 5,6,8,9-tetrahydro- 7H- [1,2,4]triazolo[4,3- d][1,4]diazepin-7- yl]carbonyl}benzene- 1,3-diol | G1 | (500 MHz, DMSO) δ ppm 2.89-2.98 (m, 2 H) 3.52 (br. s., 1 H) 3.66 (s, 3 H) 3.91 (s, 2 H) 3.96-4.04 (m, 3 H) 6.20 (dd, J = 8.38, 1.79 Hz, 1 H) 6.25 (s, 1 H) 6.81 (d, J = 8.24 Hz, 2 H) 6.90 (d, J = 8.24 Hz, 1 H) 7.05 (d, J = 7.97 Hz, 2 H) 9.49 (s, 1 H) 9.62 (s, 1 H) |
| 117 | 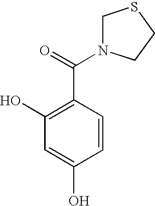 | 4-(1,3-thiazolidin-3- ylcarbonyl)benzene- 1,3-diol | G1 | (500 MHz, DMSO) δ ppm 2.94 (t, 2 H) 3.65 (t, 2 H) 4.47 (s, 2 H) 6.21 (dd, 1 H) 6.26 (d, 1 H) 7.03 (d, 1 H) 9.65 (s, 1 H) 10.05 (s, 1 H) |
| 118 | 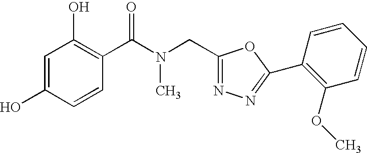 | 2,4-Dihydroxy-N-{[5- (2-methoxyphenyl)- 1,3,4-oxadiazol-2- yl]methyl}-N- methylbenzamide | G1 | (500 MHz, DMSO) δ ppm 2.96 (s, 3 H) 3.83 (s, 3 H) 4.80 (s, 2 H) 6.21 (dd, 1 H) 6.27 (d, 1 H) 6.92 (d, 1 H) 7.08 (t, 1 H) 7.22 (d, 1 H) 7.56 (t, 1 H) 7.76 (d, 1 H) 9.54 (s, 1 H) 9.73 (s, 1 H) |
| 119 | 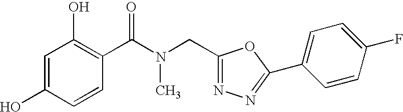 | N-{[5-(4- fluorophenyl)- 1,3,4-oxadiazol-2- yl]methyl}-2,4- dihydroxy-N- benzamide | G1 | (500 MHz, DMSO) δ ppm 2.96 (s, 3 H) 4.81 (s, 2 H) 6.21 (dd, 1 H) 6.27 (d, 1 H) 6.94 (d, 1 H) 7.41 (t, 2 H) 7.99 (dd, 2 H) 9.54 (s, 1 H) 9.74 (s, 1 H) |
| 120 | 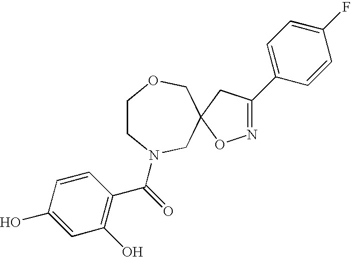 | 4-{[3-(4- fluorophenyl)-1,7- dioxa-2,10- diazaspiro[4.6]undec- 2-en-10- yl]carbonyl}benzene- 1,3-diol | G1 | (500 MHz, DMSO) δ ppm 2.98-3.07 (m, 2 H) 3.49 (br. s., 2 H) 3.59 (d, J = 13.46 Hz, 2 H) 3.76-3.85 (m, J = 12.36 Hz, 4 H) 6.20 (d, J = 7.97 Hz, 1 H) 6.24 (s, 1 H) 6.93 (d, J = 7.97 Hz, 1 H) 7.23 (t, J = 8.52 Hz, 2 H) 7.60 (br. s., 2 H) 9.47 (s, 1 H) 9.68 (s, 1 H) |
| 121 | 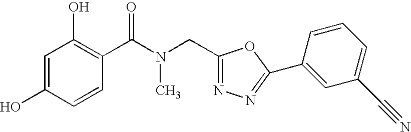 | N-{[5-(3- cyanophenyl)- 1,3,4-oxadiazol-2- yl]methyl}-2,4- dihydroxy-N- methylbenzamide | G1 | (500 MHz, DMSO) δ ppm 2.98 (s, 3 H) 4.84 (s, 2 H) 6.21 (dd, 1 H) 6.27 (d, 1 H) 6.95 (d, 1 H) 7.78 (t, 1 H) 8.07 (d, 1 H) 8.24 (d, 1 H) 8.33 (s, 1 H) 9.55 (s, 1 H) 9.74 (s, 1 H) |
| 122 | 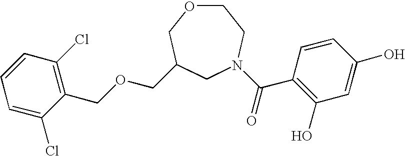 | 4-[(6-{[(2,6- dichlorobenzyl)oxy] methyl}-1,4- oxazepan-4- yl)carbonyl]benzene- 1,3-diol | G1 | (500 MHz, DMSO) δ ppm 3.00-3.07 (m, 1 H) 3.42-3.57 (m, 3 H) 3.60 (d, J = 11.81 Hz, 2 H) 4.55 (s, 2 H) 6.17 (dd, J = 8.24, 1.37 Hz, 1 H) 6.24 (d, J = 1.65 Hz, 1 H) 6.84 (d, J = 8.24 Hz, 1 H) 7.33 (t, J = 7.97 Hz, 1 H) 7.43 (d, J = 7.69 Hz, 2 H) 9.42 (s, 1 H) 9.59 (s, 1 H) |
| 123 | 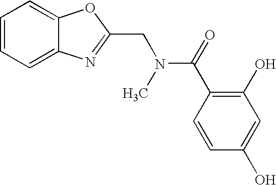 | N-(1,3-benzoxazol- 2-ylmethyl)-2,4- dihydroxy-N- methylbenzamide | G1 | (500 MHz, DMSO) δ ppm 3.00 (s, 3 H) 4.80 (s, 2 H) 6.19 (d, 1 H) 6.26 (d, 1 H) 6.93 (d, 1 H) 7.29 -7.36 (m, 2 H) 7.66 (d, 1 H) 7.68 (d, 1 H) 9.53 (s, 1 H) 9.74 (s, 1 H) |
| 124 | 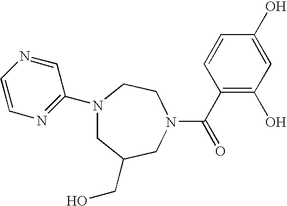 | 4-{[6- (hydroxymethyl)-4- pyrazin-2-yl-1,4- diazepan-1- yl]carbonyl}benzene- 1,3-diol | G1 | (500 MHz, DMSO) δ ppm 3.01-3.07 (m, 2 H) 3.61 (br. s., 2 H) 3.95-4.04 (m, 2 H) 4.60 (d, J = 1.65 Hz, 1 H) 6.06 (d, J = 7.97 Hz, 1 H) 6.20 (d, J = 1.65 Hz, 1 H) 7.71 (d, J = 2.20 Hz, 1 H) 7.90 (br. s., 1 H) 8.08 (br. s., 1 H) 8.78 (br. s., 1 H) 9.39 (s, 1 H) 9.50 (s, 1 H) |
| 125 | 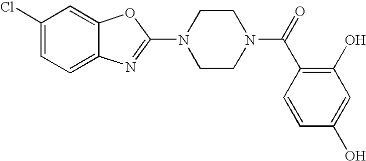 | 4-{[4-(6-chloro-1,3- benzoxazol-2- yl)piperazin-1- yl]carbonyl}benzene- 1,3-diol | G1 | (500 MHz, DMSO) δ ppm 3.01-3.07 (m, 4 H) 3.50-3.59 (m, 4 H) 6.22 (dd, 1 H) 6.27 (d, 1 H) 6.94 (d, 1 H) 7.15 (dd, 1 H) 7.24 (d, 1 H) 7.53 (d, 1 H) 9.54 (s, 1 H) 9.71 (s, 1 H) |
| 126 | 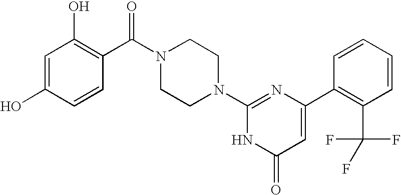 | 2-[4-(2,4- dihydroxybenzoyl) piperazin-1-yl]-6-[2- (trifluoromethyl) phenyl]pyrimidin-4(3H)- one | G1 | (500 MHz, DMSO) δ ppm 3.01 (s, 4 H) 3.63 (s, 4 H) 5.77 (s, 1 H) 6.21 (dd, 1 H) 6.25 (d, 1 H) 6.93 (d, 1 H) 7.50 (d, 1 H) 7.59 (t, 1 H) 7.67 (t, 1 H) 7.76 (d, 1 H) 9.53 (s, 1 H) 9.70 (s, 1 H) |
| 127 | 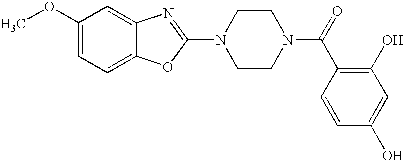 | 4-{[4-(5-methoxy- 1,3-benzoxazol-2- yl)piperazin-1- yl]carbonyl}benzene- 1,3-diol | G1 | (500 MHz, DMSO) δ ppm 3.02-3.07(m, 4 H) 3.49-3.58 (m, 4 H) 3.69 (s, 3 H) 6.22 (dd, 1 H) 6.27 (d, 1 H) 6.53 (dd, 1 H) 6.84 (d, 1 H) 6.94 (d, 1 H) 7.24 (d, 1 H) 9.54 (s, 1 H) 9.71 (s, 1 H) |
| 128 | 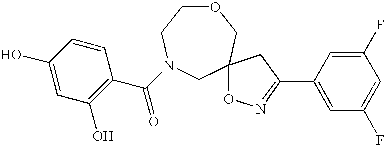 | 4-{[3-(3,5- difluorophenyl)-1,7- dioxa-2,10- diazaspiro[4.6]undec- 2-en-10- yl]carbonyl}benzene- 1,3-diol | G1 | (500 MHz, DMSO) δ ppm 3.03 (s, 2 H) 3.52-3.61 (m, 3 H) 3.76-3.85 (m, 3 H) 6.19 (d, J = 7.97 Hz, 1 H) 6.25 (s, 1 H) 6.93 (d, J = 8.24 Hz, 1 H) 7.25 (br. s., 2 H) 7.32 (t, J = 9.20 Hz, 1 H) 9.47 (s, 1 H) 9.68 (s, 1 H) |
| 129 | 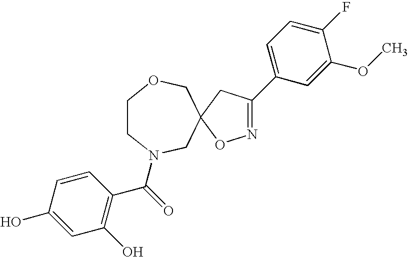 | 4-{[3-(4-fluoro-3- methoxyphenyl)-1,7- dioxa-2,10- diazaspiro[4.6]undec- 2-en-10- yl]carbonyl}benzene- 1,3-diol | G1 | (500 MHz, DMSO) δ ppm 3.52-3.60 (m, J = 13.19 Hz, 3 H) 3.76-3.84 (m, 7 H) 6.20 (d, J = 8.79 Hz, 1 H) 6.24 (s, 1 H) 6.93 (d, J = 8.24 Hz, 1 H) 7.04-7.13 (m, 1 H) 7.23 (t, J = 8.79 Hz, 2 H) 7.28-7.32 (m, 1 H) 9.47 (s, 1 H) 9.68 (s, 1 H) |
| 130 | 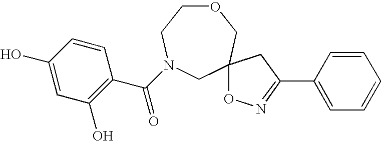 | 4-[(3-phenyl)-1,7- dioxa-2,10- diazaspiro[4.6]undec- 2-en-10- yl)carbonyl]benzene- 1,3-diol | G1 | (500 MHz, DMSO) δ ppm 3.52-3.62 (m, 3 H) 3.76-3.86 (m, J = 3.85 Hz, 3 H) 6.20 (d, J = 7.97 Hz, 2 H) 6.25 (s, 1 H) 6.93 (d, J = 8.24 Hz, 1 H) 7.40 (s, 3 H) 7.54 (br. s., 2 H) 9.47 (s, 1 H) 9.69 (s, 1 H) |
| 132 | 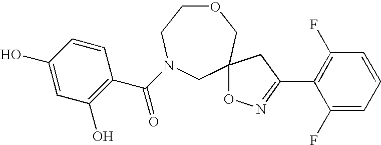 | 4-{[3-(2,6- difluorophenyl)-1,7- dioxa-2,10- diazaspiro[4.6]undec- 2-en-10- yl]carbonyl}benzene- 1,3-diol | G1 | (500 MHz, DMSO) δ ppm 3.52 (s, 2 H) 3.59 (d, J = 13.46 Hz, 1 H) 3.76-3.86 (m, J = 3.57 Hz, 3 H) 6.17 (s, 1 H) 6.24 (s, 1 H) 6.89 (d, J = 8.52 Hz, 1 H) 7.17 (t, J = 8.79 Hz, 2 H) 7.48-7.54 (m, J = 6.87 Hz, 1 H) 9.46 (s, 1 H) 9.67 (s, 1 H) |
| 132 | 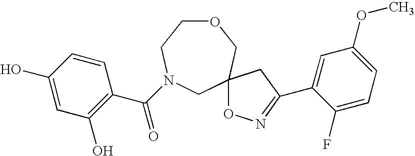 | 4-{[3-(2-fluoro-5- methoxyphenyl)-1,7- dioxa-2,10- diazaspiro[4.6]undec- 2-en-10- yl]carbonyl}benzene- 1,3-diol | G1 | (500 MHz, DMSO) δ ppm 3.54-3.61 (m, 3 H) 3.72 (s, 3 H) 3.76-3.86 (m, 3 H) 6.19 (d, J = 8.52 Hz, 1 H) 6.25 (s, 1 H) 6.91 (d, J = 8.24 Hz, 1 H) 6.97-7.03 (m, 1 H) 7.10 (br. s., 1 H) 7.19 (t, J = 10.03 Hz, 1 H) 9.46 (s, 1 H) 9.68 (s, 1 H) |
| 133 | 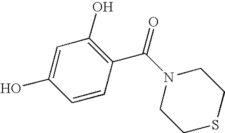 | 4-(thiomorpholin-4- ylcarbonyl)benzene- 1,3-diol | G1 | (500 MHz, DMSO) δ ppm 3.59 (s, 4 H) 6.19 (dd, 1 H) 6.24 (d, 1 H) 6.87 (d, 1 H) 9.47 (s, 1 H) 9.60 (s, 1 H) |
| 134 | 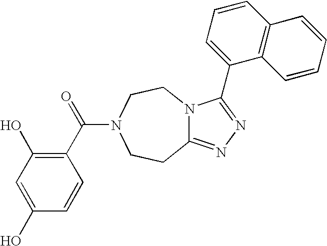 | (2,4-Dihydroxy- phenyl)-(3- naphthalen-1-yl- 4,5,7,8-tetrahydro- 1,2,3a,6-tetraaza- azulen-6-yl)- methanone | G1 | (500 MHz, DMSO) δ ppm 3 .66 (s, 3 H) 3.81 (s, 2 H) 6.19 (d, J = 5.77 Hz, 1 H) 6.24 (s, 1 H) 6.92 (d, J = 9.07 Hz, 1 H) 7.50-7.58 (m, 4 H) 7.61 (t, J = 6.87 Hz, 1 H) 7.65 (d, J = 8.24 Hz, 1 H) 8.01 (d, J = 7.97 Hz, 1 H) 8.08 (d, J = 8.24 Hz, 1 H) 9.47 (s, 1 H) 9.64 (s, 1 H) |
| 135 | 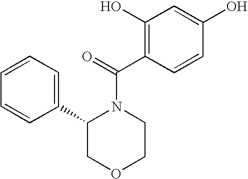 | 4-[(3- phenylmorpholin-4- yl)carbonyl]benzene- 1,3-diol | G1 | (500 MHz, DMSO) δ ppm 3.69 (d, J = 10.16 Hz, 1 H) 3.75 (d, J = 11.54 Hz, 1 H) 4.42 (d, J = 12.09 Hz, 1 H) 5.33 (br. s., 1 H) 6.21 (d, J = 8.24 Hz, 1 H) 6.26 (d, J = 1.65 Hz, 1 H) 6.95 (d, J = 8.24 Hz, 1 H) 7.21 (t, J = 7.28 Hz, 1 H) 7.31 (t, J = 7.55 Hz, 2 H) 7.38 (br. s., 2 H) 9.51 (s, 1 H) 9.79 (s, 1 H) |
| 136 | 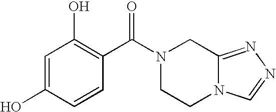 | 4-(5,6- dihydro[1,2,4]triazolo [4,3-a]pyrazin- 7(8H)- ylcarbonyl)benzene- 1,3-diol | G1 | (500 MHz, DMSO) δ ppm 3.76 (br. s., 2 H) 4.03 (t, 2 H) 4.72 (s, 2 H) 6.24 (dd, 1 H) 6.29 (d, 1 H) 6.98 (d, 1 H) 8.43 (s, 1 H) 9.61 (s, 1 H) 9.79 (s, 1 H) |

### Example 137

### 4-bromo-6-[(2-{4-[(dimethylamino)methyl]phenyl}pyrrolidin-1-yl)carbonyl]benzene-1,3-diol

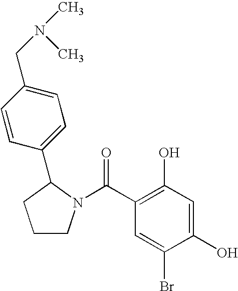

The above compound was prepared as follows using the General Procedure G2. N,N-dimethyl-1-[4-(2-pyrrolidinyl)phenyl]methanamine (270 mg, 1.3 mmol) was added to a solution of 5-bromo-2,4-dihydroxybenzoic acid (233 mg, 1 mmole), diisopropylethyl amine (0.9 ml, 5 mmol), and O-(7-azabenzotriazol-1-yl)-N,N,N′,N′-tetramethyluronium phosphorus pentafloride (HATU) (420 mg, 1.1 mmol) in 5 mL of DMF under a nitrogen atmosphere. The reaction was allowed to stir at room temperature for 12 hours. Saturated NaHCO₃ (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 0→10% MeOH in DCM) to give the desired product (74.7 mg, 37% yield). 1H NMR (400 MHz, MeOD) d ppm 1.74-2.24 (m, 3H) 2.34-2.59 (m, 1H) 2.57-2.91 (m, 6H) 4.16 (d, J=5.05 Hz, 2H) 4.99-5.55 (m, 1H) 6.48 (s, 1H) 6.66-7.26 (m, 1H) 7.21-7.86 (m, 4H): Anal. Calcd for C₂₀H₂₃BrN₂O₃.0.4PF₆.1H₂O.1.75 CH₃OH: C, 47.38; H, 5.85; N, 5.08. Found: C, 47.82; H, 5.63; N, 4.65.

### Example 138

### 2-(5-tert-butyl-2-methoxybenzoyl)isoindoline

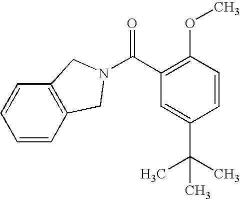

The above compound was prepared as follows using the General Procedure G2. Isoindoline (220 mg, 1.73 mmol) was added to a solution of 5-tert-butyl-2-methoxybenzoic acid (commercially available from VWR) (300 mg, 1.44 mmol), diisopropylethyl amine (1.3 mL, 5 mmol), and O-(7-azabenzotriazol-1-yl)-N,N,N′,N′-tetramethyluronium phosphorus pentafloride (HATU) (610 mg, 1.6 mmol) in 5 mL of DMF under a nitrogen atmosphere. The reaction was allowed to stir at room temperature for 12 hours. Saturated NaHCO₃ (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 35→40% EtOAc in hexanes) to give the desired product (412.7 mg, 92.6% yield). ¹H NMR (400 MHz, CHLOROFORM-D) δ ppm 1.23 (s, 9H) 3.75 (s, 3H) 4.54 (s, 2H) 4.93 (d, 2H) 6.83 (d, J=8.59 Hz, 1H) 7.06 (d, J=7.33 Hz, 1H) 7.15-7.22 (m, 2H) 7.22-7.29 (m, 2H) 7.33 (dd, J=8.84, 2.53 Hz, 1H): Anal. Calcd for C₂₀H₂₃NO₂.0.5H₂O: C, 75.44; H, 7.60; N, 4.40. Found: C, 75.81; H, 7.25; N, 4.41.

### Example 139

### 4-{[3-(4-bromophenyl)piperazin-1-yl]carbonyl}-6-chlorobenzene-1,3-diol

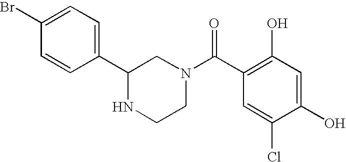

The above compound was prepared as follows using the General Procedure G10. 2-(4-bromophenyl)piperazine (100 mg, 0.41 mmol) was added to a solution of 5-chloro-2,4-dihydroxybenzoic acid (compound C in General Procedure G10) (70 mg, 0.37 mmol), diisopropylethyl amine (1.3 mL, 5 mmol), and O-(7-azabenzotriazol-1-yl)-N,N,N′,N′-tetramethyluronium phosphorus pentafloride (HATU) (170 mg, 0.44 mmol) in 5 mL of DMF under a nitrogen atmosphere. The reaction was allowed to stir at room temperature for 12 hours. Saturated NaHCO₃ (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 0→5% MeOH in DCM) to give the desired product (37.8 mg, 25% yield). ¹H NMR (300 MHz, DMSO-D6) δ ppm 2.57-2.76 (m, 3H) 2.83-3.01 (m, 3H) 3.57-3.69 (m, 1H) 6.51 (s, 1H) 7.04 (s, 1H) 7.21-7.41 (m, 2H) 7.51 (d, J=8.29 Hz, 2H) 9.95 (s, 1H) 10.31 (s, 1H): Anal. Calcd for C₁₇H₁₆BrClN₂O₃.0.05 PF₆.0.75H₂O: C, 47.22; H, 4.08; N, 6.48. Found: C, 47.39; H, 4.03; N, 6.50.

### Example 140

### 4-chloro-6-{[5-(hydroxymethyl)-1,3-dihydro-2H-isoindol-2-yl]carbonyl}benzene-1,3-diol

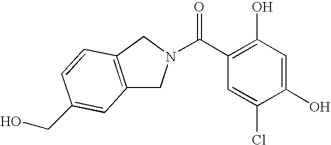

The above compound was prepared as follows using the General Procedure G10. Hydrogen chloride (2.5 mL, 10 mmol) was added to a solution of tert-butyl 5-(hydroxymethyl)isoindoline-2-carboxylate (156 mg, 0.63 mmol, as prepared via the method reported in WO2005018557A2) in MeOH (3 mL). After stirring at room temperature for 12 hours, the reaction mixture was evaporated to give a purple solid residue. This solid residue was used for the next step reaction without further purification.

The solid residue from above (0.63 mmol) was added to a solution of compound C in general procedure G10 (180 mg, 0.81 mmol), 4-methylmorpholine (0.8 mL, 6.5 mmol), N-(3-dimethylaminopropyl)-N′-ethylcarbodiimide hydrochloride (270 mg, 1.4 mmol), and 1-hydroxy benzotriazole (200 mg, 1.4 mmol) in 4 mL of DMF under a nitrogen atmosphere. The reaction was allowed to stir at room temperature for 12 hours. H₂O (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 60→70% EtOAc in hexanes) to give the desired product (59 mg, 29.6% yield). ¹H NMR (400 MHz, DMSO-D6) δ ppm 4.47 (s, 2H) 4.61-4.86 (m, J=26.02 Hz, 4H) 5.19 (s, 1H) 6.58 (s, 1H) 7.10-7.40 (m, 4H) 10.31 (s, 1H) 10.41 (s, 1H). Anal. Calcd for C₁₆H₁₄ClNO₄.0.25H₂O: C, 59.27; H, 4.51; N, 4.32. Found: C, 59.09; H, 4.66; N, 4.05.

### Example 141

### 4-tert-butyl-2-(1,3-dihydro-2H-isoindol-2-ylcarbonyl)phenol

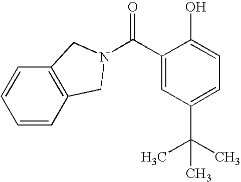

The above compound was prepared as follows using the General Procedure G2b. Boron tribromide (6.5 mL, 6.4 mmol, 1 M in DCM) was added to a solution of the product obtained from Example 138 (397 mg, 1.3 mmol) in DCM (10 mL) at −78° C. The reaction was allowed to warm to room temperature and stirred for 12 hours. The reaction mixture was neutralized with Na₂CO₃ and then EtOAc was added to extract the aqueous layer. Dry EtOAc layer over Na₂SO₄. The Na₂SO₄ was filtered off and the filtrate was evaporated to give a brown oil residue. The residue was purified by silica gel chromatography (gradient elution 10→20% EtOAc in hexanes) to give the desired product (164 mg, 43.4% yield). ¹H NMR (400 MHz, DMSO-D6) δ ppm 1.24 (s, 9H) 4.61 (s, 2H) 4.75-4.85 (m, 2H) 6.84 (d, J=8.59 Hz, 1H) 7.19 (d, J=2.53 Hz, 1H) 7.23-7.33 (m, 4H) 7.38 (d, J=6.82 Hz, 1H) 9.74 (s, 1H): Anal. Calcd for C₁₉H₂₁NO₂.0.25H₂O: C, 76.10; H, 7.23; N, 4.67. Found: C, 75.84; H, 7.16; N, 4.55.

### Example 142

### 3-bromo-2-(1,3-dihydro-2H-isoindol-2-ylcarbonyl)phenol

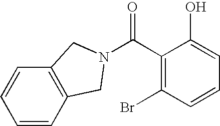

The above compound was prepared as follows using the General Procedure G3. 3-bromo-2-(1,3-dihydro-2H-isoindol-2-ylcarbonyl)phenol was synthesized according to General Procedure G2 to afford product that was 90% pure (221 mg, 48%) and then re-purified to afford pure 3-bromo-2-(1,3-dihydro-2H-isoindol-2-ylcarbonyl)phenol (118 mg). ¹H NMR (400 MHz, DMSO-d₆) δ ppm 4.41-4.52 (m, 2H) 4.76-4.85 (m, 2H) 6.94 (d, J=8.08 Hz, 1H) 7.09-7.14 (m, 1H) 7.19 (t, J=8.08 Hz, 1H) 7.26-7.34 (m, 3H) 7.40 (d, J=7.07 Hz, 1H) 10.31 (s, 1H).

### Preparation G3-1-a

### 2-bromo-6-hydroxybenzoic acid

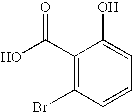

To a solution of methyl 2-bromo-6-hydroxybenzoate (0.547 mg, 2.01 mmol) in MeOH (10 mL) was added 1M NaOH (10 mL). Excess solid NaOH was added to saturate the solution. The resulting mixture was heated to 80° C. for 5 hours and then allowed to cool to ambient temperature. The solution was concentrated to half of its volume and then carefully acidified with 1M HCl to a pH ˜1.0. The product was extracted with EtOAc (3×), dried (Na₂SO₄), and concentrated to afford 2-bromo-6-hydroxybenzoic acid as a white solid (409 mg, 94%). ¹H NMR (400 MHz, DMSO-d₆) δ ppm 6.87 (dd, J=8.34, 0.76 Hz, 1H) 7.03 (dd, J=8.08, 0.76 Hz, 1H) 7.13 (t, J=8.08 Hz, 1H) 11.75 (s, 1H).

### Preparation G3-1-b

### methyl 2-bromo-6-hydroxybenzoate

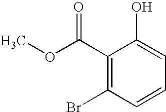

To a solution of 2-bromo-6-{[tert-butyl(dimethyl)silyl]oxy}-N,N-diethylbenzamide (2.905 g, 7.52 mmol) in anhydrous CH₃CN (50 mL) under a nitrogen atmosphere was added Na₂HPO₄ (1.601 g, 11.28 mmol) and then followed by Me₃OBF₄ (3.337 g, 22.56 mmol). The resulting solution was stirred for 16 hours at ambient temperature and then saturated aqueous NaHCO₃ (50 mL) was added followed by solid NaHCO₃. The resulting solution was then stirred at ambient temperature for 16 hours. The product was then extracted with EtOAc (3×), dried (Na₂SO₄), concentrated, and purified by flash chromatography (0% EtOAc/Hexanes-20% EtOAc/Hexanes) to afford methyl 2-bromo-6-hydroxybenzoate as a clear liquid (1.362 g, 78%). ¹H NMR (400 MHz, DMSO-d₆) δ ppm 3.80 (s, 3H) 6.91 (d, J=8.34 Hz, 1H) 7.07 (d, J=7.07 Hz, 1H) 7.19 (t, J=8.08 Hz, 1H) 10.45 (s, 1H).

### Preparation G3-1-c

### 2-bromo-6-{[tert-butyl(dimethyl)silyl]oxy}-N,N-diethyl benzamide

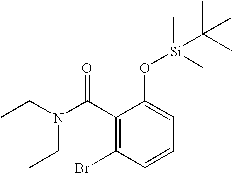

A solution of 2-{[tert-butyl(dimethyl)silyl]oxy}-N,N-diethylbenzamide (2.791 g, 9.08 mmol) in anhydrous THF (100 mL) under a nitrogen atmosphere was cooled to −78° C. and then t-BuLi (1.7M in Pentane, 8.01 mL, 13.62 mmol) was slowly added dropwise over 30 minutes. The solution was stirred at −78° C. for an additional 30 minutes and then bromine (0.7 mL, 13.62 mmol) was slowly added over 30 additional minutes. The bath was removed and the solution allowed to warm to ambient temperature. After 20 hours, saturated aqueous sodium thiosulfate and EtOAc was added and the organic layer separated. The organic layer was then washed with brine, dried (Na₂SO₄), concentrated, and purified by flash chromatography (0% EtOAc/Hexanes-15% EtOAc/Hexanes) to afford 2-bromo-6-{[tert-butyl(dimethyl)silyl]oxy}-N,N-diethylbenzamide as a white solid (2.905 g, 83%). ¹H NMR (400 MHz, DMSO-d₆) δ ppm 0.17 (s, 3H) 0.24 (s, 3H) 0.91 (s, 9H) 1.01 (t, J=7.07 Hz, 3H) 1.15 (t, J=7.07 Hz, 3H) 3.00-3.11 (m, 2H) 3.23-3.33 (m, J=7.20, 7.01, 6.80, 6.80, 6.80 Hz, 1H) 3.53-3.63 (m, J=13.64, 7.04, 7.04, 6.88 Hz, 1H) 6.93 (dd, J=6.95, 2.15 Hz, 1H) 7.18-7.24 (m, 2H).

### Preparation G3-1-d

### 2-{[tert-butyl(dimethyl)silyl]oxy}-N,N-diethylbenzamide

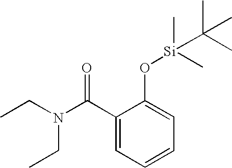

To a solution of N,N-diethyl-2-hydroxybenzamide (commercially available from Aldrich, 2.0 g, 10.35 mmol) in DMF (50 mL) was added DIEA (6.49 mL, 37.26 mmol) and TBDMSCI (3.9 g, 25.87 mmol) under a nitrogen atmosphere at ambient temperature. After 16 hours, the solution was washed with H₂O (3×), dried (Na₂SO₄), concentrated, and purified by flash chromatography (0% EtOAc/Hexanes-10% EtOAc/Hexanes) to afford quantitative yield of 2-{[tert-butyl(dimethyl)silyl]oxy}-N,N-diethylbenzamide. ¹H NMR (400 MHz, DMSO-d₆) δ ppm 0.19 (d, J=17.18 Hz, 5H) 0.84 (s, 5H) 0.90-0.97 (m, 12H) 1.13 (t, J=7.07 Hz, 3H) 6.89 (d, J=8.34 Hz, 1H) 6.98 (t, J=7.45 Hz, 1H) 7.12 (dd, J=7.58, 1.77 Hz, 1H) 7.27 (td, J=7.83, 1.77 Hz, 1H).

### Example 143

### 3-(1,3-dihydro-2H-isoindol-2-ylcarbonyl)-2′-(trifluoromethyl)biphenyl-4-ol

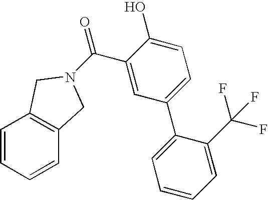

The above compound was prepared as follows using the General Procedures G2 and G5. A reaction solution of 4-Bromo-2-(1,3-dihydro-2H-isoindol-2-ylcarbonyl)phenol (145 mg, 0.46 mmol, product from Example 10) and 2-(trifluoromethyl)benzeneboronic acid (113 mg, 0.59 mmol) in 4 mL of DME was purged with N₂ for 15 minutes, then Pd(dppf)₂Cl₂ (15 mg, 0.02 mmol) was added, then another 2.0N Cs₂CO₃ solution after being purged with N₂ for 15 minutes (0.7 mL, 1.38 mmol) was added to the mixture. The resulting mixture was stirred at 90° C. for 4 hours. The reaction is completed by LCMS & TLC. The reaction mixture was filtered through Celite pad and washed well with MeOH. The filtrate was concentrated by vacuum. The residue was partitioned between EtOAc (200 mL) and sat. NaHCO₃ solution (2×50 mL) and brine (50 mL). The organic layer was dried (Na₂SO₄) and then concentrated by vacuum. The residue was purified by silica gel chromatography (eluting with EtOAc and hexanes) to give the desired product of 3-(1,3-dihydro-2H-isoindol-2-ylcarbonyl)-2′-(trifluoromethyl)biphenyl-4-ol as a white solid (122 mg, 69% yield). ¹H NMR (400 MHz, DMSO-D6) δ ppm 4.58-4.68 (m, 2H) 4.76-4.85 (m, 2H) 6.94-7.05 (m, J=8.59 Hz, 1H) 7.11-7.16 (m, J=2.02 Hz, 1H) 7.18-7.34 (m, 4H) 7.35-7.46 (m, 2H) 7.51-7.62 (m, J=7.71, 7.71 Hz, 1H) 7.64-7.74 (m, J=7.58, 7.58 Hz, 1H) 7.75-7.85 (m, J=7.83 Hz, 1H) 10.22 (s, 1H). LCMS: Calcd. For C₂₂H₁₆F₃NO₂: MW: 383; found: (M+1): 384. Anal. Calcd for C₂₂H₁₆F₃NO₂×0.18MeCl₂×0.08EtOAc: C, 66.61; H, 4.22; N, 3.45. Found: C, 66.57; H, 4.20; N, 3.69.

### Example 144

### 3-(1,3-dihydro-2H-isoindol-2-ylcarbonyl)biphenyl-4-ol

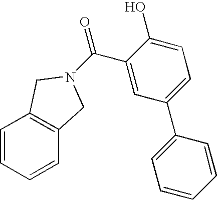

The above compound was prepared as follows using the General Procedures G2 and G5. A reaction solution of 4-Bromo-2-(1,3-dihydro-2H-isoindol-2-ylcarbonyl)phenol (100 mg, 0.31 mmol, product from Example 10) and phenylboronic acid (50 mg, 0.41 mmol) in 3 mL of DME was purged with N₂ for 15 minutes, then Pd(dppf)₂Cl₂ (10 mg, 0.01 mmol) was added, then another 2.0N Cs₂CO₃ solution after being purged with N₂ for 15 minutes (0.5 mL, 1.0 mmol) was added to the mixture. The resulting mixture was stirred at 90° C. for 4 hours. The reaction was completed by LCMS & TLC. The reaction mixture was filtered through Celite pad and washed well with MeOH. The filtrate was concentrated by vacuum. The residue was partitioned between EtOAc (200 mL) and saturated NaHCO₃ solution (2×50 mL) and brine (50 mL). The organic layer was dried (Na₂SO₄) and then concentrated by vacuum. The residue was purified by silica gel chromatography (eluting with EtOAc and hexanes) to give the desired product of 3-(1,3-dihydro-2H-isoindol-2-ylcarbonyl)biphenyl-4-ol as white solid (64 mg, 66% yield). ¹H NMR (400 MHz, DMSO-D6) δ ppm 4.61-4.72 (m, 2H) 4.76-4.91 (m, 2H) 7.02 (d, J=8.34 Hz, 1H) 7.21-7.34 (m, 4H) 7.34-7.47 (m, 4H) 7.48-7.67 (m, 3H) 10.13 (s, 1H). LCMS: Calcd. For C₂₂H₁₆NO₂: MW: 315; found: (M+1): 316. Anal. Calcd for C₂₂H₁₆F₃NO₂×0.29MeCl₂: C, 75.21; H, 5.21; N, 4.12. Found: C, 75.14; H, 5.14; N, 4.36.

### Example 145

### 2-(1,3-dihydro-2H-isoindol-2-ylcarbonyl)-4-(1-methyl-1H-pyrazol-4-yl)phenol

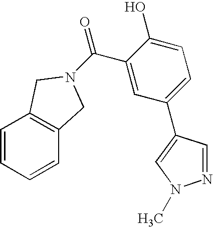

The above compound was prepared as follows using the General Procedures G2 and G5. A reaction solution of 4-Bromo-2-(1,3-dihydro-2H-isoindol-2-ylcarbonyl)phenol (100 mg, 0.31 mmol, product from Example 10) and 1-Methyl-4-(4,4,5,5-tetratmethyl-1,3,2-dioxaborolan-2yl)-1H-pyrazole (85 mg, 0.41 mmol) in 3 mL of DME was purged with N₂ for 15 minutes, then Pd(dppf)₂Cl₂ (10 mg, 0.01 mmol) was added, then another 2.0N Cs₂CO₃ solution after being purged with N₂ for 15 minutes (0.5 mL, 1.0 mmol) was added to the mixture. The resulting mixture was stirred at 90° C. for 4 hours. The reaction was completed by LCMS & TLC. The reaction mixture was filtered through Celite pad and washed well with MeOH. The filtrate was concentrated by vacuum. The residue was partitioned between EtOAc (200 mL) and saturated NaHCO₃ solution (2×50 mL) and brine (50 mL). The organic layer was dried (Na₂SO₄) and then concentrated by vacuum. The residue was purified by silica gel chromatography (eluting with EtOAc and hexanes) to give the desired product of 2-(1,3-dihydro-2H-isoindol-2-ylcarbonyl)-4-(1-methyl-1H-pyrazol-4-yl)phenol as white solid (35 mg, 35% yield). ¹H NMR (400 MHz, DMSO-D6) δ ppm 3.82 (s, 3H) 4.65 (s, 2H) 4.81 (s, 2H) 6.91 (d, J=8.08 Hz, 1H) 7.20-7.34 (m, 3H) 7.36-7.50 (m, J=14.02, 14.02 Hz, 3H) 7.76 (s, 1H) 8.02 (s, 1H) 9.90 (s, 1H). LCMS: Calcd. For C₁₉H₁₇N₃O₂: MW: 319; found: (M+1): 320. Anal. Calcd for C₂₂H₁₆F₃NO₂×0.28EtOAc: C, 70.24; H, 5.44; N, 12.21. Found: C, 70.25; H, 5.50; N, 12.22.

### Example 146

### 2-(5-chloro-2,4-dihydroxybenzoyl)-N-ethylisoindoline-1-carboxamide

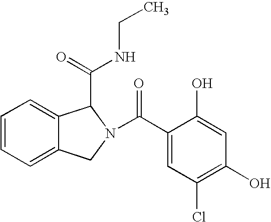

The above compound (Ex. 146-a), prepared as a racemic mixture in Example 156, was separated into two separate enantiomers as follows. 2-(5-chloro-2,4-dihydroxybenzoyl)-N-ethylisoindoline-1-carboxamide (543 mg) was resolved by Chiralpak AD-H, 35% MeOH (260 nm) at 50 mL/min under 140 bar to give peak #1, (R)-2-(5-chloro-2,4-dihydroxybenzoyl)-N-ethylisoindoline-1-carboxamide (Ex. 146-b, 233 mg, white solid);

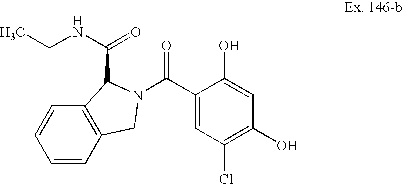

and peak #2, (S)-2-(5-chloro-2,4-dihydroxybenzoyl)-N-ethylisoindoline-1-carboxamide (Ex. 146-c, 230 mg, white solid).

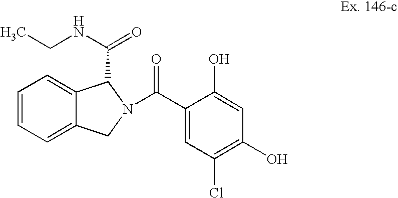

Both peaks gave ee %: 100%.

Anal. Calcd for Ex. 146-b: C₁₈H₁₇ClN₂O₄.0.25H₂O: C, 59.18; H, 4.83; N, 7.67. Found: C, 59.31; H, 4.76; N, 7.56.

Anal. Calcd for Ex. 146-c: C₁₈H₁₇ClN₂O₄.0.5H₂O: C, 58.46; H, 4.91; N, 7.58. Found: C, 58.72; H, 4.76; N, 7.62.

### Example 147

### 1-[3-(1,3-dihydro-2H-isoindol-2-ylcarbonyl)-4-hydroxyphenyl]ethanone

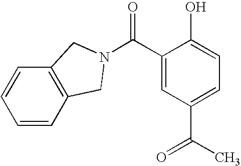

The above compound was prepared as follows using the General Procedure G6. Tri-butyl (1-ethoxyvinyl) tin (372 mg, 1 mmol) and tetrakis (triphenylphosphine) palladium (60 mg, 0.05 mmol) were added to a solution of the product obtained from Example 10 (251 mg, 0.8 mmole) in dioxane (6 mL) at room temperature. The reaction was purged with nitrogen several times and was heated to 90° C. The reaction was allowed to stir at 90° C. for 12 hours. The reaction mixture was added to H₂O and then EtOAc was added to extract the aqueous layer. Dry EtOAc layer over Na₂SO₄. The Na₂SO₄ was filtered off and the filtrate was evaporated to give a brown oil residue. The residue was purified by silica gel chromatography (gradient elution 30→40% EtOAc in hexanes) to give the desired product (117 mg, 67.2% yield). ¹H NMR (400 MHz, CHLOROFORM-D) δ ppm 2.59 (s, 3H) 5.04-5.17 (m, 4H) 7.04 (d, J=8.59 Hz, 1H) 7.27-7.33 (m, 4H) 7.96 (dd, J=8.72, 2.15 Hz, 1H) 8.32-8.52 (m, J=2.27 Hz, 1H) 11.90 (s, 1H): Anal. Calcd for C₁₇H₁₅NO₃.0.25 CH₃OH: C, 71.61; H, 5.57; N, 4.84. Found: C, 71.49; H, 5.47; N, 4.79.

### Example 148

### 2-(1,3-dihydro-2H-isoindol-2-ylcarbonyl)-4-(1-hydroxyethyl)phenol

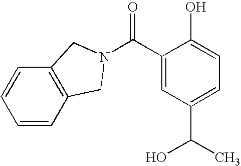

The above compound was prepared as follows using the General Procedure G6. Sodium borohydride (100 mg, 1.5 mmol) was added to a solution of the product obtained from Example 147 (31.8 mg, 0.113 mmol) in EtOH (5 mL) at room temperature. The reaction was stirred at room temperature for 12 hours. The reaction mixture was acidified with NaOAc and NaOAc buffer (20 mL) and then EtOAc was added to extract the aqueous layer. Dry EtOAc layer over Na₂SO₄. The Na₂SO₄ was filtered off and the filtrate was evaporated to give a brown oil residue. The residue was purified by silica gel chromatography (gradient elution 50→60% EtOAc in hexanes) to give the desired product (10 mg, 31% yield). ¹H NMR (400 MHz, MeOD) δ ppm 1.39-1.45 (m, J=6.57 Hz, 3H) 4.73-4.76 (m, 2H) 4.78 (q, J=6.48 Hz, 1H) 4.90-4.98 (m, 2H) 6.89 (d, J=8.34 Hz, 1H) 7.14-7.22 (m, 1H) 7.24-7.48 (m, 5H): Anal. Calcd for C₁₇H₁₇NO₃.0.15H₂O: C, 71.39; H, 6.10; N, 4.90. Found: C, 71.43; H, 6.26; N, 4.69.

### Example 149

### 3-(1,3-dihydro-2H-isoindol-2-ylcarbonyl)-4-hydroxybenzonitrile

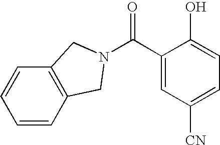

The above compound was prepared as follows using the General Procedure G6. Chloromethyl methyl ether (0.5 mL, 6.3 mmol) and diisopropylethyl amine (1.1 ml, 6.2 mmol) were added to a solution of the product obtained from Example 137 (658 mg, 2.07 mmole) in DMF (5 mL) at room temperature. The reaction was stirred at room temperature for 12 hours. To the reaction mixture was added H₂O (50 mL), and then EtOAc was added to extract the aqueous layer (50 mL×2). Dry EtOAc layer over Na₂SO₄. The Na₂SO₄ was filtered off and the filtrate was evaporated to give a brown oil residue. The residue was purified by silica gel chromatography (gradient elution 30→40% EtOAc in hexanes) to give the desired intermediate product (635 mg, 84.6% yield) 2-[5-bromo-2-(methoxymethoxy)benzoyl]isoindoline. ¹H NMR (300 MHz, CHLOROFORM-D) δ ppm 3.33 (s, 3H) 4.55 (s, 2H) 4.88 (s, 2H) 5.04-5.10 (m, 2H) 6.98-7.10 (m, 2H) 7.11-7.26 (m, 3H) 7.35 (s, 1H) 7.36-7.41 (m, 1H).

Then, potassium cyanide (80 mg, 1.1 mmol) and tetrakis (triphenylphosphine) palladium (60 mg, 0.05 mmol) were added to a solution of 2-[5-bromo-2-(methoxymethoxy)benzoyl]isoindoline as prepared above (200 mg, 0.55 mmole) in THF (5 mL) at room temperature. The reaction was heated and stirred at 80° C. for 12 hours. The reaction mixture was evaporated to give a brown oil residue. The residue was purified by silica gel chromatography (gradient elution 35→40% EtOAc in hexanes) to give the desired intermediate product (3-(1,3-dihydro-2H-isoindol-2-ylcarbonyl)-4-(methoxymethoxy)benzonitrile (130 mg, 76.7% yield). ¹H NMR (400 MHz, CHLOROFORM-D) δ ppm 3.38 (s, 3H) 4.53 (s, 2H) 4.92 (s, 2H) 5.19 (s, 2H) 7.08 (d, J=7.33 Hz, 1H) 7.26 (d, J=3.03 Hz, 2H) 7.39 (dd, J=7.83, 2.78 Hz, 2H) 7.43-7.50 (m, 1H) 7.52-7.65 (m, 2H).

Hydrogen chloride (1 mL, 4 mmol; 4 M in dioxane) was then added to a solution of 3-(1,3-dihydro-2H-isoindol-2-ylcarbonyl)-4-(methoxymethoxy)benzonitrile (130 mg, 0.42 mmole) in DCM (5 mL) at room temperature. The reaction was heated and stirred at room temperature for 12 hours. The reaction mixture was evaporated to give an oil residue. The residue was purified by silica gel chromatography (gradient elution 45→50% EtOAc in hexanes) to give the desired final product, 3-(1,3-dihydro-2H-isoindol-2-ylcarbonyl)-4-hydroxybenzonitrile (50.8 mg, 45.5% yield). ¹H NMR (400 MHz, DMSO-D6) δ ppm 3.32 (s, 1H) 4.58 (s, 2H) 4.80 (s, 2H) 7.05-7.09 (m, 1H) 7.22-7.34 (m, 3H) 7.38 (d, J=7.07 Hz, 1H) 7.68-7.84 (m, 2H) 11.21 (s, 1H): Anal. Calcd for C₁₆H₁₂N₂O₂: C, 72.72; H, 4.58; N, 10.60. Found: C, 72.50; H, 4.59; N, 10.39.

### Example 150

### 4-chloro-6-(1,3-dihydro-2H-isoindol-2-ylcarbonyl)-5-methylbenzene-1,3-diol

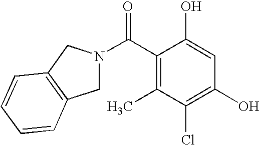

The above compound was prepared as follows using General Procedure G9. Isoindoline (130 mg, 1 mmol) was added to a solution of 3-chloro-4,6-dimethoxy-2-methylbenzoic acid (compound A in general procedure G9, and prepared via the method reported by Clevenger et al. Organic Letters (2004), 6 (24), p4459)) (200 mg, 0.87 mmole), diisopropylethyl amine (0.8 mL, 4.5 mmol), and O-(7-azabenzotriazol-1-yl)-N,N,N′,N′-tetramethyluronium phosphorus pentafloride (HATU) (380 mg, 1 mmol) in 4 mL of DMF under a nitrogen atmosphere. The reaction was allowed to stir at room temperature for 12 hours. Saturated NaHCO₃ (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 45 →50% EtOAc in hexanes) to give the desired intermediate product (3-chloro-4,6-dimethoxy-2-methylphenyl)(isoindolin-2-yl)methanone (265 mg, 92% yield). ¹H NMR (400 MHz, DMSO-D6) δ ppm 2.18 (s, 3H) 3.81 (s, 3H) 3.92 (s, 3H) 4.39 (d, J=7.07 Hz, 2H) 4.80 (s, 2H) 6.76 (s, 1H) 7.17-7.35 (m, 3H) 7.38 (d, J=7.07 Hz, 1H): Anal. Calcd for C₁₈H₁₈ClNO₃: C, 65.16; H, 5.47; N, 4.22. Found: C, 65.05; H, 5.48; N, 4.22.

Boron tribromide (2.4 mL, 2.4 mmol; 1 M in heptane) was added to a solution of (3-chloro-4,6-dimethoxy-2-methylphenyl)(isoindolin-2-yl)methanone as prepared above (131.5 mg, 0.4 mmole) in DCM (7 mL) at −78° C. The reaction was stirred at room temperature for 12 hours. The reaction mixture was neutralized with Na₂CO₃ and then EtOAc was added to extract the aqueous layer. Dry EtOAc layer over Na₂SO₄. The Na₂SO₄ was filtered off and the filtrate was evaporated to give a brown oil residue. The residue was purified by silica gel chromatography (gradient elution 10→20% EtOAc in hexanes) to give the desired product (164 mg, 43.4% yield). The residue was purified by silica gel chromatography (gradient elution 70→75% EtOAc in hexanes) to give the desired final product (76.5 mg, 63.6% yield) 4-chloro-6-(1,3-dihydro-2H-isoindol-2-ylcarbonyl)-5-methylbenzene-1,3-diol. ¹H NMR (400 MHz, DMSO-D6) δ ppm 2.12 (s, 3H) 4.39 (d, J=16 Hz, 1H) 4.52 (d, J=12 Hz, 1H) 4.77 (s, 2H) 6.47 (s, 1H) 7.19-7.32 (m, 3H) 7.33-7.50 (m, J=7.07 Hz, 1H) 9.74 (s, 1H) 10.08 (s, 1H): Anal. Calcd for C₁₆H₁₄ClNO₃.0.15H₂O: C, 58.90; H, 5.10; N, 4.29. Found: C, 59.32; H, 5.08; N, 4.23.

### Example 151

### 4-chloro-6-{[(2R)-2-(pyrrolidin-1-ylmethyl)pyrrolidin-1-yl]carbonyl}benzene-1,3-diol

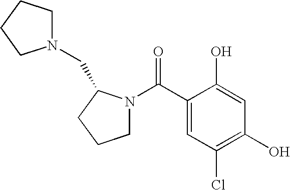

The above compound was prepared as follows using the General Procedure 10. (R)-(−)-1-(2-pyrrolidinylmethyl)pyrrolidine (110 mg, 0.7 mmol) was added to a solution of 5-chloro-2,4-bis(methoxymethoxy)benzoic acid (compound B in general procedure G10) (150 mg, 0.54 mmole), 4-methylmorpholine (0.6 ml, 5.4 mmol), N-(3-dimethylaminopropyl)-N′-ethylcarbodiimide hydrochloride (215 mg, 1.1 mmol), and 1-hydroxy benzotriazole (150 mg, 1.1 mmol) in 4 mL of DMF under a nitrogen atmosphere. The reaction was allowed to stir at room temperature for 12 hours. H₂O (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 20→30% MeOH in DCM to give the desired intermediate product (126 mg, 56.5% yield) as shown below.

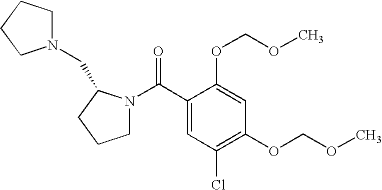

Hydrogen chloride (1.5 mL, 6 mmol; 4 M in dioxane) was then added to a solution of (R)-(5-chloro-2,4-bis(methoxymethoxy)phenyl)(2-(pyrrolidin-1-ylmethyl)pyrrolidin-1-yl)methanone (as shown above) (126 mg, 0.31 mmole) in MeOH (5 mL). The reaction was stirred at room temperature for 12 hours. The reaction mixture was evaporated to give the desired final product as a pinkish solid (110 mg, 90.7% yield). ¹H NMR (400 MHz, DMSO-D6) δ ppm 1.60-2.23 (m, 9H) 3.02-3.27 (m, 3H) 3.36-3.48 (m, 1H) 3.57-3.91 (m, 2H) 4.26-4.47 (m, 1H) 6.60 (s, 1H) 7.19 (s, 1H) 9.86 (s, 1H) 10.25 (s, 1H) 10.53 (s, 1H). Anal. Calcd for C₁₆H₂₁ClN2O₃.HCl.0.25H₂O: C, 52.54; H, 6.20; N, 7.66. Found: C, 52.17; H, 6.50; N, 7.30.

### Example 152

### 2-(5-chloro-2,4-dihydroxybenzoyl)-N-ethylisoindoline-5-carboxamide

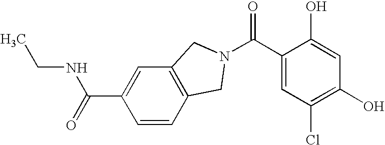

The above compound was prepared as follows using General Procedure G11. Methyl isoindoline-5-carboxylate (110 mg, 0.56 mmol, prepared via the method reported by Devadas et al. in WO2005018557A2) was added to a solution of 5-chloro-2,4-bis(methoxymethoxy)benzoic acid (compound B in general procedure G10) (160 mg, 0.56 mmole), 4-methylmorpholine (0.9 ml, 8.5 mmol), N-(3-dimethylaminopropyl)-N′-ethylcarbodiimide hydrochloride (230 mg, 1.2 mmol), and 1-hydroxy benzotriazole (165 mg, 1.2 mmol) in 5 mL of DMF under a nitrogen atmosphere. The reaction was allowed to stir at room temperature for 12 hours. H₂O (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 30→40% EtOAc in hexanes to give the intermediate compound A (68.4 mg, 27.8% yield) shown below.

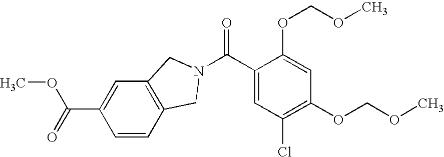

¹H NMR (400 MHz, DMSO-D6) δ ppm 2.78 (s, 1H) 2.94 (s, 1H) 3.17 (d, J=1.26 Hz, 1H) 3.32 (s, 3H) 3.41 (s, 3H) 3.51 (t, J=4.67 Hz, 1H) 3.59 (s, 2H) 5.14-5.25 (m, 2H) 5.26-5.46 (m, 2H) 7.03 (s, 1H) 7.18-7.44 (m, 1H).

Lithium hydroxide monohydrate (40 mg, 0.8 mmol) was added to a solution of the intermediate compound A shown above (68.4 mg, 0.16 mmol) in H₂O (1 mL) and MeOH (2 mL). The reaction mixture was stirred at 40° C. for 12 hours and was neutralized with NaOAc—HOAc buffer solution. EtOAc (2×50 mL) was then added to extract the aqueous layer. Dry EtOAc layer over Na2SO₄. The Na₂SO₄ was filtered off and the filtrate was evaporated to give a white solid residue as the desired intermediate compound B (65.2 mg, 98%) shown below.

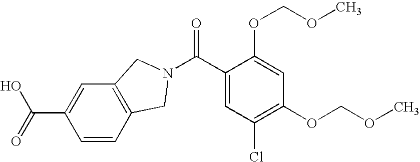

Intermediate compound B was used in the next step without further purification. Ethylamine (0.1 mL, 0.2 mmol, 2 M in THF) was added to a solution of intermediate compound B shown above (65.2 mg, 0.15 mmol), 4-methylmorpholine (0.2 ml, 1.54 mmol), N-(3-dimethylaminopropyl)-N′-ethylcarbodiimide hydrochloride (60 mg, 0.31 mmol), and 1-hydroxy benzotriazole (50 mg, 0.31 mmol) in 3 mL of DMF under a nitrogen atmosphere. The reaction was allowed to stir at room temperature for 12 hours. H₂O (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 80→90% EtOAc in hexanes to give the desired intermediate compound C (55.6 mg, 80.4% yield) shown below.

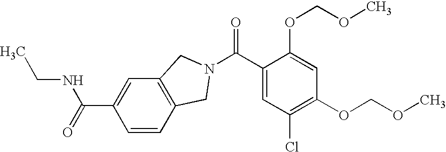

¹H NMR (400 MHz, DMSO-D6) δ ppm 2.78 (s, 1H) 2.94 (s, 1H) 3.17 (d, J=1.26 Hz, 1H) 3.32 (s, 3H) 3.41 (s, 3H) 3.51 (t, J=4.67 Hz, 1H) 3.59 (s, 2H) 5.14-5.25 (m, 2H) 5.26-5.46 (m, 2H) 7.03 (s, 1H) 7.18-7.44 (m, 1H).

Hydrogen chloride (2.5 mL, 10 mmol; 4 M in dioxane) was then added to a solution of compound C as shown above (55.6 mg, 0.124 mmole) in MeOH (3 mL). The reaction was stirred at room temperature for 12 hours. The reaction mixture was evaporated, neutralized with sat. NaHCO₃ (aq), and then extracted with EtOAc (2×50 mL). The combined organic layer was dried, filtered, and evaporated to give the desired final product, 2-(5-chloro-2,4-dihydroxybenzoyl)-N-ethylisoindoline-5-carboxamide, as a white solid (48.4 mg, quantitative yield). ¹H NMR (400 MHz, DMSO-D6) δ ppm 1.10 (t, J=7.07 Hz, 3H) 3.24-3.29 (m, 1H) 4.67-4.87 (m, 4H) 7.22 (s, 1H) 7.30-7.50 (m, 1H) 7.74 (d, J=7.83 Hz, 1H) 7.82 (s, 1H) 8.45 (s, 1H) 10.34 (s, 1H) 10.44 (s, 1H). Anal. Calcd for C₁₈H₁₇ClN₂O₄.0.25 CH₃OH: C, 59.44; H, 4.92; N, 7.60. Found: C, 59.91; H, 5.01; N, 7.11.

### Example 153

### 5-chloro-2,4-bis(methoxymethoxy)benzoic acid

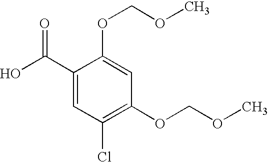

The above compound was prepared as follows using General Procedure G10. Chloromethyl methyl ether (13 mL, 171 mmol) and DIEA (42 mL, 240 mmol) were added to a reaction solution of methyl-2,4-dihroxybenzoate (6.7 g, 40 mmol) in 40 mL of DMF under N₂ atmosphere. The resulting mixture was stirred at room temperature for 12 hours. The reaction mixture was partitioned between EtOAc (3×500 mL) and water (200 mL), saturated NaHCO₃ solution (2×200 mL) and brine (200 mL). The organic layer was dried (Na₂SO₄), filtered, and concentrated by vacuum to give an oil residue. The residue was purified by silica gel chromatography (gradient elution 0→30% EtOAc in hexanes to give the desired intermediate product Methyl 2,4-bis(methoxymethoxy)benzoate (9.45 g, 92% yield). ¹H NMR (400 MHz, CHLOROFORM-D) δ ppm 3.47 (s, 3H) 3.51 (s, 3H) 3.85 (s, 3H) 5.18 (s, 2H) 5.23 (s, 2H) 6.70 (dd, J=8.84, 2.27 Hz, 1H) 6.83 (d, J=2.27 Hz, 1H) 7.80 (d, J=8.84 Hz, 1H).

Methyl 5-chloro-2,4-bis(methoxymethoxy)benzoate was then prepared as follows. Preparation of 0.7 M Calcium hypochlorite solution in 10% HOAc: Calcium hypochlorite (9.743 g, 44.29 mmol) was dissolved into 64 mL of 10% HOAc in an ice-bath with stirring. Calcium hypochlorite solution was added dropwise to a reaction solution of Methyl 2,4-bis(methoxymethoxy)benzoate (as prepared above) (9.45 g, 36.9 mmol) in 50.0 mL of acetone. The resulting mixture was stirred at room temperature for 12 hours. The reaction mixture was partitioned between EtOAc (3×500 mL) and saturated NaHCO₃ solution (200 mL). The organic layer was dried (Na₂SO₄), filtered, and then concentrated by vacuum. The residue was purified by silica gel chromatography (gradient elution 0→30% EtOAc in hexanes to give the desired product Methyl 5-chloro-2,4-bis(methoxymethoxy)benzoate (6.6 g, 62% yield). ¹H NMR (400 MHz, CHLOROFORM-D) δ ppm 3.51 (d, J=1.26 Hz, 6H) 3.85 (s, 3H) 5.22 (s, 2H) 5.27 (s, 2H) 7.02 (s, 1H) 7.87 (s, 1H).

NaOH (aq) solution (40 mL, 80 mmol, 2M) was then added to a reaction solution of Methyl 5-chloro-2,4-bis(methoxymethoxy)benzoate (5.8 g, 20 mmol) in MeOH (80 mL). The resulting mixture was stirred at room temperature for 12 hours. The reaction mixture was concentrated by vacuum to remove most of the MeOH and then extracted with ether (200 mL). The aqueous layer was neutralized to pH 6.5 using 2N HCl solution and then extracted with EtOAc (3×500 mL) and CH₂Cl₂ (2×500 mL). The combined organic layers were dried (Na₂SO₄) and then concentrated by vacuum to afford 5.2 g of the desired final product, 5-chloro-2,4-bis(methoxymethoxy)benzoic acid, as a white solid (94% yield). ¹H NMR (400 MHz, DMSO-D6) δ ppm 3.39 (d, J=8.34 Hz, 6H) 5.10 (s, 2H) 5.22 (s, 2H) 6.83 (s, 1H) 7.42 (s, 1H).

### Example 154

### 2-[5-chloro-2,4-bis(methoxymethoxy)benzoyl]isoindoline-1-carboxylic acid

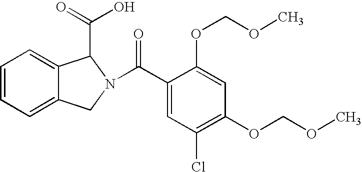

The above compound was prepared as follows using General Procedure G11a. Methyliodide (350 mg, 2.2 mmol) was added to a solution of (R,S)-Boc-1,3-dihydro-2H-isoindole carboxylic acid (500 mg, 1.9 mmol) and potassium carbonate (830 mg, 6 mmol) in DMF (5 mL). The reaction mixture was stirred at room temperature for 12 hours. H₂O (50 mL) was added to the reaction mixture and EtOAc was added to extract the aqueous solution. Dry EtOAc layer over Na₂SO₄. The Na₂SO₄ was filtered off and the filtrate was evaporated to give an oil residue. The residue was purified by silica gel chromatography (gradient elution 20→30% EtOAc in hexanes) to give the desired product (2-tert-butyl 1-methyl 1,3-dihydro-2H-isoindole-1,2-dicarboxylate) as a colorless oil (533 mg, quantitative yield). ¹H NMR (400 MHz, CHLOROFORM-D) δ ppm 1.53 (s, 9H) 3.77 (s, 3H) 4.64-4.99 (m, 2H) 5.53 (d, J=2.53 Hz, 1H) 7.29-7.51 (m, 4H).

Hydrogen chloride (8 mL, 32 mmol, 4M in dioxane) was then added to a solution of 2-tert-butyl 1-methyl 1,3-dihydro-2H-isoindole-1,2-dicarboxylate (532 mg, 1.9 mmol) in DCM (5 mL). The reaction was stirred at room temperature for 12 hours. The reaction mixture was evaporated to give an oil residue of the desired intermediate product, methyl isoindoline-1-carboxylate. The residue was used for the next step reaction without further purification.

Methyl isoindoline-1-carboxylate (1.9 mmol) was added to a solution of 5-chloro-2,4-bis(methoxymethoxy)benzoic acid, as prepared in Example 153 (536 mg, 1.9 mmol), 4-methylmorpholine (3.4 mL, 30 mmol), N-(3-dimethylaminopropyl)-N′-ethylcarbodiimide hydrochloride (770 mg, 4 mmol), and 1-hydroxy benzotriazole (550 mg, 4 mmol) in 15 mL of DMF under a nitrogen atmosphere. The reaction was allowed to stir at room temperature for 12 hours. H₂O (50 mL) was added to the reaction mixture to quench the reaction. EtOAc (2×150 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 40→45% EtOAc in hexanes to give the desired intermediate product, methyl 2-[5-chloro-2,4-bis(methoxymethoxy)benzoyl]isoindoline-1-carboxylate, as a white solid (532.3 mg, 64.2% yield).

Lithium hydroxide hydrate (720 mg, 17 mmol) was added to a solution of methyl 2-[5-chloro-2,4-bis(methoxymethoxy)benzoyl]isoindoline-1-carboxylate (532 mg, 1.2 mmol) in H₂O (3 mL) and MeOH (5 mL). The reaction mixture was heated to 40° C. for 12 hours. The mixture was evaporated and neutralized by HOAc—NaOAc buffer solution. EtOAc (2×150 mL) was added to extract the aqueous solution. The combined organic layers were dried, filtered, and concentrated to give the desired final product, 2-[5-chloro-2,4-bis(methoxymethoxy)benzoyl]isoindoline-1-carboxylic acid, as a white foam (516 mg, quantitative yield).

### Example 155

### 3-chloro-4,6-dihydroxy-N,N-dimethyl-2-(2-oxo-2-piperidin-1-ylethyl)benzamide

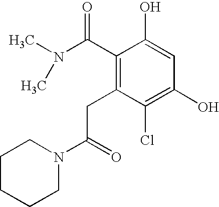

The above compound was prepared as follows using General Procedure G9b. O-(7-azabenzotriazol-1-yl)-N,N,N′,N′-tetramethyluronium phosphorus pentafloride (HATU) (326 mg, 0.9 mmol), diisopropylethyl amine (0.5 mL, 3.1 mmol), and dimethylamine HCl (2) (100 mg, 1.2 mmol) were added to a solution of 3-chloro-4,6-dimethoxy-2-(2-methoxy-2-oxoethyl)benzoic acid (226 mg, 0.78 mmol, which compound was prepared via the method reported by Henderson, et. al. J. Chem. Soc. Perkin Trans. 1 (1982), 4, p. 1111) in 10 mL of DMF under a nitrogen atmosphere. The reaction was allowed to stir at room temperature for 12 hours. Saturated NaHCO₃ was added to the reaction mixture to quench the reaction. EtOAc was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 0→30% EtOAc in hexanes) to give the desired intermediate product (Methyl {2-chloro-6-[(dimethylamino)carbonyl]-3,5-dimethoxyphenyl}acetate) as a white foam (240 mg, 97% yield). ¹H NMR (400 MHz, CHLOROFORM-D) δ ppm 2.82 (s, 3H) 3.08 (s, 3H) 3.67 (s, 3H) 3.79-3.86 (m, 6H) 3.89-3.94 (m, 2H) 6.46 (s, 1H).

NaOH (aq) (2 mL, 4 mmol, 2N) was added to a reaction solution of Methyl {2-chloro-6-[(dimethylamino)carbonyl]-3,5-dimethoxyphenyl}acetate (254 mg, 0.8 mmol) in 5 mL of MeOH and 5 mL of THF. The resulting mixture was stirred at room temperature for 12 hours. The reaction mixture was concentrated by vacuum and then acidified to pH 4.0 using 2N HCl (aq). EtOAc (400 mL) was added to extract the aqueous solution. The organic layer was dried (Na₂SO₄), filtered, and concentrated by vacuum to give the desired intermediate product, {2-chloro-6-[(dimethylamino)carbonyl]-3,5-dimethoxyphenyl}acetic acid, as a white solid (222 mg, 92% yield). ¹H NMR (400 MHz, DMSO-D6) δ ppm 2.62-2.75 (m, 3H) 2.89-3.01 (m, 3H) 3.53 (s, 2H) 3.83 (s, 3H) 3.91 (s, 3H) 6.78 (s, 1H) 12.42 (s, 1H).

N-methylmorpholine (NMM, 0.16 mL, 1.44 mmol) and 2-chloro-4,6-dimethoxy-1,3,5-triazine (CDMT, 139 mg, 0.8 mmol) were added to a suspension of {2-chloro-6-[(dimethylamino)carbonyl]-3,5-dimethoxyphenyl}acetic acid (217 mg, 0.72 mmol) in 5.0 mL of EtOAc. The resulting suspension was stirred at room temperature for 30 minutes and then piperidine (0.1 mL, 0.76 mmol) was added. The resulting mixture was stirred at room temperature for another 12 hours. The reaction mixture was portioned between EtOAc (150 mL) and water (50 mL). The organic layer was dried (Na₂SO₄), filtered, and then concentrated by vacuum. The residue was purified by silica gel chromatography (gradient elution 0→10% MeOH in DCM) to give the desired intermediate product, 3-chloro-4,6-dimethoxy-N,N-dimethyl-2-(2-oxo-2-piperidin-1-ylethyl)benzamide, as a white solid (94 mg, 35% yield). ¹H NMR (400 MHz, DMSO-D6) δ ppm 1.38-1.49 (m, J=7.58, 4.04 Hz, 3H) 1.50-1.67 (m, 3H) 2.66 (s, 3H) 2.90 (s, 3H) 3.27-3.33 (m, 2H) 3.38-3.51 (m, 4H) 3.82 (s, 3H) 3.90 (s, 3H) 6.74 (s, 1H).

Boron tribromide (1.5 mL, 1.5 mmol; 1 M in heptane) was added to a solution of 3-chloro-4,6-dimethoxy-N,N-dimethyl-2-(2-oxo-2-piperidin-1-ylethyl)benzamide (94 mg, 0.25 mmole) in DCM (10 mL) at 0° C. The reaction was heated to 40° C. and stirred at 40° C. for 12 hours. The reaction mixture was neutralized with Na₂CO₃ and then EtOAc (200 mL) was added to extract the aqueous layer. Dry EtOAc layer over Na₂SO₄. The Na₂SO₄ was filtered off and the filtrate was evaporated to give a yellow oil residue. The residue was purified by silica gel chromatography (gradient elution 0→10% MeOH in DCM) to give the desired final product, 3-chloro-4,6-dihydroxy-N,N-dimethyl-2-(2-oxo-2-piperidin-1-ylethyl)benzamide, as a white solid (23 mg, 27% yield). ¹H NMR (400 MHz, MeOD) δ ppm 1.41-1.54 (m, 2H) 1.54-1.71 (m, 2H) 2.83 (s, 3H) 2.96 (s, 3H) 3.41-3.56 (m, 6H) 3.57-3.69 (m, 1H) 3.79-3.91 (m, J=4.8 Hz, 1H) 6.36 (s, 1H).

### Example 156

### 2-(5-chloro-2,4-dihydroxybenzoyl)-N-ethylisoindoline-1-carboxamide

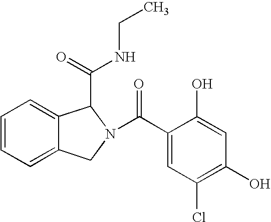

The above compound was prepared as follows using General Procedure G12. Ethylamine (2.5 mL, 5 mmol, 2M in THF) was added to a solution of Boc(R,S)-1,3-dihydro-2H-isoindole carboxylic acid (263 mg, 1 mmole), diisopropylethyl amine (0.9 ml, 5 mmol), and O-(7-azabenzotriazol-1-yl)-N,N,N′,N′-tetramethyluronium phosphorus pentafloride (HATU) (420 mg, 1.1 mmol) in 5 mL of DMF under a nitrogen atmosphere. The reaction was allowed to stir at room temperature for 12 hours. Saturated NaHCO₃ (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 40→50% EtOAc in hexanes) to give the desired intermediate product, tert-butyl 1-[(ethylamino)carbonyl]-1,3-dihydro-2H-isoindole-2-carboxylate, (267.5 mg, 92% yield). ¹H NMR (400 MHz, CHLOROFORM-D) δ ppm 1.10 (t, J=7.20 Hz, 3H) 1.49 (s, 9H) 3.10 (s, 1H) 3.42 (s, 1H) 4.68-4.93 (m, J=1.01 Hz, 2H) 5.36 (s, 1H) 7.26-7.37 (m, 3H) 7.52 (d, J=7.07 Hz, 1H). Hydrogen chloride (5 mL, 20 mmol; 4 M in dioxane) was added to a solution of tert-butyl 1-[(ethylamino)carbonyl]-1,3-dihydro-2H-isoindole-2-carboxylate (267.5 mg, 0.92 mmole) in DCM (5 mL) at room temperature. The reaction was heated and stirred at room temperature for 12 hours. The reaction mixture was evaporated to give an oil residue. The residue, (N-ethylisoindoline-1-carboxamide) was used for the next step reaction without further purification.

N-ethylisoindoline-1-carboxamide (0.92 mmol) was added to a solution of 5-chloro-2,4-dihydroxybenzoic acid (compound C in General Procedure G10; 175 mg, 0.92 mmol), 4-methylmorpholine (0.9 ml, 8 mmol), N-(3-dimethylaminopropyl)-N′-ethylcarbodiimide hydrochloride (353 mg, 1.84 mmol), and 1-hydroxy benzotriazole (250 mg, 1.84 mmol) in 4 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 70→80% EtOAc in hexanes to give the desired final product (2-(5-chloro-2,4-dihydroxybenzoyl)-N-ethylisoindoline-1-carboxamide) as a white solid (83 mg, 25% yield). ¹H NMR (400 MHz, MeOD) δ ppm 1.10-1.18 (m, 3H) 4.78-4.85 (m, 2H) 5.00-5.12 (m, 1H) 5.67-5.86 (m, 1H) 6.48-6.64 (m, 1H) 7.32 (t, J=6.19 Hz, 2H) 7.43-7.50 (m, 1H) 7.54 (t, J=7.58 Hz, 1H) 7.70-7.79 (m, 1H) 7.87 (d, J=8.34 Hz, 1 H) 8.14 (s, 1H). Anal. Calcd for C₁₈H₁₇ClN₂O₄: C, 59.92; H, 4.75; N, 7.76. Found: C, 59.81; H, 4.74; N, 7.71.

### Example 157

### 2-(5-chloro-2,4-dihydroxybenzoyl)-N-ethylisoindoline-1-carboxamide

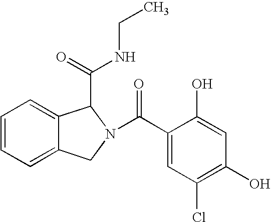

The above compound was prepared as follows using General Procedure G12. Ethylamine (10 mL, 20 mmol, 2M in THF) was added to a solution of Boc(R,S)-1,3-dihydro-2H-isoindole carboxylic acid (1 g, 3.8 mmole), diisopropylethyl amine (3.3 ml, 19 mmol), and O-(7-azabenzotriazol-1-yl)-N,N,N′,N′-tetramethyluronium phosphorus pentafloride (HATU) (1.6 g, 4.2 mmol) in 15 mL of DMF under a nitrogen atmosphere. The reaction was allowed to stir at room temperature for 12 hours. Saturated NaHCO₃ (100 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 40→50% EtOAc in hexanes) to give the desired intermediate product, tert-butyl 1-[(ethylamino)carbonyl]-1,3-dihydro-2H-isoindole-2-carboxylate, (890 mg, 80.3% yield). ¹H NMR (400 MHz, CHLOROFORM-D) δ ppm 1.10 (t, J=7.20 Hz, 3H) 1.49 (s, 9H) 3.10 (s, 1H) 3.42 (s, 1H) 4.68-4.93 (m, J=1.01 Hz, 2H) 5.36 (s, 1H) 7.26-7.37 (m, 3H) 7.52 (d, J=7.07 Hz, 1H).

Hydrogen chloride (12 mL, 48 mmol; 4 M in dioxane) was added to a solution of tert-butyl 1-[(ethylamino)carbonyl]-1,3-dihydro-2H-isoindole-2-carboxylate (788 mg, 2.7 mmole) in DCM (10 mL) at room temperature. The reaction was heated and stirred at room temperature for 12 hours. The reaction mixture was evaporated to give an oil residue. The residue (N-ethylisoindoline-1-carboxamide) was used for the next reaction step without further purification.

N-ethylisoindoline-1-carboxamide (2.7 mmol) was added to a solution of 5-chloro-2,4-bis(methoxymethoxy)benzoic acid (as prepared in Example 153) (750 mg, 2.7 mmol), 4-methylmorpholine (4.7 ml, 41 mmol), N-(3-dimethylaminopropyl)-N′-ethylcarbodiimide hydrochloride (1.05 g, 5.5 mmol), and 1-hydroxy benzotriazole (750 mg, 5.5 mmol) in 15 mL of DMF under a nitrogen atmosphere. The reaction was allowed to stir at room temperature for 12 hours. H₂O (100 mL) was added to the reaction mixture to quench the reaction. EtOAc (2×150 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 75→85% EtOAc in hexanes to give the desired intermediate product (2-[5-chloro-2,4-bis(methoxymethoxy)benzoyl]-N-ethylisoindoline-1-carboxamide) as a white solid (860 mg, 70.7% yield).

Hydrogen chloride (10 mL, 38.4 mmol; 4 M in dioxane) was added to a solution of 2-[5-chloro-2,4-bis(methoxymethoxy)benzoyl]-N-ethylisoindoline-1-carboxamide (860 mg, 1.92 mmol) in MeOH (10 mL). The reaction was stirred at room temperature for 12 hours. The reaction mixture was neutralized with saturated NaHCO₃ (aq) and then extracted with EtOAc (2×100 mL). The combined organic layers were dried, filtered, and evaporated to give the desired product as a pinkish solid as the final product (2-(5-chloro-2,4-dihydroxybenzoyl)-N-ethylisoindoline-1-carboxamide) (612 mg, 88.7% yield). ¹H NMR (400 MHz, DMSO-D6) δ ppm 1.00 (t, J=6.57 Hz, 3H) 2.69-3.18 (m, 2H) 4.57-5.05 (m, 2H) 5.54 (s, 1H) 6.56 (d, J=20.97 Hz, 1H) 7.11-7.64 (m, 5H) 8.07 (s, 1H) 10.04 (s, 1H) 10.41 (s, 1H). Anal. Calcd for C₁₈H₁₇ClN₂O₄: C, 59.92; H, 4.75; N, 7.76. Found: C, 59.58; H, 4.80; N, 7.59.

### Example 158

### 2-(5-chloro-2,4-dihydroxybenzoyl)-N-cyclobutylisoindoline-1-carboxamide

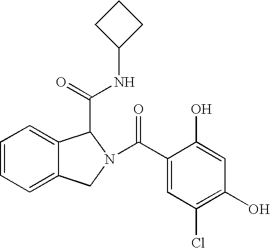

The above compound was prepared as follows using General Procedure G12. Following the procedure described in Example 146, using cyclobutylamine in place of ethylamine, tert-butyl 1-[(cyclobutylamino)carbonyl]-1,3-dihydro-2H-isoindole-2-carboxylate was prepared and used for the next step reaction without further purification.

Hydrogen chloride (2 mL, 8 mmol; 4 M in dioxane) was added to a solution of tert-butyl 1-[(cyclobutylamino)carbonyl]-1,3-dihydro-2H-isoindole-2-carboxylate (1 mmol) in DCM (3 mL) at room temperature. The reaction was heated and stirred at room temperature for 12 hours. The reaction mixture was evaporated to give an oil residue. The residue (N-cyclobutylisoindoline-1-carboxamide) was used for the next reaction step without further purification.

N-cyclobutylisoindoline-1-carboxamide (1 mmol) was added to a solution of 5-chloro-2,4-bis(methoxymethoxy)benzoic acid (as prepared in Example 153) (263 mg, 0.95 mmol), 4-methylmorpholine (1.7 mL, 15 mmol), N-(3-dimethylaminopropyl)-N′-ethylcarbodiimide hydrochloride (385 mg, 2 mmol), and 1-hydroxy benzotriazole (275 mg, 2 mmol) in 10 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 70→75% EtOAc in hexanes to give the desired intermediate product (2-[5-chloro-2,4-bis(methoxymethoxy)benzoyl]-N-cyclobutylisoindoline-1-carboxamide) as a white solid (245 mg, 52% yield).

Hydrogen chloride (3 mL, 12 mmol; 4 M in dioxane) was added to a solution of 2-[5-chloro-2,4-bis(methoxymethoxy)benzoyl]-N-cyclobutyl isoindoline-1-carboxamide (1 mmol) in MeOH (5 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give the desired final product (2-(5-chloro-2,4-dihydroxybenzoyl)-N-cyclobutylisoindoline-1-carboxamide) as a pinkish solid (330 mg, 85% yield). ¹H NMR (400 MHz, DMSO-D6) δ ppm 1.77-2.22 (m, 6H) 4.07-4.31 (m, 1H) 4.75-4.96 (m, 2H) 5.52 (s, 1H) 6.30-6.72 (m, J=17.68 Hz, 1H) 7.16-7.71 (m, 4H) 8.29 (d, J=7.58 Hz, 1H) 10.04 (s, 1H) 10.51 (s, 1H). Anal. Calcd for C₂₀H₁₉ClN₂O₄.0.25H₂O: C, 61.38; H, 5.02; N, 7.16. Found: C, 61.55; H, 4.88; N, 7.13.

### Example 159

### 2-(5-chloro-2,4-dihydroxybenzoyl)-N-(2-isocyanoethyl)isoindoline-1-carboxamide

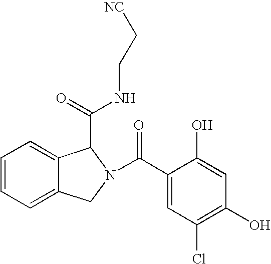

The above compound was prepared as follows using General Procedure G12. Following the procedure described in Example 146, using 3-aminopropionitrile in place of ethylamine, tert-butyl 1-{[(2-isocyanoethyl)amino]carbonyl}-1,3-dihydro-2H-isoindole-2-carboxylate was prepared and used for the next reaction step without further purification.

Hydrogen chloride (3 mL, 12 mmol; 4 M in dioxane) was added to a solution of tert-butyl 1-{[(2-isocyanoethyl)amino]carbonyl}-1,3-dihydro-2H-isoindole-2-carboxylate (1 mmole) in DCM (3 mL) at room temperature. The reaction was heated and stirred at room temperature for 12 hours. The reaction mixture was evaporated to give an oil residue. The residue (N-(2-isocyanoethyl)isoindoline-1-carboxamide) was used for the next reaction step without further purification.

N-(2-isocyanoethyl)isoindoline-1-carboxamide (0.7 mmol) was added to a solution of 5-chloro-2,4-bis(methoxymethoxy)benzoic acid (as prepared in Example 153) (200 mg, 0.7 mmol), 4-methylmorpholine (1.2 ml, 10.5 mmol), N-(3-dimethylaminopropyl)-N′-ethylcarbodiimide hydrochloride (270 mg, 1.4 mmol), and 1-hydroxy benzotriazole (200 mg, 1.4 mmol) in 8 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue (2-[5-chloro-2,4-bis(methoxymethoxy)benzoyl]-N-(2-isocyanoethyl)isoindoline-1-carboxamide) was used for the next reaction step without further purification.

Hydrogen chloride (3 mL, 12 mmol; 4 M in dioxane) was added to a solution of 2-[5-chloro-2,4-bis(methoxymethoxy)benzoyl]-N-(2-isocyanoethyl)isoindoline-1-carboxamide (0.7 mmole) in MeOH (5 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give a brown oil. The residue was purified by silica gel chromatography (gradient elution 70→75% EtOAc in hexanes to give the desired final product, 2-(5-chloro-2,4-dihydroxybenzoyl)-N-(2-isocyanoethyl)isoindoline-1-carboxamide, as a white solid (54.7 mg, 20% yield over 4 steps). ¹H NMR (400 MHz, DMSO-D6) δ ppm 2.56-2.66 (m, 1H) 3.03-3.13 (m, 1H) 3.20-3.30 (m, 1H) 3.50-3.59 (m, 1H) 4.81-4.88 (m, 2H) 5.52-5.61 (m, 1H) 6.55-6.62 (m, 1H) 7.26-7.37 (m, 4H) 7.42 (s, 1H) 8.61 (s, 1H) 10.09 (s, 1H) 10.39 (s, 1H). Anal. Calcd for C₁₉H₁₆ClN₃O₄.0.25H₂O.0.75HCl.0.3 CH₃OH: C, 54.25; H, 4.35; N, 9.83. Found: C, 54.64; H, 4.05; N, 9.41.

### Example 160

### 2-(5-chloro-2,4-dihydroxybenzoyl)-N-cyclopropylisoindoline-1-carboxamide

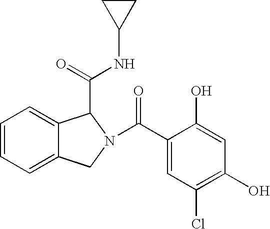

The above compound was prepared as follows using General Procedure G12. Following the procedure described in Example 146, using cyclopropylamine in place of ethylamine, the residue was purified by silica gel chromatography (gradient elution 60→70% EtOAc in hexanes) to give the desired intermediate product (292 mg, 96.7% yield) tert-butyl 1-[(cyclopropylamino)carbonyl]-1,3-dihydro-2H-isoindole-2-carboxylate.

Hydrogen chloride (4 mL, 16 mmol; 4 M in dioxane) was added to a solution of tert-butyl 1-[(cyclopropylamino)carbonyl]-1,3-dihydro-2H-isoindole-2-carboxylate (0.97 mmole) in DCM (5 mL) at room temperature. The reaction was heated and stirred at room temperature for 12 hours. The reaction mixture was evaporated to give an oil residue. The residue (N-cyclopropylisoindoline-1-carboxamide) was used for the next reaction step without further purification.

N-cyclopropylisoindoline-1-carboxamide (0.97 mmol) was added to a solution of 5-chloro-2,4-bis(methoxymethoxy)benzoic acid (as prepared in Example 153) (280 mg, 1 mmol), 4-methylmorpholine (1.7 mL, 15 mmol), N-(3-dimethylaminopropyl)-N′-ethylcarbodiimide hydrochloride (385 mg, 2 mmol), and 1-hydroxy benzotriazole (280 mg, 2 mmol) in 8 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 70→80% EtOAc in hexanes) to give the desired intermediate product 2-[5-chloro-2,4-bis(methoxymethoxy)benzoyl]-N-cyclopropylisoindoline-1-carboxamide (299 mg, 67% yield).

Hydrogen chloride (3 mL, 12 mmol; 4 M in dioxane) was then added to a solution of 2-[5-chloro-2,4-bis(methoxymethoxy)benzoyl]-N-cyclopropylisoindoline-1-carboxamide (0.65 mmol) in MeOH (2 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give a brown oil. To the residue was added DCM to give the desired final product (2-(5-chloro-2,4-dihydroxybenzoyl)-N-cyclopropylisoindoline-1-carboxamide) as a white solid (20 mg, 8% yield). ¹H NMR (400 MHz, DMSO-D6) δ ppm −0.04-0.87 (m, 4H) 2.21-2.39 (m, J=1.77 Hz, 1H) 4.74-4.99 (m, 2H) 5.40-5.71 (m, 1H) 6.23-6.77 (m, J=19.71 Hz, 1H) 6.91 (s, 1H) 7.19-7.56 (m, 4H) 8.16 (d, J=3.28 Hz, 1H) 10.06 (s, 1H) 10.37-10.88 (m, 1H). Anal. Calcd for C₁₉H₁₇ClN₂O₄.0.25H₂O: C, 60.48; H, 4.67; N, 7.42. Found: C, 60.50; H, 4.57; N, 7.15.

### Example 161

### 2-(5-chloro-2,4-dihydroxybenzoyl)-N-(2,2,2-trifluoroethyl)isoindoline-1-carboxamide

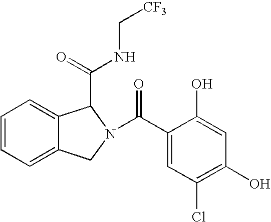

The above compound was prepared as follows using General Procedure G12. First, following the procedure described in Example 146, using trifluoroethylamine in place of ethylamine, the residue was purified by silica gel chromatography (gradient elution 50→60% EtOAc in hexanes) to give the desired intermediate product (202 mg, 84.2% yield) tert-butyl 1-{[(2,2,2-trifluoroethyl)amino]carbonyl}-1,3-dihydro-2H-isoindole-2-carboxylate.

Hydrogen chloride (2.5 mL, 10 mmol; 4 M in dioxane) was added to a solution of tert-butyl 1-{[(2,2,2-trifluoroethyl)amino]carbonyl}-1,3-dihydro-2H-isoindole-2-carboxylate (0.59 mmole) in DCM (5 mL) at room temperature. The reaction was heated and stirred at room temperature for 12 hours. The reaction mixture was evaporated to give an oil residue, N-(2,2,2-trifluoroethyl)isoindoline-1-carboxamide. The residue was used for the next reaction step without further purification.

N-(2,2,2-trifluoroethyl)isoindoline-1-carboxamide (0.59 mmol) was added to a solution of 5-chloro-2,4-bis(methoxymethoxy)benzoic acid (as prepared in Example 153) (170 mg, 0.6 mmol), 4-methylmorpholine (1 ml, 9 mmol), N-(3-dimethylaminopropyl)-N′-ethylcarbodiimide hydrochloride (250 mg, 1.2 mmol), and 1-hydroxy benzotriazole (170 mg, 1.2 mmol) in 5 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 40→50% EtOAc in hexanes) to give the desired intermediate product (212 mg, 71.5% yield) 2-[5-chloro-2,4-bis(methoxymethoxy)benzoyl]-N-(2,2,2-trifluoroethyl)isoindoline-1-carboxamide.

Hydrogen chloride (3 mL, 12 mmol; 4 M in dioxane) was added to a solution of 2-[5-chloro-2,4-bis(methoxymethoxy)benzoyl]-N-(2,2,2-trifluoroethyl)isoindoline-1-carboxamide (212 mg, 0.42 mmol) in DCM (3 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give a brown oil. To the residue was added DCM to give the desired final product, 2-(5-chloro-2,4-dihydroxybenzoyl)-N-(2,2,2-trifluoroethyl)isoindoline-1-carboxamide, as a white solid (156.5 mg, 89.4% yield). ¹H NMR (400 MHz, DMSO-D6) δ ppm 3.77-4.24 (m, 2H) 4.55-5.00 (m, 2H) 5.41-5.77 (m, 1H) 6.43-6.72 (m, 1H) 7.23 (s, 1H) 7.26-7.54 (m, 4H) 8.73-9.18 (m, 1H) 10.08 (s, 1H) 10.38 (s, 1H). Anal. Calcd for C₁₈H₁₄ClF₃N₂O₄: C, 52.13; H, 3.40; N, 6.75. Found: C, 52.25; H, 3.58; N, 6.70.

### Example 162

### N-allyl-2-(5-chloro-2,4-dihydroxybenzoyl)isoindoline-1-carboxamide

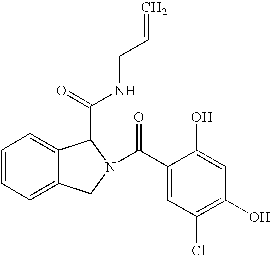

The above compound was prepared as follows using General Procedure G12. First, following the procedure described in Example 146, using allylamine in place of ethylamine, the residue was purified by silica gel chromatography (gradient elution 40→50% EtOAc in hexanes) to give the desired intermediate product (321 mg, quantitative yield) tert-butyl 1-[(allylamino)carbonyl]-1,3-dihydro-2H-isoindole-2-carboxylate.

Hydrogen chloride (3 mL, 12 mmol; 4 M in dioxane) was added to a solution of tert-butyl 1-[(allylamino)carbonyl]-1,3-dihydro-2H-isoindole-2-carboxylate (1 mmol) in DCM (5 mL) at room temperature. The reaction was heated and stirred at room temperature for 12 hours. The reaction mixture was evaporated to give an oil residue. The residue (N-allylisoindoline-1-carboxamide) was used for the next step reaction without further purification.

N-allylisoindoline-1-carboxamide (1 mmol) was then added to a solution of 5-chloro-2,4-bis(methoxymethoxy)benzoic acid (as prepared in Example 153) (340 mg, 1.2 mmol), 4-methylmorpholine (2.2 ml, 20 mmol), N-(3-dimethylaminopropyl)-N′-ethylcarbodiimide hydrochloride (460 mg, 2.4 mmol), and 1-hydroxy benzotriazole (330 mg, 2.4 mmol) in 12 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 50→60% EtOAc in hexanes) to give the desired intermediate product (423 mg, 91.8% yield) N-allyl-2-[5-chloro-2,4-bis(methoxymethoxy)benzoyl]isoindoline-1-carboxamide.

Hydrogen chloride (4 mL, 16 mmol; 4 M in dioxane) was added to a solution of N-allyl-2-[5-chloro-2,4-bis(methoxymethoxy)benzoyl]isoindoline-1-carboxamide (392 mg, 0.85 mmol) in DCM (5 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give the desired final product (N-allyl-2-(5-chloro-2,4-dihydroxybenzoyl)isoindoline-1-carboxamide) as a white solid (221 mg, 69.7% yield). ¹H NMR (400 MHz, DMSO-D6) δ ppm 3.57 (d, J=79.33 Hz, 2H) 4.65-4.93 (m, 1H) 4.97-5.19 (m, 1H) 5.42-5.70 (m, 1H) 5.68-5.95 (m, 1H) 6.40-6.71 (m, 1H) 6.92 (s, 1H) 7.15-7.67 (m, 4H) 8.28 (s, 1H) 10.06 (s, 1H) 10.40 (s, 1H). Anal. Calcd for C₁₉H₁₇ClN₂O₄: C, 61.21; H, 4.60; N, 7.51. Found: C, 61.02; H, 4.63; N, 7.36.

### Example 163

### 2-(5-chloro-2,4-dihydroxybenzoyl)-N-isopropylisoindoline-1-carboxamide

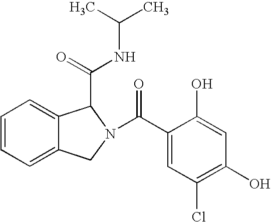

The above compound was prepared as follows using General Procedure G11. Isopropylamine (50 mg, 0.5 mmol) was added to a solution of 2-[5-chloro-2,4-bis(methoxymethoxy)benzoyl]isoindoline-1-carboxylic acid (compound F in general procedure G11a, 100 mg, 0.24 mmol), 4-methylmorpholine (0.5 ml, 4.5 mmol), N-(3-dimethylaminopropyl)-N′-ethylcarbodiimide hydrochloride (120 mg, 0.6 mmol), and 1-hydroxy benzotriazole (85 mg, 0.6 mmol) in 3 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 70→80% EtOAc in hexanes) to give the desired intermediate product (89.8 mg, 81.8% yield) 2-[5-chloro-2,4-bis(methoxymethoxy)benzoyl]-N-isopropylisoindoline-1-carboxamide.

Hydrogen chloride (1 mL, 4 mmol; 4 M in dioxane) was added to a solution of 2-[5-chloro-2,4-bis(methoxymethoxy)benzoyl]-N-isopropylisoindoline-1-carboxamide (89.8 mg, 0.19 mmole) in DCM (3 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give a white solid (60 mg, 82% yield), 2-(5-chloro-2,4-dihydroxybenzoyl)-N-isopropylisoindoline-1-carboxamide, as the final product. ¹H NMR (400 MHz, DMSO-D6) δ ppm 0.87-1.24 (m, 6H) 3.49-3.71 (m, 1H) 4.76-4.94 (m, 2H) 5.45-5.69 (m, 1H) 6.56 (d, J=23.24 Hz, 1H) 6.90 (s, 1H) 7.30 (d, J=2.02 Hz, 4H) 7.86-8.18 (m, 1H) 10.28 (s, 1H) 10.45 (s, 1H). Anal. Calcd for C₁₉H₁₉ClN₂O₄.0.2 hexane: C, 61.95; H, 5.51; N, 7.15. Found: C, 61.75; H, 5.26; N, 6.92.

### Example 164

### 2-(5-chloro-2,4-dihydroxybenzoyl)-N-[2-(dimethylamino)ethyl]isoindoline-1-carboxamide

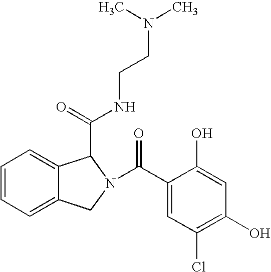

The above compound was prepared as follows according to General Procedure G11. N,N-dimethylethylenediamine (70 mg, 0.6 mmol) was added to a solution of 2-[5-chloro-2,4-bis(methoxymethoxy)benzoyl]isoindoline-1-carboxylic acid (compound F in general procedure G11a, 113 mg, 0.27 mmol), 4-methylmorpholine (0.5 ml, 4.5 mmol), N-(3-dimethylaminopropyl)-N′-ethylcarbodiimide hydrochloride (120 mg, 0.6 mmol), and 1-hydroxy benzotriazole (85 mg, 0.6 mmol) in 3 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 0→10% MeOH in DCM) to give the desired intermediate product (50 mg, 38% yield) 2-[5-chloro-2,4-bis(methoxymethoxy)benzoyl]-N-[2-(dimethylamino)ethyl]isoindoline-1-carboxamide.

Hydrogen chloride (0.7 mL, 2.8 mmol; 4 M in dioxane) was added to a solution of 2-[5-chloro-2,4-bis(methoxymethoxy)benzoyl]-N-[2-(dimethylamino)ethyl]isoindoline-1-carboxamide (50 mg, 0.1 mmole) in MeOH (3 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give a white solid (60 mg, quantitative yield) as the final product, 2-(5-chloro-2,4-dihydroxybenzoyl)-N-[2-(dimethylamino)ethyl]isoindoline-1-carboxamide. ¹H NMR (400 MHz, DMSO-D6) δ ppm 2.65 (s, 3H) 2.76 (s, 3H) 3.11 (t, J=6.19 Hz, 2H) 3.37-3.59 (m, 2H) 4.79-4.97 (m, 2H) 5.52-5.65 (m, 1H) 6.63 (s, 1H) 7.25-7.37 (m, 3H) 7.41 (d, J=7.33 Hz, 1H) 8.45-8.76 (m, 1H) 10.20 (s, 1H) 10.38 (s, 1H) 10.54 (s, 1H). Anal. Calcd for C₁₉H₁₉ClN₂O₄.1HCl.0.5H₂O.0.5 CH₃OH: C, 52.91; H, 5.63; N, 9.03. Found: C, 52.85; H, 5.63; N, 8.69.

### Example 165

### 2-(5-chloro-2,4-dihydroxybenzoyl)-N-(2,2-difluoroethyl)isoindoline-1-carboxamide

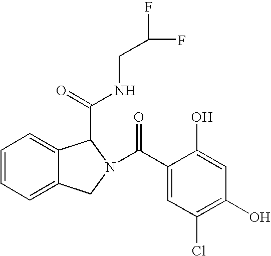

The above compound was prepared as follows using General Procedure G11. 2,2-difluoroethylamine (50 mg, 0.6 mmol) was added to a solution of 2-[5-chloro-2,4-bis(methoxymethoxy)benzoyl]isoindoline-1-carboxylic acid (compound F in general procedure G11a, 118 mg, 0.28 mmol), 4-methylmorpholine (0.5 mL, 4.5 mmol), N-(3-dimethylaminopropyl)-N′-ethylcarbodiimide hydrochloride (120 mg, 0.6 mmol), and 1-hydroxy benzotriazole (85 mg, 0.6 mmol) in 3 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 50→60% EtOAc in hexanes) to give the desired intermediate product (100.5 mg, 74% yield) 2-[5-chloro-2,4-bis(methoxymethoxy)benzoyl]-N-(2,2-difluoroethyl)isoindoline-1-carboxamide.

Hydrogen chloride (0.7 mL, 2.8 mmol; 4 M in dioxane) was added to a solution of 2-[5-chloro-2,4-bis(methoxymethoxy)benzoyl]-N-(2,2-difluoroethyl)isoindoline-1-carboxamide (64.5 mg, 0.13 mmol) in MeOH (4 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give a white solid (50 mg, 94.7% yield) as the final product, 2-(5-chloro-2,4-dihydroxybenzoyl)-N-(2,2-difluoroethyl)isoindoline-1-carboxamide. ¹H NMR (400 MHz, DMSO-D6) δ ppm 3.40-3.79 (m, 2H) 4.82 (s, 2H) 5.34-5.79 (m, 1H) 6.57 (s, 1H) 6.92 (s, 1H) 7.16-7.53 (m, 4H) 8.25-8.91 (m, 1H) 10.08 (s, 1H) 10.39 (s, 1H). Anal. Calcd for C₁₈H₁₅ClF₂N₂O₄.1 Hexane: C, 55.11; H, 4.08; N, 6.91. Found: C, 55.09; H, 4.02; N, 6.64.

### Example 166

### 2-(5-chloro-2,4-dihydroxybenzoyl)-N-(4-hydroxycyclohexyl)isoindoline-1-carboxamide

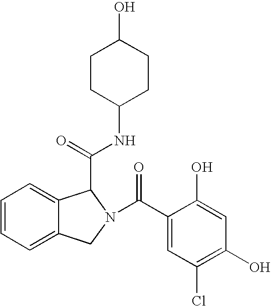

The above compound was prepared as follows using General Procedure G11. Trans-4-aminocyclohexanol hydrochloride (90 mg, 0.54 mmol) was added to a solution of 2-[5-chloro-2,4-bis(methoxymethoxy)benzoyl]isoindoline-1-carboxylic acid (compound F in general procedure G11a, 114 mg, 0.27 mmol), 4-methylmorpholine (0.5 ml, 4.5 mmol), N-(3-dimethylaminopropyl)-N′-ethylcarbodiimide hydrochloride (110 mg, 0.54 mmol), and 1-hydroxy benzotriazole (75 mg, 0.54 mmol) in 5 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 0→10% MeOH in EtOAc) to give the desired intermediate product (80 mg, 57% yield) 2-[5-chloro-2,4-bis(methoxymethoxy)benzoyl]-N-(4-hydroxycyclohexyl)isoindoline-1-carboxamide.

Hydrogen chloride (0.8 mL, 3.2 mmol; 4 M in dioxane) was added to a solution of 2-[5-chloro-2,4-bis(methoxymethoxy)benzoyl]-N-(4-hydroxycyclohexyl)isoindoline-1-carboxamide (80 mg, 0.15 mmole) in MeOH (4 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give white solid (60 mg, 90.4% yield) as the final product, 2-(5-chloro-2,4-dihydroxybenzoyl)-N-(4-hydroxycyclohexyl)isoindoline-1-carboxamide. ¹H NMR (400 MHz, DMSO-D6) δ ppm 1.04-1.25 (m, 4H) 1.59-1.85 (m, 4H) 4.35-4.55 (m, J=13.64 Hz, 2H) 4.59-4.95 (m, 2H) 5.53 (s, 1H) 6.55 (d, J=25.01 Hz, 1H) 6.87 (d, J=7.58 Hz, 1H) 7.10-7.55 (m, J=1.77 Hz, 4H) 7.91 (s, 1H) 10.00 (s, 1H) 10.46 (s, 1H). Anal. Calcd for C₂₂H₂₃ClN₂O₅.0.25H₂O: C, 60.69; H, 5.44; N, 6.43. Found: C, 60.42; H, 5.50; N, 6.18.

### Example 167

### 2-(5-chloro-2,4-dihydroxybenzoyl)-N-propylisoindoline-1-carboxamide

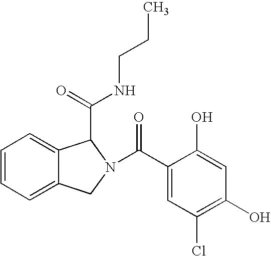

The compound above was prepared as follows using the General Procedure G11. Propylamine (40 mg, 0.6 mmol) was added to a solution of 2-[5-chloro-2,4-bis(methoxymethoxy)benzoyl]isoindoline-1-carboxylic acid (compound F in general procedure G11a, 104 mg, 0.25 mmol), 4-methylmorpholine (0.34 ml, 3 mmol), N-(3-dimethylaminopropyl)-N′-ethylcarbodiimide hydrochloride (100 mg, 0.5 mmol), and 1-hydroxy benzotriazole (70 mg, 0.5 mmol) in 5 mL of DMF under a nitrogen atmosphere. The reaction was allowed to stir at room temperature for 12 hours. H₂O (50 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a pale yellow oil residue. The residue (2-[5-chloro-2,4-bis(methoxymethoxy)benzoyl]-N-propylisoindoline-1-carboxamide) was used for the next reaction step without further purification.

Hydrogen chloride (1 mL, 4 mmol; 4 M in dioxane) was added to a solution of 2-[5-chloro-2,4-bis(methoxymethoxy)benzoyl]-N-propylisoindoline-1-carboxamide (110 mg, 0.25 mmol) in MeOH (3 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give an oil residue. The residue was purified by silica gel chromatography (gradient elution 70→80% EtOAc in hexanes) to give the desired final product (2-(5-chloro-2,4-dihydroxybenzoyl)-N-propylisoindoline-1-carboxamide) as a white solid (15 mg, 16% yield over two steps). ¹H NMR (400 MHz, DMSO-D6) δ ppm 0.72-0.95 (m, 3H) 1.16-1.57 (m, 2H) 2.91 (dd, J=13.01, 6.95 Hz, 2H) 4.54-4.92 (m, 2H) 5.49-5.67 (m, 1H) 6.56 (s, 1H) 6.92 (s, 1H) 7.14-7.55 (m, 2H) 8.10 (s, 1H) 10.07 (s, 1H) 10.48 (s, 1H). Anal. Calcd for C₁₉H₁₉ClN₂O₄.1H₂O: C, 58.09; H, 5.39; N, 7.13. Found: C, 57.87; H, 5.12; N, 7.34.

TABLE 2

| No. | Structure | Name | Proc. | ¹H NMR |
|---|---|---|---|---|
| 168 | 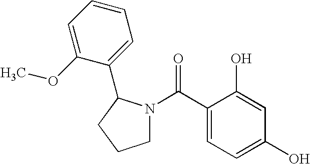 | 4-{[2-(2- methoxyphenyl)pyrrolidin- 1- yl]carbonyl}benzene- 1,3-diol | G2 | (400 MHz, DMSO-d6) δ ppm 1.53-1.91 (m, 3 H) 2.19-2.38 (m, 1 H) 3.44-3.62 (m, 1 H) 3.68-3.92 (m, 4 H) 5.35 (s, 1 H) 6.19-6.36 (m, 2 H) 6.87 (t, J = 7.33 Hz, 1 H) 6.92-7.04 (m, 1 H) 7.12-7.31 (m, 3 H) 9.64-9.78 (m, 1 H) 10.62 (s, 1 H) |
| 169 | 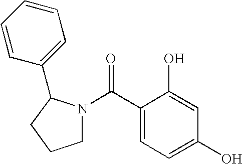 | 4-[(2-phenylpyrrolidin- 1- yl)carbonyl]benzene- 1,3-diol | G2 | (400 MHz, DMSO-d6) δ ppm 1.67-1.77 (m, 1 H) 1.77-1.87 (m, 2 H) 2.29-2.38 (m, 1 H) 3.53-3.64 (m, 1 H) 3.75-3.86 (m, 1 H) 5.14 (s, 1 H) 6.22-6.32 (m, 2 H) 7.15-7.24 (m, J = 5.05 Hz, 2 H) 7.24-7.35 (m, 4 H) 9.76 (s, 1 H) 10.68 (s, 1 H) |
| 170 | 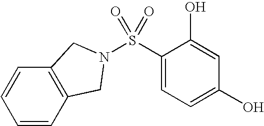 | 4-(1,3-dihydro-2H- isoindol-2- ylsulfonyl)benzene- 1,3-diol | G2 | (400 MHz, DMSO-d6) δ ppm 4.61 (s, 4 H) 6.30-6.35 (m, 2 H) 7.22-7.29 (m, 4 H) 7.52-7.57 (m, 1 H) 10.21 (s, 1 H) 10.50 (s, 1 H) |
| 171 | 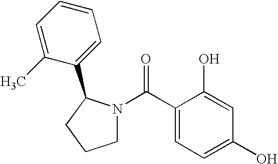 | 4-{[(2S)-2-(2- methylphenyl)pyrrolidin-1- yl]carbonyl}benzene- 1,3-diol | G2 | (400 MHz, DMSO-d6) δ ppm 1.34-2.12 (m, 4 H) 2.18-2.40 (m, 3 H) 3.40-3.96 (m, 2 H) 5.07-5.32 (m, 1 H) 5.64-6.63 (m, 2 H) 6.83-7.35 (m, 5 H) 9.27-9.96 (m, 1 H) 10.22-10.86 (m, 1 H) |
| 172 | 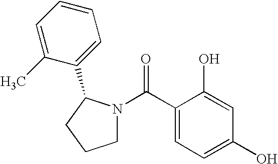 | 4-{[(2R)-2-(2- methylphenyl)pyrrolidin-1- yl]carbonyl}benzene- 1,3-diol | G2 | (400 MHz, DMSO-d6) δ ppm 1.45-2.16 (m, 4 H) 2.23-2.45 (m, 3 H) 3.48-3.97 (m, 2 H) 5.14-5.40 (m, 1 H) 5.72-6.41 (m, 2 H) 6.88-7.39 (m, 5 H) 9.31-9.91 (m, 1 H) 10.37-10.84 (m, 1 H) |
| 173 | 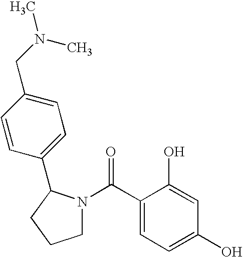 | 4-[(2-{4- [(dimethylamino) methyl]phenyl}pyrrolidin-1- yl)carbonyl]benzene- 1,3-diol | G2 | (400 MHz, DMSO-d6) δ ppm 1.70-1.75 (m, 1 H) 1.75-1.87 (m, 3 H) 1.89 (s, 2 H) 2.11 (s, 6 H) 2.26-2.37 (m, J = 10.74, 10.74 Hz, 1 H) 3.52-3.63 (m, 1 H) 3.74-3.86 (m, 1 H) 5.07-5.18 (m, 1 H) 6.21-6.32 (m, 2 H) 7.16-7.28 (m, 5 H) |
| 174 | 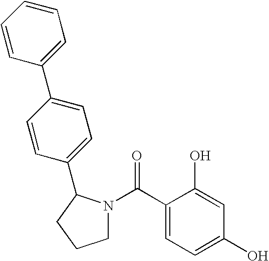 | 4-[(2-biphenyl-4- ylpyrrolidin-1- yl)carbonyl]benzene- 1,3-diol | G2 | (400 MHz, DMSO-d6) δ ppm 1.68-1.97 (m, 3 H) 2.39 (s, 1 H) 3.61 (s, 1 H) 3.86 (s, 1 H) 5.18 (s, 1 H) 6.29 (s, 2 H) 7.22-7.53 (m, 6 H) 7.52-7.75 (m, J = 22.86, 7.71 Hz, 4 H) 9.78 (s, 1 H) 10.73 (s, 1 H) |
| 175 | 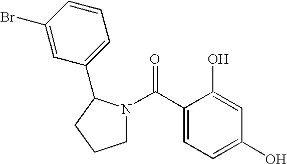 | 4-{[(2-(3- bromophenyl) pyrrolidin-1- yl]carbonyl}benzene- 1,3-diol | G2 | (400 MHz, DMSO-d6) δ ppm 1.58-1.92 (m, 3 H) 2.27-2.44 (m, 1 H) 3.43-3.60 (m, 1 H) 3.69-3.93 (m, 1 H) 5.03-5.17 (m, 1 H) 6.15-6.63 (m, 2 H) 7.09-7.64 (m, 5 H) 9.63-9.84 (m, 1 H) 10.42-10.67 (m, 1 H) |
| 176 | 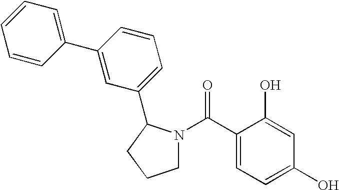 | 4-[(2-biphenyl-3- ylpyrrolidin-1- yl)carbonyl]benzene- 1,3-diol | G8 | (400 MHz, DMSO-d6) δ ppm 1.69-1.95 (m, 3 H) 2.24-2.46 (m, 1 H) 3.42-3.69 (m, J = 13.14 Hz, 1 H) 3.75-3.95 (m, J = 5.81 Hz, 1 H) 5.10-5.31 (m, 1 H) 6.15-6.39 (m, 2 H) 7.15-7.43 (m, 4 H) 7.42-7.54 (m, 3 H) 7.54-7.71 (m, 3 H) 9.41-9.88 (m, 1 H) 10.25-10.69 (m, 1 H) |
| 177 | 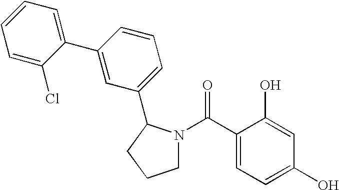 | 4-{[2-(2′- chlorobiphenyl-3- yl)pyrrolidin-1- yl]carbonyl}benzene- 1,3-diol | G8 | (400 MHz, DMSO-d6) δ ppm 1.76-1.88 (m, 4 H) 2.31-2.42 (m, 1 H) 3.50-3.62 (m, 1 H) 3.75-3.86 (m, 1 H) 5.13-5.25 (m, 1 H) 6.27 (s, 2 H) 7.18-7.30 (m, 2 H) 7.36-7.45 (m, 5 H) 7.52-7.58 (m, 1 H) 9.74 (s, 1 H) 10.58 (s, 1 H) |
| 178 | 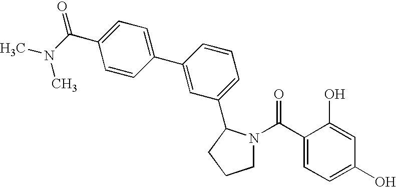 | 3′-[1-(2,4- dihydroxybenzoyl) pyrrolidin-2-yl]-N,N- dimethylbiphenyl-4- carboxamide | G8 | (400 MHz, DMSO-d6) δ ppm 1.83 (s, 3 H) 2.38 (s, 1 H) 2.93-3.04 (m, J = 11.37 Hz, 6 H) 3.54 (s, 1 H) 3.85 (s, 1 H) 5.22 (s, 1 H) 6.30 (s, 2 H) 7.17-7.29 (m, 1 H) 7.29-7.46 (m, 2 H) 7.46-7.58 (m, 3 H) 7.59-7.76 (m, 3 H) 9.69- 9.77 (m, 1 H) 10.56 (s, 1 H) |
| 179 | 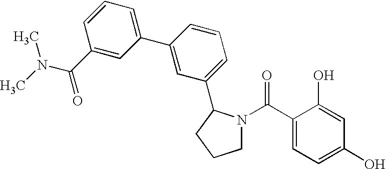 | 3′-[1-(2,4- dihydroxybenzoyl) pyrrolidin-2-yl]-N,N- dimethylbiphenyl-3- carboxamide | G8 | (400 MHz, DMSO-d6) δ ppm 1.74-1.93 (m, 3 H) 2.30-2.45 (m, 1 H) 2.91-2.97 (m, 3 H) 2.98-3.04 (m, 3 H) 3.50-3.62 (m, 1 H) 3.79-3.92 (m, 1 H) 5.16-5.27 (m, 1 H) 6.22-6.36 (m, 2 H) 7.18-7.28 (m, 1 H) 7.29-7.36 (m, 1 H) 7.36-7.43 (m, 2 H) 7.49-7.57 (m, 2 H) 7.59-7.68 (m, 2 H) 7.68-7.76 (m, 1 H) 9.69-9.80 (m, 1 H) 10.55-10.65 (m, 1 H) |
| 180 | 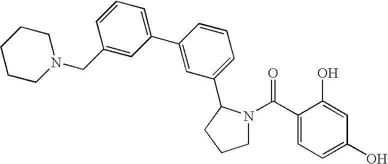 | 4-({2-[3′-(piperidin-1- ylmethyl)biphenyl-3- yl]pyrrolidin-1- yl}carbonyl)benzene- 1,3-diol | G8 | |
| 181 | 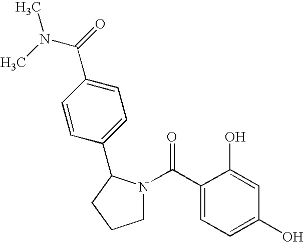 | 4-[1-(2,4- dihydroxybenzoyl) pyrrolidin-2-yl]-N,N- dimethylbenzamide | G7 | (400 MHz, DMSO-d6) δ ppm 1.60-1.94 (m, 3 H) 2.27-2.44 (m, 1 H) 2.83-3.03 (m, 6 H) 3.46-3.66 (m, 1 H) 3.72-3.92 (m, 1 H) 5.01-5.24 (m, 1 H) 6.07-6.42 (m, 2 H) 7.15-7.48 (m, 5 H) 9.60-9.89 (m, 1 H) 10.50-10.81 (m, 1 H) |
| 182 | 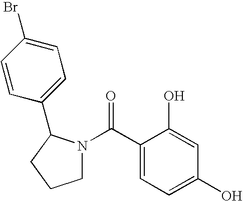 | 4-{[2-(4- bromophenyl) pyrrolidin-1-yl]- carbonyl}benzene- 1,3-diol | G2 | (400 MHz, DMSO-d6) δ ppm 1.58-1.91 (m, 3 H) 2.21-2.43 (m, 1 H) 3.43-3.68 (m, 1 H) 3.68-3.91 (m, 1 H) 4.80-5.29 (m, 1 H) 5.71-6.44 (m, 2 H) 6.46-7.38 (m, 3 H) 7.38-7.65 (m, 2 H) 9.24-9.99 (m, 1 H) 10.01-10.91 (m, 1 H) |
| 183 | 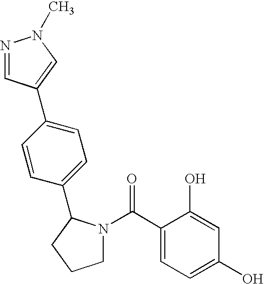 | 4-({2-[4-(1-methyl-1H- pyrazol-4- yl)phenyl]pyrrolidin-1- yl}carbonyl)benzene- 1,3-diol | G8 | (400 MHz, DMSO-d6) δ ppm 1.63-1.95 (m, 3 H) 2.22-2.41 (m, 1 H) 3.48-3.65 (m, 1 H) 3.77-3.92 (m, 4 H) 5.04-5.20 (m, 1 H) 5.79-6.40 (m, 2 H) 6.54-7.36 (m, 3 H) 7.39-7.52 (m, J = 7.58 Hz, 2 H) 7.81 (s, 1 H) 8.08 (s, 1 H) 9.35-10.02 (m, 1 H) 10.26-10.91 (m, 1 H) |
| 184 | 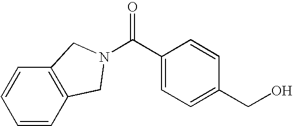 | [4-(1,3-dihydro-2H- isoindol-2- ylcarbonyl)phenyl] methanol | G2 | (400 MHz, DMSO-d6) δ ppm 4.56 (s, 2 H) 4.77 (s, 2 H) 4.86 (s, 2 H) 7.23-7.34 (m, 3 H) 7.35-7.46 (m, 3 H) 7.58 (d, J = 8.08 Hz, 2 H) |
| 185 | 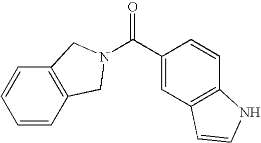 | 5-(1,3-dihydro-2H- isoindol-2- ylcarbonyl) 1H-indole | G2 | (400 MHz, DMSO-d6) δ ppm 4.87 (d, J = 16.67 Hz, 4 H) 6.49-6.54 (m, J = 2.27 Hz, 1 H) 7.24-7.33 (m, 3 H) 7.36 (dd, J = 8.34, 1.52 Hz, 1 H) 7.40 (d, J = 6.82 Hz, 1 H) 7.43-7.47 (m, 2 H) 7.87 (s, 1 H) 11.32 (s, 1 H) |
| 186 | 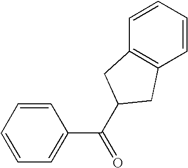 | 2-benzoylisoindoline | G2 | (400 MHz, DMSO-d6) δ ppm 4.75 (s, 2 H) 4.86 (s, 2 H) 7.25-7.33 (m, 3 H) 7.40 (d, J = 6.82 Hz, 1 H) 7.45-7.52 (m, 3 H) 7.60 (ddd, J = 4.93, 2.40, 2.27 Hz, 2 H) |
| 187 | 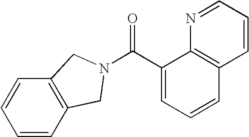 | 8-(1,3-dihydro-2H- isoindol-2- ylcarbonyl) quinoline | G2 | (400 MHz, DMSO-D6) δ ppm 4.36 (s, 2 H) 4.95 (s, 2 H) 7.12 (d, J = 6.82 Hz, 1 H) 7.21 (s, 1 H) 7.29 (s, 1 H) 7.41 (d, J = 6.06 Hz, 1 H) 7.54-7.66 (m, 1 H) 7.70 (s, 1 H) 7.78 (d, J = 5.81 Hz, 1 H) 8.09 (d, J = 7.33 Hz, 1 H) 8.46 (d, J = 7.83 Hz, 1 H) 8.92 (s, 1 H) |
| 188 | 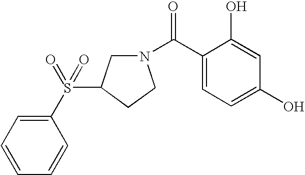 | 4-{[3- (phenylsulfonyl) pyrrolidin-1- yl]carbonyl}benzene- 1,3-diol | G2 | (400 MHz, DMSO-D6) δ ppm 2.11-2.25 (m, 2 H) 3.43-3.54 (m, 2 H) 3.60-3.72 (m, 1 H) 3.72-3.83 (m, 1 H) 4.11-4.25 (m, 1 H) 6.18-6.32 (m, 2 H) 7.00-7.12 (m, J = 8.34 Hz, 1 H) 7.62-7.73 (m, 2 H) 7.73-7.83 (m, J = 7.45, 7.45 Hz, 1 H) 7.82-7.95 (m, J = 6.32 Hz, 2 H) 10.19 (s, 2 H) |
| 189 | 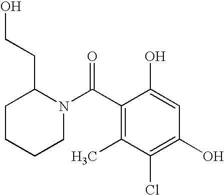 | 4-chloro-6-{[2-(2- hydroxyethyl)piperidin- 1-yl]carbonyl}-5- methylbenzene-1,3- diol | G9a | (400 MHz, DMSO-D6) δ ppm 1.28-1.62 (m, 3 H) 1.66-1.79 (m, 2 H) 1.80-1.95 (m, 2 H) 1.94-2.09 (m, 1 H) 2.20 (s, 3 H) 2.77-2.91 (m, 1 H) 3.08-3.21 (m, 3 H) 4.21-4.39 (m, 2 H) 5.74 (s, 1 H) 6.44 (s, 1 H) |
| 190 | 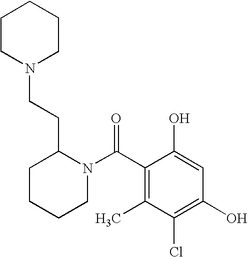 | 4-chloro-5-methyl-6- {[2-(2-piperidin-1- ylethyl)piperidin-1- yl]carbonyl}benzene- 1,3-diol | G9a | (400 MHz, DMSO-D6) δ ppm 1.20 (s, 1 H) 1.38 (dd, J = 12.13, 3.54 Hz, 1 H) 1.50-1.73 (m, 8 H) 1.77-1.91 (m, J = 13.64 Hz, 3 H) 2.07-2.17 (m, 3 H) 2.23 (d, J = 11.62 Hz, 1 H) 2.79-2.96 (m, 2 H) 2.98-3.15 (m, 4 H) 3.19 (t, J = 13.64 Hz, 2 H) 4.78 (s, 1 H) 6.42 (s, 1 H) 9.93 (s, 1 H) 10.07-10.17 (m, 1 H) |
| 191 | 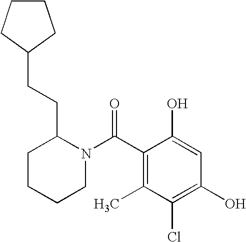 | 4-chloro-6-{[2-(2- cyclopentylethyl) piperidin- 1-yl]carbonyl}-5- methylbenzene-1,3- diol | G9a | (400 MHz, DMSO-D6) δ ppm 0.84-1.13 (m, 3 H) 1.17-1.36 (m, 2 H) 1.34-1.68 (m, 10 H) 1.66-1.85 (m, 2 H) 2.00-2.16 (m, 3 H) 2.58-2.75 (m, 1 H) 2.84-3.06 (m, 1 H) 3.07-3.25 (m, 1 H) 4.44 (d, J = 3.79 Hz, 1 H) 4.70 (d, J = 4.55 Hz, 1 H) 6.32-6.48 (m, 1 H) 9.54 (s, 1 H) 9.98 (s, 1 H) |
| 192 | 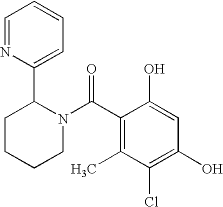 | 4-chloro-5-methyl-6- [(2-pyridin-2- ylpiperidin-1- yl)carbonyl]benzene- 1,3-diol | G9a | (400 MHz, DMSO-D6) δ ppm 1.93-1.99 (m, 3 H) 1.99-2.09 (m, 4 H) 2.13-2.24 (m, 1 H) 3.09-3.28 (m, 2 H) 3.97-4.21 (m, 2 H) 6.67 (s, 1 H) 7.01-7.17 (m, 1 H) 7.22 (dd, J = 9.60, 6.57 Hz, 1 H) 7.85-8.00 (m, 2 H) 10.64 (s, 1 H) 11.00 (s, 1 H) |
| 193 | 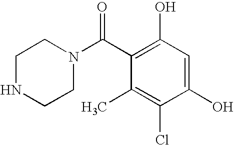 | 4-chloro-5-methyl-6- (piperazin-1- ylcarbonyl)benzene- 1,3-diol | G9a | (400 MHz, DMSO-D6) δ ppm 2.09 (s, 3 H) 2.94-3.07 (m, 2 H) 3.13 (d, J = 4.04 Hz, 2 H) 3.66-3.79 (m, 2 H) 3.78-3.93 (m, 2 H) 6.43 (s, 1 H) 8.87 (s, 1 H) 9.87 (s, 1 H) 10.20 (s, 1 H) |
| 194 | 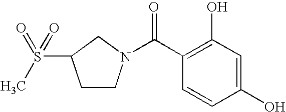 | 4-{[3- (methylsulfonyl) pyrrolidin-1- yl]carbonyl}benzene- 1,3-diol | G2 | (400 MHz, DMSO-D6) δ ppm 2.20-2.31 (m, 2 H) 2.72 (s, 3 H) 3.13 (dd, J = 7.33, 4.29 Hz, 1 H) 3.57-3.68 (m, 2 H) 3.74-3.85 (m, J = 6.82 Hz, 1 H) 3.90-4.03 (m, 1 H) 6.19-6.32 (m, 1 H) 7.11 (d, J = 8.34 Hz, 1 H) 7.94 (s, 1 H) 9.73 (s, 1 H) 10.47 (s, 1 H) |
| 195 | 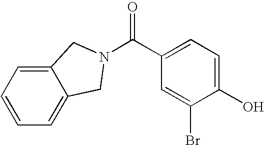 | 2-bromo-4-(1,3- dihydro-2H-isoindol-2- ylcarbonyl)phenol | G2 | (400 MHz, DMSO-D6) δ ppm 4.76-4.93 (m, 4 H) 7.01 (d, J = 8.08 Hz, 1 H) 7.19-7.43 (m, 4 H) 7.49 (s, 1 H) 7.75 (s, 1 H) |
| 196 | 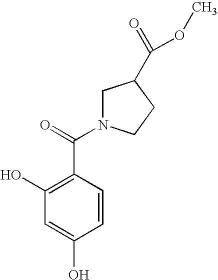 | methyl 1-(2,4- dihydroxybenzoyl) pyrrolidine-3-carboxylate | G2 | (400 MHz, MeOD) δ ppm 1.97-2.15 (m, 2 H) 3.01-3.13 (m, 1 H) 3.17-3.21 (m, 1 H) 3.45-3.54 (m, 1 H) 3.58 (s, 3 H) 3.65 (d, J = 7.33 Hz, 2 H) 6.12-6.24 (m, 2 H) 7.06 (d, J = 8.34 Hz, 1 H) |
| 197 | 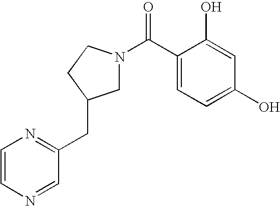 | 4-{[3-(pyrazin-2- ylmethyl)pyrrolidin-1- yl]carbonyl}benzene- 1,3-diol | G2 | (400 MHz, DMSO-D6) δ ppm 1.61 (s, 1 H) 1.90 (d, J = 1.26 Hz, 2 H) 2.87 (s, 3 H) 3.49-3.61 (m, 3 H) 6.14-6.29 (m, 2 H) 7.17 (s, 1 H) 8.42-8.49 (m, 1 H) 8.57 (s, 2 H) 9.76 (s, 1 H) 10.88 (s, 1 H) |
| 198 | 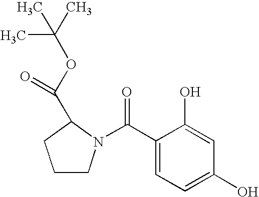 | tert-butyl 1-(2,4- dihydroxybenzoyl)-D- prolinate | G2 | (400 MHz, MeOD) δ ppm 1.37 (s, 9 H) 1.75-2.00 (m, 4 H) 2.16-2.31 (m, 1 H) 3.59 (d, J = 6.32 Hz, 2 H) 4.36 (s, 1 H) 6.12-6.28 (m, 2 H) 7.13 (d, J = 8.34 Hz, 1 H) |
| 199 | 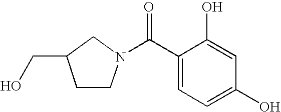 | 4-{[3- (hydroxymethyl) pyrrolidin-1- yl]carbonyl}benzene- 1,3-diol | G2 | (400 MHz, MeOD) δ ppm 1.55-1.72 (m, 1 H) 1.92 (dd, J = 11.62, 5.31 Hz, 1 H) 2.32 (d, J = 6.82 Hz, 1 H) 3.22-3.33 (m, J = 11.49, 7.45 Hz, 1 H) 3.35-3.70 (m, 5 H) 6.15-6.28 (m, 2 H) 7.09 (d, J = 8.08 Hz, 1 H) |
| 200 | 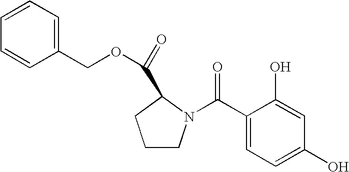 | benzyl 1-(2,4- dihydroxybenzoyl)-L- prolinate | G2 | (400 MHz, MeOD) δ ppm 1.87 (s, 3 H) 2.16-2.29 (m, 1 H) 3.51-3.66 (m, 2 H) 4.51 (dd, J = 8.46, 4.17 Hz, 1 H) 5.07 (s, 2 H) 6.19 (s, 2 H) 7.10 (s, 1 H) 7.17-7.32 (m, 5 H) |
| 201 | 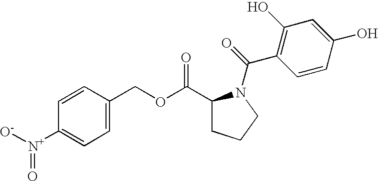 | 4-nitrobenzyl 1-(2,4- dihydroxybenzoyl)-L- prolinate | G1 | (500 MHz, deuterium oxide) δ ppm 1.86 (d, J = 4.94 Hz, 4 H) 2.24 (s, 1 H) 3.55 (s, 1 H) 4.50 (s, 1 H) 5.25 (s, 2 H) 6.24 (s, 3 H) 7.14 (s, 1 H) 7.62 (s, 2 H) 8.17 (s, 2 H) 9.77 (s, 1 H) |
| 202 | 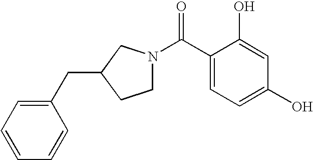 | 4-[(3-benzylpyrrolidin- 1- yl)carbonyl]benzene- 1,3-diol | G1 | (500 MHz, deuterium oxide) δ ppm 1.53 (s, 2 H) 1.84 (s, 2 H) 2.63 (s, 1 H) 6.17-6.21 (m, 3 H) 7.14 (s, 6 H) 7.23 (s, 3 H) 9.68 (s, 1 H) |
| 203 | 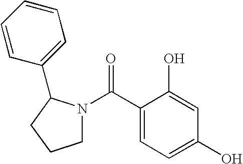 | 4-[(2-phenylpyrrolidin- 1- yl)carbonyl]benzene- 1,3-diol | G1 | (500 MHz, deuterium oxide) δ ppm 1.71 (s, 3 H) 2.64 (s, 1 H) 2.92 (s, 1 H) 3.77 (s, 1 H) 5.07 (s, 1 H) 6.20 (s, 1 H) 6.32 (s, 1 H) 6.40 (s, 1 H) 7.04 (s, 2 H) 7.11 (s, 1 H) 7.14 (s, 1 H) 7.22 (s, 4 H) 7.34 (s, 1 H) 7.54 (s, 1 H) 7.73 (s, 1 H) 10.23 (s, 1 H) |
| 204 | 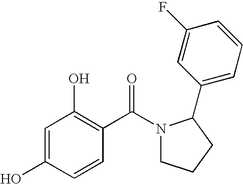 | 4-{[2-(3- fluorophenyl) pyrrolidin-1- yl]carbonyl}benzene- 1,3-diol | G1 | (500 MHz, deuterium oxide) δ ppm 1.68 (s, 1 H) 1.76 (s, 3 H) 2.02 (s, 1 H) 2.28 (s, 1 H) 3.75 (s, 1 H) 5.10 (s, 1 H) 6.24 (s, 2 H) 6.96 (s, 2 H) 7.11 (s, 2 H) 7.28 (s, 1 H) |
| 205 | 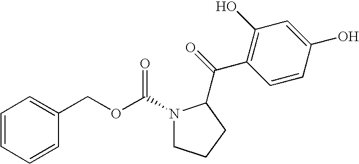 | benzyl 1-(2,4- dihydroxybenzoyl)-D- prolinate | G1 | (500 MHz, deuterium oxide) δ ppm 1.82 (s, 4 H) 2.21 (s, 1 H) 3.53 (s, 1 H) 4.47 (d, J = 4.67 Hz, 1 H) 5.09 (s, 2 H) 6.23 (s, 3 H) 7.14 (s, 1 H) 7.31 (s, 5 H) |
| 206 | 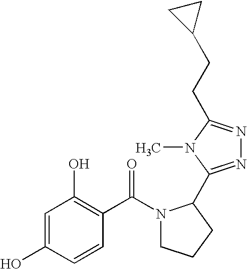 | 4-[(2-{5- [(cyclopropylmethyl) thio]-4-methyl-4H-1,2,4- triazol-3-yl}pyrrolidin- 1- yl)carbonyl]benzene- 1,3-diol | G1 | (500 MHz, deuterium oxide) δ ppm 0.14 (d, J = 4.12 Hz, 3 H) 0.45 (d, J = 6.87 Hz, 3 H) 1.02 (s, 1 H) 1.86 (s, 2 H) 1.99 (s, 1 H) 2.05 (d, J = 6.87 Hz, 2 H) 2.24 (s, 2 H) 2.94 (s, 3 H) 3.64 (s, 1 H) 5.19 (s, 1 H) 6.21 (s, 3 H) 7.10 (s, 1 H) |
| 207 | 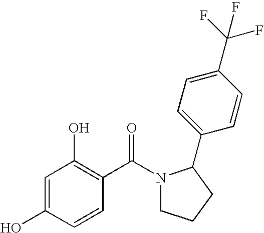 | 4-({2-[4- (trifluoromethyl)phenyl] pyrrolidin-1- yl}carbonyl)benzene- 1,3-diol | G1 | (500 MHz, deuterium oxide) δ ppm 1.67 (s, 1 H) 1.79 (s, 3 H) 2.02 (s, 1 H) 3.54 (s, 1 H) 3.78 (s, 1 H) 5.14 (s, 1 H) 6.24 (s, 2 H) 7.19 (s, 1 H) 7.29 (s, 1 H) 7.49 (s, 2 H) 7.60 (s, 2 H) |
| 208 | 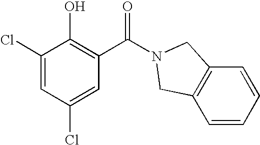 | 2,4-dichloro-6-(1,3- dihydro-2H-isoindol-2- ylcarbonyl)phenol | G1 | (400 MHz, DMSO-D6) δ ppm 4.61 (s, 2 H) 4.81 (s, 2 H) 7.24-7.33 (m, 3 H) 7.33-7.44 (m, 2 H) 7.62 (d, J = 2.53 Hz, 1 H) 10.36 (s, 1 H) |
| 209 | 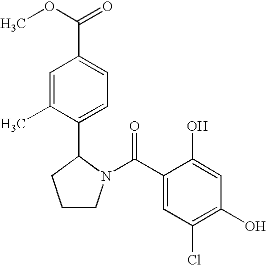 | methyl 4-[1-(5-chloro- 2,4- dihydroxybenzoyl) pyrrolidin-2-yl]-3- methylbenzoate | G14a | (400 MHz, DMSO-D6) δ ppm 1.55-1.67 (m, 1 H) 1.78-1.93 (m, 2 H) 2.07 (s, 1 H) 2.41 (s, 3 H) 3.45-3.61 (m, 1 H) 3.71-3.80 (m, J = 7.33 Hz, 1 H) 3.80-3.87 (m, 3 H) 5.25 (t, J = 6.69 Hz, 1 H) 7.23 (s, 1 H) 7.44 (d, J = 8.08 Hz, 1 H) 7.71 (d, J = 7.83 Hz, 1 H) 7.76 (s, 1 H) 10.48 (s, 2 H) |
| 210 | 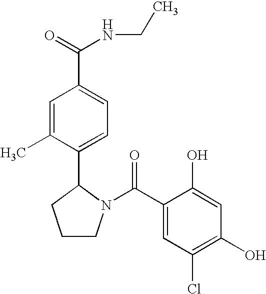 | 4-(1-(5-chloro-2,4- dihydroxybenzoyl) pyrrolidin-2-yl)- N-ethyl-3- methylbenzamide | G14a | (400 MHz, DMSO-D6) δ ppm 1.09 (t, J = 7.20 Hz, 3 H) 1.52-1.66 (m, J = 6.57 Hz, 1 H) 1.82 (d, J = 6.57 Hz, 2 H) 2.04-2.11 (m, 1 H) 2.33-2.42 (m, 3 H) 3.20-3.28 (m, 2 H) 3.47-3.56 (m, 1 H) 3.71-3.85 (m, 1 H) 5.16-5.30 (m, 1 H) 6.54 (s, 1 H) 7.21 (s, 1 H) 7.35 (d, J = 7.83 Hz, 1 H) 7.55 (d, J = 7.33 Hz, 1 H) 7.60 (s, 1 H) 10.47 (s, 2 H) |
| 211 | 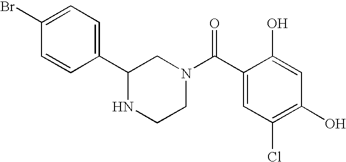 | 4-{[3-(4- bromophenyl)piperazin- 1-yl]carbonyl}-6- chlorobenzene-1,3- diol | G2 | (300 MHz, DMSO-D6) δ ppm 2.57-2.76 (m, 3 H) 2.83-3.01 (m, 3 H) 3.57-3.69 (m, 1 H) 6.51 (s, 1 H) 7.04 (s, 1 H) 7.21-7.41 (m, 2 H) 7.51 (d, J = 8.29 Hz, 2 H) 9.95 (s, 1 H) 10.31 (s, 1 H) |
| 212 | 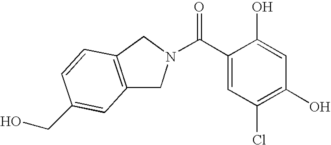 | 4-chloro-6-{[5- (hydroxymethyl)-1,3- dihydro-2H-isoindol-2- yl]carbonyl}benzene- 1,3-diol | G2 | (400 MHz, DMSO-D6) δ ppm 4.47 (s, 2 H) 4.61-4.86 (m, J = 26.02 Hz, 4 H) 5.19 (s, 1 H) 6.58 (s, 1 H) 7.10-7.40 (m, 4 H) 10.31 (s, 1 H) 10.41 (s, 1 H) |
| 213 | 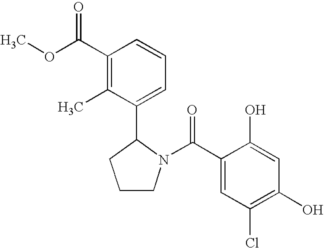 | methyl 3-[1-(5-chloro- 2,4- dihydroxybenzoyl) pyrrolidin-2-yl]-2- methylbenzoate | G14b | (400 MHz, DMSO-D6) δ ppm 1.56 (dd, J = 12.25, 5.94 Hz, 1 H) 1.69-1.99 (m, 2 H) 2.36-2.47 (m, 2 H) 3.49 (d, J = 10.11 Hz, 1 H) 3.73-3.81 (m, 1 H) 3.82 (s, 3 H) 5.23-5.42 (m, 1 H) 6.50-6.69 (m, 1 H) 7.08-7.28 (m, 2 H) 7.51 (d, J = 7.83 Hz, 2 H) 10.47 (s, 2 H). |
| 214 | 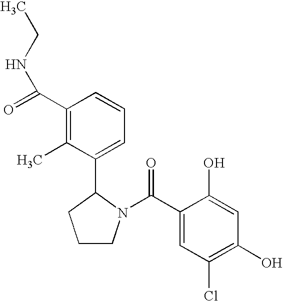 | 3-[1-(5-chloro-2,4- dihydroxybenzoyl) pyrrolidin-2-yl]- N-ethyl-2- methylbenzamide | G14b | (400 MHz, DMSO-D6) δ ppm 1.10 (t, J = 7.20 Hz, 3 H) 1.59 (d, J = 5.81 Hz, 1 H) 1.75-1.92 (m, 2 H) 2.30 (s, 3 H) 2.39 (d, J = 12.38 Hz, 1 H) 3.18-3.29 (m, 2 H) 3.44-3.58 (m, 1 H) 3.72-3.94 (m, 1 H) 5.29 (dd, J = 7.58, 5.05 Hz, 1 H) 6.55 (s, 1 H) 7.04-7.11 (m, 1 H) 7.15 (t, J = 7.45 Hz, 1 H) 7.23 (s, 1 H) 7.36 (d, J = 7.33 Hz, 1 H) 8.16-8.44 (m, 1 H) 10.50 (s, 2 H) |

### Example 215

### General Procedure G14a

### General Procedure for Preparation of Compound G1

### Synthesis of Compound 2

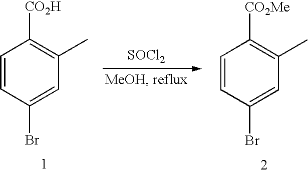

To a solution of compound 1 (208.8 g, 0.97 mol) in MeOH (1200 mL) was added dropwise SOCl₂ (210 mL, 2.91 mol) at 0° C. The mixture was heated to reflux for 60 hours. TLC (petroleum ether:ethyl acetate=10:1) showed the reaction was complete, then the mixture was concentrated in vacuo. The residue was purified by column chromatography (eluting with petroleum ether) to afford compound 2 (179 g, 81%) as a yellow oil.

¹H NMR (400 MHz, CDCl₃) δ 7.75-7.77 (d, J=8.0 Hz, 1H), 7.34-7.39 (m, 2H), 3.87 (s, 3H), 2.56 (s, 3H).

### Synthesis of Compound 3

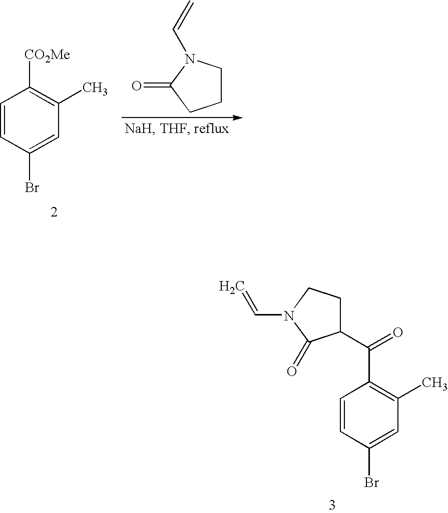

Compound 2 (50 g, 0.219 mol) and 1-vinyl-2-pyrrolidinone (27.0 g, 0.241 mol) were dissolved in anhydrous THF (200 mL). The solution was added slowly to a gently refluxing mixture of sodium hydride (13.0 g, 60% in mineral oil, 0.307 mol) and THF (100 mL). After the addition, the mixture was stirred at reflux for an additional 2 hours. TLC (petroleum ether:ethyl acetate=10:1) showed the reaction was complete, then the mixture was cooled and the excess sodium hydride was quenched by the addition of saturated aq. ammonium chloride. The mixture was concentrated in vacuo. The residue was extracted with ethyl acetate (200 mL×3). The combined organic layers were washed with brine, dried over anhydrous sodium sulfate and concentrated in vacuo to give the crude product 3, which was used in the next step without further purification.

Synthesis of Compound 4

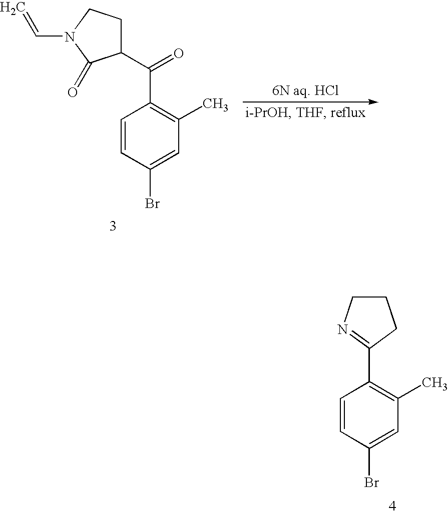

Compound 3 (0.526 mol) was dissolved in i-PrOH/THF (360 mL/180 mL). Aqueous 6N HCl (1300 mL) was added and the mixture was stirred at reflux for 14 hours. TLC (petroleum ether:ethyl acetate=10:1) showed the reaction was complete, then the mixture was cooled and basified to pH>10 with K₂CO₃. The alkaline aqueous layer was then extracted with ethyl acetate (1000 mL×3). The combined organic layers were washed with brine, dried over anhydrous sodium sulfate and concentrated in vacuo to give the crude product, which was purified by column chromatography (eluting with petroleum ether) to afford compound 4 (70 g, yield: 56% for two steps) as a brown oil. ¹H NMR (400 MHz, CDCl₃): δ 7.28-7.38 (m, 3H), 4.05-4.09 (t, J=8.0 Hz, 2H), 2.85-2.90 (m, 2H), 2.48 (s, 3H), 1.95-2.04 (m, 2H).

### Synthesis of Compound 5

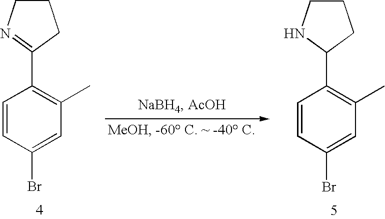

To a solution of compound 4 (38 g, 0.16 mol) in methanol/acetic acid (370 mL, 80:20) was added NaBH₄ (13.4 g, 0.352 mol) in portions over 30 minutes with vigorous stirring at −60° C. The mixture was stirred at −40° C. for 2 hours, TLC (petroleum ether:ethyl acetate=10:1) showed the reaction was complete. The mixture was warmed to room temperature and evaporated to remove most of the solvent. The residue was taken up with 500 mL of H₂O, and the mixture was basified to pH 9 with K₂CO₃. The resulting mixture was extracted with CH₂Cl₂ (200 mL×3). The combined extracts were washed with brine, dried over anhydrous potassium carbonate and concentrated in vacuo to give the crude product 5, which was used in the next step without further purification. MS (ESI) 240 (Z⁺).

### Synthesis of Compound 6

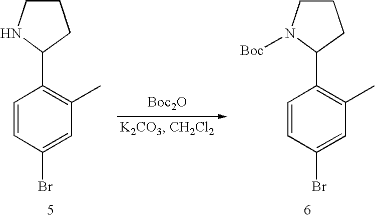

A mixture of compound 5 (0.16 mol) and K₂CO₃ (33.0 g, 0.24 mol) in dry CH₂Cl₂ (300 mL) was chilled to 5° C., then Boc₂O (43.0 g, 0.24 mol) was added dropwise. The reaction mixture was stirred at room temperature overnight. TLC (petroleum ether:ethyl acetate=10:1) showed the reaction was complete, then the mixture was concentrated in vacuo. The residue was taken up with 500 mL ethyl acetate and 200 mL water. The organic layer was separated and the aqueous layer was extracted with ethyl acetate (150 mL×2). The combined organic layers were washed with brine, dried over anhydrous sodium sulfate and concentrated in vacuo to give the crude product, which was purified by column chromatography (petroleum ether:ethyl acetate=1:0˜10:1) to afford compound 6 (44 g, yield: 81% for two steps) as a white solid.

¹H NMR (400 MHz, CDCl₃) δ 7.24-7.36 (d, J=8.0 Hz, 2H), 6.88-6.94 (m, 1H), 4.87-5.01 (m, 1H), 3.47-3.61 (m, 2H), 2.27 (s, 3H), 1.64-1.86 (m, 4H), 1.46 (br, 3H), 1.11 (br, 6H).

### Synthesis of Compound G1

### tert-butyl 2-[4-(methoxycarbonyl)-2-methylphenyl]pyrrolidine-1-carboxylate

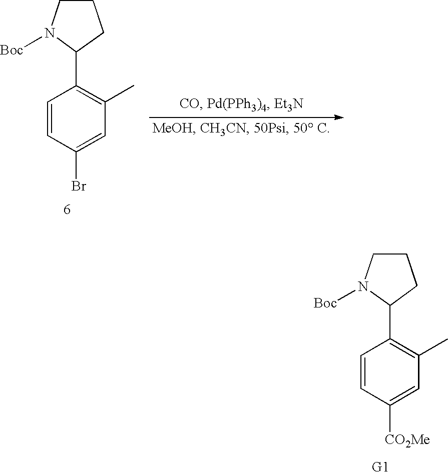

A mixture of compound 6 (21 g, 0.061 mol), Pd(PPh₃)₄ (2.1 g, 0.002 mol), Et₃N (13 mL, 0.093 mol), 105 mL of acetonitrile and 50 mL of methanol was stirred under 100 psi of carbon monoxide at 65° C. for 62 hours. The reaction mixture was filtered and concentrated in vacuo. The residue was dissolved in 200 mL of ethyl acetate, then washed with water and brine, dried over anhydrous sodium sulfate and concentrated in vacuo to give the crude product, which was purified by column chromatography (petroleum ether:ethyl acetate=1:0˜30:1) to afford compound G1 (4.9 g, 65%) as a brown oil. ¹H NMR (400 MHz, CDCl₃): δ 7.79-7.81 (d, J=8.0 Hz, 2H), 7.14-7.16 (m, 1H), 4.95-5.16 (m, 1H), 3.89 (s, 3H), 3.50-3.58 (m, 2H), 2.28-2.37 (m, 4H), 1.86-1.92 (m, 2H), 1.68-1.70 (m, 1H), 1.44 (br, 3H), 1.13 (br, 6H).

### Synthesis of Compound H1

### methyl 3-methyl-4-pyrrolidin-2-ylbenzoate

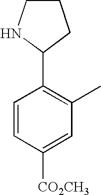

Hydrogen chloride (16 mL, 64 mmol; 4 M in dioxane) was added to a solution of compound G1 (2 g, 6.26 mmole) in DCM (30 mL). The reaction was stirred at room temperature for 12 hours. The reaction mixture was evaporated to give a white solid residue. The residue was used for the next step reaction without further purification.

### Compound H1

### Methyl 4-{1-[5-chloro-2,4-bis(methoxymethoxy)benzoyl]pyrrolidin-2-yl}-3-methylbenzoate

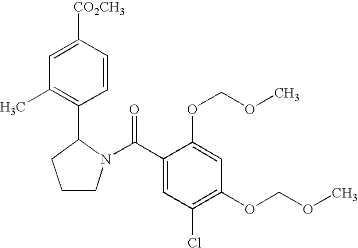

Methyl 3-methyl-4-pyrrolidin-2-ylbenzoate (6.26 mmol) was added to a solution of 5-chloro-2,4-bis(methoxymethoxy)benzoic acid (1.75 g, 6.26 mmol), 4-methylmorpholine (14 mL, 125.2 mmol), N-(3-dimethylaminopropyl)-N′-ethylcarbodiimide hydrochloride (2.5 g, 13 mmol), and 1-hydroxy benzotriazole 1.76 g, 13 mmol) in 25 mL of DMF under a nitrogen atmosphere. The reaction was allowed to stir at room temperature for 12 hours. H₂O (100 mL) was added to the reaction mixture to quench the reaction. EtOAc (2×200 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 50→60% EtOAc in hexanes to give the desired product (3.4 g, quantitative yield).

### Synthesis of Compound I1

### methyl 4-[1-(5-chloro-2,4-dihydroxybenzoyl)pyrrolidin-2-yl]-3-methylbenzoate

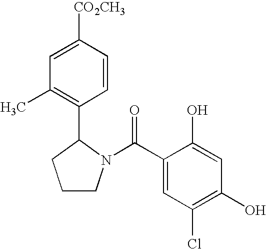

Hydrogen chloride (1.2 mL, 5 mmol; 4 M in dioxane) was added to a solution of compound H1 (116.2 mg, 0.24 mmole) in MeOH (3 mL). The reaction was stirred at room temperature for 12 hours. The reaction mixture neutralized with sat. NaHCO₃ (aq) and then extracted with EtOAc (2×50 mL). The combined organic layers were dried, filtered, and evaporated to give white solid (30 mg, 32.7% yield). ¹H NMR (400 MHz, DMSO-D6) δ ppm 1.55-1.67 (m, 1H) 1.78-1.93 (m, 2H) 2.07 (s, 1H) 2.41 (s, 3H) 3.45-3.61 (m, 1H) 3.71-3.80 (m, J=7.33 Hz, 1H) 3.80-3.87 (m, 3H) 5.25 (t, J=6.69 Hz, 1H) 7.23 (s, 1H) 7.44 (d, J=8.08 Hz, 1H) 7.71 (d, J=7.83 Hz, 1H) 7.76 (s, 1H) 10.48 (s, 2H).

### Synthesis of Compound J1

### 4-{1-[5-chloro-2,4-bis(methoxymethoxy)benzoyl]pyrrolidin-2-yl}-3-methylbenzoic acid

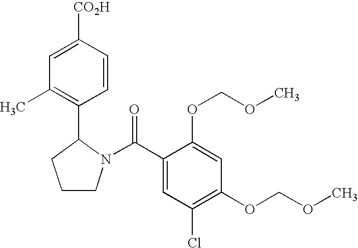

Lithium hydroxide hydrate (6 g, 142.3 mmol) was added to a solution of compound I1 (3.4 g, 7.1 mmol) in H₂O (20 mL) and MeOH (15 mL). The reaction mixture was heated to 40° C. for 12 hours. The mixture was evaporated and neutralized by HOAc—NaOAc buffer solution. EtOAc (2×200 mL) was added to extract the aqueous solution. The combined organic layers were dried, filtered, and concentrated to give the desired product as a white solid (3.03 g, 91.1% yield). ¹H NMR (400 MHz, DMSO-D6) δ ppm 1.59-1.68 (m, 2H) 1.77-1.86 (m, 2H) 2.39 (s, 3H) 3.31-3.38 (m, 6H) 3.40 (s, 2H) 3.43 (s, 2H) 3.53-3.66 (m, 2H) 5.26 (dd, J=7.71, 4.67 Hz, 1H) 5.32 (s, 3H) 7.34 (s, 1H) 7.39 (d, J=7.83 Hz, 1H) 7.74 (s, 2H) 7.76 (s, 1H) 12.80 (s, 1H).

### Synthesis of 4-{1-[5-chloro-2,4-bis(methoxymethoxy)benzoyl]pyrrolidin-2-yl}-N-ethyl-3-methylbenzamide

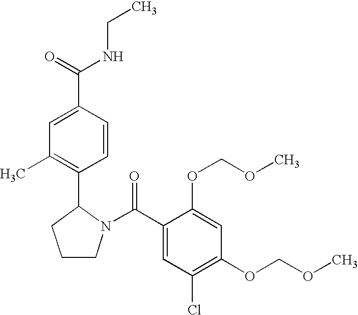

Ethylamine (1 mL, 0.7 mmol) was added to a solution of compound J1 (150 mg, 0.32 mmole), 4-methylmorpholine (0.8 ml, 7 mmol), N-(3-dimethylaminopropyl)-N′-ethylcarbodiimide hydrochloride (140 mg, 0.7 mmol), and 1-hydroxy benzotriazole (100 mg, 0.7 mmol) in 4 mL of DMF under a nitrogen atmosphere. The reaction was allowed to stir at room temperature for 12 hours. H₂O (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 0→20% MeOH in EtOAc to give the desired product (100 mg, 63.6% yield).

### Synthesis of 4-[1-(5-chloro-2,4-dihydroxybenzoyl)pyrrolidin-2-yl]-N-ethyl-3-methylbenzamide

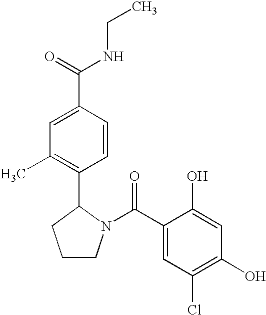

Hydrogen chloride (1 mL, 4 mmol; 4 M in dioxane) was added to a solution of 4-{1-[5-chloro-2,4-bis(methoxymethoxy)benzoyl]pyrrolidin-2-yl}-N-ethyl-3-methylbenzamide (100 mg, 0.2 mmole) in MeOH (3 mL). The reaction was stirred at room temperature for 12 hours. The reaction mixture neutralized with saturated NaHCO₃ (aq) and then extracted with EtOAc (2×50 mL). The combined organic layers were dried, filtered, and evaporated to give white solid (72 mg, 89.5% yield). ¹H NMR (400 MHz, DMSO-D6) δ ppm 1.09 (t, J=7.20 Hz, 3H) 1.52-1.66 (m, J=6.57 Hz, 1H) 1.82 (d, J=6.57 Hz, 2H) 2.04-2.11 (m, 1H) 2.33-2.42 (m, 3H) 3.20-3.28 (m, 2H) 3.47-3.56 (m, 1H) 3.71-3.85 (m, 1H) 5.16-5.30 (m, 1H) 6.54 (s, 1H) 7.21 (s, 1H) 7.35 (d, J=7.83 Hz, 1H) 7.55 (d, J=7.33 Hz, 1H) 7.60 (s, 1H) 10.47 (s, 2H). Anal. Calcd for C₂₁H₂₃ClN₂O₄: C, 62.61; H, 5.75; N, 6.95. Found: C, 62.21; H, 5.79; N, 6.75.

### Example 216

### General Procedure G14b

### General Procedure for Preparation of Compound G2

### Synthesis of Compound 2

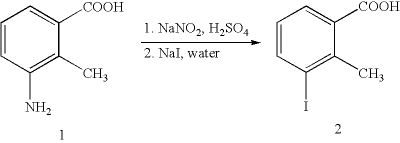

To a solution of compound 1 (50.0 g, 0.331 mol) in 20% aqueous H₂SO₄ (350 mL) was added dropwise a solution of NaNO₂ (24.0 g, 0.348 mol) in H₂O (100 mL) at 0° C. The mixture was stirred at 0° C. for 1 hour. A solution of NaI (64.0 g, 0.424 mol) in water (100 mL) was added dropwise into the mixture at 0-5° C. The mixture was stirred at room temperature for 12 hours and filtered. The filter cake was washed with water to pH 7 and dried to afford crude compound 2 (63 g, 72%) as a black solid, which was used in the next step without further purification.

### Synthesis of Compound 3

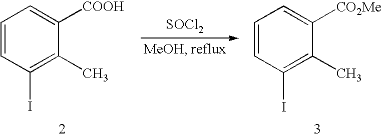

To a solution of compound 2 (63.0 g, 0.24 mol) in MeOH (300 mL) was added dropwise a freshly prepared solution of SOCl₂ (52 mL, 0.72 mol) in MeOH (100 mL) at 0° C. The mixture was heated to reflux for 3 hours. TLC (petroleum ether:ethyl acetate=10:1) showed the reaction was complete, then the mixture was concentrated in vacuo. The residue was purified by column chromatography (petroleum ether) to afford compound 3 (60 g, 92%) as brown oil.

¹H NMR (400 MHz, CDCl₃): δ 7.94-7.96 (d, J=8.0 Hz, 1H), 7.71-7.73 (d, J=8.0 Hz, 1H), 6.87-6.91 (t, J=8.0 Hz, 1H), 3.88 (s, 3H), 2.65 (s, 3H).

### Synthesis of Compound 4

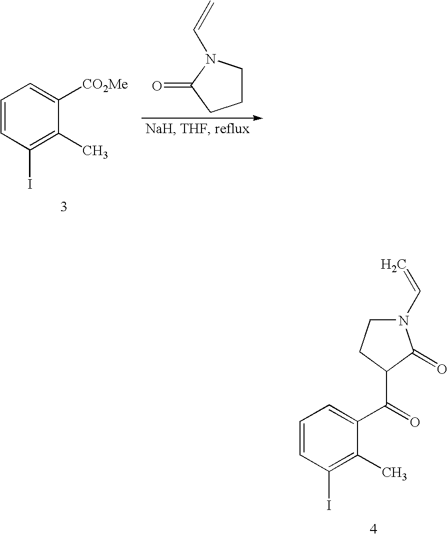

A solution of compound 3 (60 g, 0.217 mol) and 1-vinyl-2-pyrrolidinone (26.5 g, 0.239 mol) in anhydrous THF (200 mL) was added slowly to a gently refluxing suspension of sodium hydride (12.2 g, 60% in mineral oil, 0.304 mol) in THF (100 mL). After the addition, the mixture was stirred at reflux for additional 3 hours. TLC (petroleum ether:ethyl acetate=10:1) showed the reaction was complete. The mixture was cooled and the excess sodium hydride was quenched with saturated aqueous ammonium chloride. The mixture was concentrated in vacuo to remove THF. The residue was extracted with ethyl acetate (600 mL×2). The combined organic layers were washed with brine, dried over anhydrous sodium sulfate and concentrated in vacuo to give crude product 4, which was used in the next step without further purification.

### Synthesis of Compound 5

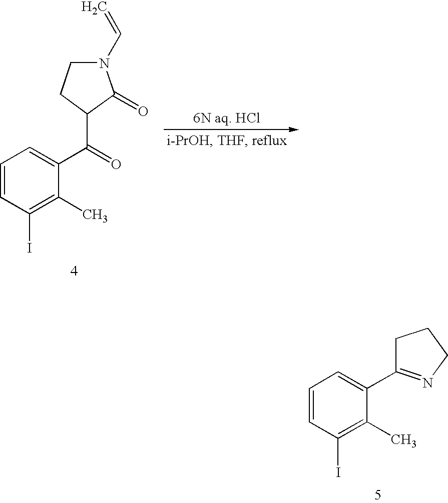

To a solution of compound 4 (crude product from H01707-042-1, 0.081 mol) in i-PrOH/THF (162 mL/81 mL) was added 6 N aq. HCl (450 mL). The mixture was stirred at reflux for 14 hours. TLC (petroleum ether:ethyl acetate=10:1) showed the reaction was complete. The mixture was cooled and extracted with ethyl acetate (200 mL×3). The majority of the product was remained in the acidic aqueous layer. The combined organic layers were washed with 3 N HCl (200 mL×2). The acidic aqueous layers were basified to pH>10 by gradual addition of K₂CO₃. The aqueous layer was extracted with ethyl acetate (600 mL×3). The combined organic layers were washed with brine, dried over anhydrous sodium sulfate and concentrated in vacuo to give crude product, which was purified by column chromatography (petroleum ether) to afford compound 5 (24 g, yield: 35% for two steps) as brown oil. ¹H NMR (400 MHz, CDCl₃): δ 7.82-7.84 (d, J=8.0 Hz, 1H), 7.26-7.28 (d, J=8.0 Hz, 1H), 6.85-6.89 (t, J=8.0 Hz, 1H), 4.05-4.09 (t, J=8.0 Hz, 2H), 2.80-2.84 (t, J=8.0 Hz, 2H), 2.51 (s, 3H), 1.97-2.05 (m, 2H).

### Synthesis of Compound 6

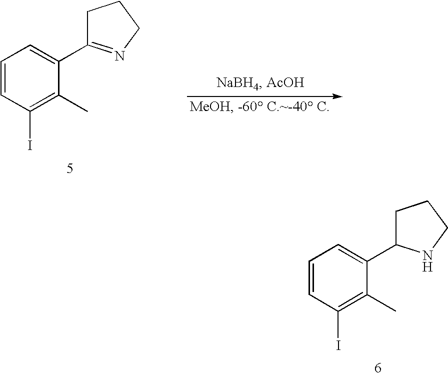

To a solution of compound 5 (23 g, 0.081 mol) in 190 mL of methanol/acetic acid (80:20) was added sodium borohydride (6.1 g, 0.161 mol) in portions at −60° C. over 30 minutes. During the addition, the temperature was allowed to keep at about −40° C. The mixture was stirred at −40° C. for 2 hours. TLC (petroleum ether:ethyl acetate=10:1) showed the reaction was complete. The mixture was warmed to room temperature and evaporated to remove most of the solvent. 450 mL of H₂O was added to the residue, and the mixture was basified to PH 9 with K₂CO₃. The resulting mixture was extracted with CH₂Cl₂ (250 mL×2). The combined organic layers were washed with brine, dried over anhydrous potassium carbonate and concentrated in vacuo to give crude product 6, which was used in the next step without further purification. MS (ESI) 288 (Z⁺).

### Synthesis of Compound 7

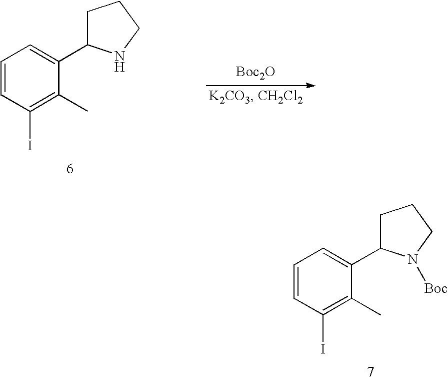

A suspension of compound 6 (0.081 mol) and K₂CO₃ (17.0 g, 0.121 mol) in dry CH₂Cl₂ (120 mL) was cooled to 5° C., then Boc₂O (21.9 g, 0.121 mol) was added dropwise. The reaction mixture was stirred at room temperature overnight. TLC (petroleum ether:ethyl acetate=10:1) showed the reaction was complete. The mixture was concentrated in vacuo. The residue was dissolved in 250 mL of ethyl acetate and 100 mL of water. The resulting mixture was extracted with ethyl acetate (100 mL×2). The combined organic layers were washed with brine, dried over anhydrous sodium sulfate and concentrated in vacuo to give crude product, which was purified by column chromatography (petroleum ether:ethyl acetate 1:0 to 10:1) to afford compound 7 (28 g, yield: 90% for two steps) as a white solid. ¹H NMR (400 MHz, CDCl₃): δ 7.70-7.72 (d, J=8.0 Hz, 1H), 7.04-7.06 (d, J=8.0 Hz, 1H), 6.81-6.85 (t, J=8.0 Hz, 1H), 4.98-5.18 (m, 1H), 3.48-3.68 (m, 2H), 2.46 (s, 3H), 2.22-2.38 (m, 1H), 1.80-1.90 (m, 2H), 1.62-1.74 (m, 1H), 1.45 (br, 3H), 1.17 (br, 6H).

### Synthesis of Compound G2

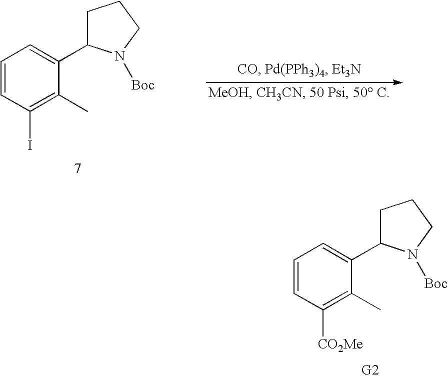

A mixture of compound 7 (26 g, 0.067 mol), Pd(PPh₃)₄ (2.4 g, 0.002 mol), Et₃N (14.3 mL, 0.1 mol), 110 mL of acetonitrile and 50 mL of methanol was stirred under 50 Psi of carbon monoxide at 50° C. for 8 hours. The reaction mixture was concentrated in vacuo. The residue was dissolved in 250 mL of ethyl acetate, then washed with water and brine, dried over anhydrous sodium sulfate and concentrated in vacuo to give crude product, which was purified by column chromatography (petroleum ether:ethyl acetate 30:1 to 10:1) to afford compound G2 (20 g, 93%) as a gray solid. ¹H NMR (300 MHz, CDCl₃): ¹H NMR δ 7.60-7.63 (d, J=9.0 Hz, 1H), 7.14-7.25 (m, 2H), 5.07-5.25 (m, 1H), 3.87 (s, 3H), 3.46-3.70 (m, 2H), 2.49 (s, 3H), 2.31-2.37 (m, 1H), 1.66-1.86 (m, 3H), 1.44 (br, 3H), 1.14 (br, 6H). MS (ESI) 342 (Z⁺+Na), 220 (Z⁺−100), 264 (Z⁺−56).

### Synthesis of Compound H2

### methyl 2-methyl-3-pyrrolidin-2-ylbenzoate

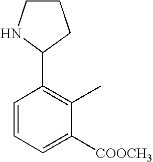

Hydrogen chloride (16 mL, 64 mmol; 4 M in dioxane) was added to a solution of compound G2 (2 g, 6.26 mmole) in DCM (30 mL). The reaction was stirred at room temperature for 12 hours. The reaction mixture was evaporated to give a white solid residue. The residue was used for the next reaction step without further purification.

### methyl 3-{1-[5-chloro-2,4-bis(methoxymethoxy)benzoyl]pyrrolidin-2-yl}-2-methylbenzoate

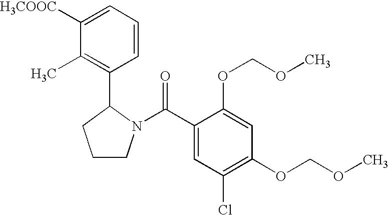

Methyl 2-methyl-3-pyrrolidin-2-ylbenzoate (6.26 mmol) was added to a solution of 5-chloro-2,4-bis(methoxymethoxy)benzoic acid (as prepared in Example 153) (1.75 g, 6.26 mmol), 4-methylmorpholine (14 ml, 125.2 mmol), N-(3-dimethylaminopropyl)-N′-ethylcarbodiimide hydrochloride (2.5 g, 13 mmol), and 1-hydroxy benzotriazole 1.76 g, 13 mmol) in 25 mL of DMF under a nitrogen atmosphere. The reaction was allowed to stir at room temperature for 12 hours. H₂O (100 mL) was added to the reaction mixture to quench the reaction. EtOAc (2×200 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 50→60% EtOAc in hexanes to give the desired product (3.2 g, quantitative yield).

### Synthesis of Compound I2

### methyl 3-[1-(5-chloro-2,4-dihydroxybenzoyl)pyrrolidin-2-yl]-2-methylbenzoate

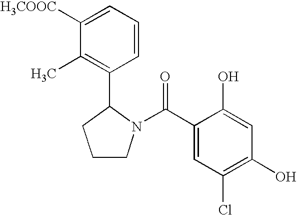

Hydrogen chloride (1 mL, 4 mmol; 4 M in dioxane) was added to a solution of compound H2 (100 mg, 0.24 mmole) in MeOH (3 mL). The reaction was stirred at room temperature for 12 hours. The reaction mixture neutralized with saturated NaHCO₃ (aq) and then extracted with EtOAc (2×50 mL). The combined organic layers were dried, filtered, and evaporated to give white solid (30 mg, 92.8% yield). ¹H NMR (400 MHz, DMSO-D6) δ ppm 1.56 (dd, J=12.25, 5.94 Hz, 1H) 1.69-1.99 (m, 2H) 2.36-2.47 (m, 2H) 3.49 (d, J=10.11 Hz, 1H) 3.73-3.81 (m, 1H) 3.82 (s, 3H) 5.23-5.42 (m, 1H) 6.50-6.69 (m, 1H) 7.08-7.28 (m, 2H) 7.51 (d, J=7.83 Hz, 2H) 10.47 (s, 2H). Anal. Calcd for C₂₀H₂₀ClNO₅.0.75H₂O: C, 59.56; H, 5.37; N, 3.47. Found: C, 59.47; H, 5.21; N, 3.48.

### Synthesis of Compound J2

### 3-{1-[5-chloro-2,4-bis(methoxymethoxy)benzoyl]pyrrolidin-2-yl}-2-methylbenzoic acid

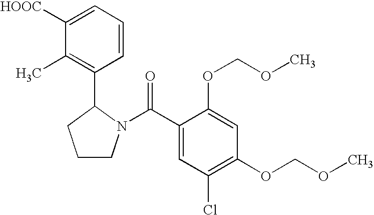

Lithium hydroxide hydrate (6 g, 142.3 mmol) was added to a solution of compound I2 (3.4 g, 7.1 mmol) in H₂O (20 mL) and MeOH (15 mL). The reaction mixture was heated to 40° C. for 12 hours. The mixture was evaporated and neutralized by HOAc—NaOAc buffer solution. EtOAc (2×200 mL) was added to extract the aqueous solution. The combined organic layers were dried, filtered, and concentrated to give the desired product as a white solid (3.03 g, 91.1% yield). ¹H NMR (400 MHz, DMSO-D6) δ ppm 1.56-1.70 (m, 1H) 1.76-1.86 (m, 2H) 2.32-2.44 (m, 1H) 3.39 (s, 3H) 3.43 (s, 3H) 3.76 (t, J=7.07 Hz, 2H) 5.11-5.24 (m, 1H) 5.27-5.42 (m, 4H) 7.06 (s, 1H) 7.26 (t, J=7.71 Hz, 1H) 7.34 (s, 1H) 7.46 (d, J=7.83 Hz, 1H) 7.52 (d, J=7.58 Hz, 1H) 12.86 (s, 1H). Anal. Calcd for C₂₃H₂₆ClNO₇: C, 59.55; H, 5.65; N, 3.02. Found: C, 59.18; H, 5.72; N, 3.16.

### 3-{1-[5-chloro-2,4-bis(methoxymethoxy)benzoyl]pyrrolidin-2-yl}-N-ethyl-2-methylbenzamide

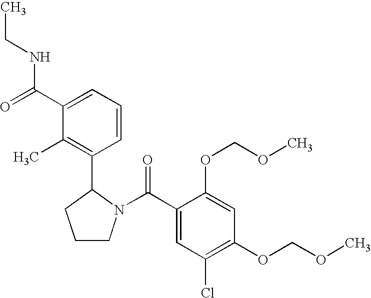

Ethylamine (1 mL, 0.7 mmol) was added to a solution of compound J2 (150 mg, 0.32 mmole), 4-methylmorpholine (0.8 ml, 7 mmol), N-(3-dimethylaminopropyl)-N′-ethylcarbodiimide hydrochloride (140 mg, 0.7 mmol), and 1-hydroxy benzotriazole (100 mg, 0.7 mmol) in 4 mL of DMF under a nitrogen atmosphere. The reaction was allowed to stir at room temperature for 12 hours. H₂O (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 80%→90% EtOAc in hexanes to give the desired product (72 mg, 45.8% yield).

### 3-[1-(5-chloro-2,4-dihydroxybenzoyl)pyrrolidin-2-yl]-N-ethyl-2-methylbenzamide

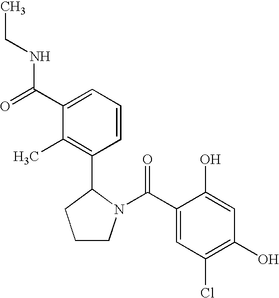

Hydrogen chloride (1 mL, 4 mmol; 4 M in dioxane) was added to a solution of 3-{1-[5-chloro-2,4-bis(methoxymethoxy)benzoyl]pyrrolidin-2-yl}-N-ethyl-2-methylbenzamide (72 mg, 0.147 mmole) in MeOH (2 mL). The reaction was stirred at room temperature for 12 hours. The reaction mixture neutralized with saturated NaHCO₃ (aq) and then extracted with EtOAc (2×50 mL). The combined organic layers were dried, filtered, and evaporated to give white solid (55.5 mg, 93.7% yield). ¹H NMR (400 MHz, DMSO-D6) δ ppm 1.10 (t, J=7.20 Hz, 3H) 1.59 (d, J=5.81 Hz, 1H) 1.75-1.92 (m, 2H) 2.30 (s, 3H) 2.39 (d, J=12.38 Hz, 1H) 3.18-3.29 (m, 2H) 3.44-3.58 (m, 1H) 3.72-3.94 (m, 1H) 5.29 (dd, J=7.58, 5.05 Hz, 1H) 6.55 (s, 1H) 7.04-7.11 (m, 1H) 7.15 (t, J=7.45 Hz, 1H) 7.23 (s, 1H) 7.36 (d, J=7.33 Hz, 1H) 8.16-8.44 (m, 1H) 10.50 (s, 2H).

Anal. Calcd for C₂₁H₂₃ClN₂O₄: C, 62.61; H, 5.75; N, 6.95. Found: C, 62.39; H, 5.949; N, 6.57.

### Example 217

### methyl 3-[1-(5-chloro-2,4-dihydroxybenzoyl)pyrrolidin-2-yl]-4-methylbenzoate

### General Procedure G14c

### Synthesis of Compound 2

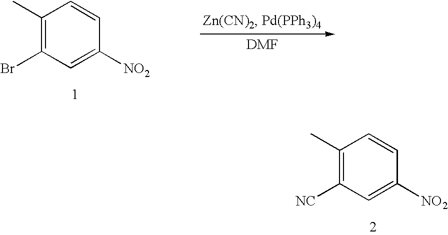

A mixture of compound 1 (250.0 g, 1.16 mol), Zn(CN)₂ (135.0 g, 1.16 mol) and Pd(PPh₃)₄ (25 g, 0.02 mol) in dry DMF (1500 mL) was stirred at 80° C. for 24 hours under N₂. TLC (petroleum ether:ethyl acetate=10:1) showed the reaction was complete, then the mixture was poured into toluene (1500 mL) and washed with 2 N NH₃H₂O (800 mL×2). The aqueous layer was extracted with toluene (800 mL×2). The combined organic layers were washed with brine, dried over anhydrous sodium sulfate and concentrated in vacuo to give crude product, which was purified by column chromatography (first petroleum ether, then petroleum ether:ethyl acetate 10:1) to afford compound 2 (25.5 g, yield: 42%) as a grey solid and recover compound 1 (170 g) as yellow solid.

¹H NMR (300 MHz, CDCl₃): δ 8.46-8.47 (d, J=3.0 Hz, 1H), 8.31-8.34 (dd, J=9.0 Hz, 1H), 7.53-7.56 (d, J=9.0 Hz, 1H), 2.68 (s, 3H).

### Synthesis of Compound 3

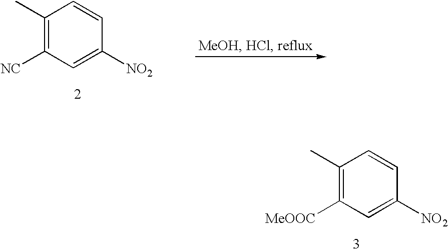

To a solution of compound 2 (64.0 g, 0.39 mol) in MeOH (1500 mL), H₂O (50 mL) and CH₂Cl₂ (500 mL) was purged with HCl gas till the solution was saturated. The mixture was left standing for 48 hours, then refluxed for 48 hours. TLC (petroleum ether:ethyl acetate=10:1) showed the reaction was complete, then the mixture was concentrated in vacuo to give crude product, which was purified by column chromatography (first petroleum ether, then petroleum ether:ethyl acetate 1:1) to afford compound 3 (33 g, yield: 82%) as a yellow solid and recover compound 2 (42 g) as a grey solid. ¹H NMR (400 MHz, CDCl₃): ¹H NMR δ 8.76 (S, 1H), 8.22-8.24 (dd, J=8.0 Hz, 1H), 7.42-7.44 (d, J=8.0 Hz, 1H), 3.95 (s, 3H), 2.71 (s, 3H).

### Synthesis of Compound 4

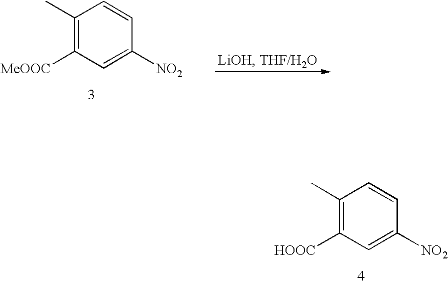

To a solution of compound 3 (75.0 g, 0.46 mol) in THF (500 mL) was added 1 N aq. LiOH (1 L, 0.92 mol), then the mixture was stirred at 30° C. for 4 hours. TLC (petroleum ether:ethyl acetate=10:1) showed the reaction was complete, then the mixture was concentrated in vacuo and the residue was extracted with ethyl acetate (200 mL×2). The combined organic layers were washed with brine, dried over anhydrous sodium sulfate and concentrated in vacuo to give compound 4 (67 g, 98%) as a brown solid., which was used in the next step without further purification. ¹H NMR (400 MHz, CDCl₃): δ 8.93 (S, 1H), 8.30-8.32 (d, J=8.0 Hz, 1H), 7.48-7.50 (d, J=8.0 Hz, 1H), 2.79 (s, 3H).

### Synthesis of Compound 5

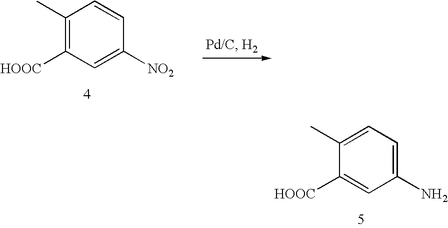

A mixture of compound 4 (30.0 g, 0.166 mol) and Pd/C (1.8 g) in dry methanol (1600 mL) was stirred under 50 Psi of H₂ at 40° C. for 3 to 4 hours. TLC (petroleum ether:ethyl acetate=1:2) showed the reaction was complete, then the mixture was filtered and the filtrate was concentrated in vacuo to give compound 5 (24 g, 96%) as a white solid. ¹H NMR (400 MHz, CDCl₃): δ 7.10 (S, 1H), 6.89-6.91 (d, J=8.0 Hz, 1H), 6.64-6.66 (dd, J=8.0 Hz, 1H), 2.31 (s, 3H).

### Synthesis of Compound 6

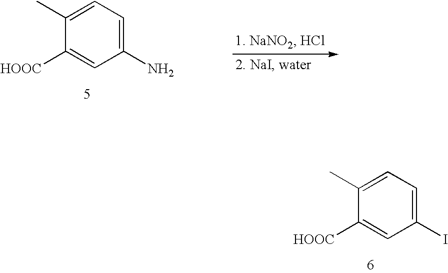

To a solution of compound 5 (50.0 g, 0.331 mol) in 12 N aqueous HCl (1310 mL) and ice-water (650 g) was added dropwise a solution of NaNO₂ (41 g, 0.58 mol) in H₂O (1 L) at 0° C. The mixture was stirred at 30° C. for 1 hour. A solution of KI (560 g, 3.4 mol) in water (800 mL) was added dropwise into the mixture at 0° C. The mixture was warmed to 25° C. and stirred for 2 hours. TLC (CH₂Cl₂: MeOH=15:1) showed the reaction was complete. The mixture was extracted with ethyl acetate (4 L). The organic layer was washed with saturate aqueous Na₂S₂O₃ (5 L×2) and brine, dried over Na₂SO₄ and concentrated in vacuo to give compound 6 (80 g, 90%) as a yellow solid. ¹H NMR (400 MHz, CDCl₃): δ 8.37 (S, 1H), 7.75-7.77 (dd, J=8.0 Hz, 1H), 7.02-7.04 (d, J=8.0 Hz, 1H), 2.60 (s, 3H).

### Synthesis of Compound 7

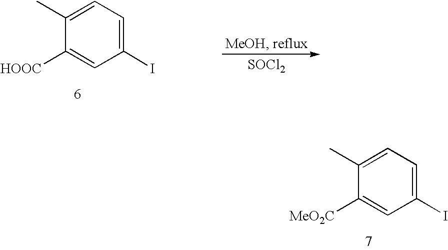

To a solution of compound 6 (40.0 g, 0.153 mol) in MeOH (400 mL) was added dropwise a freshly prepared solution of SOCl₂ (22 mL, 0.305 mol) in MeOH (400 mL) at 0° C. The mixture was heated to reflux for 4 hours. TLC (petroleum ether:ethyl acetate=10:1) showed the reaction was complete, then the mixture was concentrated in vacuo. The residue was desolved in ethyl acetate (800 mL) and washed with water (200 mL×2), saturated aqueous sodium carbonate (200 mL×2) and brine, dried over anhydrous sodium sulfate and concentrated in vacuo to give compound 7 as a yellow solid. ¹H NMR (300 MHz, CDCl₃): δ 8.21 (S, 1H), 7.67-7.70 (dd, J=9.0 Hz, 1H), 6.97-6.99 (d, J=6.0 Hz, 1H), 3.88 (s, 3H), 2.53 (s, 3H).

### Synthesis of Compound 8

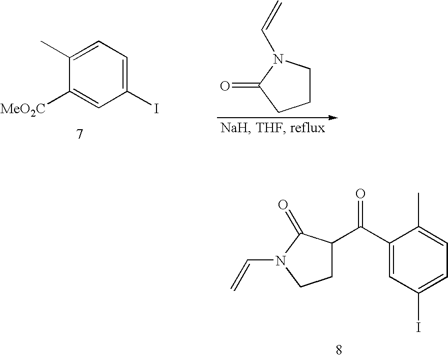

A solution of compound 7 (39.6 g, 0.143 mol) and 1-vinyl-2-pyrrolidinone (18.5 g, 0.143 mol) in anhydrous THF (200 mL) was added slowly to a gently refluxing suspension of sodium hydride (9.2 g, 60% in mineral oil, 0.229 mol) in THF (70 mL). After the addition, the mixture was stirred at reflux for additional 3 hours. TLC (petroleum ether:ethyl acetate=10:1) showed the reaction was complete. The mixture was cooled and the excess sodium hydride was quenched with saturated aqueous ammonium chloride. The mixture was concentrated in vacuo to remove THF. The residue was extracted with ethyl acetate (400 mL×2). The combined organic layers were washed with brine, dried over anhydrous sodium sulfate and concentrated in vacuo to give crude product 8, which was used in the next step without further purification.

### Synthesis of Compound 9

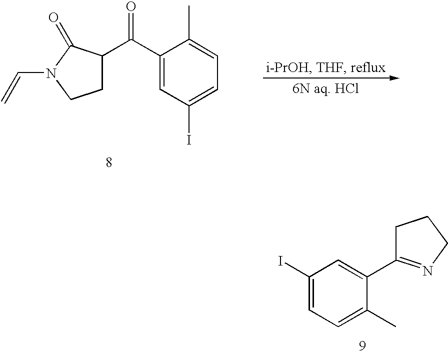

To a solution of compound 8 (0.143 mol) in i-PrOH/THF (100 mL/50 mL) was added 6 N aqueous HCl (360 mL). The mixture was stirred at reflux for 14 hours. TLC (petroleum ether:ethyl acetate=10:1) showed the reaction was complete. The mixture was cooled and extracted with ethyl acetate (100 mL×2). The majority of the product remained in the acidic aqueous layer. The combined organic layers were washed with 3 N HCl (200 mL×2). The acidic aqueous layers were basified to pH>10 by gradual addition of K₂CO₃. The aqueous layer was extracted with ethyl acetate (600 mL×3). The combined organic layers were washed with brine, dried over anhydrous sodium sulfate and concentrated in vacuo to give crude product, which was purified by column chromatography (first petroleum ether, then petroleum ether:ethyl acetate 20:1) to afford compound 9 (11.8 g, yield: 29% for two steps) as a yellow oil. ¹H NMR (400 MHz, CDCl₃): δ 7.74 (S, 1H), 7.55-7.57 (d, J=8.0 Hz, 1H), 6.96-6.98 (d, J=8.0 Hz, 1H), 4.06-4.10 (t, J=8.0 Hz, 2H), 2.85-2.90 (t, J=10.0 Hz, 2H), 2.43 (s, 3H), 1.97-2.06 (m, 2H). MS: ES-MS: 286 (M+1)⁺

### Synthesis of Compound 10

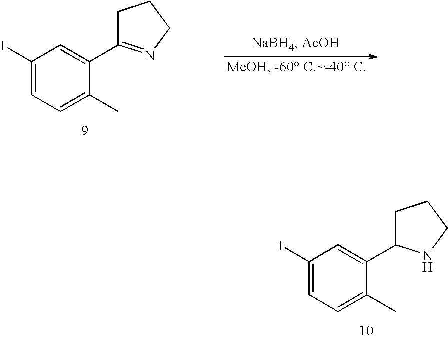

To a solution of compound 9 (18 g, 0.063 mol) in 150 mL of methanol/acetic acid (80:20) was added sodium borohydride (4.8 g, 0.126 mol) in portions at −60° C. over 30 minutes. During the addition, the temperature was allowed to keep at about −40° C. The mixture was stirred at −40° C. for 2 hours. TLC (petroleum ether:ethyl acetate=10:1) showed the reaction was complete. The mixture was warmed to room temperature and evaporated to remove most of the solvent. 500 mL of H₂O was added to the residue, and the mixture was basified to pH 9 with K₂CO₃. The resulting mixture was extracted with CH₂Cl₂ (800 mL×2). The combined organic layers were washed with brine, dried over anhydrous potassium carbonate and concentrated in vacuo to give crude product 10, which was used in the next step without further purification. MS: ES-MS: 288 (M+1)⁺.

### Synthesis of Compound 11

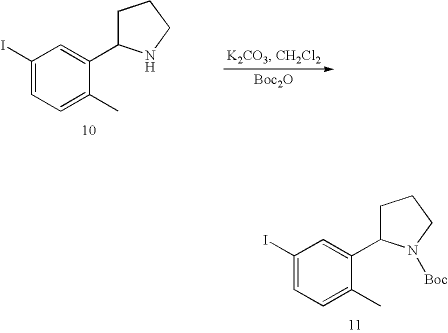

A suspension of compound 10 (0.063 mol) and Na₂CO₃ (13.0 g, 0.123 mol) in dry CH₂Cl₂ (160 mL) was cooled to 5° C., then Boc₂O (18.6 g, 0.085 mol) was added dropwise. The reaction mixture was stirred at room temperature overnight. TLC (petroleum ether:ethyl acetate=10:1) showed the reaction was complete. The mixture was concentrated in vacuo. The residue was dissolved in 250 mL of ethyl acetate and 100 mL of water. The organic layer was separated, and the aqueous layer was extracted with ethyl acetate (100 mL×2). The combined organic layers were washed with brine, dried over anhydrous sodium sulfate and concentrated in vacuo to give crude product, which was purified by column chromatography (first petroleum ether, then petroleum ether:ethyl acetate 30:1) to afford compound 11 (21 g, yield: 84% for two steps) as a white solid. ¹H NMR (300 MHz, CDCl₃): ¹H NMR δ 7.39-7.43 (m, 2H), 6.83-6.85 (d, J=6.0 Hz, 1H), 4.83-5.18 (m, 1H), 3.48-3.66 (m, 2H), 2.32-2.36 (m, 4H), 1.83-1.95 (m, 2H), 1.65-1.72 (m, 1H), 1.45 (br, 3H), 1.16 (br, 6H). MS: ES-MS: 410 (M+23)⁺, 288 (M−100)⁺, 332 (M−56)⁺.

### Synthesis of Compound G3

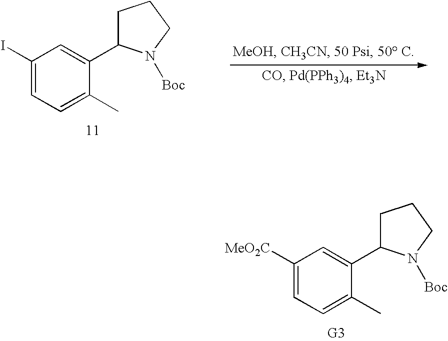

A mixture of compound 11 (18 g, 0.048 mol), Pd(PPh₃)₄ (2.8 g, 0.002 mol), Et₃N (10 mL, 0.071 mol), 80 mL of acetonitrile and 40 mL of methanol was stirred under 50 psi of carbon monoxide at 50° C. for 12 hours. The reaction mixture was concentrated in vacuo. The residue was dissolved in 250 mL of ethyl acetate, then washed with water and brine, dried over anhydrous sodium sulfate and concentrated in vacuo to give crude product, which was purified by column chromatography (first petroleum ether, then petroleum ether:ethyl acetate 40:1 to 10:1) to afford compound G3 (14 g, 94%) as a yellow solid. ¹H NMR (400 MHz, CDCl₃): δ 7.71-7.80 (m, 2H), 7.17-7.19 (d, J=8.0 Hz, 1H), 4.93-5.25 (m, 1H), 3.87 (s, 3H), 3.46-3.74 (m, 2H), 2.32-2.39 (m, 4H), 1.87-1.96 (m, 2H), 1.68-1.72 (m, 1H), 1.45 (br, 3H), 1.12 (br, 6H). MS: ES-MS: 342 (M+23)⁺, 220 (M−100)⁺, 264 (M−56)⁺.

### Synthesis of Compound H3

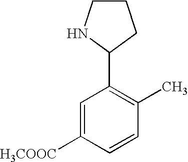

methyl 4-methyl-3-pyrrolidin-2-ylbenzoate Hydrogen chloride (8 mL, 32 mmol; 4 M in dioxane) was added to a solution compound G3 (0.5 g, 2 mmole) in DCM (5 mL). The reaction was stirred at room temperature for 12 hours. The reaction mixture was evaporated to give a white solid residue. The residue was used for the next reaction step without further purification.

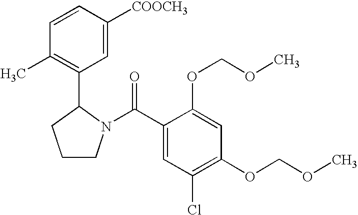

### Methyl 3-{1-[5-chloro-2,4-bis(methoxymethoxy)benzoyl]pyrrolidin-2-yl}-4-methylbenzoate

Methyl 4-methyl-3-pyrrolidin-2-ylbenzoate (1.56 mmol) was added to a solution of 5-chloro-2,4-bis(methoxymethoxy)benzoic acid (432 mg, 1.56 mmol), 4-methylmorpholine (3.4 mL, 31 mmol), N-(3-dimethylaminopropyl)-N′-ethylcarbodiimide hydrochloride (0.6 g, 3.1 mmol), and 1-hydroxy benzotriazole (0.42 g, 3.1 mmol) in 12 mL of DMF under a nitrogen atmosphere. The reaction was allowed to stir at room temperature for 12 hours. H₂O (50 mL) was added to the reaction mixture to quench the reaction. EtOAc (2×100 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 55→60% EtOAc in hexanes to give the desired product (0.61 g, 81.9% yield).

### Synthesis of Compound I3

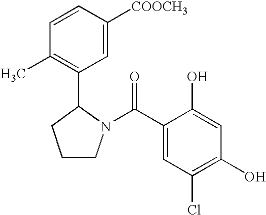

### methyl 3-[1-(5-chloro-2,4-dihydroxybenzoyl)pyrrolidin-2-yl]-4-methylbenzoate

Hydrogen chloride (0.8 mL, 3 mmol; 4 M in dioxane) was added to a solution of Methyl 3-{1-[5-chloro-2,4-bis(methoxymethoxy)benzoyl]pyrrolidin-2-yl}-4-methylbenzoate (18.4 mg, 0.04 mmole) in MeOH (2 mL). The reaction was stirred at room temperature for 12 hours. The reaction mixture neutralized with saturated NaHCO₃ (aq) and then extracted with EtOAc (2×20 mL). The combined organic layers were dried, filtered, and evaporated to give a white solid (15 mg, 96.2% yield). ¹H NMR (400 MHz, DMSO-D6) δ ppm 1.58-1.68 (m, 1H) 1.83 (s, 2H) 2.10-2.29 (m, 1H) 2.41 (s, 3H) 3.43-3.59 (m, 1H) 3.70-3.79 (m, 1H) 3.83 (s, 3H) 5.14-5.34 (m, 1H) 6.58 (s, 1H) 7.13 (s, 1H) 7.29 (s, 1H) 7.56-7.77 (m, 1H) 7.92 (s, 1H). Anal. Calcd for C₂₀H₂₀ClNO₅.0.1 hexane: C, 62.10; H, 5.41; N, 3.52. Found: C, 62.11; H, 5.46; N, 3.45.

### Example 218

### 3-[1-(5-chloro-2,4-dihydroxybenzoyl)pyrrolidin-2-yl]-N-ethyl-4-methylbenzamide

### Synthesis of Compound J3

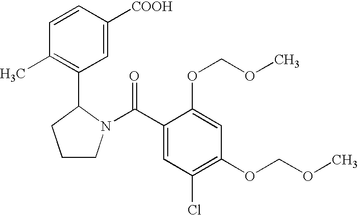

### 3-{1-[5-chloro-2,4-bis(methoxymethoxy)benzoyl]pyrrolidin-2-yl}-4-methylbenzoic acid

Lithium hydroxide hydrate (0.8 g, 15 mmol) was added to a solution of methyl 3-[1-(5-chloro-2,4-dihydroxybenzoyl)pyrrolidin-2-yl]-4-methylbenzoate (from Example 217) (0.61 g, 1.28 mmol) in H₂O (5 mL) and MeOH (4 mL). The reaction mixture was heated to 40° C. for 12 hours. The mixture was evaporated and neutralized by HOAc—NaOAc buffer solution. EtOAc (2×50 mL) was added to extract the aqueous solution. The combined organic layers were dried, filtered, and concentrated to give the desired product as a white solid (0.55 g, 93.7% yield). ¹H NMR (400 MHz, DMSO-D6) δ ppm 1.59-1.67 (m, 1H) 1.78-1.87 (m, 2H) 2.36-2.39 (m, 1H) 2.41 (s, 3H) 3.38 (s, 3H) 3.44 (s, 3H) 3.56-3.67 (m, 2H) 5.26-5.39 (m, 4H) 5.44 (d, J=6.82 Hz, 1H) 7.11 (s, 1H) 7.26-7.34 (m, 2H) 7.71 (dd, J=7.71, 1.64 Hz, 1H) 7.91 (s, 1H) 12.86 (s, 1H).

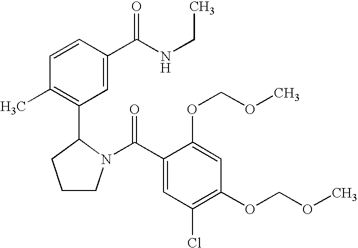

### 3-{1-[5-chloro-2,4-bis(methoxymethoxy)benzoyl]pyrrolidin-2-yl}-N-ethyl-4-methylbenzamide

Ethylamine (1 mL, 0.7 mmol) was added to a solution of compound J3 as prepared above (150 mg, 0.32 mmol), 4-methylmorpholine (0.8 ml, 7 mmol), N-(3-dimethylaminopropyl)-N′-ethylcarbodiimide hydrochloride (140 mg, 0.7 mmol), and 1-hydroxy benzotriazole (100 mg, 0.7 mmol) in 4 mL of DMF under a nitrogen atmosphere. The reaction was allowed to stir at room temperature for 12 hours. H₂O (30 mL) was added to the reaction mixture to quench the reaction. EtOAc (2×50 mL) was then added to extract the aqueous solution. Dry EtOAc layer over Na₂SO₄. The Na₂SO₄ was filtered off and the filtrate was evaporated to give a brown oil residue. The residue was purified by silica gel chromatography (gradient elution 60%→65% EtOAc in hexanes to give the desired product (92.4 mg, 83.6% yield).

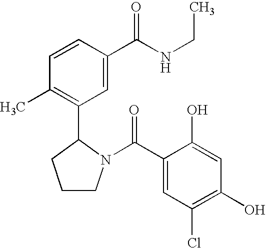

### 3-[1-(5-chloro-2,4-dihydroxybenzoyl)pyrrolidin-2-yl]-N-ethyl-4-methylbenzamide

Hydrogen chloride (1.5 mL, 6 mmol; 4 M in dioxane) was added to a solution of 3-{1-[5-chloro-2,4-bis(methoxymethoxy)benzoyl]pyrrolidin-2-yl}-N-ethyl-4-methylbenzamide (92.4 mg, 0.27 mmol) in MeOH (2 mL). The reaction was stirred at room temperature for 12 hours. The reaction mixture was neutralized with saturated NaHCO₃ (aq) and then extracted with EtOAc (2×50 mL). The combined organic layers were dried, filtered, and evaporated to give a white solid (70 mg, 64.8% yield). ¹H NMR (400 MHz, DMSO-D6) δ ppm 1.11 (t, J=7.20 Hz, 3H) 1.59-1.69 (m, 1H) 1.78-1.95 (m, 2H) 2.02-2.11 (m, 1H) 2.39 (s, 3H) 3.21-3.31 (m, 2H) 3.48-3.61 (m, 1H) 3.74-3.99 (m, 1H) 5.13-5.32 (m, 1H) 6.56 (s, 1H) 7.13-7.30 (m, 2H) 7.57 (dd, J=7.83, 1.77 Hz, 1H) 7.67-7.84 (m, 1H) 8.19-8.51 (m, 1H) 10.49 (s, 2H). Anal. Calcd for C₂₁H₂₃ClN₂O₄: C, 62.61; H, 5.75; N, 6.95. Found: C, 62.82; H, 6.01; N, 6.52.

### Example 219

### HSP-90 Biochemical Assay

Compounds of the present invention were evaluated for potency against HSP-90 using a SPA (scintillation proximity assay) competition binding assay. Briefly, either full length or N-terminal HSP-90 that contains a 6-His tag on its C-terminus binds to copper on Yttrium-silicate scintillant beads via the His-tag. Tritiated propyl-Geldanamycin (pGA), whose structure is shown below, is an analog of a natural inhibitor of HSP-90 called Geldanamycin.

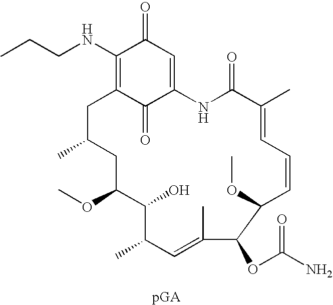

Tritiated pGA, which contains a tritiated propyl-amine group added at the #17 position, binds HSP-90 and brings the isotope into proximity with the beads. 17-n-propylamino-Geldanamycin can be prepared as described in U.S. Pat. No. 4,261,989, which is incorporated herein by reference. The beta signal emitted from the isotope excites the scintillant, which creates a measurable signal. As competitive compounds are added to the assay mixture, they compete with bound tritiated pGA at the ATP-binding site on the N-terminal of HSP-90. When a compound displaces the labeled pGA, the signal is reduced (the beta-particles are no longer in proximity with the bead). This reduction in signal is used to quantify the extent to which the inhibitor/compound is competitive with pGA.

The SPA assay for ³H-pGA binding to HSP-90 was performed in 96-well flat bottom white plates (Corning #3604). Typical reaction solutions contained 30 nM HSP-90 and 200 nM ³H-pGA in binding buffer (100 mM Hepes, pH 7.5 and 150 mM KCl). The ³H-pGA was first diluted to 33% label with unlabeled pGA that was synthesized and purified to give a final concentration of 200 nM. Inhibitors were added to the HSP-90/³H-pGA solutions at eleven different concentrations for Kᵢ determinations. The range of inhibitor concentrations were 100 μM, or an appropriate range, for solid samples and 10 μM for targeted library compounds and 4 mM liquid stocks. To determine percent inhibition, the compound was tested at 1 and 10 μM. The final DMSO in the samples was 4%. Copper-Ysi beads (Amersham, #RPNQ0096) that have been diluted in binding buffer were added to each well to give a final concentration of 100 μg/well. The plates were sealed, covered with a foil-covered lid and shaken for 30 minutes at room temperature. The beads were allowed to settle for 30 minutes after which the plates were counted using a Packard TopCount NXT instrument. This procedure has also been adapted for medium throughput using a Beckman Biomek FX. Samples were run in duplicate and on two separate days to assure an accurate value of Kᵢ.

For Kᵢ determinations, the corrected cpm's (actual cpm's minus background) were plotted vs. inhibitor concentration using GraphPad Prism software. The data were fit to a generic IC₅₀ equation, Y=YI/(1+[X]/IC₅₀), where YI=Y-intercept and [X] is the competing ligand/inhibitor. The IC₅₀ was then used to calculate the Ki by using the Cheng-Prusoff equation:

$$Ki \{ cl \} = \frac{{IC}_{50} \{ cl \}}{1 + ( [ hl ] / Kd \{ hl \} )}$$

Where cl=cold ligand concentration (varies), [hl]=concentration of hot ligand (200 nM) and Kd{hl}=240 nM. Error was calculated as follows: IC₅₀ error/IC₅₀ value=fractional error and fractional error*Kᵢ value=Kᵢ error.

In the cases in which inhibitor binds to HSP-90 so tightly that the population of free inhibitor molecules is significantly depleted by formation of the enzyme-inhibitor complex, the above equation is no longer valid. This is normally true when the observed IC₅₀ is about the same as the HSP-90 concentration. For a tight binding inhibitor, the following equation can be applied:

$$\frac{EL}{{EL}_{o}} = \frac{- ( {K}_{I}^{app} + {I}_{o} - {E}_{o} ) + \sqrt{{( {K}_{I}^{app} + {I}_{o} - {E}_{o} )}^{2} + 4 \times {E}_{o} \times {K}_{I}^{app}}}{2 \times {E}_{o}}$$

### Where

$${K}_{I}^{app} = {K}_{I} \times ( 1 + \frac{{L}_{o}}{{K}_{L}} )$$

EL and ELₒ are the radioligand-HSP-90 complexes in the presence and absence of inhibitor, respectively. EL/ELo represents the fractional signal in the presence of inhibitor. Io, Eₒ, and Lₒ are the inhibitor, HSP-90, and radioligand concentrations, respectively.

### Example 220

### HSP-90 Biochemical Assay Data for Compounds Shown in Examples 1 to 136

| | Ki | % inhibition | % inhibition |
|---|---|---|---|
| Example | (μM) | @ 1 μM | @ 10 μM |
| 1 | 8.20 | | |
| 2 | 0.03 | | |
| 3 | 1.98 | | |
| 4 | 0.06 | | |
| 5 | 0.02 | | |
| 6 | 0.36 | | |
| 7 | 0.04 | | |
| 8 | 0.08 | | |
| 9 | 2.34 | | |
| 10 | 0.23 | | |
| 11 | 15.0 | | |
| 12 | 0.26 | | |
| 13 | 10.9 | | |
| 14 | 0.003 | | |
| 15 | 0.88 | | |
| 16 | 0.65 | | |
| 17 | 36.0 | | |
| 18 | 1.80 | | |
| 19 | 1.90 | | |
| 20 | 10.1 | | |
| 21 | 0.38 | | |
| 22 | 0.31 | | |
| 23 | 0.86 | | |
| 24 | | 5.4 | 34.9 |
| 25 | | 9.9 | 13.7 |
| 26 | | 1.0 | 39.0 |
| 27 | | 0.2 | 26.2 |
| 28 | 0.50 | 50.8 | 76.2 |
| 29 | | 0.0 | 26.6 |
| 30 | | 5.8 | 61.2 |
| 31 | | 8.1 | 12.9 |
| 32 | | −11.9 | 14.2 |
| 33 | | 2.0 | 20.9 |
| 34 | | −3.1 | 43.0 |
| 35 | | 6.1 | 32.8 |
| 36 | 5.37 | | |
| 37 | | 24.6 | 64.1 |
| 38 | | 27.4 | 68.4 |
| 39 | | 11.3 | 52.3 |
| 40 | | 13.2 | 49.1 |
| 41 | 0.30 | 64.8 | 86.8 |
| 42 | 0.47 | 59.9 | 86.5 |
| 43 | | 35.9 | 73.6 |
| 44 | 0.06 | 82.4 | 90.5 |
| 45 | | 9.6 | 38.3 |
| 46 | | 44.9 | 77.1 |
| 47 | | 43.4 | 80.0 |
| 48 | 0.31 | 63.3 | 87.5 |
| 49 | 0.36 | 61.2 | 86.3 |
| 50 | | 34.6 | 77.7 |
| 51 | | 22.1 | 70.9 |
| 52 | | 39.1 | 80.2 |
| 53 | | −7.8 | 7.3 |
| 54 | 0.58 | 50.3 | 85.5 |
| 55 | 0.11 | 77.4 | 87.0 |
| 56 | | 27.0 | 73.0 |
| 57 | 0.15 | 73.7 | 88.2 |
| 58 | | 29.3 | 72.5 |
| 59 | 0.47 | 58.6 | 82.9 |
| 60 | 0.12 | 77.6 | 88.1 |
| 61 | | 37.7 | 75.6 |
| 62 | | 12.0 | 64.4 |
| 63 | | 32.2 | 72.0 |
| 64 | | 5.9 | 59.2 |
| 65 | 0.32 | 69.7 | 87.0 |
| 66 | 0.44 | 55.5 | 84.8 |
| 67 | | −0.8 | 35.4 |
| 68 | | 37.2 | 78.6 |
| 69 | | 33.9 | 76.6 |
| 70 | | 40.2 | 79.1 |
| 71 | | 37.3 | 79.3 |
| 72 | | 13.2 | 62.4 |
| 73 | 0.06 | 82.7 | 91.6 |
| 74 | | 47.7 | 80.7 |
| 75 | 0.53 | 51.6 | 82.0 |
| 76 | | 48.4 | 81.0 |
| 77 | | 44.6 | 79.6 |
| 78 | | 17.7 | 68.1 |
| 79 | 0.44 | 57.7 | 85.1 |
| 80 | | 42.0 | 81.3 |
| 81 | | 29.0 | 73.8 |
| 82 | 0.38 | 62.8 | 85.3 |
| 83 | | 10.1 | 35.0 |
| 84 | 0.13 | 76.2 | 89.8 |
| 85 | 0.60 | 52.1 | 79.6 |
| 86 | | 23.9 | 59.9 |
| 87 | | 36.7 | 65.0 |
| 88 | | 4.3 | 42.9 |
| 89 | | 45.7 | 77.8 |
| 90 | | 23.2 | 65.4 |
| 91 | | −7.3 | 12.2 |
| 92 | | 9.0 | 22.4 |
| 93 | | 34.5 | 76.1 |
| 94 | | 21.1 | 62.1 |
| 95 | | 22.7 | 70.3 |
| 96 | | 20.8 | 67.6 |
| 97 | 0.39 | 62.7 | 82.6 |
| 98 | | 26.7 | 65.9 |
| 99 | 0.65 | 52.2 | 80.0 |
| 100 | | 35.9 | 79.1 |
| 101 | | 21.8 | 70.2 |
| 102 | | 9.8 | 54.4 |
| 103 | | 64.5 | 87.8 |
| 104 | | 33.9 | 74.3 |
| 105 | | 30.2 | 69.3 |
| 106 | | 26.9 | 72.4 |
| 107 | 0.69 | 51.1 | 83.4 |
| 108 | | 40.5 | 83.1 |
| 109 | 0.32 | 68.0 | 87.2 |
| 110 | 0.41 | 55.3 | 82.8 |
| 111 | | 5.9 | 64.2 |
| 112 | | 48.1 | 80.5 |
| 113 | 0.61 | 53.0 | 81.9 |
| 114 | 0.51 | 60.2 | 83.2 |
| 115 | 0.50 | 60.3 | 83.7 |
| 116 | | 5.0 | 62.1 |
| 117 | 0.26 | 65.0 | 89.2 |
| 118 | | 15.4 | 57.0 |
| 119 | 0.68 | 50.9 | 82.8 |
| 120 | 0.10 | 76.2 | 89.4 |
| 121 | | 10.6 | 64.6 |
| 122 | | 48.2 | 79.0 |
| 123 | | 25.5 | 65.6 |
| 124 | | −4.8 | 18.1 |
| 125 | | 22.4 | 59.8 |
| 126 | | 35.9 | 71.0 |
| 127 | | 21.1 | 60.3 |
| 128 | | 31.2 | 73.1 |
| 129 | 0.34 | 60.5 | 88.5 |
| 130 | | 46.2 | 82.4 |
| 131 | | 24.7 | 69.6 |
| 132 | | 33.3 | 73.9 |
| 133 | | 42.6 | 80.0 |
| 134 | | 3.7 | 37.6 |
| 135 | | 15.9 | 44.7 |
| 136 | | 10.4 | 47.8 |

### Example 221

### HSP-90 Biochemical Assay Data for Compounds Shown in Examples 137 to 214, and 217-218

| | Ki | % inhibition | % inhibition |
|---|---|---|---|
| Example | (μM) | @ 1 μM | @ 10 μM |
| 137 | 0.002 | | |
| 138 | | | |
| 139 | 0.056 | | |
| 140 | 0.005 | | |
| 141 | 0.44 | | |
| 142 | 7.3 | | |
| 143 | 0.88 | | |
| 144 | 0.65 | | |
| 145 | 36 | | |
| 146-a | 0.005 | | |
| 146-b | 0.5 | | |
| 146-c | 0.001 | | |
| 147 | 2.3 | | |
| 148 | 1.7 | | |
| 149 | 20 | | |
| 150 | 0.004 | | |
| 151 | 0.5 | | |
| 152 | 0.002 | | |
| 153 | | | |
| 154 | | | |
| 155 | 4.5 | | |
| 156 | 0.005 | | |
| 157 | 0.005 | | |
| 158 | 0.006 | | |
| 159 | 0.01 | | |
| 160 | 0.01 | | |
| 161 | 0.005 | | |
| 162 | 0.005 | | |
| 163 | 0.007 | | |
| 164 | 0.008 | | |
| 165 | 0.003 | | |
| 166 | 0.043 | | |
| 167 | 0.006 | | |
| 168 | 0.2 | | |
| 169 | 0.13 | | |
| 170 | | | |
| 171 | 3 | | |
| 172 | 0.03 | | |
| 173 | 0.1 | | |
| 174 | 0.02 | | |
| 175 | 0.06 | | |
| 176 | 0.4 | | |
| 177 | 0.2 | | |
| 178 | 4.2 | | |
| 179 | 0.7 | | |
| 180 | 0.8 | | |
| 181 | 0.08 | | |
| 182 | 0.06 | | |
| 183 | 0.01 | | |
| 184 | | | 0 |
| 185 | 25 | | |
| 186 | | | |
| 187 | | 0 | |
| 188 | 0.6 | | |
| 189 | 1.2 | | |
| 190 | 30 | | |
| 191 | 1 | | |
| 192 | 20 | | |
| 193 | 2.1 | | |
| 194 | 9.3 | | |
| 195 | 0.6 | | |
| 196 | 1.6 | | |
| 197 | 2.8 | | |
| 198 | 0.1 | | |
| 199 | 1.6 | | |
| 200 | 0.28 | | |
| 201 | 15 | | |
| 202 | 0.29 | | |
| 203 | 0.13 | | |
| 204 | 0.13 | | |
| 205 | 0.27 | | |
| 206 | | | 0 |
| 207 | 0.19 | | |
| 208 | | | 0 |
| 209 | 0.002 | | |
| 210 | 0.02 | | |
| 211 | 0.06 | | |
| 212 | 0.004 | | |
| 213 | 0.004 | | |
| 214 | 0.01 | | |
| 217 | 0.14 | | |
| 218 | 0.66 | | |

1. A compound of formula (I) 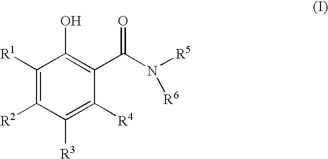
wherein:
R¹ is H, —CH₃, or halogen;
R², R³, and R⁴ are each independently H, —OH, (C₁ to C₆) alkyl, (C₂ to C₈) alkenyl, (C₂ to C₈) alkynyl, (C₁ to C₈) alkoxy, (C₁ to C₈) heteroalkyl, halogen, —CF₃, cyano, —Xₘ—C(O)R⁷, —Xₘ—S(O)₂R⁷, —Xₘ—(NR^{8a})—S(O)₂R^{8b}, —Xₘ—(NR^{8a})—C(O)R^{8b}, (C₂ to C₉) cycloheteroalkyl, (C₃ to C₈) cycloalkyl, (C₆ to C₁₄) aryl, or (C₂ to C₉) heteroaryl, each of which is optionally substituted with at least one R⁹ group; or
R³ together with either R² or R⁴, together with the atoms to which they are attached, form a (C₆ to C₁₄) aryl, (C₂ to C₉) heteroaryl, (C₂ to C₉) cycloheteroalkyl, or a (C₃ to C₈) cycloalkyl group, each of which is optionally substituted with at least one R⁹ group;
R⁵ and R⁶ taken together with the nitrogen atom to which they are attached in formula (I) form a (C₂ to C₉) heteroaryl, or a (C₂ to C₉) cycloheteroalkyl group, wherein each of said (C₂ to C₉) heteroaryl and (C₂ to C₉) cycloheteroalkyl is optionally substituted with at least one R¹² group, and each is optionally spiro-fused to an R¹³ group;
each R⁷ is independently H, halogen, —CF₃, cyano, —N(R^{8a}R^{8b}), (C₁ to C₈) alkyl, (C₂ to C₈) alkenyl, (C₂ to C₈) alkynyl, (C₁ to C₈) alkoxy, (C₆ to C₁₄) aryl, (C₂ to C₉) heteroaryl, (C₂ to C₈) cycloheteroalkyl, or (C₃ to C₈) cycloalkyl;
each R^{8a} and R^{8b} is independently H, (C₁ to C₆) alkyl, (C₂ to C₈) alkenyl, (C₂ to C₈) alkynyl, —(CH₂)ₙCN, —(CH₂)ₙN(R^{10a}R^{10b}), —(CH₂)ₙCF₃, —(CH₂)ₙCHF₂, (C₃ to C₈) cycloalkyl, (C₂ to C₉) cycloheteroalkyl, or (C₁ to C₈) heteroalkyl, wherein said (C₃ to C₈) cycloalkyl and (C₂ to C₉) cycloheteroalkyl are each optionally substituted with at least one R^{10a} group; or when R^{8a} and R^{8b} are both bound to a nitrogen atom, R^{8a} and R^{8b} together with the nitrogen atom to which they are attached, can form a (C₂ to C₉) cycloheteroalkyl group;
each R⁹ is independently —OH, halogen, —CF₃, cyano, (C₁ to C₈) alkyl, (C₂ to C₈) alkenyl, (C₂ to C₈) alkynyl, (C₁ to C₈) heteroalkyl, (C₁ to C₈) alkoxy, —Xₘ—S(O)₂R⁷, —Xₘ(NR^{8a})—S(O)₂R^{8b}, —N(R^{8a}R^{8b}, —NR^{8a}C(O)₂R^{8b}, —(CH₂)ₙC(O)₂R^{8a}, —C(O)N(R^{8a}R^{8b}), —Xₘ—(C₆ to C₁₄) aryl, —Xₘ—(C₂ to C₉) heteroaryl, —Xₘ—(C₂ to C₉) cycloheteroalkyl, or —Xₘ—(C₃ to C₉) cycloalkyl, wherein said (C₆ to C₁₄) aryl, (C₂ to C₉) heteroaryl (C₂ to C₉) cycloheteroalkyl, or (C₃ to C₈) cycloalkyl is optionally further substituted with at least one R¹⁴ group;
each R^{10a} and R^{10b} is independently H, —OH, (C₁ to C₆) alkyl, (C₂ to C₈) alkenyl, (C₂ to C₈) alkynyl, (C₁ to C₈) heteroalkyl, or (C₁ to C₈) alkoxy;
each R¹¹ is independently (C₁ to C₈) alkyl, (C₂ to C₈) alkenyl, (C₂ to C₈) alkynyl, (C₁ to C₈) alkoxy, (C₁ to C₈) heteroalkyl, cyano, —CF₃, halogen, —N(R^{8a}R^{8b}), —(CH₂)ₙC(O)₂R^{8a} —(CH₂)ₙ—S(CH₂)ₙR⁹, —C(O)N(R^{8a}R^{8b}), (C₆ to C₁₄) aryl, (C₂ to C₉) heteroaryl, (C₂ to C₉) cycloheteroalkyl, or (C₃ to C₈) cycloalkyl, each of which is optionally substituted with at least one R⁸ group;
each R¹² is independently —OH, halogen, —CF₃, cyano, (C₁ to C₆) alkyl, (C₂ to C₈) alkenyl, (C₂ to C₈) alkynyl, (C₁ to C₈) heteroalkyl, (C₁ to C₈) alkoxy, —(CH₂)ₙ—SCH₃, —N(R^{8a}R^{8b}), —NR^{8a}C(O)₂R^{8b}, —C(O)N(R^{8a}R^{8b}), —(CH₂)ₙC(O)₂R^{8a}, —(CH₂)ₙC(O)₂R¹¹, —XₘS(O)₂R⁷, —Xₘ—(C₆ to C₁₄) aryl, —Xₘ—(C₂ to C₈) heteroaryl, —Xₘ—(C₂ to C₉) cycloheteroalkyl, or —Xₘ—(C₃ to C₈) cycloalkyl, wherein said (C₆ to C₁₄) aryl, (C₂ to C₈) heteroaryl, (C₂ to C₉) cycloheteroalkyl, and (C₃ to C₈) cycloalkyl are optionally further substituted with at least one R¹¹ group;
each R¹³ is independently (C₆ to C₁₄) aryl, (C₂ to C₉) heteroaryl, (C₂ to C₉) cycloheteroalkyl, or (C₃ to C₈) cycloalkyl, each of which is optionally substituted with at least one R¹² group;
each R¹⁴ is independently —OH, halogen, —CF₃, cyano, —NO₂, (C₁ to C₈) alkyl, (C₂ to C₈) alkenyl, (C₂ to C₈) alkynyl, (C₁ to C₈) heteroalkyl, (C₁ to C₈) alkoxy, —(CH₂)ₙ—SCH₃, —N(R^{8a}R^{8b}), —NR^{8a}C(O)₂R^{8b}, —(CH₂)ₙC(O)₂R^{8a}, —Xₘ—(C₆ to C₁₄) aryl, —Xₘ—(C₂ to C₉) heteroaryl, —Xₘ—(C₂ to C₉) cycloheteroalkyl, or —Xₘ—(C₃ to C₈) cycloalkyl;
X is —O—, —S—, —NH—, (C₁ to C₈) heteroalkyl, (C₁ to C₈) alkoxy, (C₁ to C₆) alkyl, (C₂ to C₈) alkenyl, or (C₂ to C₈) alkynyl;
each m is independently 0 or 1; and
each n is independently 0, 1, 2, 3, or 4; or
a pharmaceutically acceptable salt or solvate thereof.

2. (canceled)

3. The compound according to claim 1, wherein:
R² is —OH; and
R³ and R⁴ are each independently H, —OH, (C₁ to C₆) alkyl, (C₁ to C₈) alkoxy, (C₁ to C₈) heteroalkyl, halogen, —CF₃, cyano, (C₂ to C₈) cycloheteroalkyl, (C₃ to C₈) cycloalkyl, (C₆ to C₁₄) aryl, or (C₂ to C₉) heteroaryl, each of which is optionally substituted with at least one R⁹ group.

4. The compound according to claim 1, wherein:
R⁵ and R⁶ taken together with the nitrogen atom to which they are attached in formula (I) form a group selected from: 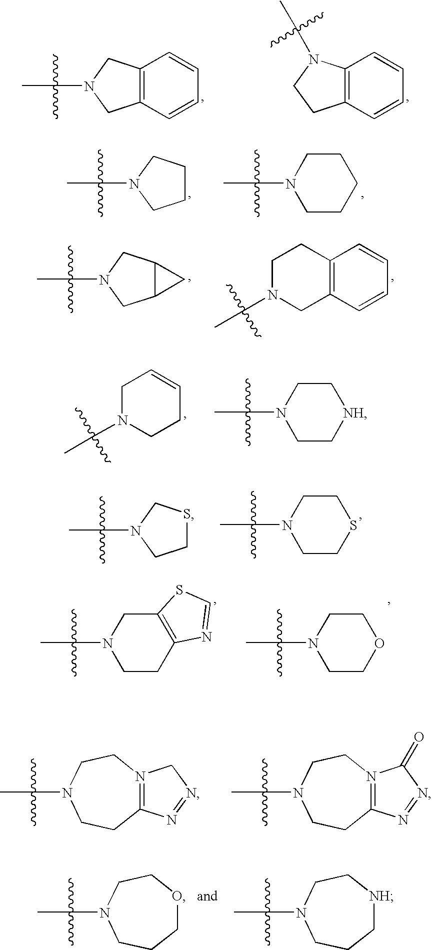
wherein each of said groups is optionally substituted with at least one R¹² group, and each of which is optionally spiro-fused to an R¹³ group.

5. The compound according to claim 1 wherein the compound of formula (I) has the following structure:

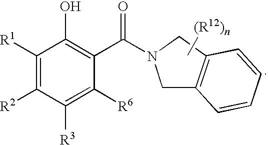

6. The compound according to claim 1 wherein the compound of formula (I) has the following structure:

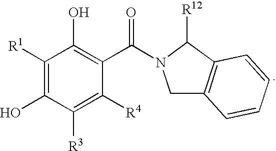

7. The compound according to claim 1 wherein the compound of formula (I) has the following structure:

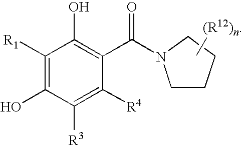

8. The compound according to claim 1 which is selected from the group consisting of: 3-bromo-2-(1,3-dihydro-2H-isoindol-2-ylcarbonyl)phenol; 4-(1,3-dihydro-2H-isoindol-2-ylcarbonyl)benzene-1,3-diol; tert-butyl [3-(2,4-dihydroxybenzoyl)-3-azabicyclo[3.1.0]hex-6-yl]carbamate; 4-{[2-(2-methylphenyl)pyrrolidin-1-yl]carbonyl}benzene-1,3-diol; 4-{[2-(1-naphthyl)pyrrolidin-1-yl]carbonyl}benzene-1,3-diol; 4-{[2-(3,5-dichlorophenyl)pyrrolidin-1-yl]carbonyl}benzene-1,3-diol; 4-{[2-(2-chlorophenyl)pyrrolidin-1-yl]carbonyl}benzene-1,3-diol; 4-[(2-phenylpyrrolidin-1-yl)carbonyl]benzene-1,3-diol; 2-(1,3-dihydro-2H-isoindol-2-ylcarbonyl)-4-fluorophenol; 4-bromo-2-(1,3-dihydro-2H-isoindol-2-ylcarbonyl)phenol; 2-(1,3-dihydro-2H-isoindol-2-ylcarbonyl)-5-(trifluoromethyl)phenol; 4-chloro-2-(1,3-dihydro-2H-isoindol-2-ylcarbonyl)phenol; 4-[(6-amino-3-azabicyclo[3.1.0]hex-3-yl)carbonyl]benzene-1,3-diol; 4-bromo-6-(1,3-dihydro-2H-isoindol-2-ylcarbonyl)benzene-1,3-diol; 3-(1,3-dihydro-2H-isoindol-2-ylcarbonyl)-2′-(trifluoromethyl)biphenyl-4-ol; 3-(1,3-dihydro-2H-isoindol-2-ylcarbonyl)biphenyl-4-ol; and 2-(1,3-dihydro-2H-isoindol-2-ylcarbonyl)-4-(1-methyl-1H-pyrazol-4-yl)phenol; 2-(1,3-dihydro-2H-isoindol-2-ylcarbonyl)-4-methoxyphenol; 2-(1,3-dihydro-2H-isoindol-2-ylcarbonyl)-5-methoxyphenol; 4-(1,3-dihydro-2H-isoindol-2-ylcarbonyl)-2-methylbenzene-1,3-diol; 4-(2,3-dihydro-1H-indol-1-ylcarbonyl)benzene-1,3-diol; 4-(1,3-dihydro-2H-isoindol-2-ylcarbonyl)benzene-1,3-diol; 1-(1,3-dihydro-2H-isoindol-2-ylcarbonyl)-2-naphthol; 3-(1,3-dihydro-2H-isoindol-2-ylcarbonyl)-2-naphthol; 3-(1,3-dihydro-2H-isoindol-2-ylcarbonyl)-6-methoxy-2-naphthol; 5-chloro-2-(1,3-dihydro-2H-isoindol-2-ylcarbonyl)phenol; 2-(1,3-dihydro-2H-isoindol-2-ylcarbonyl)-5-methylphenol; 3-(1,3-dihydro-2H-isoindol-2-ylcarbonyl)-2′,4′-difluorobiphenyl-4-ol; 2-(1,3-dihydro-2H-isoindol-2-ylcarbonyl)-5-fluorophenol; 2-(1,3-dihydro-2H-isoindol-2-ylcarbonyl)-4-methylphenol; 4-(2-aminopyrimidin-4-yl)-2-(1,3-dihydro-2H-isoindol-2-ylcarbonyl)phenol; 2-(1,3-dihydro-2H-isoindol-2-ylcarbonyl)-4-pyrimidin-4-ylphenol; 2-(1,3-dihydro-2H-isoindol-2-ylcarbonyl)-4-(1H-pyrazol-3-yl)phenol; 2-(1,3-dihydro-2H-isoindol-2-ylcarbonyl)-4-(2-ethylpyrimidin-4-yl)phenol; 2-(1,3-dihydro-2H-isoindol-2-ylcarbonyl)-4-(2-methylpyrimidin-4-yl)phenol; 2-(1,3-dihydro-2H-isoindol-2-ylcarbonyl)phenol; 4-({6-[(cyclopropylmethoxy)methyl]-1,4-oxazepan-4-yl}carbonyl)benzene-1,3-diol; 4-({6-[(cyclopropylmethoxy)methyl]-6-hydroxy-1,4-oxazepan-4-yl}carbonyl)benzene-1,3-diol; 2,4-dihydroxy-N-isobutyl-N-methylbenzamide; 2,4-dihydroxy-N-(2-hydroxycyclohexyl)-N-methylbenzamide; 4-{[2-(2,2-dimethylpropyl)piperidin-1-yl]carbonyl}benzene-1,3-diol; 4-[(4-methylpiperidin-1-yl)carbonyl]benzene-1,3-diol; 4-{[4-(4-chlorobenzyl)piperidin-1-yl]carbonyl}benzene-1,3-diol; 4-{[2-(2-cyclopentylethyl)piperidin-1-yl]carbonyl}benzene-1,3-diol; 2,4-dihydroxy-N-(2-hydroxycyclohexyl)-N-methylbenzamide; 4-{[4-(2-pyrimidin-2-ylethyl)piperidin-1-yl]carbonyl}benzene-1,3-diol; 4-{[4-(2-pyrazin-2-ylethyl)piperidin-1-yl]carbonyl}benzene-1,3-diol; 4-({4-[2-(3-chlorophenyl)ethyl]piperidin-1-yl}carbonyl)benzene-1,3-diol; 4-{[4-(2-phenylethyl)piperidin-1-yl]carbonyl}benzene-1,3-diol; 4-{[4-(2-pyrimidin-5-ylethyl)piperidin-1-yl]carbonyl}benzene-1,3-diol; 4-({3-[(2-fluorobenzyl)oxy]-1-oxa-8-azaspiro[4.5]dec-8-yl}carbonyl)benzene-1,3-diol; ethyl 1-(2,4-dihydroxybenzoyl)piperidine-3-carboxylate; 2,4-dihydroxy-N-(2-hydroxy-1-methyl-2-phenylethyl)-N-methylbenzamide; 4-[(4,4-diphenylpiperidin-1-yl)carbonyl]benzene-1,3-diol; 4-{[3-(2-phenoxyethyl)piperidin-1-yl]carbonyl}benzene-1,3-diol; 4-[(6,7-diethoxy-3,4-dihydroisoquinolin-2(1H)-yl)carbonyl]benzene-1,3-diol; 4-{[2-(3-fluorophenyl)piperidin-1-yl]carbonyl}benzene-1,3-diol; 4-[(3-{[(5-ethyl-1,2,4-oxadiazol-3-yl)methoxy]methyl}pyrrolidin-1-yl)carbonyl]benzene-1,3-diol; 4 ({3-[(3-chlorophenoxy)methyl]piperidin-1-yl}carbonyl)benzene-1,3-diol; 4-[(3-{[(6-methylpyridin-3-yl)oxy]methyl}piperidin-1-yl)carbonyl]benzene-1,3-diol; 4-({6-[(cyclopentyloxy)methyl]-6-hydroxy-1,4-oxazepan-4-yl}carbonyl)benzene-1,3-diol; 4-{[2-(1,3-thiazol-2-yl)piperidin-1-yl]carbonyl}benzene-1,3-diol; 4-(piperidin-1-ylcarbonyl)benzene-1,3-diol; 4-({3-[2-(methoxymethyl)pyrimidin-4-yl]piperidin-1-yl}carbonyl)benzene-1,3-diol; 4-{[3-(2 phenylethyl)pyrrolidin-1-yl]carbonyl}benzene-1,3-diol; 2,4-dihydroxy-N-methyl-N-(1-phenylethyl)benzamide; 2,4-dihydroxy-N-methyl-N-(1-phenylethyl)benzamide; 4-{[4-(4-chlorophenoxy)piperidin-1-yl]carbonyl}benzene-1,3-diol; 4-{[4-(3-chlorophenoxy)piperidin-1-yl]carbonyl}benzene-1,3-diol; 4-{[4-(4-fluorophenyl)piperidin-1-yl]carbonyl}benzene-1,3-diol; 4-[(4-phenoxypiperidin-1-yl)carbonyl]benzene-1,3-diol; 4-{[4-(2-methoxyphenoxy)piperidin-1-yl]carbonyl}benzene-1,3-diol; 4-{[2-(2-methylphenyl)pyrrolidin-1-yl]carbonyl}benzene-1,3-diol; 4-{[4-(4-methylphenoxy)piperidin-1-yl]carbonyl}benzene-1,3-diol; 4-[(6,8-dimethyl-3,4-dihydro-1′H-spiro[chromene-2,4′-piperidin]-1′-yl)carbonyl]benzene-1,3-diol; 4-{[4-(4-chlorophenyl)-4-hydroxypiperidin-1-yl]carbonyl}benzene-1,3-diol; 4-[(4-hydroxy-4-phenylpiperidin-1-yl)carbonyl]benzene-1,3-diol; 4-({4-[4-chloro-3-(trifluoromethyl)phenyl]-4-hydroxypiperidin-1-yl}carbonyl)benzene-1,3-diol; 4-[(6-chloro-1′H-spiro[chromene-2,4′-piperidin]-1′-yl)carbonyl]benzene-1,3-diol; 4-(1′H,3H-spiro[2-benzofuran-1,4′-piperidin]-1′-ylcarbonyl)benzene-1,3-diol; 2,4-dihydroxy-N-methyl-N-[1-(1-naphthyl)ethyl]benzamide; 4-[(6-methyl-1′H-spiro[chromene-2,4′-piperidin]-1′-yl)carbonyl]benzene-1,3-diol; 4-({4-[2-(trifluoromethyl)pyrimidin-4-yl]piperidin-1-yl}carbonyl)benzene-1,3-diol; 4-{[2-(4-chlorophenyl)pyrrolidin-1-yl]carbonyl}benzene-1,3-diol; 1′-(2,4-dihydroxybenzoyl)spiro[chromene-2,4′-piperidin]-4(3H)-one; 4-[(4-pyrimidin-2-ylpiperindin-1-yl)carbonyl]benzene-1,3-diol; 4-({3-[(3-chlorophenoxy)methyl]pyrrolidin-1-yl}carbonyl)benzene-1,3-diol; 4-{[4-(5-chloro-1,3-benzoxazol-2-yl)-1,4-diazepan-1-yl]carbonyl}benzene-1,3-diol; 4-({3-[(2-chloro-4-fluorophenoxy)methyl]pyrrolidin-1-yl}carbonyl)benzene-1,3-diol; 4-(pyrrolidin-1-ylcarbonyl)benzene-1,3-diol; 1-{[7-(2,4-dihydroxybenzoyl)-6,7,8,9-tetrahydro-5H-[1,2,4]triazolo[4,3-d][1,4]diazepin-3-yl]methyl}pyrrolidin-2-one; 4-({2-[5-(2-methoxyphenyl)-1,3,4-oxadiazol-2-yl]pyrrolidin-1-yl}carbonyl)benzene-1,3-diol; 4-[(3-phenylpyrrolidin-1-yl)carbonyl]benzene-1,3-diol; methyl 1-(2,4-dihydroxybenzoyl)pyrrolidine-3-carboxylate; 4-{[3-(4-fluorophenyl)pyrrolidin-1-yl]carbonyl}benzene-1,3-diol; 4-{[3-(3-chlorophenyl)pyrrolidin-1-yl]carbonyl}benzene-1,3-diol; 4-(3,6-dihydropyridin-1(2H)-ylcarbonyl)benzene-1,3-diol; 4-[(6-{[(3,5-dimethylisoxazol-4-yl)methoxy]methyl}-1,4-oxazepan-4-yl)carbonyl]benzene-1,3-diol; 4-{[(6-(3,4-dichlorobenzyl)-1,4-oxazepan-4-yl]carbonyl}benzene-1,3-diol; 4-{[3-hydroxy-3-(2-methylphenyl)pyrrolidin-1-yl]carbonyl}benzene-1,3-diol; 4-{[4-(6-methylpyridin-2-yl)piperazin-1-yl]carbonyl}benzene-1,3-diol; N-[2-(3,4-dimethoxyphenyl)ethyl]-2,4-dihydroxy-N-methylbenzamide; (2,4-Dihydroxy-phenyl)-(1-phenyl-3,4-dihydro-1H-isoquinolin-2-yl)-methanone; 4-[(2-methyl-6,7-dihydro[1,3]thiazolo[5,4-c]pyridin-5(4H)-yl)carbonyl]benzene-1,3-diol; N-(4-fluorobenzyl)-2,4-dihydroxy-N-methylbenzamide, N-(4-chlorobenzyl)-2,4-dihydroxy-N-methylbenzamide; 2,4-dihydroxy-N-methyl-N-(4-phenoxybenzyl)benzamide; 2,4-dihydroxy-N-methyl-N-(2-phenylethyl)benzamide; 4-(3,4-dihydroisoquinolin-2(1H)-ylcarbonyl)benzene-1,3-diol; 4-({2-[(3-chlorophenoxy)methyl]morpholin-4-yl}carbonyl)benzene-1,3-diol; 4-[(4-pyrazin-2-ylpiperazin-1-yl)carbonyl]benzene-1,3-diol; 4-{[2-(phenoxymethyl)morpholin-4-yl]carbonyl}benzene-1,3-diol; N-(2-chlorobenzyl)-2,4-dihydroxy-N-methylbenzamide; 4-({2-[(3,5-difluorophenoxy)methyl]morpholin-4-yl}carbonyl)benzene-1,3-diol; 4-({2-[(2-chloro-4-fluorophenoxy)methyl]morpholin-4-yl}carbonyl)benzene-1,3-diol, 4-{[3-(4-methoxybenzyl)-5,6,8,9-tetrahydro-7H-[1,2,4]triazolo[4,3-d][1,4]diazepin-7-yl]carbonyl}benzene-1,3-diol; 4-(1,3-thiazolidin-3-ylcarbonyl)benzene-1,3-diol; 2,4-dihydroxy-N-{[5-(2-methoxyphenyl)-1,3,4-oxadiazol-2-yl]methyl}-N-methylbenzamide: N-{[5-(4-fluorophenyl)-1,3,4-oxadiazol-2-yl]methyl}-2,4-dihydroxy-N-methylbenzamide; 4-{[3-(4-fluorophenyl)-1,7-dioxa-2,10-diazaspiro[4.6]undec-2-en-10-yl]carbonyl}benzene-1,3-diol; N-{[5-(3-cyanophenyl)-1,3,4-oxadiazol-2-yl]methyl}-2,4-dihydroxy-N-methylbenzamide; 4-[(6-{[(2,6-dichlorobenzyl)oxy]methyl}-1,4-oxazepan-4-yl)carbonyl]benzene-1,3-diol; N-(1,3-benzoxazol-2-ylmethyl)-2,4-dihydroxy-N-methylbenzamide; 4-{[6-(hydroxymethyl)-4-pyrazin-2-yl-1,4-diazepan-1-yl]carbonyl}benzene-1,3-diol; 4-{[4-(6-chloro-1,3-benzoxazol-2-yl)piperazin-1-yl]carbonyl}benzene-1,3-diol; 2-[4-(2,4-dihydroxybenzoyl)piperazin-1-yl]-6-[2-(trifluoromethyl)phenyl]pyrimidin-4(3H)-one; 4-{[4-(5-methoxy-1,3-benzoxazol-2-yl)piperazin-1-yl]carbonyl}-benzene-1,3-diol; 4-{[3-(3,5-difluorophenyl)-1,7-dioxa-2,10-diazaspiro[4.6]undec-2-en-10-yl]carbonyl}benzene-1,3-diol; 4-{[3-(4-fluoro-3-methoxyphenyl)-1,7-dioxa-2,10-diazaspiro[4.6]undec-2-en-10-yl]carbonyl}benzene-1,3-diol; 4-[(3-phenyl-1,7-dioxa-2,10-diazaspiro[4.6]undec-2-en-10-yl)carbonyl]benzene-1,3-diol; 4-{[3-(2,6-difluorophenyl)-1,7-dioxa-2,10-diazaspiro[4.6]undec-2-en-10-yl]carbonyl}benzene-1,3-diol; 4-{[3-(2-fluoro-5-methoxyphenyl)-1,7-dioxa-2,10-diazaspiro[4.6]undec-2-en-10-yl]carbonyl}benzene-1,3-diol; 4-(thiomorpholin-4-ylcarbonyl)benzene-1,3-diol; (2,4-Dihydroxy-phenyl)-(3-naphthalen-1-yl-4,5,7,8-tetrahydro-1,2,3a,6-tetraaza-azulen-6-yl)-methanone; 4-[(3-phenylmorpholin-4-yl)carbonyl]benzene-1,3-diol; 4-(5,6-dihydro[1,2,4]triazolo[4,3-a]pyrazin-7(8H)-ylcarbonyl)benzene-1,3-diol; 4-bromo-6-[(2-{4-[(dimethylamino)methyl]phenyl}pyrrolidin-1-yl)carbonyl]benzene-1,3-diol; 4-{[3-(4-bromophenyl)piperazin-1-yl]carbonyl}-6-chlorobenzene-1,3-diol; 4-chloro-6-{[5-(hydroxymethyl)-1,3-dihydro-2H-isoindol-2-yl]carbonyl}benzene-1,3-diol; 4-tert-butyl-2-(1,3-dihydro-2H-isoindol-2-ylcarbonyl)phenol; 3-bromo-2-(1,3-dihydro-2H-isoindol-2-ylcarbonyl)phenol; -(1,3-dihydro-2H-isoindol-2-ylcarbonyl)-2′-(trifluoromethyl)biphenyl-4-ol; 3-(1,3-dihydro-2H-isoindol-2-ylcarbonyl)biphenyl-4-ol; 2-(1,3-dihydro-2H-isoindol-2-ylcarbonyl)-4-(1-methyl-1H-pyrazol-4-yl)phenol; 2-(5-chloro-2,4-dihydroxybenzoyl)-N-ethylisoindoline-1-carboxamide; 1-[3-(1,3-dihydro-2H-isoindol-2-ylcarbonyl)-4-hydroxyphenyl]ethanone; 2-(1,3-dihydro-2H-isoindol-2-ylcarbonyl)-4-(1-hydroxyethyl)phenol; 3-(1,3-dihydro-2H-isoindol-2-ylcarbonyl)-4-hydroxybenzonitrile; 4-chloro-6-(1,3-dihydro-2H-isoindol-2-ylcarbonyl)-5-methylbenzene-1,3-diol; 4-chloro-6-{[(2R)-2-(pyrrolidin-1-ylmethyl)pyrrolidin-1-yl]carbonyl}benzene-1,3-diol; 2-(5-chloro-2,4-dihydroxybenzoyl)-N-ethylisoindoline-5-carboxamide; 3-chloro-4,6-dihydroxy-N,N-dimethyl-2-(2-oxo-2-piperidin-1-ylethyl)benzamide; 2-(5-chloro-2,4-dihydroxybenzoyl)-N-ethylisoindoline-1-carboxamide; 2-(5-chloro-2,4-dihydroxybenzoyl)-N-ethylisoindoline-1-carboxamide; 2-(5-chloro-2,4-dihydroxybenzoyl)-N-cyclobutylisoindoline-1-carboxamide; 2-(5-chloro-2,4-dihydroxybenzoyl)-N-(2 isocyanoethyl)isoindoline-1-carboxamide; 2-(5-chloro-2,4-dihydroxybenzoyl)-N-cyclopropylisoindoline-1-carboxamide; 2-(5-chloro-2,4-dihydroxybenzoyl)-N-(2,2,2-trifluoroethyl)isoindoline-1-carboxamide; N-allyl-2-(5-chloro-2,4-dihydroxybenzoyl)isoindoline-1-carboxamide; 2-(5-chloro-2,4-dihydroxybenzoyl)-N-isopropylisoindoline-1-carboxamide; 2-(5-chloro-2,4-dihydroxybenzoyl)-N-[2-(dimethylamino)ethyl]isoindoline-1-carboxamide; 2-(5-chloro-2,4-dihydroxybenzoyl)-N-(2,2-difluoroethyl)isoindoline-1-carboxamide; 2-(5-chloro-2,4-dihydroxybenzoyl)-N-(4-hydroxycyclohexyl)isoindoline-1-carboxamide; 2-(5-chloro-2,4-dihydroxybenzoyl)-N-propylisoindoline-1-carboxamide; 4-{[2-(2-methoxyphenyl)pyrrolidin-1-yl]carbonyl}benzene-1,3-diol; 4-[(2-phenylpyrrolidin-1-yl)carbonyl]benzene-1,3-diol; 4-(1,3-dihydro-2H-isoindol-2-ylsulfonyl)benzene-1,3-diol; 4-{[(2S)-2-(2-methylphenyl)pyrrolidin-1-yl]carbonyl}benzene-1,3-diol; 4-{[(2R)-2-(2-methylphenyl)pyrrolidin-1-yl]carbonyl}benzene-1,3-diol; 4-[(2-{4-[(dimethylamino)methyl]phenyl}pyrrolidin-1-yl)carbonyl]benzene-1,3-diol; 4-[(2-biphenyl-4-ylpyrrolidin-1-yl)carbonyl]benzene-1,3-diol; 4-{[2-(3-bromophenyl)pyrrolidin-1-yl]carbonyl}benzene-1,3-diol; 4-[(2-biphenyl-3-ylpyrrolidin-1-yl)carbonyl]benzene-1,3-diol; 4-{[2-(2′-chlorobiphenyl-3-yl)pyrrolidin-1-yl]carbonyl}benzene-1,3-diol; 3′-[1-(2,4-dihydroxybenzoyl)pyrrolidin-2-yl]-N,N-dimethylbiphenyl-4-carboxamide; 3′-[1-(2,4-dihydroxybenzoyl)pyrrolidin-2-yl]-N,N-dimethylbiphenyl-3-carboxamide; 4-({2-[3′-(piperidin-1-ylmethyl)biphenyl-3-yl]pyrrolidin-1-yl}carbonyl)benzene-1,3-diol; 4-[1-(2,4-dihydroxybenzoyl)pyrrolidin-2-yl]-N,N-dimethylbenzamide; 4-{[2-(4-bromophenyl)pyrrolidin-1-yl]carbonyl}benzene-1,3-diol; 4-({2-[4-(1-methyl-1H-pyrazol-4-yl)phenyl]pyrrolidin-1-yl}carbonyl)benzene-1,3-diol; 4-{[3-(phenylsulfonyl)pyrrolidin-1-yl]carbonyl}benzene-1,3-diol; 4 chloro-6-{[2-(2-hydroxyethyl)piperidin-1-yl]carbonyl}-5-methylbenzene-1,3-diol; 4-chloro-5-methyl-6-{[2-(2-piperidin-1-ylethyl)piperidin-1-yl]carbonyl}benzene-1,3-diol; 4-chloro-6-{[2-(2-cyclopentylethyl)piperidin-1-yl]carbonyl}-5-methylbenzene-1,3-diol; 4-chloro-5-methyl-6-[(2-pyridin-2-ylpiperidin-1-yl)carbonyl]benzene-1,3-diol; 4-chloro-5-methyl-6-(piperazin-1-ylcarbonyl)benzene-1,3-diol; 4-{[3-(methylsulfonyl)pyrrolidin-1-yl]carbonyl}benzene-1,3-diol; methyl 1-(2,4-dihydroxybenzoyl)pyrrolidine-3-carboxylate; 4-{[3-(pyrazin-2-ylmethyl)pyrrolidin-1-yl]carbonyl}benzene-1,3-diol; tert-butyl 1-(2,4-dihydroxybenzoyl)-D-prolinate; 4-{[3-(hydroxymethyl)pyrrolidin-1-yl]carbonyl}benzene-1,3-diol; benzyl 1-(2,4-dihydroxybenzoyl)-L-prolinate; 4-nitrobenzyl 1-(2,4-dihydroxybenzoyl)-L-prolinate; 4-[(3-benzylpyrrolidin-1-yl)carbonyl]benzene-1,3-diol; 4-[(2-phenylpyrrolidin-1-yl)carbonyl]benzene-1,3-diol; 4-{[2-(3-fluorophenyl)pyrrolidin-1-yl]carbonyl}benzene-1,3-diol; benzyl 1-(2,4-dihydroxybenzoyl)-D-prolinate; 4-[(2-{5-[(cyclopropylmethyl)thio]-4-methyl-4H-1,2,4-triazol-3-yl}pyrrolidin-1-yl)carbonyl]benzene-1,3-diol; 4-({2-[4-(trifluoromethyl)phenyl]pyrrolidin-1-yl}carbonyl)benzene-1,3-diol; 2,4-dichloro-6-(1,3-dihydro-2H-isoindol-2-ylcarbonyl)phenol; 4-{[3-(4-bromophenyl)piperazin-1-yl]carbonyl}-6-chlorobenzene-1,3-diol; 4-chloro-6-{[5-(hydroxymethyl)-1,3-dihydro-2H-isoindol-2-yl]carbonyl}benzene-1,3-diol; methyl 4-(1-(5-chloro-2,4-dihydroxybenzoyl)pyrrolidin-2-yl)-3-methylbenzoate; 3-[1-(5-chloro-2,4-dihydroxybenzoyl)pyrrolidin-2-yl]-N-ethyl-4-methylbenzamide; methyl 3-[1-(5-chloro-2,4-dihydroxybenzoyl)pyrrolidin-2-yl]-4-methylbenzoate; and 4-(1-(5-chloro-2,4-dihydroxybenzoyl)pyrrolidin-2-yl)-N-ethyl-3-methylbenzamide;
or a pharmaceutically acceptable salt or solvate thereof.

9. A pharmaceutical composition, comprising at least one compound according to any one of claims 1 to 8, or a pharmaceutically acceptable salt or solvate thereof, and a pharmaceutically acceptable carrier or diluent.

10. A method of treating cancer in a mammal, comprising the step of administering to said mammal a therapeutically effective amount of at least one compound according to any one of claims 1 to 8, or a pharmaceutically acceptable salt or solvate thereof.
